(19) United States
(12) Patent Application Publication
(10) Pub. No.: US 2026/0285877 A1
(43) Pub. Date: Sep. 24, 2026

(54) Macrocyclic EGFR Inhibitor, and Preparation Method Therefor and Pharmaceutical Use Thereof

(71) Applicant: Abbisko Therapeutics Co., Ltd., Pudong New Area, Shanghai (CN)

(72) Inventors: Fei YANG, Pudong New Area, Shanghai (CN); Haibing DENG, Pudong New Area, Shanghai (CN); Yong YU, Pudong New Area, Shanghai (CN); Hongping YU, Pudong New Area, Shanghai (CN); Zhui CHEN, Pudong New Area, Shanghai (CN); Yaochang XU, Pudong New Area, Shanghai (CN)

(21) Appl. No.: 18/873,958

(22) PCT Filed: Jul. 24, 2023
(86) PCT No.: PCT/CN2023/108872

§ 371 Date: Dec. 11, 2024

(30) Foreign Application Priority Data
Jul. 27, 2022 (CN) 202210890232.3
Dec. 23, 2022 (CN) 202211663752.7

(51) Int. Cl. C07D 498/22; A61K 31/529; A61P 35/00

(52) U.S. Cl. CPC C07D 498/22; A61K 31/529; A61P 35/00

(57) ABSTRACT

The present invention relates to a macrocyclic EGFR inhibitor and preparation method therefor and pharmaceutical use thereof. In particular, the present invention relates to an EGFR inhibitor having a structure of formula (I), a pharmaceutical composition thereof, a use thereof as an EGFR inhibitor, and a use thereof in the preparation of a drug for treating and/or preventing tumors or metastatic diseases associated at least partially with EGFR L858R/C797S double mutation, EGFR Del19/C797S double mutation, L858R/T790M/C797S triple mutation and Del19/T790M/C797S triple mutation, especially in the preparation of a drug for treating and/or preventing hyperproliferative diseases and disorders inducing cell death. Each substituent of formula (I) is as defined in the description.

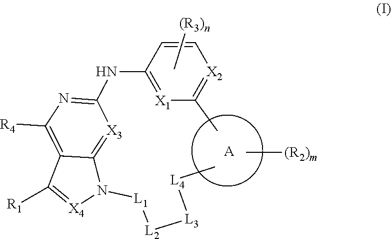

### TECHNICAL FIELD

The present invention belongs to the field of pharmaceutical synthesis, and particularly relates to a macrocyclic EGFR inhibitor, preparation method therefor, and pharmaceutical use thereof.

### BACKGROUND

Lung cancer is a malignant tumor with the highest mortality rate worldwide, posing a serious threat to human health. Non-small-cell Lung Cancer (NSCLC) accounts for approximately 85% of all lung cancer cases. Currently, molecular mechanisms such as multiple gene mutations and abnormal gene expression have been confirmed to be related to the pathogenesis of NSCLC, among which, EGFR is the primary driver gene. EGFR activating mutations occur in approximately 17% of NSCLC patients worldwide and are more prevalent in East Asian populations, with a mutation frequency of about 50%. EGFR activating mutations typically occur in exons 18-21. The most common mutation subtypes are deletion mutations in exon 19 (Del19) and L858R point mutations in exon 21, accounting for more than 80% of all mutation types. These mutations can lead to ligand-independent receptor activation and promote tumor cell survival and proliferation. The first- and second-generation EGFR Tyrosine Kinase Inhibitors (TKIs) are mainly targeted at these two activating mutations. Compared with platinum-based chemotherapy, the first- and second-generation EGFR TKIs can significantly prolong progression-free survival, but resistance tends to emerge after 10-12 months of treatment. The EGFR T790M mutation is the main mechanism of resistance to the first- and second-generation EGFR TKIs, and subsequently, the third-generation EGFR TKI, Osimertinib, was developed to selectively inhibit the EGFR T790M mutation and other common EGFR mutations. The results of the phase III FLAURA clinical trial demonstrated that Osimertinib exhibited superior efficacy compared with the first-generation EGFR TKIs, significantly improving progression-free survival (18.9 months vs. 10.2 months) and overall survival (38.6 months vs. 31.8 months), which directly promoted Osimertinib to be included in the first-line treatment options.

Despite the significant efficacy of Osimertinib, resistance inevitably emerges and leads to disease progression. Currently, no targeted drugs are approved for patients who are resistant to Osimertinib. Several EGFR-dependent and -independent mechanisms of resistance have been reported in both preclinical and clinical studies. The C797X (with the C797S mutation being the most frequent) mutation in exon 20 has emerged as the most common EGFR-dependent mechanism of resistance to Osimertinib. The C797X mutation is located in the tyrosine kinase region of EGFR, making it impossible for Osimertinib to form a covalent bond within the ATP binding domain of EGFR, which results in resistance. In the phase 3 AURA3 clinical trial, the probability of C797X mutation in patients receiving Osimertinib in the second-line setting was 15%, while in the phase 3 FLAURA clinical trial, the probability of C797X mutation in patients receiving Osimertinib in the first-line setting was 7%. Therefore, it is crucial to develop a next-generation EGFR inhibitor with improved activity and selectivity, targeting both a double mutation consisting of an EGFR activating mutation/C797S and a triple mutation consisting of an EGFR activating mutation/T790M/C797S, for patients who have developed resistance to the first-line or second-line Osimertinib therapy.

### SUMMARY

The objective of the present invention is to provide a macrocyclic EGFR inhibitor, a preparation method therefor, and a pharmaceutical use thereof. The series of compounds of the present invention have a strong inhibitory effect on cytological activity of EGFR L858R/C797S double mutation, EGFR Del19/C797S double mutation, L858R/T790M/C797S triple mutation and Del19/T790M/C797S triple mutation, and can be widely used in the preparation of drugs for treatment and/or prevention of tumors or metastatic diseases associated at least partially with EGFR L858R/C797S double mutation, EGFR Del19/C797S double mutation, L858R/T790M/C797S triple mutation and Del19/T790M/C797S triple mutation, especially in the preparation of drugs for treating hyperproliferative diseases and disorders inducing cell death, thereby promising the development of a new-generation EGFR inhibitor.

The first aspect of the present invention provides a compound of formula (I), a stereoisomer or pharmaceutically acceptable salt thereof:

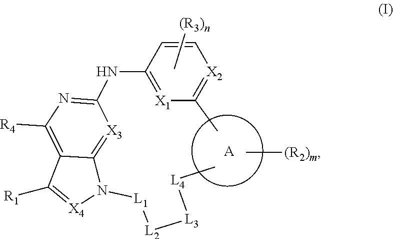

wherein, X₁ is CRₐ or N; X₂ is CR_{b} or N; X₃ is CR or N; X₄ is CR_{d} or N;
L₁ is CR₅ₐR_{5b};
L₂ is (CR_{5c}R_{5d})ₚ, NR₆ₐ, O, S, S(O), S(O)₂ or C(O);
L₃ is a bond, CR₅ₑR_{5f}, NR_{6b}, O, S, S(O), S(O)₂ or C(O); or
L₂ and L₃ together form a C₄₋₁₂ cycloalkyl, 4-12 membered heterocyclyl, C₆₋₁₀ aryl or 5-10 membered heteroaryl, and the C₄₋₁₂ cycloalkyl, 4-12 membered heterocyclyl, C₆₋₁₀ aryl or 5-10 membered heteroaryl is independently optionally further substituted by one or more substituents selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
L₄ is CR_{5g}R₅ₕ, O, S, S(O), S(O)₂, NR_{6c} or C(O);
ring A is C₄₋₁₂ cycloalkyl, 4-12 membered heterocyclyl, C₆₋₁₀ aryl or 5-10 membered heteroaryl;
R₁ is selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N-R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂, and the above groups are each independently optionally further substituted by one or more substituents selected from the group consisting of deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₁₋₁₀ alkyl substituted by C₃₋₁₂ cycloalkyl, C₁₋₁₀ alkyl substituted by 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-N(R₁₃)—S(O)₂R₁₀, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₄ and —C₀₋₈ alkyl-N(R₁₃)—C(O)R₁₂, and the above groups are more independently optionally further substituted by one or more substituents selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-N(R₁₃)—S(O)₂R₁₀, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
each R₂ is independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂, and the above groups are independently optionally further substituted by one or more substituents selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
each R₃, each Rₐ and each R_{b} are independently selected from the group consisting of hydrogen, deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
R₄ and R_{c} are each independently selected from the group consisting of hydrogen, deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
R_{d} is selected from the group consisting of hydrogen, deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₂, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
R₅ₐ and R_{5b} are each independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂, or, R₅ₐ and R_{5b}, together with the carbon atom to which they are directly attached, form a C(O), C₃₋₆ cycloalkyl, 4-10 membered heterocyclyl, C₆₋₁₀ aryl or 5-10 membered heteroaryl, and the above groups are each independently optionally further substituted by one or more substituents selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
each R_{5c} and each R_{5d} are independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂, or, R_{5c} and R_{5d}, together with the carbon atom to which they are directly attached, form a C₃₋₆ cycloalkyl, 4-10 membered heterocyclyl, C₆₋₁₀ aryl or 5-10 membered heteroaryl, and the above groups are each independently optionally further substituted by one or more substituents selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂, provided that when R_{5c} or R_{5d} is H or C₁₋₄ alkyl, R₁ is optionally substituted C₂₋₁₀ alkynyl, or, L₃ is O, S, S(O) or S(O)₂;
R₅ₑ and R_{5f} are each independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂, or, R₅ₑ and R_{5f}, together with the carbon atom to which they are directly attached, form a C₃₋₆ cycloalkyl, 4-10 membered heterocyclyl, C₆₋₁₀ aryl or 5-10 membered heteroaryl, and the above groups are each independently optionally further substituted by one or more substituents selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
R_{5g} and R₅ₕ are each independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₂)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂, or, R_{5g} and R₅ₕ, together with the carbon atom to which they are directly attached, form a C₃₋₆ cycloalkyl, 4-10 membered heterocyclyl, C₆₋₁₀ aryl or 5-10 membered heteroaryl, and the above groups are each independently optionally further substituted by one or more substituents selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
R₆ₐ is selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl and —O—R₁₁, and the above groups are each independently optionally further substituted by one or more substituents selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy and —NR₁₃R₁₄,
R_{6b} is selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl and —O—R₁₁, and the above groups are each independently optionally further substituted by one or more substituents selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy and —NR₁₃R₁₄;
R_{6c} is selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl and —O—R₁₁, and the above groups are each independently optionally further substituted by one or more substituents selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy and —NR₁₃R₁₄;
each R₇ is independently selected from the group consisting of hydrogen, deuterium, C₁₋₁₀ alkyl, C₂₋₁₀ alkenyl, C₂₋₁₀ alkynyl, C₃₋₁₀ cycloalkyl, 3-10 membered heterocyclyl, C₆₋₁₀ aryl, 5-10 membered heteroaryl, —C₀₋₈ alkyl-S(O)ᵣR₁₀, —C₀₋₈ alkyl-C(O)R₁₁, —C₀₋₈ alkyl-C(O)R₁₂ or —C₀₋₈ alkyl-C(O)NR₁₃R₁₄, and the above groups are optionally further substituted by one or more substituents selected from the group consisting of deuterium, halogen, cyano, nitro, azido, C₁₋₁₀ alkyl, C₂₋₁₀ alkenyl, C₂₋₁₀ alkynyl, C₁₋₁₀ haloalkyl, C₁₋₁₀ deuteroalkyl, C₃₋₁₀ cycloalkyl, 3-10 membered heterocycyl, C₆₋₁₀ aryl, 5-10 membered heteroaryl, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)R₁₂, —C₀₋₈ alkyl-O—C(O)R₁₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ or —C₀₋₈ alkyl-N(R₁₃)—C(O)R₁₂;
each R₈ and each R₉ are independently selected from the group consisting of hydrogen, deuterium, hydroxy, C₁₋₁₀ alkyl, C₂₋₁₀ alkenyl, C₂₋₁₀ alkynyl, C₃₋₁₀ cycloalkyl, 3-10 membered heterocyclyl, C₆₋₁₀ aryl or 5-10 membered heteroaryl, or, R₈ and R₉, together with the sulfur atom to which they are directly attached, form a 3-10 membered heterocyclyl, and the above groups are optionally further substituted by one or more substituents selected from the group consisting of deuterium, halogen, cyano, nitro, azido, C₁₋₁₀ alkyl, C₂₋₁₀ alkenyl, C₂₋₁₀ alkynyl, C₁₋₁₀ haloalkyl, C₁₋₁₀ deuteroalkyl, C₃₋₁₀ cycloalkyl, 3-10 membered heterocyclyl, C₆₋₁₀ aryl, 5-10 membered heteroaryl, —C₀₋₈ alkyl-S(O)R₁₀, —C₀₋₈ alkyl-O—R₁₁, —C₀₋₈ alkyl-C(O)OR₁₁, —C₀₋₈ alkyl-C(O)R₁₂, —C₀₋₈ alkyl-O—C(O)R₁₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ or —C₀₋₈ alkyl-N(R₁₃)—C(O)R₁₂,
each R₁₀ is independently selected from the group consisting of hydrogen, deuterium, hydroxy, C₆₋₁₀ alky, C₂₋₁₀ alkenyl, C₃₋₁₂ cycloalkyl, 3-12 membered heterocyclyl, C₆₋₁₀ aryl, 5-10 membered heteroaryl and —NR₁₃R₁₄, and the above groups are each independently optionally further substituted by one or more substituents selected from the group consisting of deuterium, halogen, hydroxy, ═O, C₁₋₁₀ alkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy and —NR₁₃R₁₄;
each R₁₁ is independently selected from the group consisting of hydrogen, deuterium, C₁₋₁₀ alkyl, C₂₋₁₀ alkenyl, C₃₋₁₂ cycloalkyl, 3-12 membered heterocyclyl, C₆₋₁₀ aryl and 5-10 membered heteroaryl, and the above groups are each independently optionally further substituted by one or more substituents selected from the group consisting of deuterium, halogen, hydroxy, ═O, cyano, C₁₋₁₀ alkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy and —NR₁₃R₁₄;
each R₁₂ is independently selected from the group consisting of hydrogen, deuterium, hydroxy, C₁₋₁₀ alkyl, C₁₋₁₀ alkoxy, C₂₋₁₀ alkenyl, C₂₋₁₀ alkynyl, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy and —NR₁₃R₁₄, and the above groups are each independently optionally further substituted by one or more substituents selected from the group consisting of deuterium, halogen, hydroxy, ═O, cyano, C₁₋₁₀ alkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy and —NR₁₃R₁₄;
each R₁₃ and each R₁₄ are independently selected from the group consisting of hydrogen, deuterium, hydroxy, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, sulfinyl, sulfonyl, methanesulfonyl, isopropylsulfonyl, cyclopropylsulfonyl, p-toluenesulfonyl, aminosulfonyl, dimethylaminosulfonyl and C₁₋₁₀ alkanoyl, and the above groups are each independently optionally further substituted by one or more substituents selected from the group consisting of deuterium, halogen, hydroxy, ═O, C₁₋₁₀ alkyl, C₂₋₁₀ alkenyl, C₂₋₁₀ alkynyl, C₁₋₁₀ haloalkyl, C₁₋₁₀ deuteroalkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy, amino, monoC₁₋₁₀ alkylamino, diC₁₋₁₀ alkylamino and C₁₋₁₀ alkanoyl; or,
R₁₃ and R₁₄, together with the nitrogen atom to which they are directly attached, form a 4-10 membered heterocyclyl or 5-10 membered heteroaryl, and the 4-10 membered heterocyclyl or 5-10 membered heteroaryl is optionally further substituted by one or more substituents selected from the group consisting of deuterium, halogen, hydroxy, ═O, C₁₋₁₀ alkyl, C₂₋₁₀ alkenyl, C₂₋₁₀ alkynyl, C₁₋₁₀ haloalkyl, C₁₋₁₀ deuteroalkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy, methanesulfonylmethyl, amino, monoC₁₋₁₀ alkylamino, diC₁₋₁₀ alkylamino and C₁₋₁₀ alkanoyl;
m is 0, 1, 2, 3 or 4; n is 0, 1 or 2; p is 1 or 2; and
each r is independently 0, 1 or 2.

As a preferred embodiment, in the compound of formula (I), the stereoisomer or pharmaceutically acceptable salt thereof, X₁ is CRₐ or N; X₂ is CR_{b} or N; X₃ is CR_{c} or N;
X₄ is CR_{c} or N;
L₁ is CR₅ₐR_{5b};
L₂ is (CR_{5c}R_{5d})ₚ, NR₆ₐ, O, S, S(O), S(O)₂ or C(O);
L₃ is a bond, CR₅ₑR_{5f}, NR_{6b}, O, S, S(O), S(O)₂ or C(O); or
L₂ and L₃ together form a C₄₋₆ cycloalkyl, 4-6 membered heterocyclyl, C₆₋₈ aryl or 5-8 membered heteroaryl, and the C₄₋₆ cycloalkyl, 4-6 membered heterocyclyl, C₆₋₈ aryl or 5-8 membered heteroaryl is independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₄ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₄ alkyl-C(O)SR₁₁, —C₀₋₈ alkyl-S—C(O)R₁₂, —C₀₋₈ alkyl-C(O)R₁₂, —C₀₋₄ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₄ alkyl-C(O)NR₁₃R₁₄ and —C₀₋₈ alkyl-N(R₁₃)—C(O)R₁₂;
L₄ is CR_{5g}R₅ₕ, O, S, S(O), S(O)₂, NR_{6c} or C(O);
ring A is C₄₋₆ cycloalkyl, 4-6 membered heterocyclyl, C₆₋₈ aryl or 5-8 membered heteroaryl;
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, and the above groups are each independently optionally further substituted by one or more substituents selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, C₆₋₈ aryl, 5-8 membered heteroaryl, ═O, ═S, —C₀₋₄ alkyl-SF₅, —C₀₋₈ alkyl-S(O)(═N—R₇)R₈, —C₀₋₄ alkyl-N═S(O)R₈R₉, —C₀₋₈ alkyl-N═SR₈R₉, —C₀₋₄ alkyl-N(R₁₃)—S(O)₂R₁₀, —C₀₋₄ alkyl-O—S(O)₂R₁₀, —C₀₋₈ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, and the above groups are independently optionally further more substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-N(R₁₃)—S(O)₂R₁₀, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₄ and —C₀₋₈ alkyl-N(R₁₃)—C(O)R₁₂;
each R₂ is independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₈ alkyl-C(═NR)R₁₂, —C₀₋₄ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂;
each R₃, each Rₐ and each R_{b} are independently selected from the group consisting of hydrogen, deuterium, halogen, cyano, C₁₋₄ alkyl, C₁₋₄ haloalkyl, C₀₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heterocyclyl, —C₀₋₈ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₈ alkyl-O—S(O)₂R₁₀, —C₀₋₈ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₄ alkyl-C(═NR)R₂, —C₀₋₈ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂;
R₄ and R_{c} are each independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄— alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₂, —C₀₋₄ alkyl-N(R₁₃)—C(═NR₁₁)R₁₂, —C₀₋₄ alkyl-C(O)NR₁₃R₁₄ and —C₀₋₄ alkyl-N(R₁₃)—C(O)R₁₂;
R_{d} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₂)₂, —C₀₋₄ alkyl-NR₁₃R₁₄, —C₀₋₈ alkyl-C(═NR₁₃)R₁₂, —C₀₋₄ alkyl-N(R₁₃)—C(═NR₁₄)R₁₂, —C₀₋₈ alkyl-C(O)NR₁₃R₁₄ and —C₀₋₄ alkyl-N(R₁₃)—C(O)R₁₂;
R₅ₐ and R_{5b} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₈ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, or, R₅ₐ and R_{5b}, together with the carbon atom to which they are directly attached, form a C(O), C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₃)R₁₂, —C₀₋₄ alkyl-N(Ru)—C(═NR₁₄)R₁₂, —C₀₋₄ alkyl-C(O)NR₁₃R₁₄ and —C₀₋₄ alkyl-N(R₁₃)—C(O)R₁₂;
each R_{5c} and each R_{5d} are independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₂, —C₀₋₄ alkyl-C(O)R₁₂, —C₀₋₄ alkyl-O—C(O)R₁₂, —C₀₋₄ alkyl-P(O)(R₂)₂, —C₀₋₄ alkyl-NR₁₃R₁₄, —C₀₋₈ alkyl-C(═NR₁₃)R₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, or, R_{5c} and R_{5d}, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, provided that when R_{5c} or R_{5d} is H or C₁₋₄ alkyl, R₁ is optionally substituted C₂₋₁₀ alkynyl, or, L₃ is O, S, S(O) or S(O)₂;
R₅ₑ and R_{5f} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, or, R₅ₑ and R_{5f}, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₈ alkyl-N═SR₈R₉, —C₀₋₄ alkyl-O—S(O)₂R₁₀, —C₀₋₄ alkyl-S(O)ᵣR₁₀, —C₀₋₈ alkyl-O—R₁₁, —C₀₋₄ alkyl-C(O)OR₁₁, —C₀₋₄ alkyl-C(O)SR₁₁, —C₀₋₈ alkyl-S—C(O)R₁₂, —C₀₋₄ alkyl-C(O)R₁₂, —C₀₋₈ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂;
R_{5g} and R₅ₕ are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₈ alkyl-SF₅, —C₀₋₄ alkyl-S(O)(═N—R₇)R₈, —C₀₋₈ alkyl-N═S(O)R₈R₉, —C₀₋₈ alkyl-N═SR₈R₉, —C₀₋₄ alkyl-O—S(O)₂R₁₀, —C₀₋₄ alkyl-S(O)ᵣR₁₀, —C₀₋₈ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₈ alkyl-C(═NR₃)R₁₂, —C₀₋₄ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂, or, R_{5g} and R₅ₕ, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl—O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₈ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ and —C₀₋₈ alkyl-N(R₁₃)—C(O)R₁₂;
R₆ₐ, R_{6b}, R_{6c}, R₇, R₈, R₉, R₁₀, R₁₁, R₁₂, R₁₃, R₁₄, m, n, p and r are as defined in the compound of formula (I).

As a preferred embodiment, in the compound of formula (I), the stereoisomer or pharmaceutically acceptable salt thereof, each R₇ is independently selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-S(O)ᵣR₁₀, —C₀₋₄ alkyl-C(O)OR₁₁, —C₀₋₄ alkyl-C(O)R₁₂ or —C₀₋₄ alkyl-C(O)NR₁₃R₁₄, and the above groups are optionally further substituted by one or more substituents selected from the group consisting of deuterium, halogen, cyano, C₁₋₄ alkyl, C₂₋₄ alkenyl, C₂₋₄ alkynyl, C₁₋₄ haloalkyl, C₁₋₄ deuteroalkyl, C₃₋₆ cycloalkyl, 3-6 membered heterocyclyl, C₆₋₈ aryl, 5-8 membered heteroaryl, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)R₁₂, —C₀₋₄ alkyl-O—C(O)R₁₂, —C₀₋₄ alkyl-NR₁₃R₁₄, —C₀₋₄ alkyl-C(═NR₁₃)R₁₂, —C₀₋₈ alkyl-N(R₁₃)—C(═NR₁₄)R₁₂, —C₀₋₄ alkyl-C(O)NR₁₃R₁₄ or —C₀₋₄ alkyl-N(R₁₃)—C(O)R₁₂;
each R₈ and each R₉ are independently selected from the group consisting of hydrogen, deuterium, hydroxy, C₁₋₄ alkyl, C₂₋₄ alkenyl, C₂₋₄ alkynyl, C₃₋₆ cycloalkyl, 3-6 membered heterocyclyl, C₆₋₈ aryl or 5-8 membered heteroaryl, or, R₈ and R₉, together with the sulfur atom to which they are directly attached, form a 3-6 membered heterocyclyl, and the above groups are optionally further substituted by one or more substituents selected from the group consisting of deuterium, halogen, cyano, C₁₋₄ alkyl, C₂₋₄ alkenyl, C₂₋₄ alkynyl, C₁₋₄ haloalkyl, C₁₋₄ deuteroalkyl, C₃₋₆ cycloalkyl, 3-6 membered heterocyclyl, C₆₋₈ aryl, 5-8 membered heteroaryl, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)R₁₂, —C₀₋₄ alkyl-O—C(O)R₁₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄ or —C₀₋₄ alkyl-N(R₁₃)—C(O)R₁₂;
each R₁₀ is independently selected from the group consisting of hydrogen, deuterium, hydroxy, C₁₋₄ alkyl, C₂₋₄ alkenyl, C₃₋₆ cycloalkyl, 3-6 membered heterocyclyl, C₆₋₈ aryl, 5-8 membered heteroaryl and —NR₁₃R₁₄, and the above groups are each independently optionally further substituted by one or more substituents selected from the group consisting of deuterium, halogen, hydroxy, ═O, C₁₋₄ alkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy and —NR₁₃R₁₄;
each R₁₁ is independently selected from the group consisting of hydrogen, deuterium, C₁₋₄ alkyl, C₂₋₄ alkenyl, C₃₋₆ cycloalkyl, 3-6 membered heterocyclyl, C₆₋₈ aryl and 5-8 membered heteroaryl, and the above groups are each independently optionally further substituted by one or more substituents selected from the group consisting of deuterium, halogen, hydroxy, ═O, cyano, C₁₋₄ alkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy and —NR₁₃R₁₄;
each R₁₂ is independently selected from the group consisting of hydrogen, deuterium, hydroxy, C₁₋₄ alkyl, C₁₋₄ alkoxy, C₂₋₄ alkenyl, C₂₋₄ alkynyl, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy and —NR₁₃R₁₄, and the above groups are each independently optionally further substituted by one or more substituents selected from the group consisting of deuterium, halogen, hydroxy, ═O, cyano, C₁₋₄ alkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy and —NR₁₃R₁₄;
each R₁₃ and each R₁₄ are independently selected from the group consisting of hydrogen, deuterium, hydroxy, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, sulfinyl, sulfonyl, methanesulfonyl, isopropylsulfonyl, cyclopropylsulfonyl, p-toluenesulfonyl, aminosulfonyl, dimethylaminosulfonyl and C₁₋₄ alkanoyl, and the above groups are each independently optionally further substituted by one or more substituents selected from the group consisting of deuterium, halogen, hydroxy, ═O, C₁₋₄ alkyl, C₂₋₄ alkenyl, C₂₋₄ alkynyl, C₁₋₄ haloalkyl, C₁₋₄ deuteroalkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, amino, monoC₁₋₄ alkylamino, diC₁₋₄ alkylamino and C₁₋₄ alkanoyl;
R₁₃ and R₁₄, together with the nitrogen atom to which they are directly attached, form a 4-6 membered heterocyclyl or 5-8 membered heteroaryl, and the 4-6 membered heterocyclyl or 5-8 membered heteroaryl is optionally further substituted by one or more substituents selected from the group consisting of deuterium, halogen, hydroxy, ═O, C₁₋₄ alkyl, C₂₋₄ alkenyl, C₂₋₄ alkynyl, C₁₋₄ haloalkyl, C₁₋₄ deuteroalkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, methanesulfonylmethyl, amino, monoC₁₋₄ alkylamino, diC₁₋₄ alkylamino and C₁₋₄ alkanoyl; and
each r is independently 0, 1 or 2.

As a further preferred embodiment, in the compound of formula (I), the stereoisomer or pharmaceutically acceptable salt thereof, the compound of formula (I) is a compound having formula (II) as shown below:

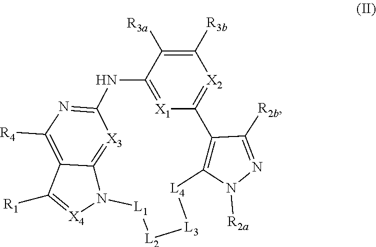

wherein, X₁ is CRₐ or N; X₂ is CR_{b} or N; X₃ is CR_{c} or N; X₄ is CR_{d} or N;
L₁ is CR₅ₐR_{5b};
L₂ is (CR₅ₑR_{5d})ₚ, NR₆ₐ, O, S, S(O), S(O)₂ or C(O);
L₃ is a bond, CR₅ₑR_{5f} or C(O);
L₄ is CR_{5g}R₅ₕ, O, S, S(O), S(O)₂, NR_{6c} or C(O);
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)R₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, and the above groups are each independently optionally further substituted by one or more substituents selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₁₃)—S(O)₂R₁₀, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl. C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₈ alkyl-N═S(O)R₈R₉, —C₀₋₄ alkyl-N═SR₈R₉, —C₀₋₄ alkyl-N(R₁₃)—S(O)₂R₁₀, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₂, —C₀₋₄ alkyl-P(O)(R₁₂)₂, —C₀₋₈ alkyl-NR₁₃R₁₄, —C₀₋₄ alkyl-C(═NR₁₃)R₁₂, —C₀₋₈ alkyl-N(R₁₃)—C(═NR₁₄)R₁₂, —C₀₋₄ alkyl-C(O)NR₁₃R₁₄ and —C₀₋₈ alkyl-N(R₁₃)—C(O)R₁₂;
R₂ₐ is selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —S(O)ᵣR₁₀, —O—R₁₁, —C(O)OR₁₁, —C(O)SR₁₁, —C(O)R₁₂ and —C(O)NR₁₃R₁₄, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R_{2b} is selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₂;
R₃ₐ, R_{3b}, Rₐ and R_{b} are each independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R₄ and R_{c} are each independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R_{d} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R₅ₐ and R_{5b} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, or, R₅ₐ and R_{5b}, together with the carbon atom to which they are directly attached, form a C(O), C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
each R_{5c} and each R_{5d} are independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)₂R₁₀, —O—R₁₁, —C(O)OR₁₁, —C(O)SR₁₁, —S—C(O)R₁₂, —C(O)R₁₂, —O—C(O)R₁₂, —P(O)R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, or, R_{5c} and R_{5d}, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, provided that when se or R_{1d} is H or C₁₋₄ alkyl, R₁ is optionally substituted C₂₋₁₀ alkynyl;
R₅ₑ and R_{5f} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, or, R₅ₑ and R_{5f}, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R)—C(═NR₁₄)R₁₂, —C(O)NR₁₃R₁₄ and —N(R₁₃)—C(O)R₁₂;
R_{5g} and R₅ₕ are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, or, R_{5g} and R₅ₕ, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(Ru)—C(═NR₁₄)R₁₂, —C(O)NR₁₃R₁₄ and —N(R₁₃)—C(O)R₁₂; and
R₆ₐ, R_{6c}, R₇, R₈, R₉, R₁₀, R₁₁, R₁₂, R₁₃, R₁₄, p and r are as defined in the compound of formula (I).

As a further preferred embodiment, in the compound of formula (I), the stereoisomer or pharmaceutically acceptable salt thereof, the compound of formula (I) is a compound having formula (III) as shown below:

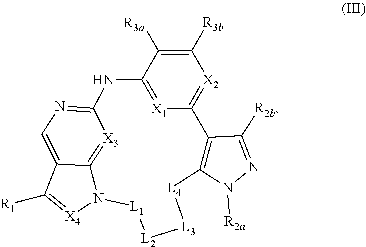

wherein, X₁ is CRₐ or N; X₂ is CR_{b} or N; X₃ is CR_{c} or N; X₄ is CRₐ or N;
L₁ is CR₅ₐR_{5b}; L₂ is (CR_{5c}R_{5d})ₚ; L₃ is a bond, CR₅ₑR_{5f} or C(O); L₃ is CR_{5g}R₅ₕ, O, NR_{6c} or C(O);
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, and the above groups are each independently optionally further substituted by one or more substituents selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₁₃)—S(O)₂R₁₀, —O—S(O)₂R₁₀, —S(O)R₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₄ alkyl-N═SR₈R₉, —C₀₋₄ alkyl-N(R₁₃)—S(O)₂R₁₀, —C₀₋₈ alkyl-O—S(O)₂R₁₀, —C₀₋₄ alkyl-S(O)₂R₁₀, —C₀₋₄ alkyl-O—R₁₁, —C₀₋₈ alkyl-C(O)OR₁₁, —C₀₋₈ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₈ alkyl-P(O)(R₁₂)₂, —C₀₋₄ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₄ alkyl-C(O)NR₁₃R₁₄ and —C₀₋₄ alkyl-N(R₁₃)—C(O)R₁₂;
R₂ₐ is selected from the group consisting of hydrogen, deuterium, hydroxy, C₁₋₄ alkyl, C₂₋₄ alkenyl, C₂₋₄ alkynyl, C₀₋₆ cycloalkyl and 3-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₂;
R_{2b} is selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl and —SF₅, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₂, —P(O)(R₂)₂, —NR₁₃R₁₄, —C(═NR₁₃)R₁₂, —N(R)—C(═NR₄)R₁₂, —C(O)NR₁₃R₁₄ and —N(R₁₃)—C(O)R₁₂;
R₃ₐ, R_{3b}, Rₐ and R_{b} are each independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl and —SF₅;
R_{c} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl and —SF₅;
R_{d} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl and —SF₅;
R₅ₐ and R_{5b} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl and —SF₅, or, R₅ₐ and R_{5b}, together with the carbon atom to which they are directly attached, form a C(O), C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
when p is 1, R_{5c} and R_{5d}, together with the carbon atom to which they are directly attached, form a C₃₋₆ cycloalkyl or 4-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)₂R₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₂, or,
when p is 2, one group of R_{5c} and R_{5d}, together with the carbon atom to which they are directly attached, form a C₃₋₆ cycloalkyl or 4-6 membered heterocyclyl, and when one group of R_{5c} and R_{5d}, together with the carbon atom to which they are directly attached, form a C₃₋₄ cycloalkyl or 4-6 membered heterocyclyl, the other group of R_{5c} and R_{5d} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl. —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R₅ₑ and R_{5f} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl and —SF₅, or, R₅ₑ and R_{5f}, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R_{5g} and R_{5f} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₄ aryl, 5-8 membered heteroaryl and —SF₅, or, R_{5g} and R₅ₕ, together with the carbon atom to which they are directly attached, form a C₃₋₆ cycloalkyl, 4-6 membered heterocyclyl, C₆₋₈ aryl or 5-8 membered heteroaryl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R_{6c} is selected from the group consisting of hydrogen, deuterium, C₁₋₄ alkyl, C₃₋₆ cycloalkyl, 3-6 membered heterocyclyl and —O—R₁₁, and the above groups are each independently optionally further substituted by one or more substituents selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy and —NR₁₃R₁₄; and
R₇, R₈, R₉, R₁₀, R₁₁, R₁₂, R₁₃, R₁₄, p and r are as defined in the compound of formula (I).

As a further preferred embodiment, in the compound of formula (I), the stereoisomer or pharmaceutically acceptable salt thereof, the compound of formula (I) is a compound having formula (IV) as shown below:

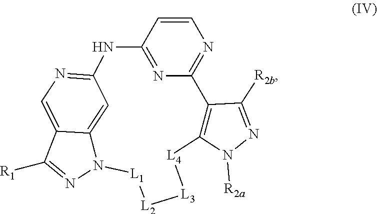

wherein, L₁ is CR₅ₐR_{5b}; L₂ is CR_{5c}R_{5d}; L₃ is CR₅ₑR_{5f}; L₄ is O;
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl and —SF₅, and the above groups are each independently optionally further substituted by one or more substituents selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₁₃)—S(O)₂R₁₀, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, and the above groups are each independently optionally further more substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₁₃)—S(O)₂R₁₀, —O—S(O)₂R₁₀, —S(O)ᵣR₁₀, —O—Ru. —C(O)OR₁₁, —C(O)SR₁₁, —S—C(O)R₁₂, —C(O)R₁₂, —O—C(O)R₁₂. —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R₂ₐ is selected from the group consisting of hydrogen, deuterium, hydroxy, C₁₋₄ alkyl, C₂₋₄ alkenyl, C₂₋₄ alkynyl, C₃-f cycloalkyl and 3-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S and —SF₅;
R_{2b} is selected from the group consisting of hydrogen, deuterium, halogen, cyano. C₁₋₄ alkyl, C₂₋₄ alkenyl, C₂₋₄ alkynyl, C₃₋₆ cycloalkyl, 3-6 membered heterocyclyl and —SF₅, and the above groups are each independently optionally further substituted by one or more substituents selected from the group consisting of deuterium, halogen, cyano. C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S and —SF₅;
R₅ₐ and R_{5b} are each independently selected from the group consisting of hydrogen, deuterium, halogen, cyano, C₁₋₄ alkyl, C₃₋₆ cycloalkyl and 3-6 membered heterocyclyl, or, R₅ₐ and R_{5b}, together with the carbon atom to which they are directly attached, form a C₃₋₆ cycloalkyl or 4-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S and —SF₅;
R_{5c} and R_{5d}, together with the carbon atom to which they are directly attached, form a C₃₋₆ cycloalkyl or 4-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S and —SF₅;
R₅ₑ and R_{5f} are each independently selected from the group consisting of hydrogen, deuterium, halogen, cyano, C₁₋₄ alkyl, C₃₋₆ cycloalkyl and 3-6 membered heterocyclyl, or, R₅ₑ and R_{5f}, together with the carbon atom to which they are directly attached, form a C₃₋₆ cycloalkyl or 4-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S and —SF₅; and
R₇, R₈, R₉, R₁₀, R₁₁, R₁₂, R₁₁, R₁₄ and r are as defined in the compound of formula (I).

As a more further preferred embodiment, in the compound of formula (I), the stereoisomer or pharmaceutically acceptable salt thereof, L₁ is CR₅ₐR_{5b}; L₂ is CR_{5c}R_{5d}; L₃ is CR₅ₑR_{5f}, L₄ is O;
R₅ₐ and R_{5b} are each independently selected from the group consisting of hydrogen, deuterium, fluorine, chlorine, bromine, cyano, methyl, ethyl, propyl, isopropyl, cyclopropyl, cyclobutyl, oxacyclopropyl, oxacyclobutyl, azacyclopropyl and azacyclobutyl, and the above groups are each independently optionally further substituted by one or more substituents selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S and —SF₅;
R_{5c} and R_{5d}, together with the carbon atom to which they are directly attached, form a C₃₋₆ cycloalkyl or 4-6 membered heterocyclyl, and the above groups are each independently optionally further substituted by one or more substituents selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S and —SF₅; and
R₅ₑ and R_{5f} are each independently selected from the group consisting of hydrogen, deuterium, fluorine, chlorine, bromine, cyano, methyl, ethyl, propyl, isopropyl, cyclopropyl, cyclobutyl, oxacyclopropyl, oxacyclobutyl, azacyclopropyl and azacyclobutyl, and the above groups are each independently optionally further substituted by one or more substituents selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S and —SF₅.

As a more further preferred embodiment, in the compound of formula (I), the stereoisomer or pharmaceutically acceptable salt thereof, R₂ₐ is selected from the group consisting of hydrogen, deuterium, hydroxy, methyl, ethyl, propyl, isopropyl, vinyl, ethynyl, cyclopropyl, cyclobutyl, oxacyclopropyl, oxacyclobutyl, azacyclopropyl and azacyclobutyl, and the above groups are each independently optionally further substituted by one or more substituents selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S and —SF₅; and
R_{2b} is selected from the group consisting of hydrogen, deuterium, fluorine, chlorine, bromine, cyano, methyl, ethyl, propyl, isopropyl, vinyl, ethynyl, cyclopropyl, cyclobutyl, oxacyclopropyl, oxacyclobutyl, azacyclopropyl, azacyclobutyl and —SF₅, and the above groups are each independently optionally further substituted by one or more substituents selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S and —SF₅.

As a further preferred embodiment, in the compound of formula (I), the stereoisomer or pharmaceutically acceptable salt thereof, the compound of formula (I) is a compound having formula (V) as shown below:

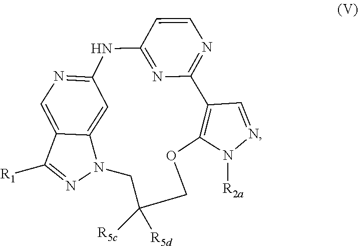

wherein, R₁ is selected from C₂₋₄ alkenyl, C₂₋₄ alkynyl and 3-8 membered heterocyclyl, and the above groups are each independently optionally further substituted by one or more substituents selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, ═O, ═S, —SF₅, —N(R)—S(O)₂R₁₀, —O—S(O)₂R₁₀, —S(O)ᵣR₁₀, —O—R₁₁, —C(O)OR₁₁, —S—C(O)R₁₂, —C(O)R₁₂, —O—C(O)R₁₂, —NR₁₃R₁₄, —C(O)NR₁₃R₁₄ and —N(R₁₃)—C(O)R₁₂, and the above groups are each independently optionally further more substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl. C₆₋₈ aryl, 5-8 membered heteroaryl, ═O, ═S, —SF₅, —N(R₁₃)—S(O)₂R₁₀, —O—S(O)₂R₁₀, —S(O)ᵣR₁₀, —O—R₁₁, —C(O)OR₁₁, —C(O)R₁₂, —O—C(O)R₁₂, —NR₁₃R₁₄, —C(O)NR₁₃R₁₄ and —N(R₁₃)—C(O)R₁₂;
R₂ₐ is selected from the group consisting of hydrogen, deuterium, C₁₋₄ alkyl, C₃₋₆ cycloalkyl and 3-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₃₋₆ cycloalkyl and 3-6 membered heterocyclyl;
R_{5c} and R_{5d}, together with the carbon atom to which they are directly attached, form a C₃₋₆ cycloalkyl or 4-6 membered heterocyclyl, and the above groups are each independently optionally further substituted by one or more substituents selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S and —SF₅;
R₁₀, R₁₁, R₁₂, R₁₃, R₁₄ and r are as defined in the compound of formula (I).

As a more further preferred embodiment, in the compound of formula (I), the stereoisomer or pharmaceutically acceptable salt thereof, R_{5c} and R_{5d}, together with the carbon atom to which they are directly attached, form a C₃₋₆ cycloalkyl or 4-6 membered heterocyclyl.

As a more further preferred embodiment, in the compound of formula (I), the stereoisomer or pharmaceutically acceptable salt thereof, R_{5c} and R_{5d}, together with the carbon atom to which they are directly attached, form a cyclopropyl, cyclobutyl, cyclopentyl, cyclohexyl, oxacyclobutyl, oxacyclopentyl or oxacyclohexyl.

As a more further preferred embodiment, in the compound of formula (I), the stereoisomer or pharmaceutically acceptable salt thereof, R₁ is C₂₋₄ alkynyl, and the C₂₋₄ alkynyl is optionally further substituted by one or more substituents selected from the group consisting of deuterium, C₁₋₄ alkyl, C₃₋₆ cycloalkyl and 3-6 membered heterocyclyl, and the above groups are each independently optionally further more substituted by one or more substituents selected from the group consisting of deuterium, halogen, cyano, C₁₋₄ alkyl, C₁₋₄ haloalkyl, C₁₋₄ deuteroalkyl, C₃₋₆ cycloalkyl, 3-6 membered heterocyclyl.

As a more further preferred embodiment, in the compound of formula (I), the stereoisomer or pharmaceutically acceptable salt thereof, R₁ is ethynyl, and the ethynyl is optionally further substituted by one or more substituents selected from the group consisting of deuterium, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl and azacyclohexyl, and the above groups are each independently optionally further more substituted by one or more substituents selected from the group consisting of deuterium, fluorine, bromine, cyano, methyl, ethyl, propyl, isopropyl, butyl, monofluoromethyl, difluoromethyl, trifluoromethyl, monodeuteriomethyl, dideuteriomethyl, trideuteriomethyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl and azacyclohexyl.

As a more further preferred embodiment, in the compound of formula (I), the stereoisomer or pharmaceutically acceptable salt thereof, R₁ is ethynyl, and the ethynyl is optionally further substituted by one or more substituents selected from the group consisting of cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl and azacyclohexyl, and the above groups are each independently optionally further more substituted by one or more substituents selected from the group consisting of deuterium, fluorine, bromine, cyano, methyl, ethyl, propyl, isopropyl, monofluoromethyl, difluoromethyl, trifluoromethyl, monodeuteriomethyl, dideuteriomethyl and trideuteriomethyl.

As a more further preferred embodiment, in the compound of formula (I), the stereoisomer or pharmaceutically acceptable salt thereof. R₁ is

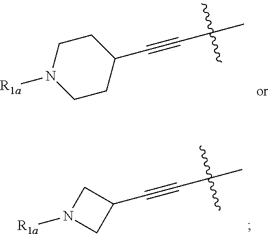

R₁ₐ is selected from hydrogen, methyl, ethyl, propyl, isopropyl, monofluoromethyl, difluoromethyl, trifluoromethyl, monodeuteriomethyl, dideuteriomethyl and trideuteriomethyl; and
R_{1b} is selected from hydrogen, methyl, ethyl, propyl, isopropyl, monofluoromethyl, difluoromethyl, trifluoromethyl, monodeuteriomethyl, dideuteriomethyl and trideuteriomethyl.

As a more further preferred embodiment, in the compound of formula (I), the stereoisomer or pharmaceutically acceptable salt thereof. R₁ is 4-8 membered nitrogen-containing heterocyclyl, and the 4-8 membered nitrogen-containing heterocyclyl is optionally further substituted by one or more substituents selected from the group consisting of deuterium, halogen, cyano, C₁₋₄ alkyl, ═O, —N(R₁₃)—S(O)₂R₁₀, —S(O)R₁₀, —O—R₁₁, —O—C(O)R₁₂, —NR₁₃R₁₄ and —N(R₁₃)—C(O)R₁₂, and the C₁₋₄ alkyl is more optionally further substituted by one or more substituents selected from the group consisting of deuterium, halogen, cyano, C₁₋₄ alkyl, C₁₋₄ haloalkyl, C₁₋₄ deuteroalkyl, —N(R₁₃)—S(O)₂R₁₀, —O—S(O)₂R₁₀, —S(O)ᵣR₁₀, —O—R₁₁, —O—C(O)R₁₂, —NR₁₃R₁₄ and —N(R₁₃)—C(O)R₁₂; and
R₁₀, R₁₁, R₁₂, R₁₃, R₁₄ and r are as defined in the compound of formula (I).

As a more further preferred embodiment, in the compound of formula (I), the stereoisomer or pharmaceutically acceptable salt thereof. R₁ is of the following structure:

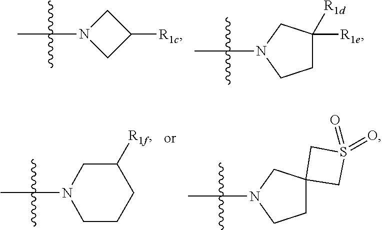

wherein,
R_{1c} is selected from the group consisting of hydrogen, deuterium, halogen, C₁₋₄ alkyl, —N(R₁₃ₐ)—S(O)₂R₁₀ₐ, —O—R₁₁ₐ and —N(R₁₃ₐ)—C(O)R₁₂, and the C₁₋₄ alkyl is more optionally further substituted by one or more substituents selected from the group consisting of deuterium, C₁₋₄ alkyl, C₁₋₄ haloalkyl, C₁₋₄ deuteroalkyl, —S(O)ᵣR_{10b}, and —O—R₁₁ₐ;
R_{1d} is selected from the group consisting of hydrogen, deuterium, halogen, C₁₋₄ alkyl, —N(R₁₃ₐ)—S(O)₂R₁₀ₐ, —O—R₁₁ₐ and —N(R₁₃ₐ)—C(O)R₁₂ₐ;
R_{1c} is selected from the group consisting of hydrogen, deuterium, halogen, C₁₋₄ alkyl, —N(R₁₃ₐ)—S(O)₂R₁₀ₐ, —O—R₁₁ₐ and —N(R₁₃ₐ)—C(O)R₁₂ₐ, and the C₁₋₄ alkyl is more optionally further substituted by one or more substituents selected from the group consisting of deuterium, C₁₋₄ alkyl, C₁₋₄ haloalkyl, C₁₋₄ deuteroalkyl, —S(O)ᵣR_{10b} and —O—R₁₁ₐ;
R_{1f} is selected from the group consisting of hydrogen, deuterium, halogen, C₁₋₄ alkyl, —N(R₁₃ₐ)—S(O)₂R₁₀ₐ, —O—R₁₁ₐ and —N(R₁₃ₐ)—C(O)R₁₂ₐ, and the C₁₋₄ alkyl is more optionally further substituted by one or more substituents selected from the group consisting of deuterium, C₁₋₄ alkyl, C₁₋₄ haloalkyl, C₁₋₄ deuteroalkyl, —S(O)ᵣR_{10b} and —O—R₁₁ₐ;
each R₁₀ₐ is independently selected from the group consisting of hydrogen, C₁₋₄ alkyl, C₃₋₆ cycloalkyl and 3-6 membered heterocyclyl;
each R_{10b} is independently selected from the group consisting of hydrogen, C₁₋₄ alkyl, C₃₋₆ cycloalkyl and 3-6 membered heterocyclyl, and the above groups are each independently optionally further substituted by one or more substituents selected from the group consisting of deuterium, halogen, hydroxy, C₁₋₄ alkyl and C₁₋₄ alkoxy;
each R₁₁ₐ is independently hydrogen or C₁₋₄ alkyl;
each R₁₂ₐ is independently selected from the group consisting of hydrogen, C₁₋₄ alkyl, C₁₋₄ alkoxy and C₃₋₆ cycloalkyl, and the above groups are each independently optionally further substituted by one or more substituents selected from the group consisting of deuterium, halogen, hydroxy and cyano;
each R₁₃ₐ is independently hydrogen or C₁₋₄ alkyl; and
each r is independently 0, 1 or 2.

As a more further preferred embodiment, in the compound of formula (I), the stereoisomer or pharmaceutically acceptable salt thereof, R₁ is of the following structure:

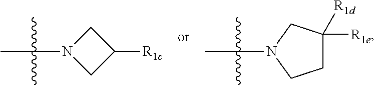

wherein, R₁ is selected from the group consisting of hydrogen, deuterium, fluorine, bromine, methyl, ethyl, propyl, isopropyl, butyl, —N(R₁₃ₐ)—S(O)₂R₁₀ₐ, —O—R₁₁ₐ and —N(R₁₃ₐ)—C(O)R₁₂ₐ, and the methyl, ethyl, propyl, isopropyl or butyl is more independently optionally further substituted by one or more substituents selected from the group consisting of deuterium, methyl, ethyl, propyl, isopropyl, butyl, monofluoromethyl, difluoromethyl, trifluoromethyl, monodeuteriomethyl, dideuteriomethyl, trideuteriomethyl, —S(O)ᵣR_{10b} and —O—R₁₁ₐ;
R_{1d} is selected from the group consisting of hydrogen, deuterium, fluorine, bromine, methyl, ethyl, propyl, isopropyl and butyl;
R_{1c} is selected from the group consisting of hydrogen, deuterium, fluorine, bromine, methyl, ethyl, propyl, isopropyl, butyl, —N(R₁₃ₐ)—S(O)₂R₁₀ₐ, —O—R₁₁ₐ and —N(R₁₃ₐ)—C(O)R₁₂ₐ, and the methyl, ethyl, propyl, isopropyl or butyl is more independently optionally further substituted by one or more substituents selected from the group consisting of deuterium, methyl, ethyl, propyl, isopropyl, butyl, monofluoromethyl, difluoromethyl, trifluoromethyl, monodeuteriomethyl, dideuteriomethyl, trideuteriomethyl, —S(O)ᵣR_{10b}, and —O—R₁₁ₐ;
each R₁₀ₐ is independently selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl and azacyclohexyl;
each R_{10b} is independently selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl and azacyclohexyl, and the above groups are each independently optionally further substituted by one or more substituents selected from the group consisting of deuterium, fluorine, chlorine, hydroxy, methyl, ethyl, propyl, isopropyl, butyl, methoxy, ethoxy and propoxy;
each R₁₁ₐ is independently selected from the group consisting of hydrogen, methyl, ethyl, propyl, isopropyl and butyl;
each R₁₂, is independently selected from the group consisting of hydrogen, methyl, ethyl, propyl, isopropyl, butyl, methoxy, ethoxy, propoxy, tert-butoxy, cyclopropyl, cyclobutyl, cyclopentyl and cyclohexyl, and the above groups are each independently optionally further substituted by one or more substituents selected from the group consisting of deuterium, fluorine, chlorine, hydroxy and cyano;
each R₁₃ₐ is independently hydrogen; and
each r is independently 0, 1 or 2.

As a more further preferred embodiment, in the compound of formula (I), the stereoisomer or pharmaceutically acceptable salt thereof, R₁ is of the following structure:

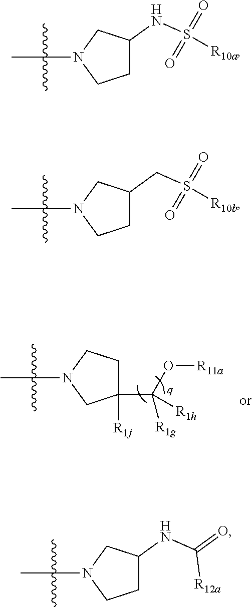

wherein,
R_{1g} is hydrogen, deuterium or methyl; R_{1b} is hydrogen, deuterium or methyl; R₁ⱼ is hydrogen, deuterium or methyl; q is 0, 1 or 2;
R₁₀ₐ is selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl and azacyclohexyl;
R_{10b} is selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl and azacyclohexyl, and the above groups are each independently optionally further substituted by one or more substituents selected from the group consisting of deuterium, fluorine, chlorine, hydroxy, methyl, ethyl, propyl, isopropyl, butyl, methoxy, ethoxy and propoxy;
each R₁₁ₐ is independently selected from the group consisting of hydrogen, methyl, ethyl, propyl, isopropyl and butyl;
R₁₂ₐ is selected from the group consisting of hydrogen, methyl, ethyl, propyl, isopropyl, butyl, methoxy, ethoxy, propoxy, tert-butoxy, cyclopropyl, cyclobutyl, cyclopentyl and cyclohexyl, and the above groups are each independently optionally further substituted by one or more substituents selected from the group consisting of deuterium, fluorine, chlorine, hydroxy and cyano.

As a more further preferred embodiment, in the compound of formula (I), the stereoisomer or pharmaceutically acceptable salt thereof, R₂ₐ is selected from the group consisting of hydrogen, deuterium, C₁₋₄ alkyl, C₁₋₄ haloalkyl, C₁₋₄ deuteroalkyl, C₃₋₆ cycloalkyl and 3-6 membered heterocyclyl.

As a more further preferred embodiment, in the compound of formula (I), the stereoisomer or pharmaceutically acceptable salt thereof, R₂ₐ is selected from the group consisting of hydrogen, deuterium, methyl, ethyl, propyl, isopropyl, butyl, monofluoromethyl, difluoromethyl, trifluoromethyl, monodeuteriomethyl, dideuteriomethyl, trideuteriomethyl, cyclopropyl, cyclobutyl, oxacyclopropyl, oxacyclobutyl, azacyclopropyl and azacyclobutyl.

As the most preferred embodiment, the compound of formula (I), the stereoisomer or pharmaceutically acceptable salt thereof includes, but is not limited to, the following compounds:

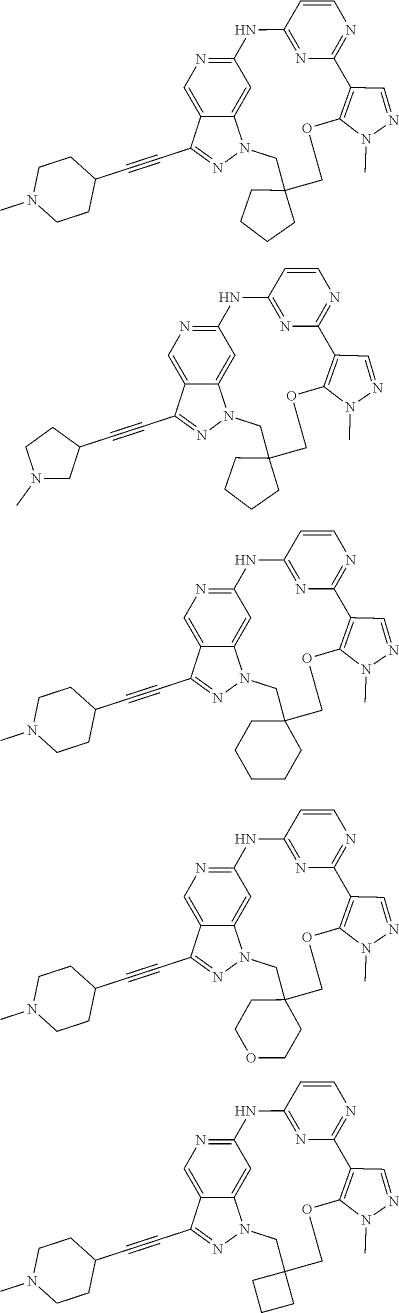

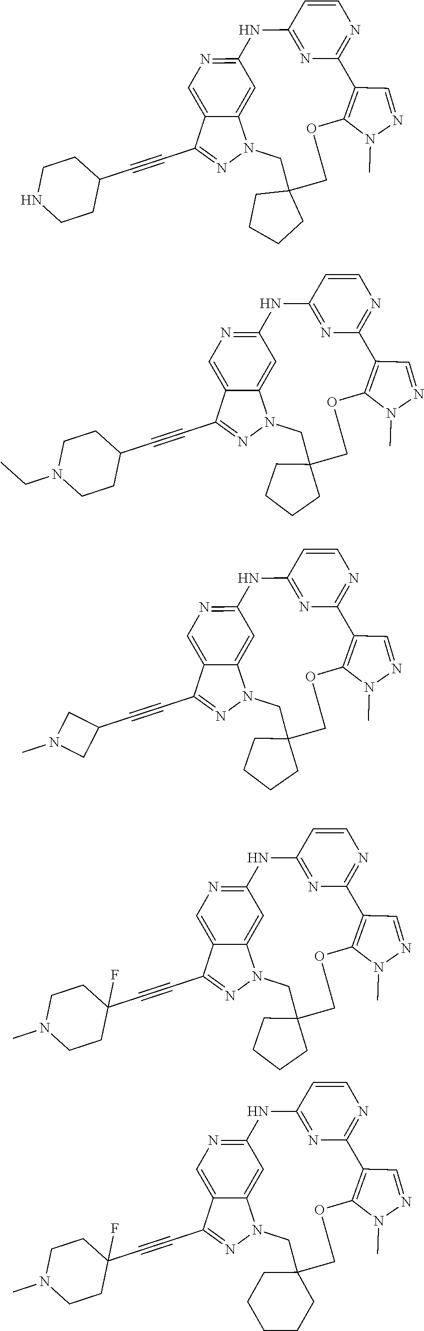

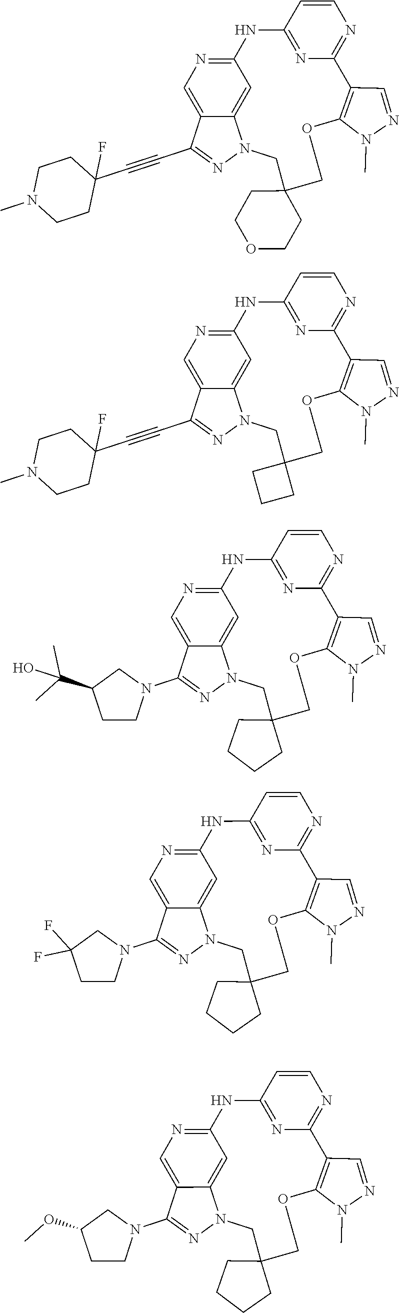

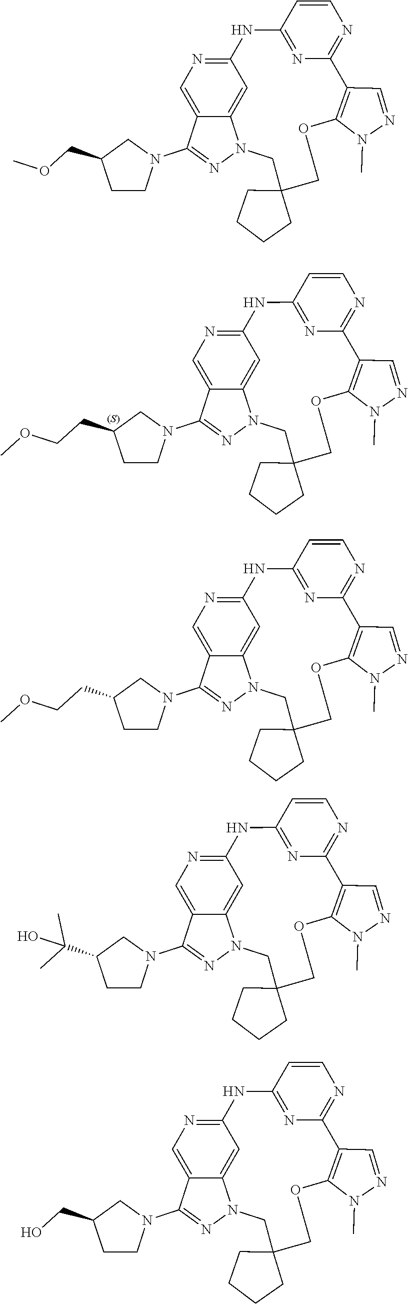

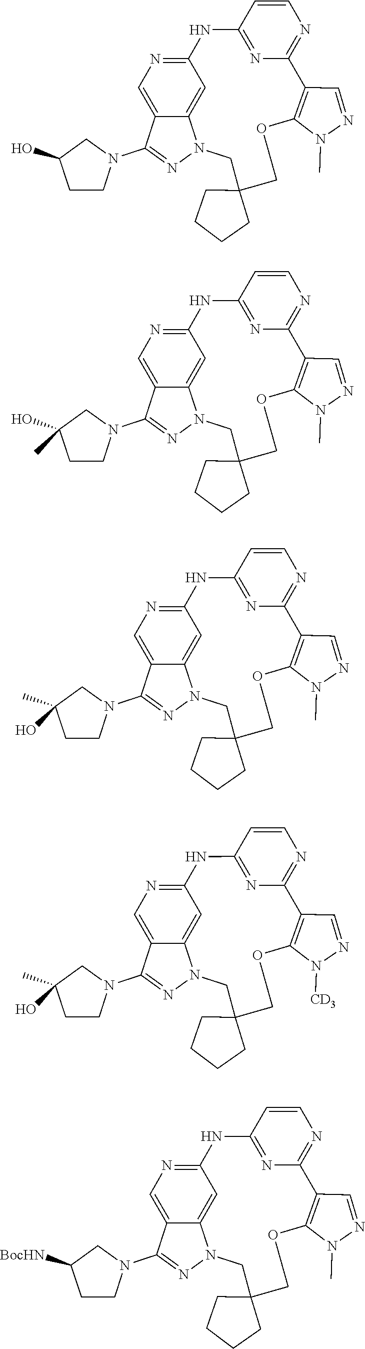

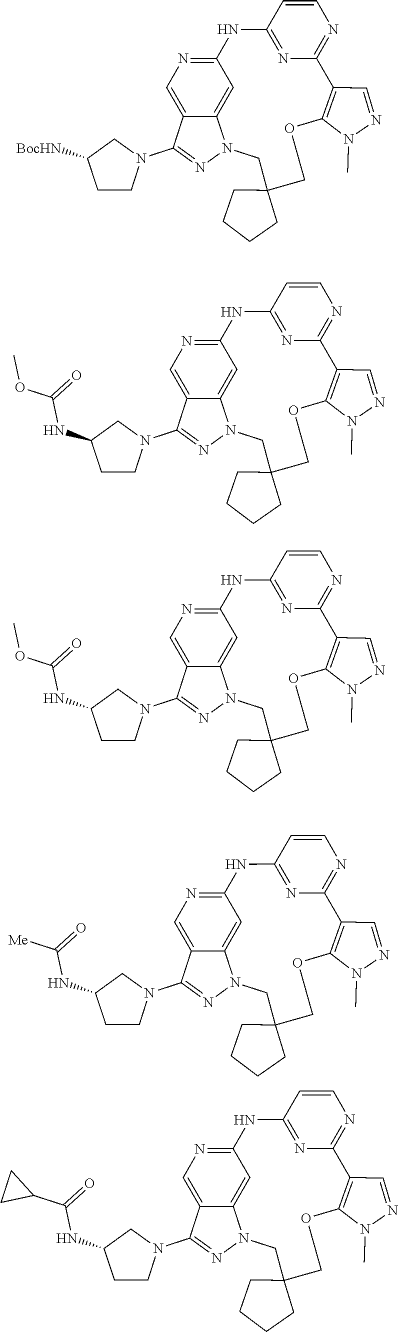

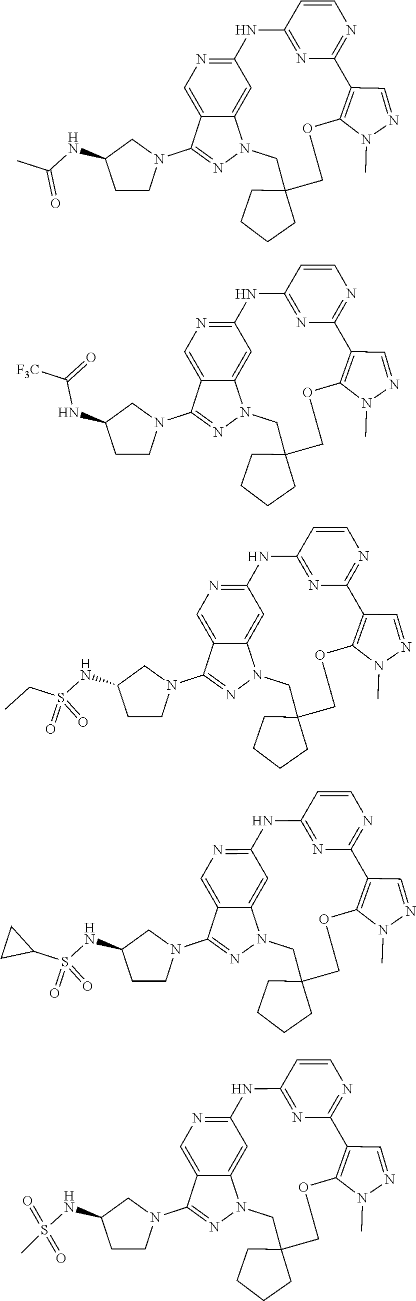

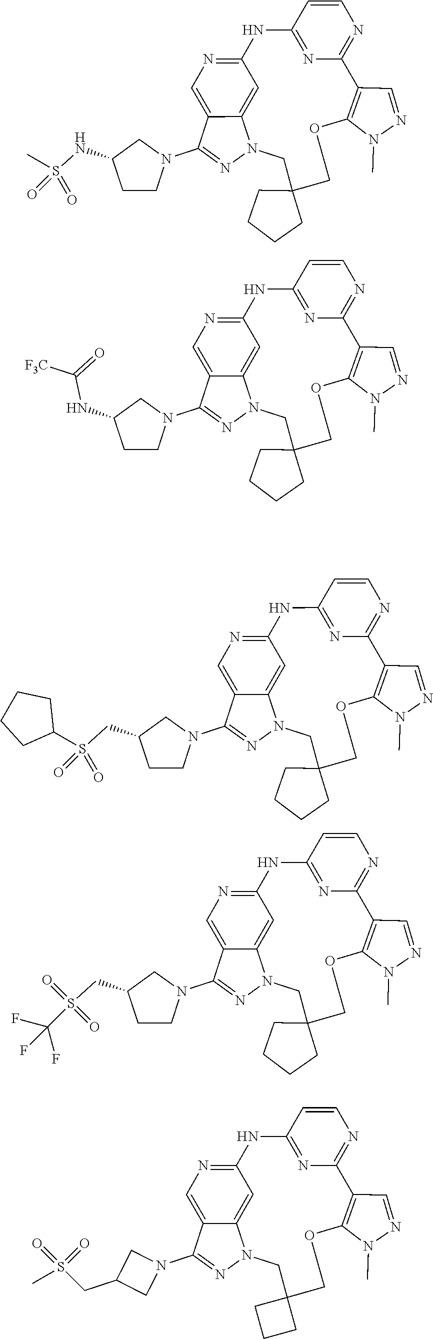

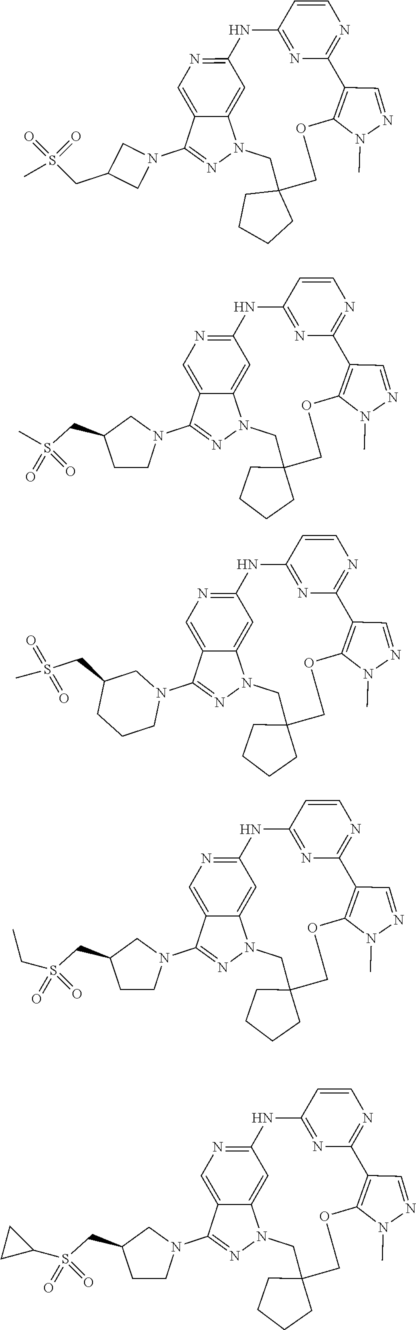

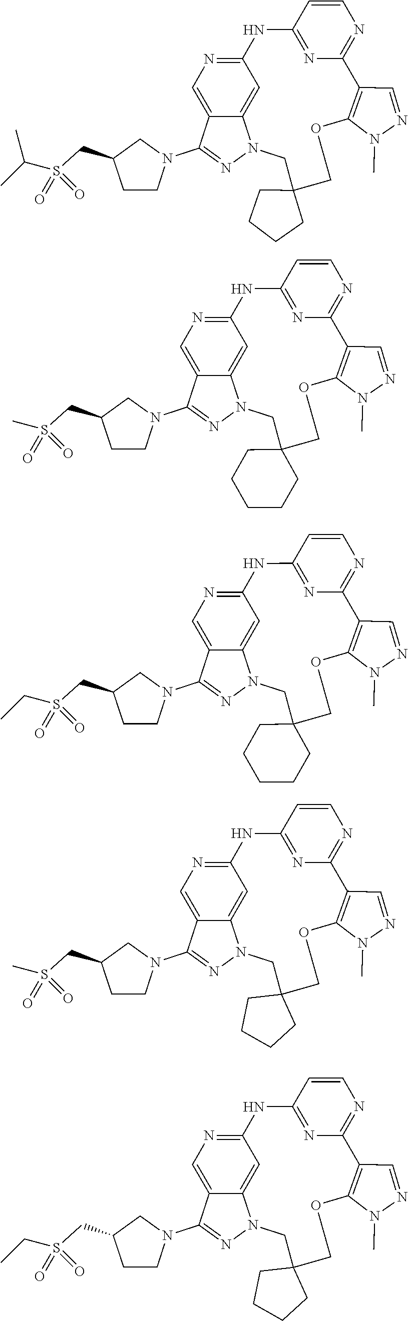

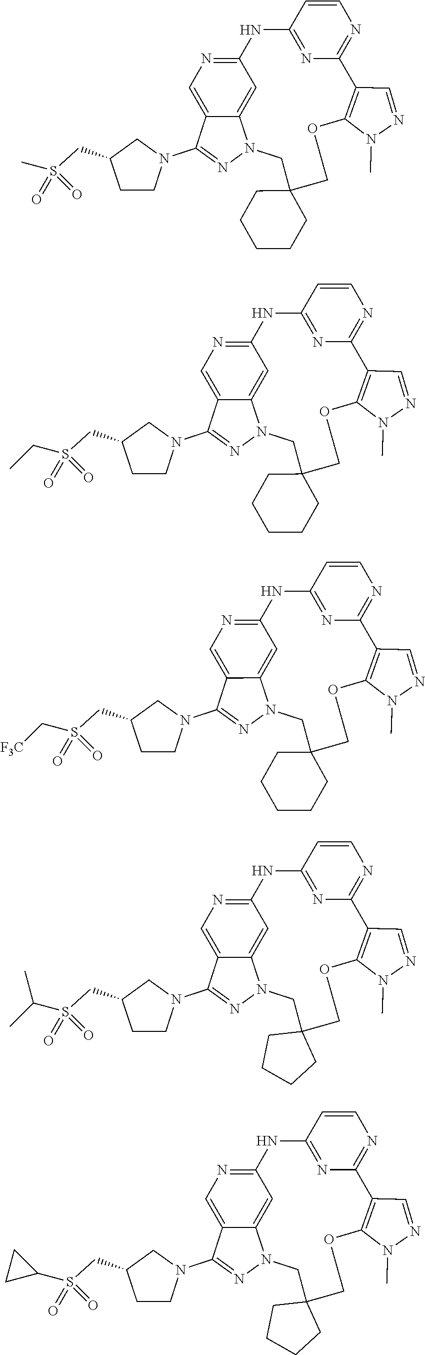

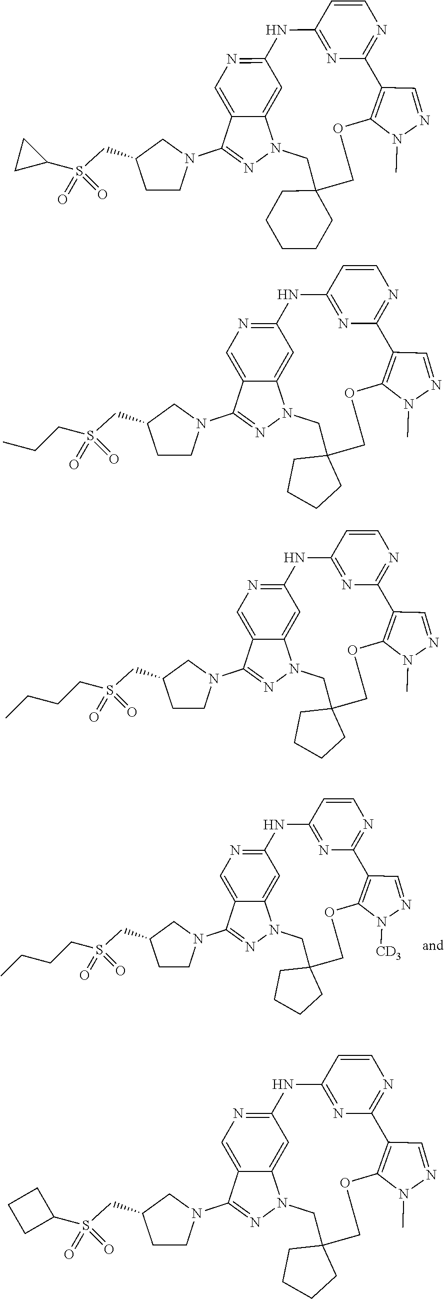

The second aspect of the present invention provides a process for preparing the compound of formula (T), the stereoisomer or pharmaceutically acceptable salt thereof, comprising the following step:

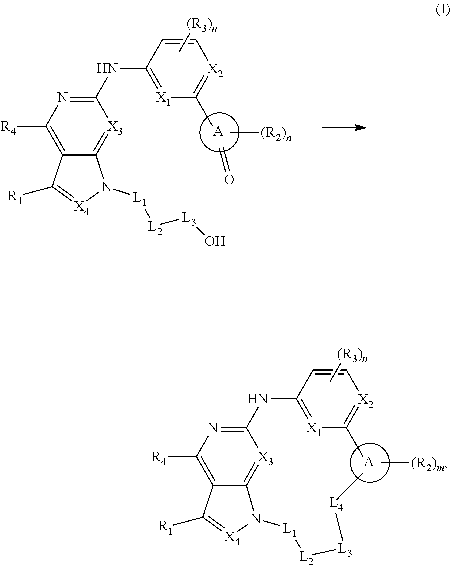

Ring A, X₁, X₂, X₃, X₄, L₁, L₂, L₃, L₄, R₁, R₂, R₃, R₄, m and n are as defined in the compound of formula (I).

The third aspect of the present invention provides a pharmaceutical composition comprising the compound of formula (I), the stereoisomer or pharmaceutically acceptable salt thereof, and a pharmaceutically acceptable carrier.

The present invention also relates to use of the compound of formula (I), the stereoisomer or pharmaceutically acceptable salt thereof in the preparation of a medicament for treating and/or preventing tumors or metastatic diseases associated at least partially with EGFR L858R/C797S double mutation, EGFR Del19/C797S double mutation, L858R/T790M/C797S triple mutation and Del19/T790M/C797S triple mutation.

The present invention also relates to a use of the compound of formula (I), the stereoisomer or pharmaceutically acceptable salt thereof in the preparation of drugs for preventing and/or treating tumors and/or metastatic diseases caused by hyperproliferation diseases and dysfunction in cell death induction.

The present invention also relates to a use of the aforementioned compound of formula (I), the stereoisomer or pharmaceutically acceptable salt thereof in the preparation of drugs for preventing and/or treating lung cancer, colon cancer, pancreatic cancer, head and neck cancer, breast cancer, ovarian cancer, uterine cancer, gastric cancer, non-small cell lung cancer, leukemia, myelodysplastic syndrome, malignant lymphoma, head and neck tumor, thoracic tumor, gastrointestinal tumor, endocrine tumor, breast and other gynecological tumor, urological tumor, skin tumor, sarcoma, sinonasal inverted papilloma or sinonasal squamous cell carcinoma associated with sinonasal inverted papilloma, all of which are associated at least partially with EGFR L858R/C797S double mutation, EGFR Del19/C797S double mutation, L858R/T790M/C797S triple mutation and Del19/T790M/C797S triple mutation.

The present invention also relates to the compound of formula (I), the stereoisomer or pharmaceutically acceptable salt thereof, for use as a drug.

The present invention also relates to the compound of formula (I), the stereoisomer or pharmaceutically acceptable salt thereof, and the use thereof for treating and/or preventing tumors or metastatic diseases associated at least partially with EGFR L858R/C797S double mutation, EGFR Del19/C797S double mutation, L858R/T790M/C797S triple mutation and Del19/T790M/C797S triple mutation.

The present invention also relates to the compound of formula (I), the stereoisomer or pharmaceutically acceptable salt thereof, and the use thereof for preventing and/or treating tumors and/or metastatic diseases caused by hyperproliferation diseases and disorders inducing cell death.

The present invention also relates to the compound of formula (I), the stereoisomer or pharmaceutically acceptable salt thereof, and the use thereof for treating and/or preventing lung cancer, colon cancer, pancreatic cancer, head and neck cancer, breast cancer, ovarian cancer, uterine cancer, gastric cancer, non-small cell lung cancer, leukemia, myelodysplastic syndrome, malignant lymphoma, head and neck tumor, thoracic tumor, gastrointestinal tumor, endocrine tumor, breast and other gynecological tumor, urological tumor, skin tumor, sarcoma, sinonasal inverted papilloma or sinonasal squamous cell carcinoma associated with inverted papilloma of the sinonasal cavity, all of which are associated at least partially with EGFR L858R/C797S double mutation. EGFR Del19/C797S double mutation, L858R/T790M/C797S triple mutation and Del19/T790M/C797S triple mutation.

The present invention also relates to a method for treating and/or preventing tumors or metastatic diseases associated at least partially with EGFR L858R/C797S double mutation, EGFR Del19/C797S double mutation, L858R/T790M/C797S triple mutation and Del19/T790M/C797S triple mutation, comprising administering to a patient in need a therapeutically effective amount of the compound of formula (I), the stereoisomer or pharmaceutically acceptable salt thereof.

The present invention also relates to a method for preventing and/or treating tumors and/or metastatic diseases caused by hyperproliferation diseases and disorders inducing cell death, comprising administering to a patient in need a therapeutically effective amount of the compound of formula (I), the stereoisomer or pharmaceutically acceptable salt thereof.

The present invention also relates to a method for treating and/or preventing lung cancer, colon cancer, pancreatic cancer, head and neck cancer, breast cancer, ovarian cancer, uterine cancer, gastric cancer, non-small cell lung cancer, leukemia, myelodysplastic syndrome, malignant lymphoma, head and neck tumor, thoracic tumor, gastrointestinal tumor, endocrine tumor, breast and other gynecological tumor, urological tumor, skin tumor, sarcoma, inverted papilloma of the sinonasal cavity or squamous cell carcinoma of the sinonasal cavity associated with inverted papilloma of the sinonasal cavity, all of which are associated at least partially with EGFR L858R/C797S double mutation, EGFR Del19/C797S double mutation, L858R/T790M/C797S triple mutation and Del19/T790M/C797S triple mutation, comprising administering to a patient in need a therapeutically effective amount of the compound of formula (I), the stereoisomer or pharmaceutically acceptable salt thereof.

### DETAILED DESCRIPTION OF EMBODIMENTS

After an extensive and intensive research, the inventors of the present invention develop an EGFR inhibitor with the structure of formula (I) for the first time. The series of compounds of the present invention can be widely used in the preparation of drugs for treating and/or preventing tumors or metastatic diseases associated at least partially with EGFR L858R/C797S double mutation, EGFR Del19/C797S double mutation, L858R/T790M/C797S triple mutation and Del19/T790M/C797S triple mutation, and are expected to be developed into a new-generation EGFR inhibitor. The present invention is achieved on this basis.

Detailed description: unless otherwise stated or specified, the following terms used in the specification and claims have the following meanings.

“Alkyl” refers to linear or branched saturated aliphatic alkyl groups, preferably including a linear or branched alkyl having 1 to 10 or 1 to 6 carbon atoms or 1 to 4 carbon atoms, including but not limited to methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl or various branched isomers thereof, etc. “C₁₋₁₀ alkyl” means a linear or branched alkyl containing 1 to 10 carbon atoms, “C₁₋₄ alkyl” means a linear or branched alkyl containing 1 to 4 carbon atoms, “C₀₋₈ alkyl” means a linear or branched alkyl containing 0 to 8 carbon atoms, and “C₀₋₄ alkyl” means a linear or branched alkyl containing 0 to 4 carbon atoms.

Alkyl can be optionally substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Cycloalkyl” or “carbocyclic ring” refers to monocyclic or polycyclic hydrocarbon substituents that are saturated or partially unsaturated, wherein the partially unsaturated cyclic hydrocarbon refers to a cyclic hydrocarbon that may contain one or more (preferably 1, 2 or 3) double bonds, but none of the rings have a fully conjugated π-electron system. Cycloalkyl may be monocyclic cycloalkyl or polycyclic cycloalkyl, preferably including a cycloalkyl containing 3 to 12 or 3 to 8 or 3 to 6 carbon atoms. For example. “C₃₋₁₂ cycloalkyl” means a cycloalkyl having 3 to 12 carton atoms, “C₄₋₁₂ cycloalkyl” means a cycloalkyl having 4 to 12 carton atoms, “C₄₋₈ cycloalkyl” means a cycloalkyl having 4 to 8 carton atoms, “C₃₋₈ cycloalkyl” means a cycloalkyl having 3 to 8 carton atoms, “C₃₋₆ cycloalkyl” means a cycloalkyl having 3 to 6 carton atoms, and “C₄₋₆ cycloalkyl” means a cycloalkyl having 4 to 6 carton atoms, wherein:

Monocyclic cycloalkyl includes but is not limited to cyclopropyl, cyclobutyl, cyclopentyl, cyclopentenyl, cyclohexyl, cyclohexenyl, cyclohexadienyl, cycloheptyl, cycloheptatrienyl, cyclooctyl, etc.

Polycyclic cycloalkyl includes spirocycloalkyl, fused cycloalkyl and bridged cycloalkyl. “Spirocycloalkyl” refers to a polycyclic group in which a carton atom (called spiro-atom) is shared among monocyclic rings, wherein these rings may contain one or more (preferably 1, 2 or 3) double bonds, but none of them have a fully conjugated π-electron system. According to the number of the spiro-atoms shared among the rings, the spirocycloalkyl may be monospirocycloalkyl, bispirocycloalkyl or polyspirocycloalkyl, including but not limited to:

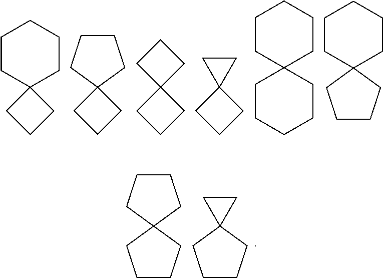

“Fused cycloalkyl” refers to an all-carbon polycyclic group in which each ring shares a pair of adjacent carbon atoms with other rings in the system, wherein one or more of the rings may contain one or more (preferably 1, 2 or 3) double bonds, but none of them have a fully conjugated π-electron system. According to the number of formed rings, the fused cycloalkyl may be bicyclic, tricyclic, tetracyclic or polycyclic, including but not limited to:

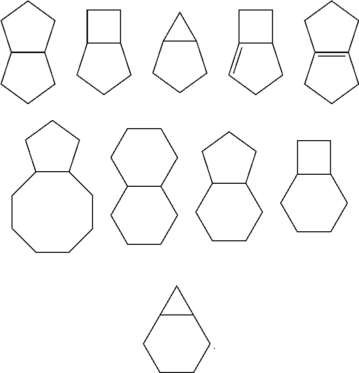

“Bridged cycloalkyl” refers to an all-carbon polycyclic group in which any two rings share two carbon atoms that are not directly connected to each other, wherein these rings may contain one or more (preferably 1, 2 or 3) double bonds, but none of them have a fully conjugated π-electron system. According to the number of formed rings, the bridged cycloalkyl may be bicyclic, tricyclic, tetracyclic or polycyclic, including but not limited to:

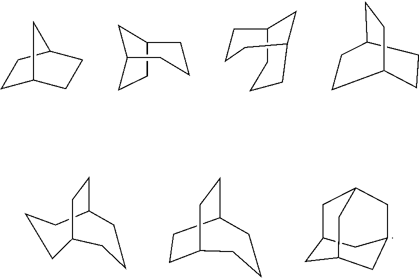

The cycloalkyl ring may be fused to an aryl, heteroaryl or heterocycloalkyl ring, wherein the ring attached to the parent structure is cycloalkyl, which includes, but is not limited to, indenyl, tetrahydronaphthyl, benzocycloheptyl, etc.

Cycloalkyl can be optionally substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Heterocyclyl” or “heterocycle” refers to a monocyclic or polycyclic hydrocarbon substituent that is saturated or partially unsaturated, wherein the partially unsaturated cyclic hydrocarbon refers to a cyclic hydrocarbon that may contain one or more (preferably 1, 2 or 3) double bonds, but none of the rings have a fully conjugated π-electron system, wherein one or more (preferably 1, 2, 3 or 4) of the rings atoms in heterocyclyl are heteroatoms selected from N, O, N·O or S(O) (wherein r is an integer of 0, 1 or 2), excluding ring portions of —O—O—, —O—S—or —S—S—, and the remaining ring atoms are carton atoms. It preferably includes heterocyclyl containing 3 to 12 or 3 to 8 or 3 to 6 ring atoms. For example, “3-6 membered heterocyclyl” means a heterocyclyl containing 3 to 6 ring atoms, “3-8 membered heterocyclyl” means a heterocyclyl containing 3 to 8 ring atoms, “4-6 membered heterocyclyl” means a heterocyclyl containing 4 to 6 ring atoms, “4-8 membered heterocyclyl” means a heterocyclyl containing 4 to 8 ring atoms, “4-10 membered heterocyclyl” means a heterocyclyl containing 4 to 10 ring atoms, “4-12 membered heterocyclyl” means a heterocyclyl containing 4 to 12 ring atoms, “5-8 membered heterocyclyl” means a heterocyclyl containing 5 to 8 ring atoms, and “3-12 membered heterocyclyl” means a heterocyclyl containing 3 to 12 ring atoms.

Monocyclic heterocyclyl includes but is not limited to pyrrolidinyl, piperidinyl, piperazinyl, morpholinyl, thiomorpholinyl, homopiperazinyl, oxacyclobutyl, tetrahydrofuranyl, etc.

Polycyclic heterocyclyl includes spiroheterocyclyl, fused heterocyclyl and bridged heterocyclyl. “Spiroheterocyclyl” refers to a polycyclic heterocyclyl group in which an atom (called spiro-atom) is shared among monocyclic rings, wherein one or more (preferably 1, 2, 3 or 4) of the ring atoms are heteroatoms selected from N, O, N·O or S(O)ᵣ (wherein r is an integer of 0, 1 or 2), and the remaining ring atoms are carbon atoms. These rings may contain one or more (preferably 1, 2 or 3) double bonds, but none of them have a fully conjugated π-electronic system. According to the number of spiro-atoms shared among the rings, spiroheterocyclyl may be monospiroheterocyclyl, bispiroheterocyclyl or polyspiroheterocyclyl. Spiroheterocyclyl includes but is not limited to:

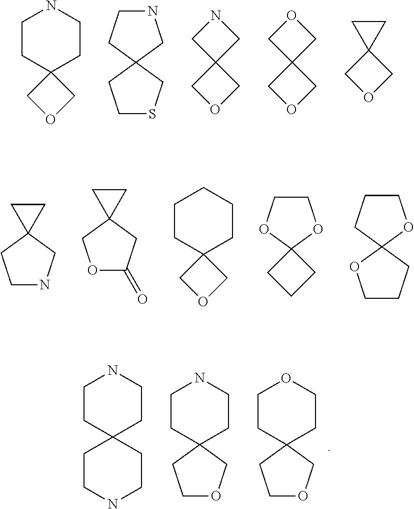

“Fused heterocyclyl” refers to a polycyclic heterocyclyl in which each ring shares a pair of adjacent atoms with the other rings in the system, where one or more (preferably 1, 2, 3 or 4) of the rings may contain one or more (preferably 1, 2 or 3) double bonds, but none of them have a fully conjugated π-electron system, where one or more (preferably 1, 2, 3 or 4) of the ring atoms are heteroatoms selected from N, O, N·O or S(O)ᵣ (where r is an integer of 0, 1 or 2), and the remaining ring atoms are carbon atoms. According to the number of formed rings, the fused heterocyclyl may be bicyclic, tricyclic tetracyclic or polycyclic including but not limited to:

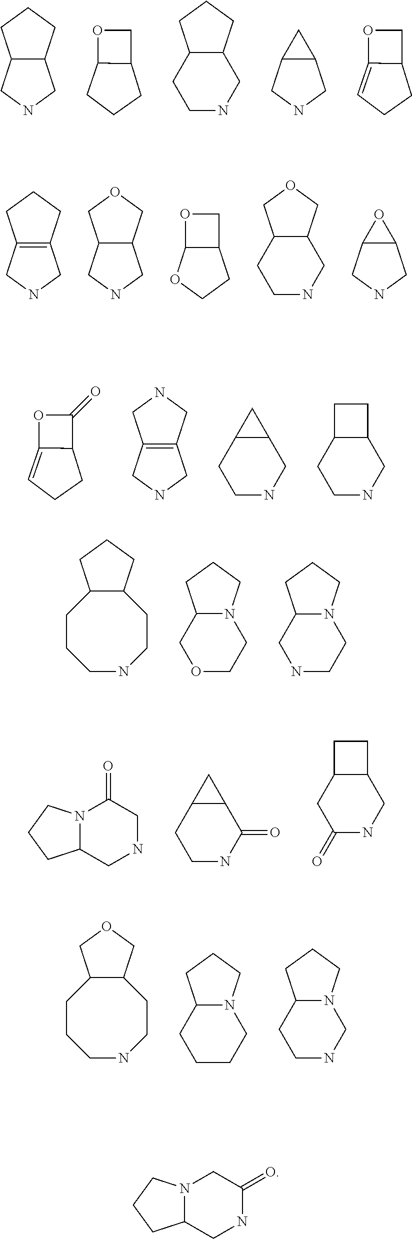

“Bridged heterocyclyl” refers to a polycyclic heterocyclyl in which any two rings share two carton atoms that are not directly attached to each other, where these rings may contain one or more (preferably 1, 2 or 3) double bonds, but none of them have a fully conjugated π-electron system, where one or more (preferably 1, 2, 3 or 4) of the ring atoms are heteroatoms selected from N, O, N·O or S(O) (where r is an integer of 0, 1 or 2), and the remaining ring atoms are carbon atoms. According to the number of formed rings, the bridged heterocyclyl may be bicyclic, tricyclic, tetracyclic or polycyclic, including but not limited to:

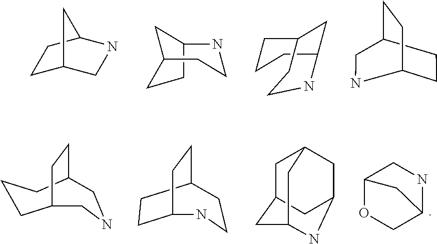

The heterocyclyl ring may be fused to an aryl, heteroaryl or cycloalkyl ring, wherein the ring attached to the parent structure is heterocyclyl, which includes but is not limited to:

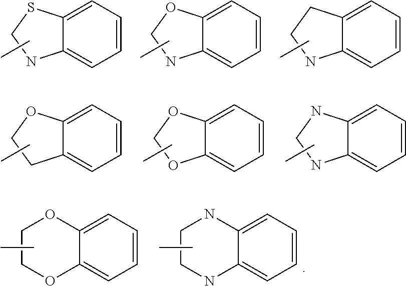

Heterocyclyl can be optionally substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl. C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Aryl” or “aromatic ring” means an all-carbon monocyclic or fused-polycyclic group (i.e., rings that share a pair of adjacent carbon atoms) and a polycyclic group having a conjugated π-electron system (i.e., rings with adjacent pairs of carbon atoms), preferably including all-carbon aryl containing 6-10 or 6-8 carbon atoms. For example, “C₆₋₁₀ aryl” means an all-carbon aryl containing 6-10 carbon atoms, including but not limited to phenyl and naphthyl, and “C₆₋₈ aryl” means an all-carbon aryl containing 6-8 carbon atoms. The aryl ring may be fused to an heteroaryl, heterocyclyl or cycloalkyl ring, wherein the ring attached to the parent structure is aryl ring, including but not limited to:

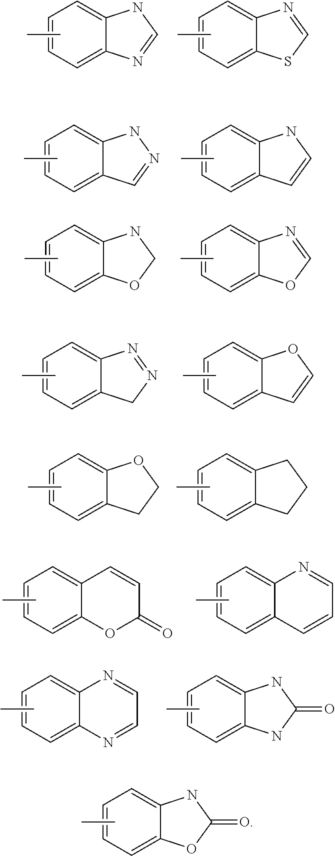

“Aryl” can be substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Heteroaryl” refers to a heteroaromatic system containing one or more (preferably 1, 2, 3 or 4) of the heteroatoms, where the heteroatoms include heteroatoms selected from N, O, N·O and S(O)ᵣ (where r is an integer of 0, 1 or 2), preferably including a heteroaromatic system containing 5-10 or 5-8 or 5-6 ring atoms. For example, “5-8 membered heteroaryl” means a heteroaromatic system containing 5-8 ring atoms, and “5-10 membered heteroaryl” means a heteroaromatic system containing 5-10 ring atoms, including but not limited to furyl, thiophenyl, pyridyl, pyrrolyl. N-alkylpyrrolyl, pyrimidinyl, pyrazinyl, imidazolyl, tetrazolyl, etc. The heteroaryl ring may be fused to an aryl, heterocyclyl or cycloalkyl ring, wherein the ring attached to the parent structure is heteroaryl ring, including but not limited to:

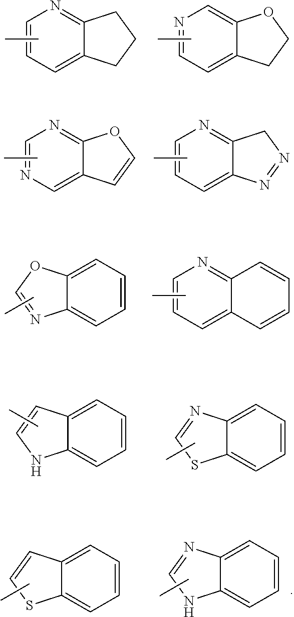

“Heteroaryl” can be optionally substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₃)—C(═NR₁₄)R₁₂, —C₀₋₈ alkyl-C(O)NR₁₃R₁₄ and —C₀₋₈ alkyl-N(R₁₃)—C(O)R₁₂.

“Alkenyl” refers to an alkyl defined as above consisting of at least two carbon atoms and at least one carbon-carbon double bond, preferably including a linear or branched alkenyl containing 2-10 or 2-4 carbon atoms. For example, “C₂₋₁₀ alkenyl” means a linear or branched alkenyl containing 2-10 carbon atoms, and “C₂₋₄ alkenyl” means a linear or branched alkenyl containing 2-4 carbon atoms. The alkenyl includes, but is not limited to, vinyl, 1-propenyl, 2-propenyl, 1-, 2- or 3-butenyl, etc.

“Alkenyl” can be substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₂, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Alkynyl” refers to an alkyl defined as above consisting of at least two carbon atoms and at least one carbon-carbon triple bond, preferably including a linear or branched alkynyl containing 2-10 or 2-4 carbon atoms. For example, “C₂₋₁₀ alkynyl” means a linear or branched alkynyl containing 2-10 carbon atoms, and “C₂₋₄ alkynyl” means a linear or branched alkynyl containing 2-4 carbon atoms. The alkynyl includes, but is not limited to, ethynyl, 1-propynyl, 2-propynyl, 1-, 2- or 3-butynyl, etc.

“Alkynyl” can be substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Alkoxy” refers to an —O-alkyl group, wherein the alkyl is defined as above. For example, “C₁₋₁₀ alkoxy” means an alkyloxy containing 1-10 carbon atoms, “C₁₋₄ alkoxy” means an alkyloxy containing 1-4 carbon atoms, and “C₁₋₂ alkoxy” means an alkyloxy containing 1-2 carbon atoms, including but not limited to, methoxy, ethoxy, propoxy, butoxy, etc.

“Alkoxy” can be optionally substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Cycloalkoxy” or “cycloalkyloxy” refers to an —O-cycloalkyl group, wherein cycloalkyl is defined as above. For example, “C₃₋₁₂ cycloalkoxy” means a cycloalkyloxy containing 3-12 carbon atoms, and “C₃₋₆ cycloalkoxy” means a cycloalkyloxy containing 3-6 carbon atoms, including but not limited to cyclopropoxy, cyclobutoxy, cyclopentoxy, cyclohexoxy, etc.

“Cycloalkoxy” or “cycloalkyloxy” can be optionally substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“Heterocycloxy” or “heterocyclyloxy” refers to an —O-heterocyclyl group, wherein the heterocyclyl is defined as above, including but not limited to, azetidyloxy, oxetanyloxy, azetidyloxy, nitrogen, oxetanyloxy, etc.

“Heterocycloxy” or “heterocyclyloxy” can be optionally substituted or unsubstituted, and when it is substituted, the substituent is preferably one or more (preferably 1, 2, 3 or 4) of the groups independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄ and —C₀₋₈ alkyl-N(R₁₃)—C(O)R₁₂.

“C₁₋₁₀ alkanoyl” refers to a monovalent atomic group which is obtained after a hydroxy is removed from the C₁₋₁₀ alkyl acid, and is also generally referred to as “C₀₋₉ alkyl-C(O)—”. For example, “C₁ alkyl-C(O)—” refers to acetyl; “C₂ alkyl-C(O)—” refers to propanoyl; “C₃ alkyl-C(O)—” refers to butanoyl or isobutanoyl.
“—C₀₋₈ alkyl-S(O)(═N—R₇)R₈” refers to the sulfur atom in —S(O)(═N—R₇)R₈ attached to C₀₋₈ alkyl, where the C₀₋₈ alkyl is defined as above.
“—C₀₋₈ alkyl-N═S(O)R₈R₉” refers to the nitrogen atom in —N═S(O)R₈R₉ attached to C₀₋₈ alkyl, where the C₀₋₈ alkyl is defined as above.
“—C₀₋₈ alkyl-N═SR₈R₉” refers to the nitrogen atom in —N═SR₈R₉ attached to C₀₋₈ alkyl, where the C₀₋₈ alkyl is defined as above.
“—C₀₋₈ alkyl-N(R₁₃)—S(O)₂R₁₀” refers to the nitrogen atom in —N(Ru)—S(O)₂R₁₀ attached to C₀₋₈ alkyl, where the C₀₋₈ alkyl is defined as above.
“—C₀₋₈ alkyl-O—S(O)₂R₁₀” refers to the oxygen atom in —O—S(O)₂R₁₀ attached to C₀₋₈ alkyl, where the C₀₋₈ alkyl is defined as above.
“—C₀₋₈ alkyl-S(O)ᵣR₁₀” refers to the sulfur atom in —S(O)ᵣR₁₀ attached to C₀₋₈ alkyl, where the C₀₋₈ alkyl is defined as above.
“—C₀₋₈ alkyl-P(O)(R₁₂)₂” refers to the phosphorus atom in —P(O)(R₁₂)₂ attached to C₀₋₈ alkyl, where the C₀₋₈ alkyl is defined as above.
“—C₀₋₈ alkyl-O—R₁₁” refers to the oxygen atom in —O—R₁₁ attached to C₀₋₈ alkyl, where the C₀₋₈ alkyl is defined as above.
“—C₀₋₈ alkyl-C(O)OR₁₁” refers to the carbonyl in —C(O)OR₁₁ attached to C₀₋₈ alkyl, where the C₀₋₈ alkyl is defined as above.
“—C₀₋₈ alkyl-C(O)SR₁₁” refers to the carbonyl in —C(O)SR₁₂ attached to C₀₋₈ alkyl, where the C₀₋₈ alkyl is defined as above.
“—C₀₋₈ alkyl-S—C(O)R₁₂” refers to the sulfur atom in —S—C(O)R₁₂ attached to C₀₋₈ alkyl, where the C₀₋₈ alkyl is defined as above.
“—C₀₋₈ alkyl-C(O)R₁₂” refers to the carbonyl in —C(O)R₁₂ attached to C₀₋₈ alkyl, where the C₀₋₈ alkyl is defined as above.
“—C₀₋₈ alkyl-O—C(O)R₁₂” refers to the oxygen atom in —O—C(O)R₁₂ attached to C₀₋₈ alkyl, where the C₀₋₈ alkyl is defined as above.
“—C₀₋₈ alkyl-NR₁₃R₁₄” refers to the nitrogen atom in —NR₁₃R₁₄ attached to C₀₋₈ alkyl, where the C₀₋₈ alkyl is defined as above.
“—C₀₋₈ alkyl-C(═NR₁₃)R₁₂” refers to the carbon atom in —C(═NR₁₃)R₁₂ attached to C₀₋₈ alkyl, where the C₀₋₈ alkyl is defined as above.
“—C₀₋₈ alkyl-N(R₁₃)—C(═NR₁₄)R₁₂” refers to the nitrogen atom in —N(R₁₃)—C(═NR₁₄)R₁₂ attached to C₀₋₈ alkyl, where the C₀₋₈ alkyl is defined as above.
“—C₀₋₈ alkyl-C(O)NR₁₃R₁₄” refers to the carbonyl in —C(O)NR₁₃R₁₄ attached to C₀₋₈ alkyl, where the C₀₋₈ alkyl is defined as above.
“—C₀₋₈ alkyl-N(Ru)—C(O)R₁₂” refers to the nitrogen atom in —N(R₁₃)—C(O)R₁₂ attached to C₀₋₈ alkyl, where the C₀₋₈ alkyl is defined as above.
“C₁₋₁₀ haloalkyl” refers to an alkyl having 1-10 carbon atoms in which hydrogens on the alkyl are optionally substituted by a fluorine, chlorine, bromine and iodine atom, including but not limited to difluoromethyl, dichloromethyl, dibromomethyl, trifluoromethyl, trichloromethyl, tribromomethyl, etc.
“C₁₋₁₀ haloalkoxy” refers to an alkoxy having 1-10 carbon atoms in which hydrogens on the alkyl are optionally substituted by a fluorine, chlorine, bromine and iodine atom, including but not limited to difluoromethoxy, dichloromethoxy, dibromomethoxy, trifluoromethoxy, trichloromethoxy, tribromomethoxy, etc.
“C₁₋₁₀ deuteroalkyl” refers to an alkyl having 1-10 carbon atoms in which hydrogens on the alkyl are optionally substituted by a deuterium atom, including but not limited to monodeuterium, dideuterium, trideuterium, etc.
“Halogen” refers to fluorine, chlorine, bromine or iodine.

The term “optional” or “optionally” means that the event or circumstance subsequently described may, but not necessarily, occur, and that the description includes instances where the event or circumstance occurs or dose not occur, i.e., both substituted or unsubstituted instances. For example. “heterocyclyl group optionally substituted by alkyl” means that alkyl may be, but not necessarily, present, and that the description includes instances where the heterocyclyl group is and is not substituted by alkyl.

The term “substituted” means that one or more “hydrogen atoms” in a group are each independently substituted by a corresponding number of substituents. It goes without saying that a substituent is only in its possible chemical position and consistent with the valence bond theory in chemistry, and those skilled in the art will be able to determine (by experiments or theories) possible or impossible substitution without undue efforts. For example, it may be unstable when an amino or hydroxy having a free hydrogen is bound to a carbon atom having an unsaturated bond (such as olefin).

“Stereoisomer” refers to isomers produced by different spatial arrangements of atoms in the molecules. They can be divided into cis-trans isomers and enantiomers and can also be divided into two categories: enantiomers and diastereomers. A stereoisomer produced by the rotation of a single bond is referred to as a conformational stereo-isomer, sometimes also referred to as a rotamer. A stereoisomer produced by bond length, bond angle, double bonds in the molecule, ring and other reasons is referred to as a configuration stereo-isomer, which can be divided into two categories.

An isomer produced by the inability of a double bond or a single bond of ring carbon atoms to rotate freely becomes a geometric isomer, also known as a cis-trans isomer, which can be divided into Z and E configurations. For example: cis-2-butene and trans-2-butene are a pair of geometric isomers. Stereoisomers with different optical properties produced by the absence of anti-axial symmetry in the molecules are called optical isomers, which can be divided into R and S configurations. “Stereoisomers” as described in the present invention, if not specifically indicated, are to be understood to include one or more of the enantiomers, configurational isomers, and conformational isomers described above.

“Pharmacologically acceptable salt” in the present invention refers to pharmaceutically acceptable acid addition salts, including inorganic and organic acid salts, which may be prepared by methods known in the art.

“Pharmaceutical composition” refers to a mixture containing one or more of the compounds described herein or a physiologically/pharmaceutically acceptable salt or pro-drug thereof, and other chemical components, for example physiologically/pharmaceutically acceptable carriers and excipients. The purpose of the pharmaceutical composition is to promote the administration to an organism, which facilitates the absorption of the active ingredient, thereby exerting biological activities.

The present invention is further explained in detail below with reference to examples, which are not intended to limit the present invention, and the present invention is not merely limited to the contents of the examples.

The compound structure of the present invention is determined by nuclear magnetic resonance (NMR) or/and liquid chromatography-mass spectrometry (LC-MS). The NMR chemical shift (δ) is given in parts per million (ppm). The NMR determination is conducted by using a Bruker AVANCE-400/500 nuclear magnetic resonance apparatus, with hexadeuterodimethyl sulfoxide (DMSO-d₆), tetradeuteromethanol (CD₃OD) and deuterated chloroform (CDCl₃) as determination solvents, and tetramethylsilane (TMS) as internal standard.

The LC-MS determination is conducted by using an Agilent 6120 mass spectrometer. The HPLC determination is conducted by using an Agilent 1200 DAD high pressure liquid chromatography (Sunfire C18 150×4.6 mm chromatographic column) and a Waters 2695-2996 high pressure liquid chromatography (Gimini C18 150×4.6 mm chromatographic column).

Yantai Yellow Sea HSGF254 or Qingdao GF254 silica gel plate is adopted as a thin layer chromatography (TLC) silica gel plate. The specification adopted by the TLC is 0.15-0.20 mm, and the specification adopted by the thin layer chromatography for the separation and purification of products is 0.4-0.5 mm. The Yantai Yellow Sea silica gel of 200-300 mesh is generally utilized as a carrier in column chromatography.

Starting materials in the examples of the present invention are known and commercially available, or may be synthesized by using or according to methods known in the art.

Unless otherwise stated, all reactions of the present invention are carried out under a dry nitrogen or argon atmosphere with continuous magnetic stirring, wherein the solvent is a dry solvent and the reaction temperature is in degree centigrade (° C.).

### I. PREPARATION OF INTERMEDIATES

### Intermediate 1-1: Preparation of (S)-3-bromo-1-(4-((tert-butyldimethylsilyl)oxy)butan-2-yl)-6-chloro-1H-pyrazolo[4,3-c]pyridine

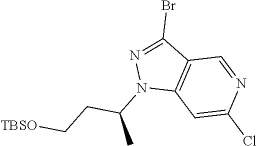 [SMILES: C[C@@H](CCO[Si](C)(C)C(C)(C)C)n1nc(Br)c2cnc(Cl)cc21]

### Step 1: Synthesis of 3-bromo-6-chloro-1H-pyrazolo[4,3-c]pyridine

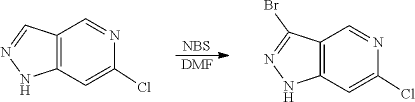

6-chloro-1H-pyrazolo[4,3-c]pyridine (5.0 g, 32.56 mmol) was dissolved in DMF (30 mL), and NBS (6.95 g, 39.07 mmol) was added. The mixture was stirred at room temperature for 2 hours. CH₂Cl₂ and saturated aqueous NaHCO₃ solution were added for extraction, the organic phase was washed with saturated aqueous NaCl solution, dried over anhydrous Na₂SO₄, filtered and concentrated, and the residue was separated by using silica gel column chromatography to obtain 3-bromo-6-chloro-1H-pyrazolo[4,3-c]pyridine (7.4 g, yield: 97.8%).

### Step 2: Synthesis of (R)-4-((tert-butyldimethylsilyl)oxy)butan-2-ol

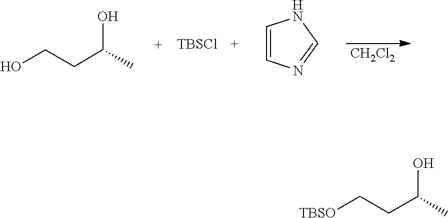

(R)-butan-1,3-diol (4.98 mL, 55.48 mmol) was dissolved in CH₂Cl₂ (80 mL), and imidazole (4.16 g, 61.03 mmol) was added. The mixture was cooled to 0° C., and then TBSCl (8.36 g, 55.48 mmol) was added. The mixture was stirred at 10° C. for 3 hours. The reaction solution was diluted with CH₂Cl₂ and water, and the organic phase was separated by extraction, washed with saturated aqueous NaCl solution, dried over anhydrous Na₂SO₄, filtered and concentrated. The residue was separated by using silica gel column chromatography to obtain (R)-4-((tert-butyldimethylsilyl)oxy)butan-2-ol (9.0 g, yield: 79.4%).

### Step 3: Synthesis of (R)-4-((tert-butyldimethylsilyl)oxy)butan-2-yl 4-methylbenzene-1-sulfonate

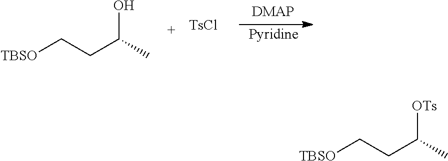 [SMILES: C[C@@H](O)CCO[Si](C)(C)C(C)(C)C ; "TsCl" ; "Pyridine" ; Cc1ccc(S(=O)(=O)O[C@H](C)CCO[Si](C)(C)C(C)(C)C)cc1]

(R)-4-((tert-butyldimethylsilyl)oxy)butan-2-ol (6.0 g, 29.36 mmol) was dissolved in pyridine (60 mL), and then DMAP (1.08 g, 8.81 mmol) was added, and TsCl (12.31 g, 64.59 mmol) was added under cooling to 0° C. After the addition, the mixture was stirred at room temperature for 18 hours. The reaction solution was diluted with CH₂Cl₂ and water, and the organic phase was separated by extraction, washed with saturated aqueous NaCl solution, dried over anhydrous Na₂SO₄, filtered and concentrated. The residue was separated by using silica gel column chromatography to obtain (R)-4-((tert-butyldimethylsilyl)oxy)butan-2-yl 4-methylbenzene-1-sulfonate (6.4 g, yield: 60.8%).

### Step 4: Synthesis of (S)-3-bromo-1-(4-((tert-butyldimethylsilyl)oxy)butan-2-yl)-6-chloro-11H-pyrazolo[4,3-c]pyridine

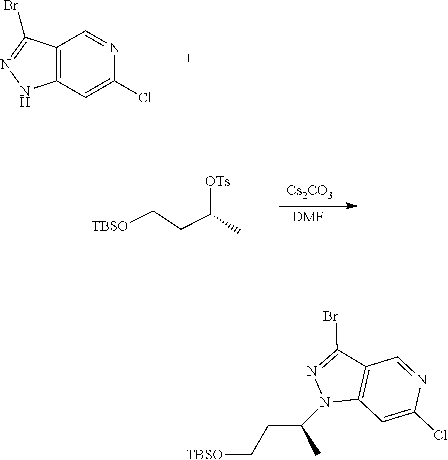

3-bromo-6-chloro-1H-pyrazolo[4,3-c]pyridine (1.0 g, 4.30 μmol) was dissolved in DMF (20 mL), and then (R)-4-((tert-butyldimethylsilyl)oxy)butan-2-yl 4-methylbenzene-1-sulfonate (1.85 g, 5.16 mmol) and Cs₂CO₃ (2.80 g, 8.60 mmol) were added in sequence. The mixture was heated to 45° C., and stirred for 18 hours. The mixture was cooled to room temperature, and EtOAc and water were added for extraction. The organic phase was washed with saturated aqueous NaCl solution, dried over anhydrous Na₂SO₄, filtered and concentrated. The residue was separated by using silica gel column chromatography to obtain (S)-3-bromo-1-(4-((tert-butyldimethylsilyl)oxy)butan-2-yl)-6-chloro-1H-pyrazolo[4,3-c]pyridine (1.5 g, yield: 83.3%). MS m/z (ESI): 418.2/420.2 [M+H]⁺.

Intermediates 1-2 to 1-6 can be prepared by selecting the corresponding raw materials with reference to the synthesis methods of intermediate 1-1.

| Intermediate | | | MS: m/z |
|---|---|---|---|
| No. | Structural Formula | Chemical Name | [M + H]⁺ |
| 1-2 | 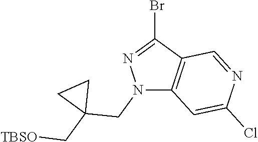 [SMILES: CC(C)(C)[Si](C)(C)OCC1(Cn2nc(Br)c3cnc(Cl)cc32)CC1] | 3-bromo-1-((1-(((tert- butyldimethylsilyl)oxy) methyl)cyclopropyl)methyl)- 6-chloro-1H-pyrazolo[4,3- c]pyridine | 431 |
| 1-3 | 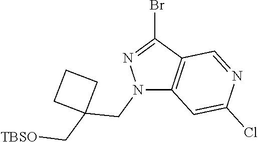 [SMILES: CC(C)(C)[Si](C)(C)OCC1(Cn2nc(Br)c3cnc(Cl)cc32)CCC1] | 3-bromo-1-((1-(((tert- butyldimethylsilyl)oxy) methyl)cyclobutyl)methyl)-6- chloro-1H-pyrazolo[4,3-c] pyridine | 445 |
| 1-4 | 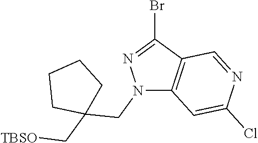 [SMILES: CC(C)(C)[Si](C)(C)OCC1(Cn2nc(Br)c3cnc(Cl)cc32)CCCC1] | 3-bromo-1-((1-(((tert- butyldimethylsilyl)oxy) methyl)cyclopentyl)methyl)-6- chloro-1H-pyrazolo[4,3-c] pyridine | 459 |
| 1-5 | 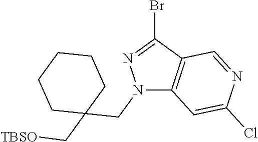 [SMILES: CC(C)(C)[Si](C)(C)OCC1(Cn2nc(Br)c3cnc(Cl)cc32)CCCCC1] | 3-bromo-1-((1-(((tert- butyldimethylsilyl)oxy) methyl)cyclohexyl)methyl)-6- chloro-1H-pyrazolo[4,3-c] pyridine | 474 |
| 1-6 | 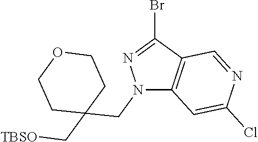 [SMILES: CC(C)(C)[Si](C)(C)OCC1(Cn2nc(Br)c3cnc(Cl)cc32)CCOCC1] | 3-bromo-1-((4-(((tert- butyldimethylsilyl)oxy) methyl)tetrahydro-2H-pyran- 4-yl)methyl)-6-chloro-1H- pyrazolo[4,3-c]pyridine | 476 |

### Intermediate 1-7: Preparation of (1-((3-bromo-6-chloro-1H-pyrazolo[4,3-c]pyridin-1-yl)methyl)cyclopentyl)methanol

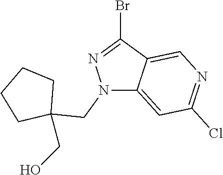 [SMILES: OCC1(Cn2nc(Br)c3cnc(Cl)cc32)CCCC1]

### Step 1: Synthesis of methyl 1-(iodomethyl)cyclopentane-1-carboxylate

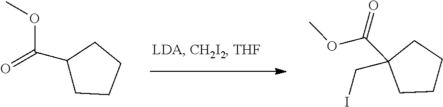

Methyl cyclopentanecarboxylate (11.7 g, 91.285 mmol) was dissolved in THF (120 mL). Under nitrogen protection, LDA/THF solution (54.8 mL, 109.542 mmol, 2 M) was slowly added dropwise at −78° C., and then CH₂I₂ (8.837 mL, 109.542 mmol) was added.

After the addition, the mixture was warmed to room temperature and reacted for 1.5 hours. The reaction was completed as detected by TLC. The solution was quenched by adding saturated aqueous NH₄Cl solution (150 mL). The solution was extracted three times with EtOAc (100 mL), and the organic phase was combined and dried over anhydrous Na₂SO₄. It was filtered and concentrated, and the residue was separated by using silica gel column chromatography [eluent. PE/EtOAc 0-15%] to obtain methyl 1-(iodomethyl)cyclopentane-1-carboxylate (23.5 g, 78.891 mmol, yield: 86.4%).

¹H NMR (400 MHz, CDCl₃) δ 3.72 (d, J=0.9 Hz, 3H), 3.41 (d, J=0.9 Hz, 2H), 2.26-2.16 (m, 2H), 1.79-1.61 (m, 6H).

### Step 2: Synthesis of methyl 1-((3-bromo-6-chloro-1H-pyrazolo[4,3-c]pyridin-1-yl)methyl)cyclopentane-1-carboxylate

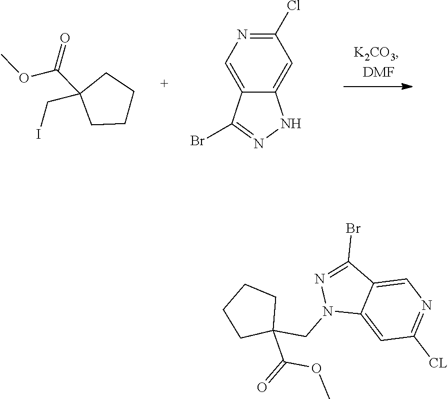

3-bromo-6-chloro-1H-pyrazolo[4,3-c]pyridine (37.46 g, 161.140 mmol) and methyl 1-(iodomethyl)cyclopentane-1-carboxylate (43.2 g, 161.14 mmol) were dissolved in DMF (800 mL), and K₂CO₃ (44.54 g, 322.28 mmol) was added, and then the mixture was heated to 100° C., and stirred for 16 hours. The reaction was completed as detected by TLC. The reaction solution was cooled to room temperature and concentrated, and the residue was separated by using silica gel column chromatography [eluent: PE/EA 5/1] to obtain methyl 1-((3-bromo-6-chloro-1H-pyrazolo[4,3-c]pyridin-1-yl)methyl)cyclopentane-1-carboxylate (38.5 g, 103.31 mmol, yield: 64.1%). MS m/z (ESI): 372.0, 374.0 [M+H]⁺.

### Step 3: Synthesis of (1-((3-bromo-6-chloro-1H-pyrazolo[4,3-c]pyridin-1-yl)methyl)cyclopentyl)methanol

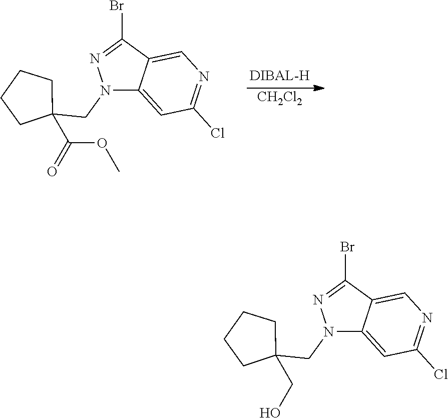

Methyl 1-((3-bromo-6-chloro-1H-pyrazolo[4,3-c]pyridin-1-yl)methyl)cyclopentane-1-carboxylate (8.5 g, 22.81 mmol) was dissolved in CH₂Cl₂ (100 mL), the mixture was cooled to −78° C., DIBAL-H (45.62 mL, 45.62 mmol) was slowly added at the low temperature, and the mixture was stirred at −78° C. for 2 hours. The reaction was completed as detected by TLC. Saturated brine was added to quench the reaction mixture, and then aqueous potassium sodium tartrate solution (50 mL) was added, and the mixture was stirred at room temperature for 10 minutes. Then, the mixture was extracted three times with CH₂Cl₂ (50 mL). The organic phase was combined, dried over anhydrous sodium sulfate, filtered and concentrated, and the resulting residue was separated by using silica gel column chromatography [eluent: PE/EA=2/1] to obtained (1-((3-bromo-6-chloro-1H-pyrazolo[4,3-c]pyridin-1-yl)methyl)cyclopentyl)methanol (5.8 g, 16.83 mmol, yield: 73.78%). MS m/z (ESI): 344.0, 346.0 [M+H]⁺.

Intermediates 1-8 to 1-10 can be prepared by selecting the corresponding raw materials with reference to the synthesis routes of intermediate 1-7.

| Intermediate | | | MS: m/z |
|---|---|---|---|
| No. | Structural Formula | Chemical Name | [M + H]⁺ |
| 1-8 | 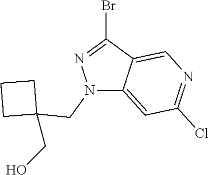 [SMILES: OCC1(Cn2nc(Br)c3cnc(Cl)cc32)CCC1] | (1-((3-bromo-6-chloro-1H- pyrazolo[4,3-c]pyridin- 1-yl)methyl)cyclobutyl) methanol | 330.0 |
| 1-9 | 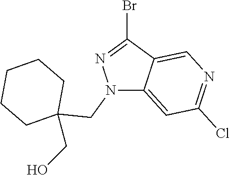 [SMILES: OCC1(Cn2nc(Br)c3cnc(Cl)cc32)CCCCC1] | (1-((3-bromo-6-chloro-1H- pyrazolo[4,3-c]pyridin- 1-yl)methyl)cyclohexyl) methanol | 358.0 |
| 1-10 | 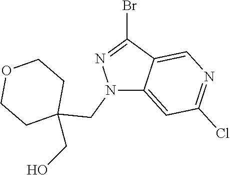 [SMILES: OCC1(Cn2nc(Br)c3cnc(Cl)cc32)CCOCC1] | (4-((3-bromo-6-chloro-1H- pyrazolo[4,3-c]pyridin- 1-yl)methyl)tetrahydro- 2H-pyran-4-yl)methanol | 360.0 |

### Intermediate 2-1: Preparation of (S)-1-(4-((tert-butyldimethylsilyl)oxy)butan-2-yl)-6-chloro-3-(prop-1-yn-1-yl)-1H-pyrazolo[4,3-c]pyridine

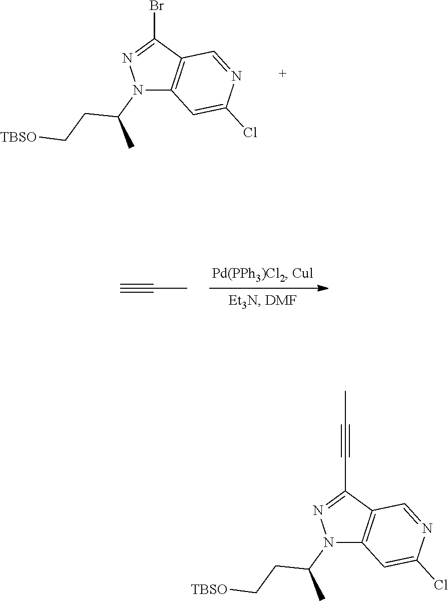

(S)-3-bromo-1-(4-((tert-butyldimethylsilyl)oxy)butan-2-yl)-6-chloro-1H-pyrazolo[4,3-c]pyridine (0.90 g, 2.14 mmol) was dissolved in DMF (10 mL), and Pd(PPh₃)₂Cl₂ (0.31 g, 0.43 mmol), CuI (164 mg, 0.86 mmol) and Et₃N (3 mL) were added. Under nitrogen protection, propyne solution (10 mL g, 10 mmol) was added with a syringe, and the mixture was heated to 70° C., and reacted overnight. After the reaction was completed, EtOAc was added to dilute the mixture, and the organic phase was washed with water and saturated brine in sequence, and dried over anhydrous sodium sulfate. It was filtered, and the filtrate was concentrated and then separated by using column chromatography to obtain (S)-1-(4-((tert-butyldimethylsilyl)oxy)butan-2-yl)-6-chloro-3-(prop-1-yn-1-yl)-1H-pyrazolo[4,3-c]pyridine (0.77 g, yield: 80%). MS m/z (ESI): 378.2 [M+H]⁺.

### Intermediate 2-2: Preparation of 1-((S)-4-((tert-butyldimethylsilyl)oxy)butan-2-yl)-6-chloro-3-(((S)-1-methylpyrrolidin-2-yl)ethynyl)-1H-pyrazolo[4,3-c]pyridine

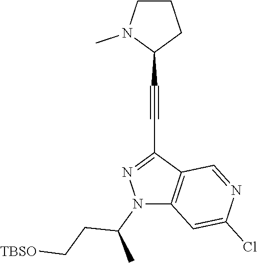 [SMILES: C[C@@H](CCO[Si](C)(C)C(C)(C)C)n1nc(C#C[C@@H]2CCCN2C)c2cnc(Cl)cc21]

### Step 1: Synthesis of tert-butyl (S)-2-ethynylpyrrolidin-1-carboxylate

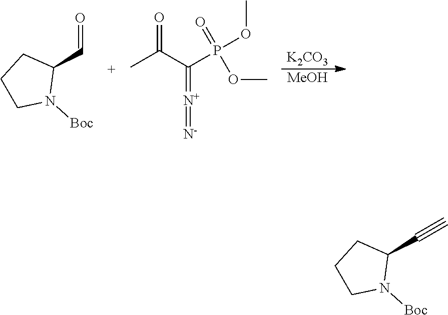

Tert-butyl (S)-2-formylpyrrolidine-1-carboxylate (5.0 g, 25.09 mmol) was dissolved in MeOH (50 mL), then K₂CO₃ (6.94 g, 50.19 mmol) was added, the mixture was cooled to 0° C., and dimethyl(1-diazo-2-oxopropyl)phosphate (5.65 mL, 37.64 mmol) was added dropwise. The mixture was reacted at room temperature for 18 hours. After the reaction was completed, the mixture was filtered, and the filtrate was concentrated and then separated by using column chromatography to obtain tert-butyl (S)-2-ethynylpyrrolidin-1-carboxylate (4.4 g, yield: 89.8%). MS m/z (ESI): 195.8 [M+H]⁺.

### Step 2: Synthesis of tert-butyl (S)-2-((1-((S)-4-((tert-butyldimethylsilyl)oxy)butan-2-yl)-6-chloro-1H-pyrazolo[4,3-c]pyridin-3-yl)ethynyl)pyrrolidin-1-carboxylate

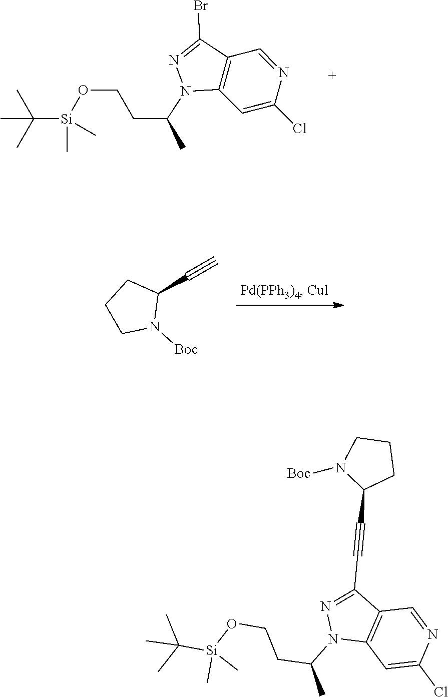 [SMILES: C[C@@H](CCO[Si](C)(C)C(C)(C)C)n1nc(Br)c2cnc(Cl)cc21 ; C#C[C@@H]1CCCN1C(=O)OC(C)(C)C ; C[C@@H](CCO[Si](C)(C)C(C)(C)C)n1nc(C#C[C@@H]2CCCN2C(=O)OC(C)(C)C)c2cnc(Cl)cc21]

(S)-3-bromo-1-(4-((tert-butyldimethylsilyl)oxy)butan-2-yl)-6-chloro-1H-pyrazolo[4,3-c]pyridine (0.6 g, 1.43 mmol) was dissolved in DMF (20 mL), and tert-butyl (S)-2-ethynylpyrrolidin-1-carboxylate (1119 mg, 5.73 mmol), CuI (0.27 g, 1.43 mmol), Et₃N (1.0 mL, 7.16 mmol) and Pd(PPh₃)₄ were added (0.50 g, 0.43 mmol). The mixture was heated to 80° C., and reacted for 2 hours under nitrogen protection. The mixture was cooled to room temperature, filtered, and the filtrate was poured into water, extracted with EtOAc, and the organic phase was washed with saturated brine and dried over anhydrous sodium sulfate. It was filtered, and the filtrate was concentrated and then separated by using column chromatography to obtain tert-butyl (S)-2-((1-((S)-4-((tert-butyldimethylsilyl)oxy)butan-2-yl)-6-chloro-1H-pyrazolo[4,3-c]pyridin-3-yl)ethynyl)pyrrolidin-1-carboxylate (720 mg, yield: 94.3%). MS m/z (ESI): 533.4 [M+H]⁺.

### Step 3: Synthesis of 1-((S)-4-((tert-butyldimethylsilyl)oxy)butan-2-yl)-6-chloro-3-(((S)-pyrrolidin-2-yl)ethynyl)-1H-pyrazolo[4,3-c]pyridine

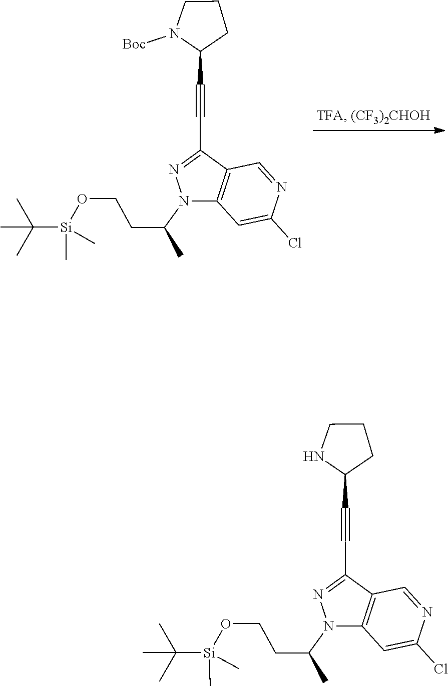 [SMILES: C[C@@H](CCO[Si](C)(C)C(C)(C)C)n1nc(C#C[C@@H]2CCCN2C(=O)OC(C)(C)C)c2cnc(Cl)cc21 ; C[C@@H](CCO[Si](C)(C)C(C)(C)C)n1nc(C#C[C@@H]2CCCN2)c2cnc(Cl)cc21]

Tert-butyl (S)-2-((1-((S)-4-((tert-butyldimethylsilyl)oxy)butan-2-yl)-6-chloro-1H-pyrazolo[4,3-c]pyridin-3-yl)ethynyl)pyrrolidin-1-carboxylate (720 mg, 1.35 mmol) was dissolved in hexafluoroisopropanol (20 mL), and then TFA (1.0 mL, 13.50 mmol) was added dropwise. The mixture was reacted at room temperature for 1 hour. After the reaction was completed, CH₂Cl₂ was added for dilution, and the mixture was washed with saturated aqueous NaHCO₃ solution, pure water, and saturated brine in sequence, and dried over anhydrous sodium sulfate. It was filtered, and the filtrate was concentrated to obtain a crude product, which was directly used for the next reaction. MS m/z (ESI): 433.2 [M+H]⁺.

### Step 4: Synthesis of 1-((S)-4-((tert-butyldimethylsilyl)oxy)butan-2-yl)-6-chloro-3-(((S)-1-methylpyrrolidin-2-yl)ethynyl)-1H-pyrazolo[4,3-c]pyridine

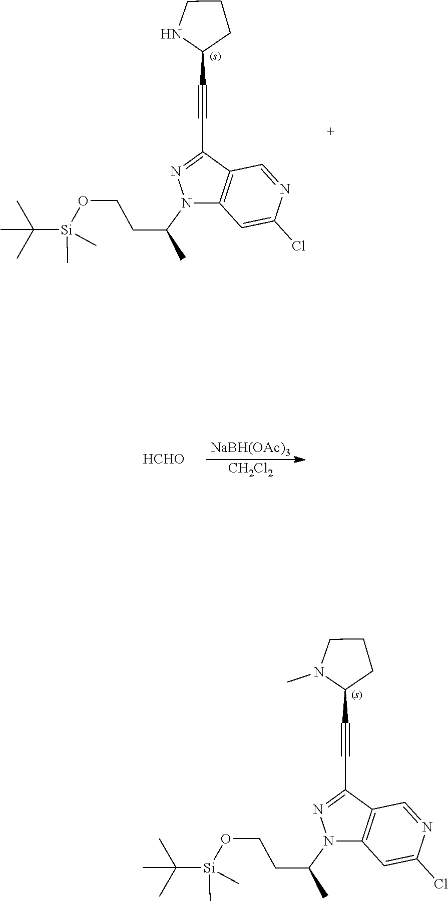

1-((S)-4-((tert-butyldimethylsilyl)oxy)butan-2-yl)-6-chloro-3-(((S)-pyrrolidin-2-yl) ethynyl)-1H-pyrazolo[4,3-c]pyridine (550 mg, 1.27 mmol) was dissolved in CH₂Cl₂ (20 mL), and then aqueous formaldehyde solution (1271 mg, 12.7 mmol) and NaBH(OAc)₃ (1346 mg, 6.35 mmol) were added. The mixture was reacted at room temperature for 3 hours. After the reaction was completed, CH₂Cl₂ was added for dilution, and the mixture was washed with saturated aqueous NaHCO₃ solution, pure water, and saturated brine in sequence, and dried over anhydrous sodium sulfate. It was filtered, and the filtrate was concentrated and then separated by using column chromatography to obtain 1-((S)-4-((tert-butyldimethylsilyl)oxy)butan-2-yl)-6-chloro-3-(((S)-1-methylpyrrolidin-2-yl)ethynyl)-1H-pyrazolo[4,3-c]pyridine (350 mg, yield: 61.6%). MS m/z (ESI): 447.2 [M+H]⁺.

### Intermediate 2-3: Preparation of (1-((6-chloro-3-((1-methylpiperidin-4-yl)ethynyl)-1H-pyrazolo[4,3-c]pyridin-1-yl)methyl)cyclopentyl)methanol

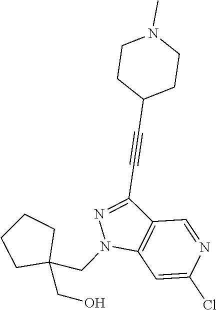 [SMILES: CN1CCC(C#Cc2nn(CC3(CO)CCCC3)c3cc(Cl)ncc23)CC1]

### Step 1: Synthesis of tert-butyl 4-((1-((1-(((tert-butyldimethylsilyl)oxy)methyl)cyclopentyl)methyl)-6-chloro-1H-pyrazolo[4,3-c]pyridin-3-yl)ethynyl)piperidine-1-carboxylate

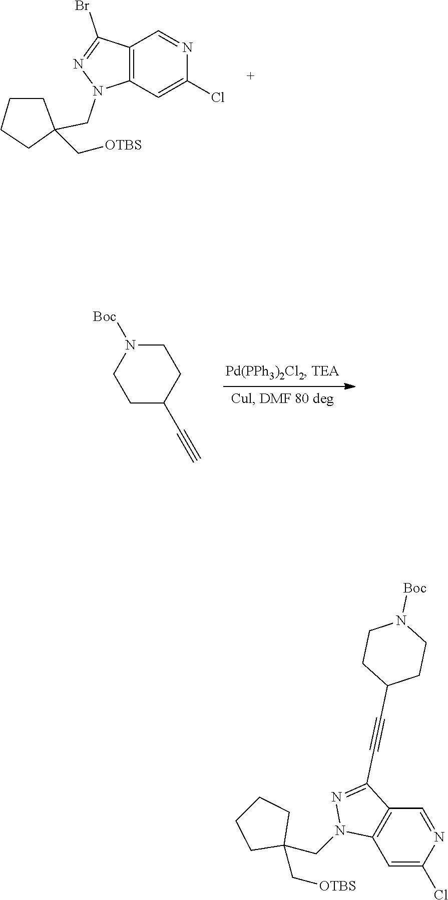 [SMILES: CC(C)(C)[Si](C)(C)OCC1(Cn2nc(Br)c3cnc(Cl)cc32)CCCC1 ; C#CC1CCN(C(=O)OC(C)(C)C)CC1 ; CC(C)(C)OC(=O)N1CCC(C#Cc2nn(CC3(CO[Si](C)(C)C(C)(C)C)CCCC3)c3cc(Cl)ncc23)CC1]

3-bromo-1-((1-(((tert-butyldimethylsilyl)oxy)methyl)cyclopentyl)methyl)-6-chloro-1H-pyrazolo[4,3-c]pyridine (375 mg, 0.817 mmol) was dissolved in DMF (20 mL), and then tert-butyl 4-ethynylpiperidine-1-carboxylate (256.5 mg, 1.226 mmol), CuI (62.3 mg, 0.327 mmol), Et₃N (1.14 mL, 8.172 mmol) and Pd(PPh₃)₂Cl₂ (114.7 mg, 0.163 mmol) were added. The mixture was heated to 80° C., and stirred overnight under nitrogen protection. After the reaction was completed, the reaction solution was concentrated and separated by using column chromatography to obtain tert-butyl 4-((1-((1-(((tert-butyldimethylsilyl)oxy)methyl)cyclopentyl)methyl)-6-chloro-1H-pyrazolo[4,3-c]pyridin-3-yl)ethynyl)piperidine-1-carboxylate (450 mg, 0.766 mmol, yield: 93.8%). MS m/z (ESI): 587.4 [M+H]⁺.

### Step 2: Synthesis of (1-((6-chloro-3-(piperidin-4-ylethynyl)-1H-pyrazolo[4,3-c]pyridin-1-yl)methyl)cyclopentyl)methanol

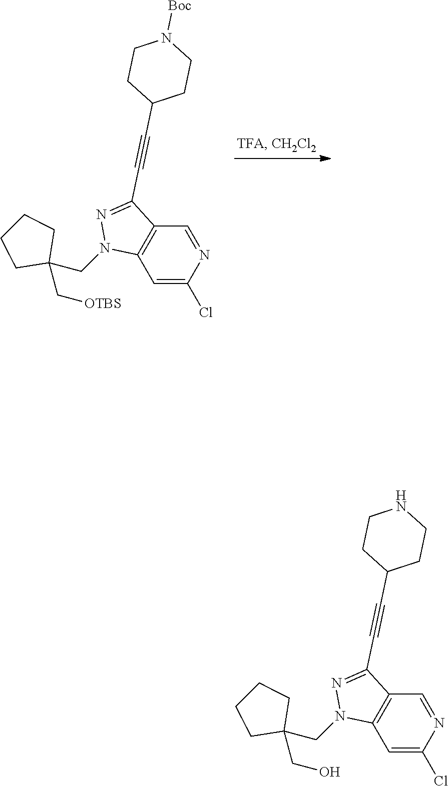 [SMILES: CC(C)(C)OC(=O)N1CCC(C#Cc2nn(CC3(CO[Si](C)(C)C(C)(C)C)CCCC3)c3cc(Cl)ncc23)CC1 ; OCC1(Cn2nc(C#CC3CCNCC3)c3cnc(Cl)cc32)CCCC1]

Tert-buty 4-((1-((1-(tert-butyldimethylsilyl)oxy)methyl)cyclopentyl)methyl)-6-chloro-1H-pyrazolo[4,3-c]pyridin-3-yl)ethynyl)piperidine-1-carboxylate (450 mg, 0.766 mmol) was dissolved in CH₂Cl₂ (15 mL), and TFA (0.57 mL, 7.662 mmol) was added dropwise. The mixture was stirred at room temperature for 2 hours. After the reaction was completed, saturated aqueous sodium bicarbonate solution was added to the reaction solution and the reaction solution was extracted with dichloromethane. The organic phase was combined, washed with water and saturated brine in sequence, and dried over anhydrous sodium sulfate. It was filtered and concentrated to obtain crude (1-((6-chloro-3-(piperidin-4-ylethynyl)-1H-pyrazolo[4,3-c]pyridin-1-yl)methyl)cyclopentyl)methanol (260 mg, 0.697 mmol, yield: 91.0%). MS m/z (ESI): 373.2 [M+H]⁺.

### Step 3: Synthesis of (1-((6-chloro-3-((1-methylpiperidin-4-yl)ethynyl)-1H-pyrazolo[4,3-c]pyridin-1-yl)methyl)cyclopentyl)methanol

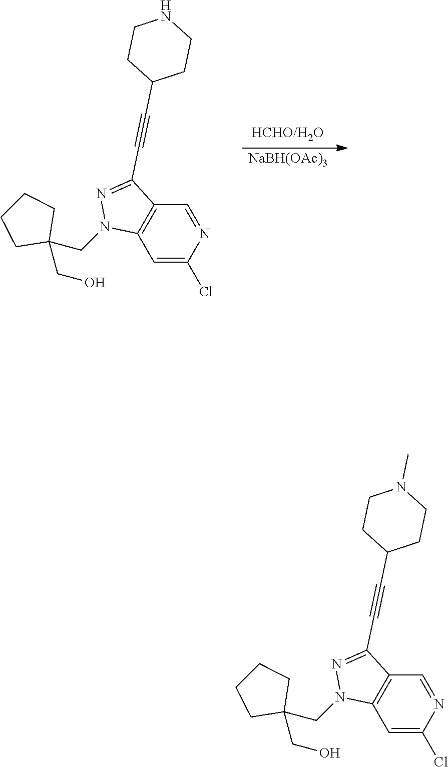

(1-((6-chloro-3-(piperidin-4-ylethynyl)-1H-pyrazolo[4,3-c]pyridin-1-yl)methyl)cyclopentyl)methanol (260 mg, 0.697 mmol) was dissolved in CH₂Cl₂ (20 mL), and then 33% formaldehyde aqueous solution (299 mg, 3.486 mmol) and NaBH(OAc)₃ (739 mg, 3.486 mmol) were added. The mixture was stirred at room temperature for 18 hours. After the reaction was completed, saturated aqueous NaHCO₃ solution was added to quench the reaction solution, and the reaction solution was extracted with dichloromethane three times. The organic phase was combined, concentrated, and then separated by using column chromatography to obtain (1-((6-chloro-3-((1-methylpiperidin-4-yl)ethynyl)-1H-pyrazolo[4,3-c]pyridin-1-yl)meth yl)cyclopentyl)methanol (260 mg, 0.672 mmol, yield: 96.4%). MS m/z (ESI): 387.2 [M+H]⁺.

Intermediates 2-4 to 2-23 can be prepared by selecting the corresponding raw materials with reference to the synthesis methods of intermediate 2-2 or 2-3.

| Intermediate | | | MS: m/z |
|---|---|---|---|
| No. | Structural Formula | Chemical Name | [M + H]⁺ |
| 2-4 | 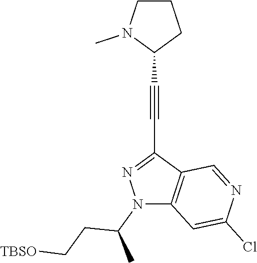 [SMILES: C[C@@H](CCO[Si](C)(C)C(C)(C)C)n1nc(C#C[C@H]2CCCN2C)c2cnc(Cl)cc21] | 1-((S)-4-((tert-butyldimeth- ylsilyl)oxy)butan-2-yl)-6- chloro-3-(((R)-1-methylpyr- rolidin-2-yl)ethynyl)-1H- pyrazolo[4,3-c]pyridine | 448 |
| 2-5 | 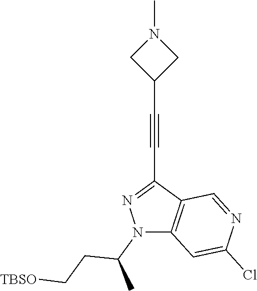 [SMILES: C[C@@H](CCO[Si](C)(C)C(C)(C)C)n1nc(C#CC2CN(C)C2)c2cnc(Cl)cc21] | (S)-1-(4-((tert-butyldimeth- ylsilyl)oxy)butan-2-yl)-6- chloro-3-((1-methylazetidin- 3-yl)ethynyl)-1H-pyrazo- lo[4,3-c]pyridine | 434 |
| 2-6 | 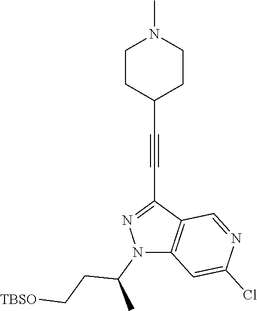 [SMILES: C[C@@H](CCO[Si](C)(C)C(C)(C)C)n1nc(C#CC2CCN(C)CC2)c2cnc(Cl)cc21] | (S)-1-(4-((tert-butyldimeth- ylsilyl)oxy)butan-2-yl)-6- chloro-3-((1-methylpiperi- din-4-yl)ethynyl)-1H-pyra- zolo[4,3-c]pyridine | 462 |
| 2-7 | 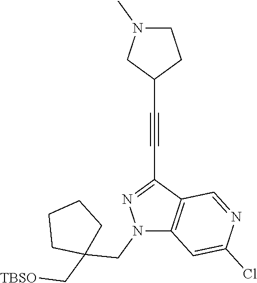 [SMILES: CN1CCC(C#Cc2nn(CC3(CO[Si](C)(C)C(C)(C)C)CCCC3)c3cc(Cl)ncc23)C1] | 1-((1-((tert-butyldimethylsil- yl)oxy)methyl)cyclopentyl) methyl)-6-chloro-3-((1- methylpyrrolidin-3-yl)eth- ynyl)-1H-pyrazolo[4,3-c]py- ridine | 487 |
| 2-8 | 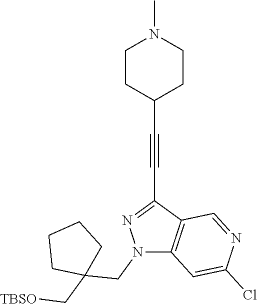 [SMILES: CN1CCC(C#Cc2nn(CC3(CO[Si](C)(C)C(C)(C)C)CCCC3)c3cc(Cl)ncc23)CC1] | 1-((1-((tert-butyldimethylsil- yl)oxy)methyl)cyclopentyl) methyl)-6-chloro-3-((1- methylpiperidin-4-yl)ethy- nyl)-1H-pyrazolo[4,3-c]py- ridine | 501 |
| 2-9 | 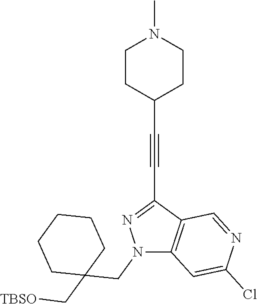 [SMILES: CN1CCC(C#Cc2nn(CC3(CO[Si](C)(C)C(C)(C)C)CCCCC3)c3cc(Cl)ncc23)CC1] | 1-((1-((tert-butyldimethylsil- yl)oxy)methyl)cyclohexyl) methyl)-6-chloro-3-((1- methylpiperidin-4-yl)ethy- nyl)-1H-pyrazolo[4,3-c]py- ridine | 515 |
| 2-10 | 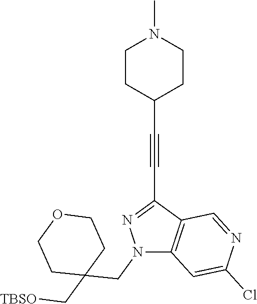 [SMILES: CN1CCC(C#Cc2nn(CC3(CO[Si](C)(C)C(C)(C)C)CCOCC3)c3cc(Cl)ncc23)CC1] | 1-((4-(((tert-butyldimethyl- silyl)oxy)methyl)tetrahydro- 2H-pyran-4-yl)methyl)- 6-chloro-3-((1-methylpiper- idin-4-yl)ethynyl)-1H-pyra- zolo[4,3-c]pyridine | 517 |
| 2-11 | 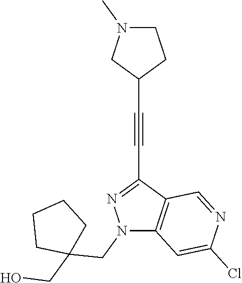 [SMILES: CN1CCC(C#Cc2nn(CC3(CO)CCCC3)c3cc(Cl)ncc23)C1] | (1-((6-chloro-3-((1-methyl- pyrrolidin-3-yl)ethynyl)-1H- pyrazolo[4,3-c]pyridin- 1-yl)methyl)cyclopentyl) methanol | 373 |
| 2-12 | 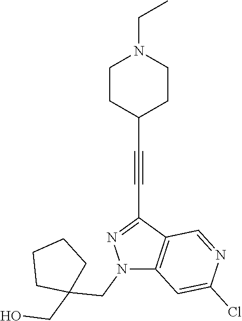 [SMILES: CCN1CCC(C#Cc2nn(CC3(CO)CCCC3)c3cc(Cl)ncc23)CC1] | (1-((6-chloro-3-((1-ethylpi- peridin-4-yl)ethynyl)-1H- pyrazolo[4,3-c]pyridin-1-yl) methyl)cyclopentyl)meth- anol | 401 |
| 2-13 | 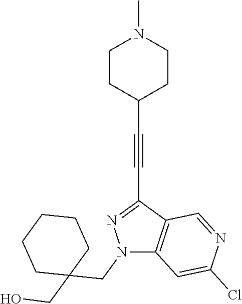 [SMILES: CN1CCC(C#Cc2nn(CC3(CO)CCCCC3)c3cc(Cl)ncc23)CC1] | (1-((6-chloro-3-((1-methyl- piperidin-4-yl)ethynyl)-1H- pyrazolo[4,3-c]pyridin- 1-yl)methyl)cyclohexyl)meth- anol | 401 |
| 2-14 | 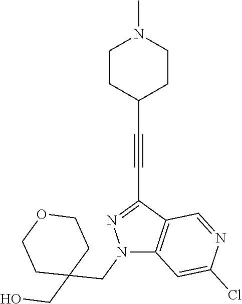 [SMILES: CN1CCC(C#Cc2nn(CC3(CO)CCOCC3)c3cc(Cl)ncc23)CC1] | (4-((6-chloro-3-((1-methyl- piperidin-4-yl)ethynyl)-1H- pyrazolo[4,3-c]pyridin- 1-yl)methyl)tetrahydro-2H- pyran-4-yl)methanol | 403 |
| 2-15 | 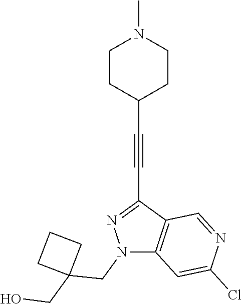 [SMILES: CN1CCC(C#Cc2nn(CC3(CO)CCC3)c3cc(Cl)ncc23)CC1] | (1-((6-chloro-3-((1-methyl- piperidin-4-yl)ethynyl)-1H- pyrazolo[4,3-c]pyridin- 1-yl)methyl)cyclobutyl)meth- nol | 373 |
| 2-16 | 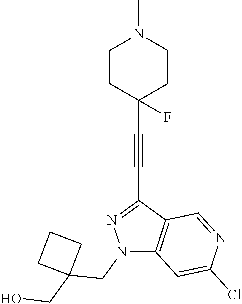 [SMILES: CN1CCC(F)(C#Cc2nn(CC3(CO)CCC3)c3cc(Cl)ncc23)CC1] | (1-((6-chloro-3-((4-fluoro- 1-methylpiperidin-4-yl)eth- ynyl)-1H-pyrazolo[4,3-c]py- ridin-1-yl)methyl)cyclobu- tyl)methanol | 391 |
| 2-17 | 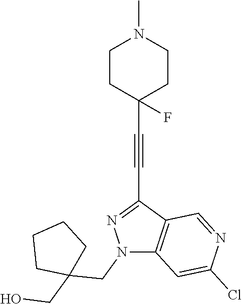 [SMILES: CN1CCC(F)(C#Cc2nn(CC3(CO)CCCC3)c3cc(Cl)ncc23)CC1] | (1-((6-chloro-3-((4-fluoro- 1-methylpiperidin-4-yl)eth- ynyl)-1H-pyrazolo[4,3-c]py- ridin-1-yl)methyl)cyclopen- tyl)methanol | 405 |
| 2-18 | 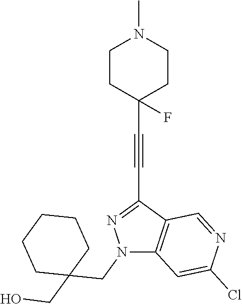 [SMILES: CN1CCC(F)(C#Cc2nn(CC3(CO)CCCCC3)c3cc(Cl)ncc23)CC1] | (1-((6-chloro-3-((4-fluoro- 1-methylpiperidin-4-yl)eth- ynyl)-1H-pyrazolo[4,3-c]py- ridin-1-yl)methyl)cyclohex- yl)methanol | 419 |
| 2-19 | 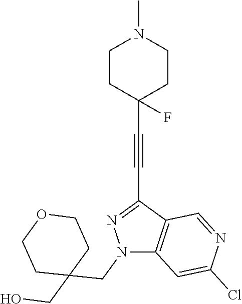 [SMILES: CN1CCC(F)(C#Cc2nn(CC3(CO)CCOCC3)c3cc(Cl)ncc23)CC1] | (4-((6-chloro-3-((4-fluoro- 1-methylpiperidin-4-yl)eth- ynyl)-1H-pyrazolo[4,3-c]py- ridin-1-y1)methyl)tetrahy- dro-2H-pyran-4-yl)methan- ol | 421 |
| 2-20 | 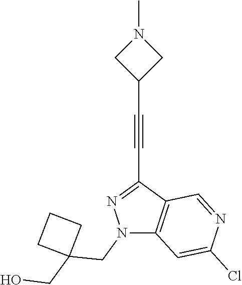 [SMILES: CN1CC(C#Cc2nn(CC3(CO)CCC3)c3cc(Cl)ncc23)C1] | (1-((6-chloro-3-((1-methyl- azetidin-3-yl)ethynyl)-1H- pyrazolo[4,3-c]pyridin-1-yl) methyl)cyclobutyl)metha- nol | 345 |
| 2-21 | 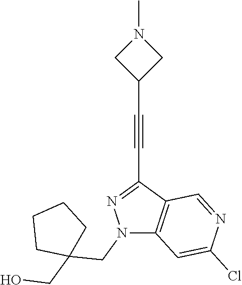 [SMILES: CN1CC(C#Cc2nn(CC3(CO)CCCC3)c3cc(Cl)ncc23)C1] | (1-((6-chloro-3-((1-methyl- azetidin-3-yl)ethynyl)-1H- pyrazolo[4,3-c]pyridin-1-yl) methyl)cyclopentyl)meth- anol | 359 |
| 2-22 | 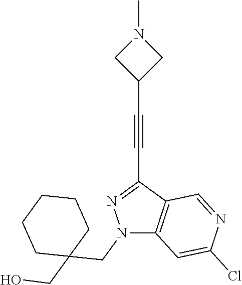 [SMILES: CN1CC(C#Cc2nn(CC3(CO)CCCCC3)c3cc(Cl)ncc23)C1] | (1-((6-chloro-3-((1-methyl- azetidin-3-yl)ethynyl)-1H- pyrazolo[4,3-c]pyridin-1-yl) methyl)cyclohexyl)meth- anol | 373 |
| 2-23 | 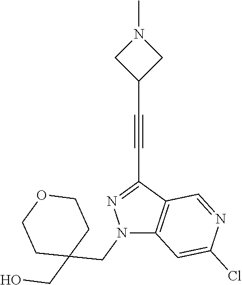 [SMILES: CN1CC(C#Cc2nn(CC3(CO)CCOCC3)c3cc(Cl)ncc23)C1] | (4-((6-chloro-3-((1-methyl- azetidin-3-yl)ethynyl)-1H- pyrazolo[4,3-c]pyridin-1-yl) methyl)tetrahydro-2H-py- ran-4-yl)methanol | 375 |

### Intermediate 3-1: Preparation of tert-butyl 3-((methylsulfonyl)methyl)azetidine-1-carboxylate

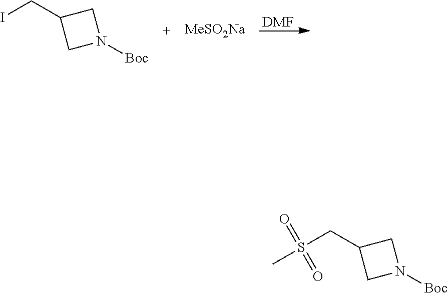

Tert-butyl 3-iodoazetidine-1-carboxylate (5.00 g, 16.83 mmol) was dissolved in DMF (30 mL), and sodium methanesulfinate (2.58 g, 25.24 mmol) was added. The mixture was heated to 70° C., and stirred for 3 hours. After the reaction was completed, ethyl acetate was added to dilute the reaction solution, and the mixture was washed with water and saturated brine in sequence and dried over anhydrous sodium sulfate. It was filtered, and the filtrate was concentrated and then separated by using column chromatography to obtain tert-butyl 3-((methylsulfonyl)methyl)azetidine-1-carboxylate (3.20 g, yield: 68%).

¹HNMR (400 MHz, DMSO-d₆) δ 3.97 (t, 2H), 3.70 (d, 2H), 3.48 (d, J=7.5 Hz, 2H), 3.04-2.99 (m, 1H), 2.95 (s, 3H), 1.37 (s, 9H).

### Intermediate 3-2: Preparation of tert-butyl (S)-3-(((trifluoromethyl)sulfonyl)methyl)pyrrolidine-1-carboxylate

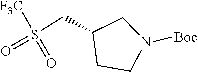 [SMILES: CC(C)(C)OC(=O)N1CC[C@H](CS(=O)(=O)C(F)(F)F)C1]

### Step 1: Synthesis of tert-butyl (S)-3-(((trifluoromethyl)thio)methyl)pyrrolidine-1-carboxylate

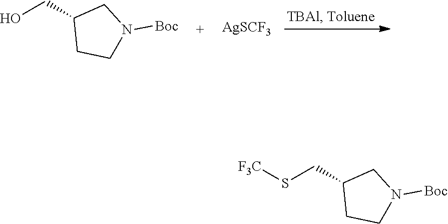

Tetrabutylammonium iodide (132.14 g, 357.73 mmol) and AgSCF₃ (25.03 g, 119.24 mmol) were added to toluene (3(0) mL), and tert-butyl (S)-3-(hydroxymethyl)pyrrolidine-1-carboxylate (6.0 g, 29.81 mmol) was added under nitrogen protection. The mixture was heated to 120° C., and reacted overnight. The reaction solution was filtered with silica gel and washed with ethyl acetate three times. The organic layer was washed with saturated brine, dried over sodium sulfate and concentrated. The product was separated by using column chromatography [petroleum ether/ethyl acetate=0-20%] to obtain tert-butyl (S)-3-(((trifluoromethyl)thio)methyl)pyrrolidine-1-carboxylate (5.0 g, purity: 93.0%, yield: 54.6%). MS m/z (ESI): 230.1 [M−56]⁺.

¹HNMR (400 MHz, CDCl₃) δ 3.67-3.38 (m, 2H), 3.38-3.25 (m, 1H), 3.04 (q, J=10.0, 9.6 Hz, 1H), 2.91 (t, J=7.6 Hz, 2H), 2.47 (q, J=7.6 Hz, 1H), 2.10 (tt, J=7.6, 3.6 Hz, 1H), 1.69-1.56 (m, 1H), 1.45 (s, 9H).

### Step 2: Synthesis of tert-butyl (S)-3-(((trifluoromethyl)sulfonyl)methyl)pyrrolidine-1-carboxylate

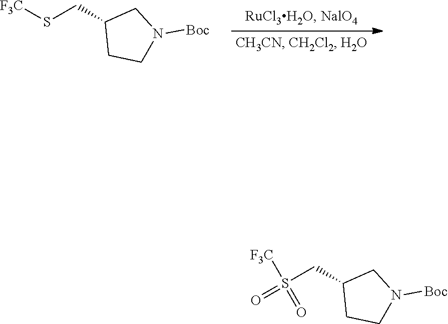 [SMILES: CC(C)(C)OC(=O)N1CC[C@H](CSC(F)(F)F)C1 ; CC(C)(C)OC(=O)N1CC[C@H](CS(=O)(=O)C(F)(F)F)C1]

Ruthenium trichloride hydrate (0.79 g, 3.51 mmol) and sodium periodate (11.24 g, 52.57 mmol) were added in sequence at room temperature to a mixed solution of tert-butyl (S)-3-(((trifluoromethyl)thio)methyl)pyrrolidine-1-carboxylate (5.0 g, 17.52 mmol) in dichloromethane (20 mL), acetonitrile (20 mL) and water (20 mL), and the mixture was reacted at room temperature for 2 hours. The reaction solution was diluted with water, extracted three times with dichloromethane, and the organic phase was combined, washed with saturated brine and dried over anhydrous sodium sulfate in sequence. It was filtered, concentrated and separated by using column chromatography to obtain tert-butyl (S)-3-(((trifluoromethyl)sulfonyl)methyl)pyrrolidine-1-carboxylate (2.9 g, yield: 52.1%). MS m/z (ESI): 262.0 [M−56]⁺.

### Intermediate 3-3: Preparation of tert-butyl (S)-3-((ethylsulfonyl)methyl)pyrrolidine-1-carboxylate

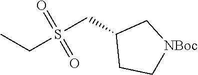 [SMILES: CCS(=O)(=O)C[C@H]1CCN(C(=O)OC(C)(C)C)C1]

### Step 1: Synthesis of tert-butyl (S)-3-((tosyloxy)methyl)pyrrolidine-1-carboxylate

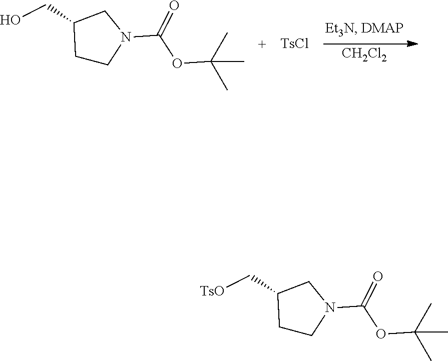 [SMILES: CC(C)(C)OC(=O)N1CC[C@H](CO)C1 ; "TsCl" ; "CH2Cl2" ; Cc1ccc(S(=O)(=O)OC[C@H]2CCN(C(=O)OC(C)(C)C)C2)cc1]

Tert-butyl (S)-3-(hydroxymethyl)pyrrolidine-1-carboxylate (15.0 g, 74.53 mmol) was dissolved in CH₂Cl₂ (200 mL), and then DMAP (4.55 g, 37.26 mmol) and Et₃N (31 mL, 223.58 mmol) were added. TsCl (21.31 g, 111.79 mmol) was added at 0° C., and the mixture was reacted at room temperature for 3 hours. Water and dichloromethane were added to the reaction solution, the solution were separated, and the aqueous phase was extracted twice with dichloromethane. The organic phase was combined, washed sequentially with saturated brine, and dried over anhydrous sodium sulfate. It was filtered, concentrated and separated by using column chromatography to obtain tert-butyl (S)-3-((tosyloxy)methyl)pyrrolidine-1-carboxylate (23.5 g, yield: 88.7%).

¹HNMR (400 MHz, CDCl₃) δ 7.78 (d, J=8.3 Hz, 2H), 7.35 (d, J=7.9 Hz, 2H), 3.95 (qd, J=9.8, 7.0 Hz, 2H), 3.45 (dd, J=11.2, 7.5 Hz, 1H), 3.40-3.22 (m, 2H), 3.0) (dd, J=11.2, 6.9 Hz, 1H), 2.53 (dq, J=14.4, 7.2 Hz, 1H), 2.45 (s, 3H), 1.94 (s, 1H), 1.65 (s, 1H), 1.43 (s, 9H).

### Step 2: Synthesis of tert-butyl (S)-3-((ethylsulfonyl)methyl)pyrrolidine-1-carboxylate

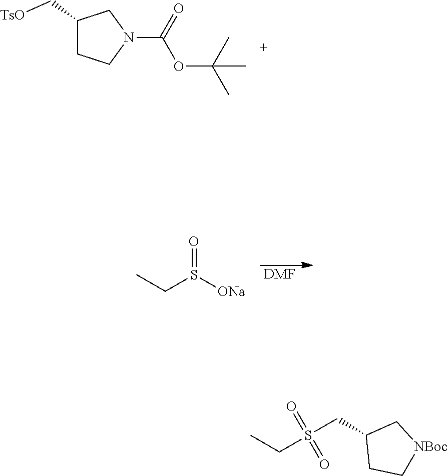

Tert-butyl (S)-3-((tosyloxy)methyl)pyrrolidine-1-carboxylate (8.0 g, 22.51 mmol) was dissolved in DMF (60 mL), potassium iodide (11.21 g, 67.52 mmol) and sodium ethanesulfinate (7.84 g, 67.52 mmol) were added in sequence, and the mixture was heated to 120° C., and reacted for 2 hours. The reaction solution was cooled to room temperature, concentrated, and separated by using column chromatography to obtain tert-butyl (S)-3-((ethylsulfonyl)methyl)pyrrolidine-1-carboxylate (5.4 g, yield: 86.5%).

### Intermediate 3-4: Preparation of tert-butyl (S)-3-((cyclobutylsulfonyl)methyl)pyrrolidine-1-carboxylate

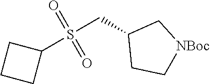 [SMILES: CC(C)(C)OC(=O)N1CC[C@H](CS(=O)(=O)C2CCC2)C1]

### Step 1: Synthesis of Cyclobutanesulfonyl Chloride

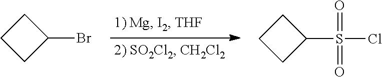 [SMILES: BrC1CCC1 ; O=S(=O)(Cl)C1CCC1]

Magnesium chips (0.41 g, 8.33 mmol) were added to anhydrous tetrahydrofuran (5 mL), and cyclobutyl bromide (0.30 g, 2.22 mmol) and iodine (0.14 g, 0.56 mmol) were added. The mixture was heated with a hair dryer while being stirred until the reaction triggered the system to become colorless. A solution of the remaining cyclobutyl bromide (1.20 g, 8.89 mmol) in tetrahydrofuran (15 mL) was slowly added dropwise. After the addition, the mixture was refluxed for 1 hour and cooled to room temperature for use as a suspension. Under nitrogen, a solution of chlorosulfonyl chloride (2.70 mL, 33.34 mmol) in anhydrous dichloromethane (10 mL) was cooled to 0° C., and the above suspension was slowly added. After the addition, the mixture was warmed to room temperature and reacted for 1 hour. The reaction solution was concentrated to dryness, and n-heptane (25 mL) was added for slurrying. It was filtered, and the filtrate was concentrated to dryness to obtain crude cyclobutanesulfonyl chloride (0.95 g, yield: 55.2%), which was directly used for the next reaction.

### Step 2: Synthesis of Sodium Cyclobutanesulfinate

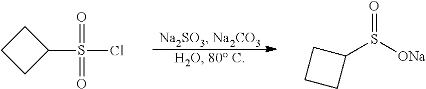

A solution of sodium sulfite (1.55 g, 12.29 mmol) in water (10 mL) was heated to 80° C., and the crude cyclobutanesulfonyl chloride (0.95 g, 6.14 mmol) and sodium carbonate (1.30 g, 12.29 mmol) were added in sequence. The mixture was kept at 80° C. for 1 hour and then concentrated to dryness. Methanol was added for slurrying, and the mixture was filtered. The filtrate was concentrated to dryness to obtain crude sodium cyclobutanesulfinate (0.86 g, yield: 98.5%), which was directly used for the next reaction.

### Step 3: Synthesis of Tert-Butyl (S)-3-((cyclobutylsulfonyl)methyl)pyrrolidine-1-carboxylate

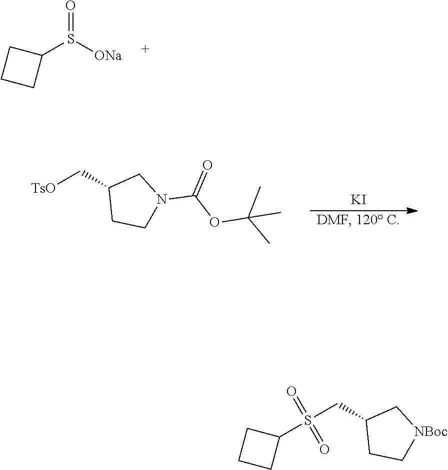

Potassium iodide (1.0 g, 6.02 mmol) and crude sodium cyclobutanesulfinate (0.86 g, 6.08 mmol) were added to a solution of tert-butyl (S)-3-((tosyloxy)methyl)pyrrolidine-1-carboxylate (0.80 g, 2.25 mmol) in N,N-dimethylformamide (10 mL) in sequence at room temperature, and the mixture was heated to 120° C., and reacted for 2 hours. The reaction solution was concentrated and separated by using column chromatography (petroleum ether/ethyl acetate=0-15% to obtain tert-butyl (S)-3-((cyclobutylsulfonyl)methyl)pyrrolidine-1-carboxylate (0.50 g, yield: 73.2%).

¹HNMR (400 MHz, CDCl₃) δ 3.80-3.65 (m, 2H), 3.47 (ddd, J=14.0, 7.3, 3.0 Hz, 1H), 3.30 (ddt, J=11.0, 8.9, 5.4 Hz, 11H), 3.05 (dd, J=10.9, 8.1 Hz, 1H), 2.89 (s, 1H), 2.82-2.66 (m, 1H), 2.65-2.51 (m, 2H), 2.35-2.17 (m, 3H), 2.07 (tt, J=9.9, 3.7 Hz, 2H), 1.84-1.60 (m, 2H), 1.44 (s, 9H).

Intermediates 3-(to 3-15 can be prepared by selecting the corresponding raw materials with reference to the partial routes of intermediate 3-3 or 3-4.

| Intermediate | | | MS: m/z |
|---|---|---|---|
| No. | Structural Formula | Chemical Name | [M + H]⁺ |
| 3-5 | 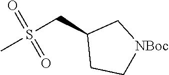 [SMILES: CC(C)(C)OC(=O)N1CC[C@@H](CS(C)(=O)=O)C1] | tert-butyl (R)-3-((methylsulfonyl)methyl) pyrrolidine-1-carboxylate | 264 |
| 3-6 | 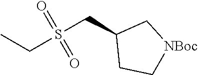 [SMILES: CCS(=O)(=O)C[C@@H]1CCN(C(=O)OC(C)(C)C)C1] | tert-butyl (R)-3-((ethylsulfonyl)methyl) pyrrolidine-1-carboxylate | 278 |
| 3-7 | 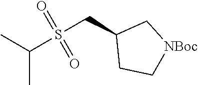 [SMILES: CC(C)S(=O)(=O)C[C@@H]1CCN(C(=O)OC(C)(C)C)C1] | tert-butyl (R)-3-((isopropylsulfonyl)meth- yl)pyrrolidine-1-carboxylate | 292 |
| 3-8 | 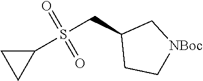 [SMILES: CC(C)(C)OC(=O)N1CC[C@@H](CS(=O)(=O)C2CC2)C1] | tert-butyl (R)-3-((cyclopropylsulfonyl) methyl)pyrrolidine-1-carboxy- late | 290 |
| 3-9 | 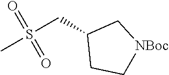 [SMILES: CC(C)(C)OC(=O)N1CC[C@H](CS(C)(=O)=O)C1] | tert-butyl (S)-3-((methylsulfonyl)methyl) pyrrolidine-1-carboxylate | 264 |
| 3-10 | 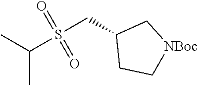 [SMILES: CC(C)S(=O)(=O)C[C@H]1CCN(C(=O)OC(C)(C)C)C1] | tert-butyl (S)-3-((isopropylsulfonyl)meth- yl)pyrrolidine-1-carboxylate | 292 |
| 3-11 | 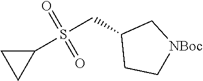 [SMILES: CC(C)(C)OC(=O)N1CC[C@H](CS(=O)(=O)C2CC2)C1] | tert-butyl (S)-3-((cyclopropylsulfonyl) methyl)pyrrolidine-1-carboxy- late | 290 |
| 3-12 | 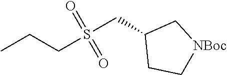 [SMILES: CCCS(=O)(=O)C[C@H]1CCN(C(=O)OC(C)(C)C)C1] | tert-butyl (S)-3-((propylsulfonyl)methyl) pyrrolidine-1-carboxylate | 292 |
| 3-13 | 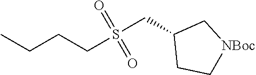 [SMILES: CCCCS(=O)(=O)C[C@H]1CCN(C(=O)OC(C)(C)C)C1] | tert-butyl (S)-3-((butylsulfonyl)methyl) pyrrolidine-1-carboxylate | 306 |
| 3-14 | 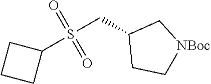 [SMILES: CC(C)(C)OC(=O)N1CC[C@H](CS(=O)(=O)C2CCC2)C1] | tert-butyl (S)-3-((cyclobutylsulfonyl)meth- yl)pyrrolidine-1-carboxylate | 304 |
| 3-15 | 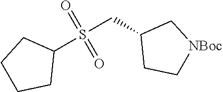 [SMILES: CC(C)(C)OC(=O)N1CC[C@H](CS(=O)(=O)C2CCCC2)C1] | tert-butyl (S)-3-((cyclopentylsulfonyl) methyl)pyrrolidine-1-carboxy- late | 318 |

### Intermediate 4-1: Preparation of 1-((1-(((tert-butyldimethylsilyl)oxy)methyl)cyclobutyl)methyl)-6-chloro-3-(3-((methylsulfonyl)methyl)azetidin-1-yl)-1H-pyrazolo[4,3-c]pyridine

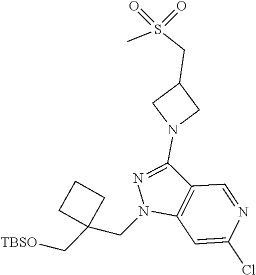 [SMILES: CC(C)(C)[Si](C)(C)OCC1(Cn2nc(N3CC(CS(C)(=O)=O)C3)c3cnc(Cl)cc32)CCC1]

### Step 1: Synthesis of 3-(methylsulfonyl)methyl)azetidine Hydrochloride

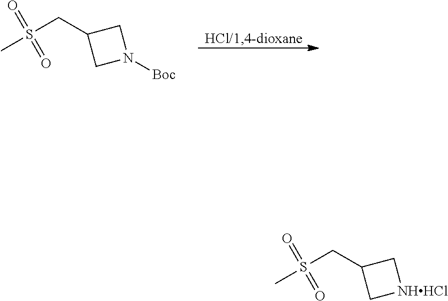

Tert-butyl 3-((methylsulfonyl)methyl)azetidine-1-carboxylate (3.90 g, 15.64 mmol) was dissolved in 1,4-dioxane (4.0 mL), hydrochloric acid (4.0 mL, 16.0 mmol) was added, and the mixture was stirred at room temperature for 1 hour. After the reaction was completed, the reaction solution was concentrated to obtain 3-((methylsulfonyl)methyl)azetidine hydrochloride (3.30 g, yield: 90%).

¹HNMR (400 MHz, MeOH-d₄) δ 4.07-3.97 (m, 2H), 3.89-3.79 (m, 2H), 3.60 (d, J=7.6 Hz, 2H), 3.31-3.23 (m, 1H), 3.00 (s, 3H).

### Step 2: Synthesis of 1-((1-(((tert-butyldimethylsilyl)oxy)methyl)cyclobutyl)methyl)-6-chloro-3-(3-((methylsulfonyl)methyl)azetidin-1-yl)-1H-pyrazolo[4,3-c]pyridine

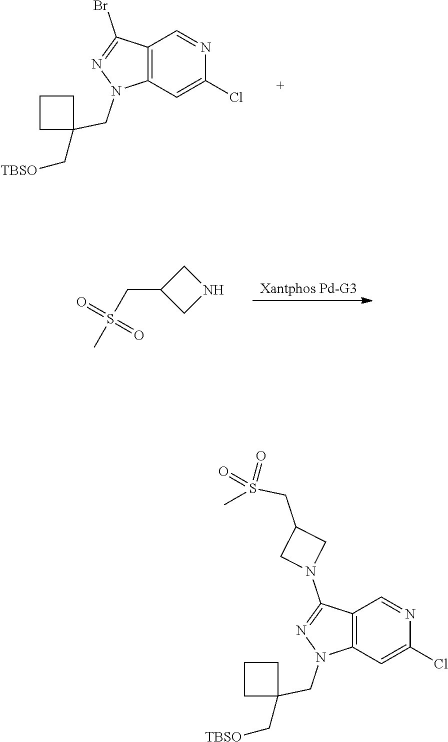

3-bromo-1-((1-(((tert-butyldimethylsilyl)oxy)methyl)cyclobutyl)methyl)-6-chloro-1H-pyrazolo[4,3-c]pyridine (0.60 g, 1.24 mmol) was added to 1,4-dioxane (15.0 mL), and then 3-((methylsulfonyl)methyl)azetidine hydrochloride (0.35 g, 1.86 mmol), Cs₂CO₃ (1.21 g, 3.72 mmol) and Xantphos Pd-G3 (0.35 g, 0.37 mmol) were added in sequence. Under nitrogen, the mixture was heated to 80° C. under nitrogen and reacted overnight. After the reaction was completed, the reaction solution was cooled to room temperature, quenched by adding saturated brine, and extracted with EtOAc. The organic layer was washed with saturated brine and dried over anhydrous sodium sulfate. It was filtered and concentrated, and the residue was separated by using column chromatography to obtain 1-((1-(((tert-butyldimethylsilyl)oxy)methyl)cyclobutyl)methyl)-6-chloro-3-(3-((methylsulfonyl)methyl)azetidine-1-yl)-1H-pyrazolo[4,3-c]pyridine (0.50 g, purity: 58.5%, yield: 45.7%). MS m/z (ESI): 513.2 [M+H]⁺.

### Intermediate 4-2: Preparation of (S)-(1-((6-chloro-3-(3-((ethylsulfonyl)methyl)pyrrolidin-1-yl)-1H-pyrazolo[4,3-c]pyridin-1-yl)methyl)cyclopentyl)methanol

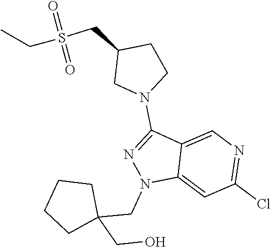 [SMILES: CCS(=O)(=O)C[C@H]1CCN(c2nn(CC3(CO)CCCC3)c3cc(Cl)ncc23)C1]

### Step 1: Synthesis of (S)-3-((ethylsulfonyl)methyl)pyrrolidine Hydrochloride

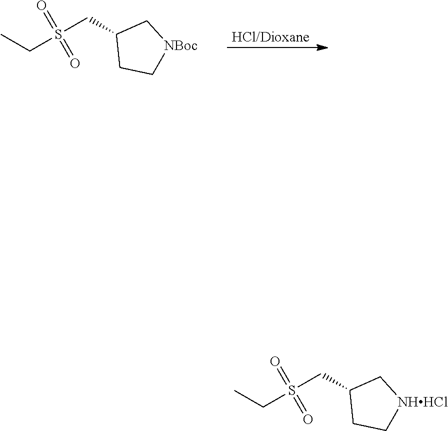

Tert-butyl (S)-3-((ethylsulfonyl)methyl)pyrrolidine-1-carboxylate (5.4 g, 19.47 mmol) was dissolved in 1,4-dioxane (15 mL), and then HCl/1,4-dioxane solution (14.6 mL, 4 M, 58.40 mmol) was slowly added dropwise and reacted at room temperature for 1 hour. After the reaction was completed, the reaction solution was concentrated to obtain crude (S)-3-((ethylsulfonyl)methyl)pyrrolidine hydrochloride (3.75 g, yield: 90.1%), which was directly used in the next reaction.

### Step 2: Synthesis of methyl (S)-1-((6-chloro-3-(3-((ethylsulfonyl)methyl)pyrrolidin-1-yl)-1H-pyrazolo[4,3-c]pyridin-1-yl)methyl)cyclopentane-1-carboxylate

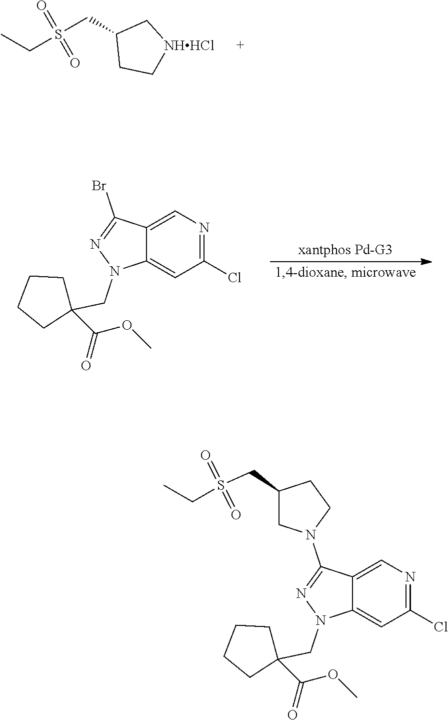

(S)-3-((ethylsulfonyl)methyl)pyrrolidine hydrochloride (0.34 g, 1.61 mmol) and methyl 1-((3-bromo-6-chloro-1H-pyrazolo[4,3-c]pyridin-1-yl)methyl)cyclopentane-1-carboxylate (0.40 g, 1.07 mmol) were dissolved in 1,4-dioxane (8 mL), and then Cs₂CO₃ (1.40 g, 4.29 mmol) and xantphos Pd-G3 (0.15 g, 0.16 mmol) were added. After the nitrogen substitution was carried out, the mixture was heated in a microwave oven at 85° C. for 4 hours. After the reaction solution was cooled, ethyl acetate was added to dilute it, and then the reaction solution was washed once with water and saturated brine respectively. The organic phase was washed with saturated brine and dried over anhydrous sodium sulfate. It was filtered and concentrated, and the crude product was separated by using column chromatography to obtain methyl (S)-1-((6-chloro-3-(3-((ethylsulfonyl)methyl)pyrrolidin-1-yl)-1H-pyrazolo[4,3-c]pyridin-1-yl)methyl)cyclopentane-1-carboxylate (0.33 g, purity: 77.5%, yield: 51.2%). MS m/z (ESI): 469.2 [M+H]⁺.

### Step 3: Synthesis of (S)-(1-((6-chloro-3-(3-((ethylsulfonyl)methyl)pyrrolidin-1-yl)-1H-pyrazolo[4,3-c]pyridin-1-yl)methyl)cyclopentyl)methanol

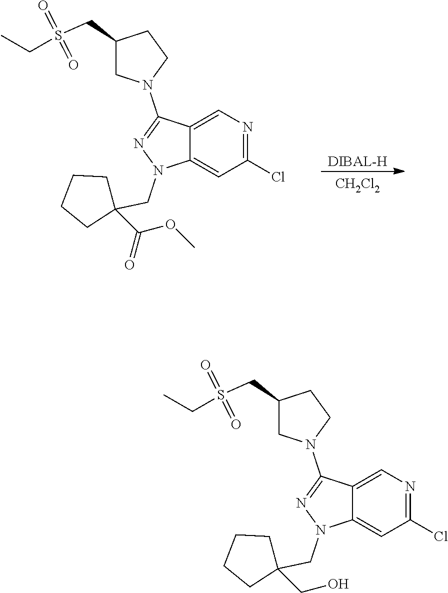 [SMILES: CCS(=O)(=O)C[C@H]1CCN(c2nn(CC3(C(=O)OC)CCCC3)c3cc(Cl)ncc23)C1 ; "CH2Cl2" ; "DIBAL-H" ; CCS(=O)(=O)C[C@H]1CCN(c2nn(CC3(CO)CCCC3)c3cc(Cl)ncc23)C1]

(S)-1-((6-chloro-3-(3-((ethylsulfonyl)methyl)pyrrolidin-1-yl)-1H-pyrazolo[4,3-c]pyridin-1-yl)methyl)cyclopentane-1-carboxylate (0.60 g, 0.99 mmol) was dissolved in dichloromethane (8 mL), the mixture was cooled to −78° C., and DIBAL-H solution (4.06 mL, 1.0 M, 4.06 mmol) was slowly added dropwise. The mixture was maintained at the low temperature and reacted for 1 hour. Saturated aqueous ammonium chloride solution was added to the reaction solution to quench the reaction, and then the solution was extracted twice with dichloromethane. The organic phase was combined, washed sequentially with saturated brine, and dried over anhydrous sodium sulfate. It was filtered and concentrated, and the crude product was separated by using column chromatography (eluent: CH₂Cl₂/MeOH 0-3%) to obtain (S)-(1-((6-chloro-3-(3-((ethylsulfonyl)methyl)pyrrolidin-1-yl)-1H-pyrazolo[4,3-c]pyridin-1-yl)methyl)cyclopentyl)methanol (0.43 g, purity: 87.0%, yield: 85.6%). MS m/z (ESI): 441.2 [M+H]⁺.

Intermediates 4-3 to 4-23 can be prepared by selecting the corresponding raw materials with reference to the synthesis methods of intermediate 4-1 or 4-2.

| Intermediate | | | MS: m/z |
|---|---|---|---|
| No. | Structural Formula | Chemical Name | [M + H]⁺ |
| 4-3 | 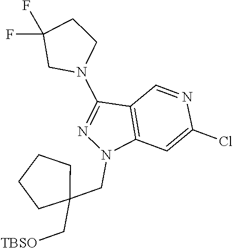 [SMILES: CC(C)(C)[Si](C)(C)OCC1(Cn2nc(N3CCC(F)(F)C3)c3cnc(Cl)cc32)CCCC1] | 1-((1-(((tert-butyldimethyl- silyl)oxy)methyl)cyclopentyl) methyl)-6-chloro-3-(3,3- difluoropyrrolidin-1-yl)-1H- pyrazolo[4,3-c]pyridine | 485 |
| 4-4 | 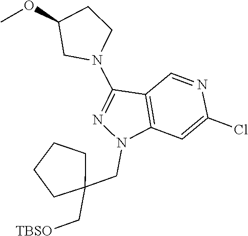 [SMILES: CO[C@H]1CCN(c2nn(CC3(CO[Si](C)(C)C(C)(C)C)CCCC3)c3cc(Cl)ncc23)C1] | (S)-1-((1-(((tert-butyldimeth- ylsilyl)oxy)methyl)cyclo- pentyl)methyl)-6-chloro-3- (3-methoxypyrrolidin-1-yl)- 1H-pyrazolo[4,3-c]pyridine | 479 |
| 4-5 | 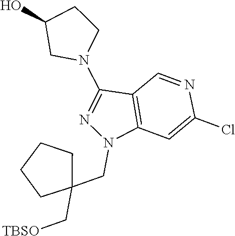 [SMILES: CC(C)(C)[Si](C)(C)OCC1(Cn2nc(N3CC[C@H](O)C3)c3cnc(Cl)cc32)CCCC1] | (S)-1-(1-((1-(((tert-butyldi- methylsilyl)oxy)methyl)cy- clopentyl)methyl)-6-chloro- 1H-pyrazolo[4,3-c]pyridin- 3-yl)pyrrolidin-3-ol | 465 |
| 4-6 | 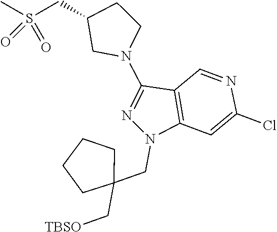 [SMILES: CC(C)(C)[Si](C)(C)OCC1(Cn2nc(N3CC[C@@H](CS(C)(=O)=O)C3)c3cnc(Cl)cc32)CCCC1] | (R)-1-((1-(((tert-butyldimeth- ylsilyl)oxy)methyl)cyclo- pentyl)methyl)-6-chloro-3- (3-((methylsulfonyl)methyl) pyrrolidin-1-yl)-1H-pyra- zolo[4,3-c]pyridine | 541 |
| 4-7 | 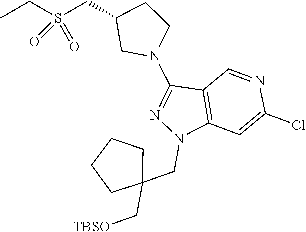 [SMILES: CCS(=O)(=O)C[C@@H]1CCN(c2nn(CC3(CO[Si](C)(C)C(C)(C)C)CCCC3)c3cc(Cl)ncc23)C1] | (R)-1-((1-(((tert-butyldimeth- ylsilyl)oxy)methyl)cyclo- pentyl)methyl)-6-chloro-3- (3-((ethylsulfonyl)methyl) pyrrolidin-1-yl)-1H-pyrazo- lo[4,3-c]pyridine | 555 |
| 4-8 | 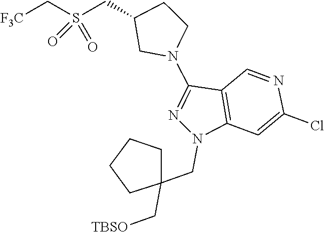 [SMILES: CC(C)(C)[Si](C)(C)OCC1(Cn2nc(N3CC[C@@H](CS(=O)(=O)CC(F)(F)F)C3)c3cnc(Cl)cc32)CCCC1] | (R)-1-((1-(((tert-butyldimeth- ylsilyl)oxy)methyl)cyclo- pentyl)methyl)-6-chloro-3- (3-(((2,2,2-trifluoroethyl)sul- fonyl)methyl)pyrrolidin- 1-yl)-1H-pyrazolo[4,3-c]py- ridine | 609 |
| 4.9 | 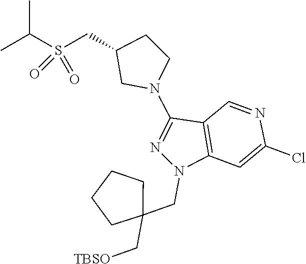 [SMILES: CC(C)S(=O)(=O)C[C@@H]1CCN(c2nn(CC3(CO[Si](C)(C)C(C)(C)C)CCCC3)c3cc(Cl)ncc23)C1] | (R)-1-((1-(((tert-butyldimeth- ylsilyl)oxy)methyl)cyclo- pentyl)methyl)-6-chloro-3- (3-((isopropylsulfonyl)meth- yl)pyrrolidin-1-yl)-1H-py- razolo[4,3-c]pyridine | 569 |
| 4-10 | 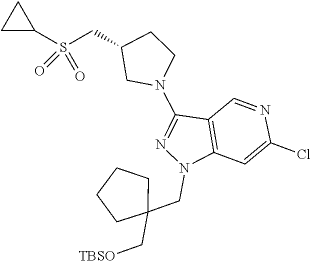 [SMILES: CC(C)(C)[Si](C)(C)OCC1(Cn2nc(N3CC[C@@H](CS(=O)(=O)C4CC4)C3)c3cnc(Cl)cc32)CCCC1] | (R)-1-((1-(((tert-butyldimeth- ylsilyl)oxy)methyl)cyclo- pentyl)methyl)-6-chloro-3- (3-((cyclopropylsulfonyl) methyl)pyrrolidin-1-yl)-1H- pyrazolo[4,3-c]pyridine | 567 |
| 4-11 | 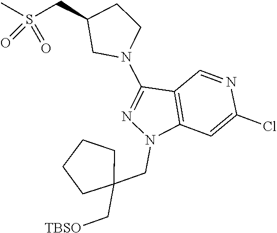 [SMILES: CC(C)(C)[Si](C)(C)OCC1(Cn2nc(N3CC[C@H](CS(C)(=O)=O)C3)c3cnc(Cl)cc32)CCCC1] | (S)-1-((1-(((tert-butyldimeth- ylsilyl)oxy)methyl)cyclo- pentyl)methyl)-6-chloro-3- (3-((methylsulfonyl)methyl) pyrrolidin-1-yl)-1H-pyra- zolo[4,3-c]pyridine | 541 |
| 4-12 | 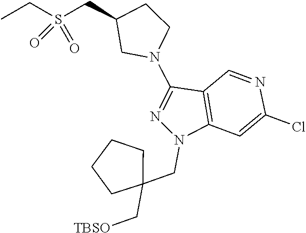 [SMILES: CCS(=O)(=O)C[C@H]1CCN(c2nn(CC3(CO[Si](C)(C)C(C)(C)C)CCCC3)c3cc(Cl)ncc23)C1] | (S)-1-((1-(((tert-butyldimeth- ylsilyl)oxy)methyl)cyclo- pentyl)methyl)-6-chloro-3- (3-((ethylsulfonyl)methyl) pyrrolidin-1-yl)-1H-pyrazo- lo[4,3-c]pyridine | 555 |
| 4-13 | 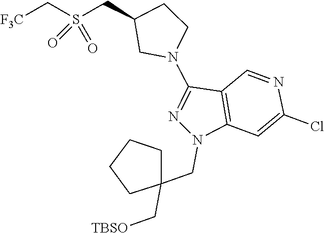 [SMILES: CC(C)(C)[Si](C)(C)OCC1(Cn2nc(N3CC[C@H](CS(=O)(=O)CC(F)(F)F)C3)c3cnc(Cl)cc32)CCCC1] | (S)-1-((1-(((tert-butyldimeth- ylsilyl)oxy )methyl)cyclo- pentyl)methyl)-6-chloro-3- (3-(((2,2,2-trifluoroethyl)sul- fonyl)methyl)pyrrolidin- 1-yl)-1H-pyrazolo[4,3-c]py- ridine | 609 |
| 4-14 | 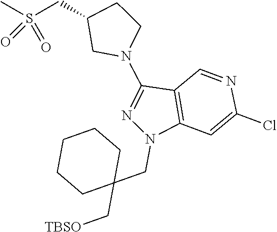 [SMILES: CC(C)(C)[Si](C)(C)OCC1(Cn2nc(N3CC[C@@H](CS(C)(=O)=O)C3)c3cnc(Cl)cc32)CCCCC1] | (R)-1-((1-(((tert-butyldimeth- ylsilyl)oxy)methyl)cyclo- hexyl)methyl)-6-chloro-3-(3- ((methylsulfonyl)methyl) pyrrolidin-1-yl)-1H-pyrazo- lo[4,3-c]pyridine | 555 |
| 4-15 | 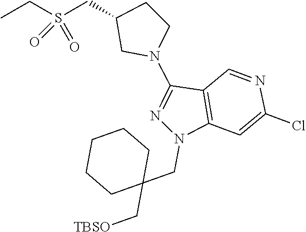 [SMILES: CCS(=O)(=O)C[C@@H]1CCN(c2nn(CC3(CO[Si](C)(C)C(C)(C)C)CCCCC3)c3cc(Cl)ncc23)C1] | (R)-1-((1-(((tert-butyldimeth- ylsilyl)oxy)methyl)cyclo- hexyl)methyl)-6-chloro-3-(3- ((ethylsulfonyl)methyl)pyr- rolidin-1-yl)-1H-pyrazolo [4,3-c]pyridine | 569 |
| 4-16 | 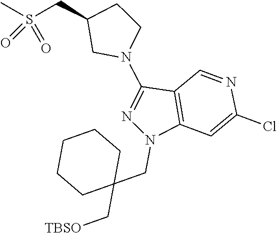 [SMILES: CC(C)(C)[Si](C)(C)OCC1(Cn2nc(N3CC[C@H](CS(C)(=O)=O)C3)c3cnc(Cl)cc32)CCCCC1] | (S)-1-((1-(((tert-butyldimeth- ylsilyl)oxy)methyl)cyclo- hexyl)methyl)-6-chloro-3-(3- ((methylsulfonyl)methyl) pyrrolidin-1-yl)-1H-pyrazo- lo[4,3-c]pyridine | 555 |
| 4-17 | 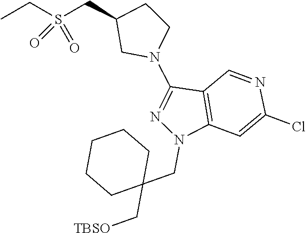 [SMILES: CCS(=O)(=O)C[C@H]1CCN(c2nn(CC3(CO[Si](C)(C)C(C)(C)C)CCCCC3)c3cc(Cl)ncc23)C1] | (S)-1-((1-(((tert-butyldimeth- ylsilyl)oxy)methyl)cyclo- hexyl)methyl)-6-chloro-3-(3- ((ethylsulfonyl)methyl)pyr- rolidin-1-yl)-1H-pyrazolo [4,3-c]pyridine | 569 |
| 4-18 | 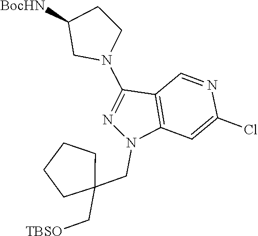 [SMILES: CC(C)(C)OC(=O)N[C@H]1CCN(c2nn(CC3(CO[Si](C)(C)C(C)(C)C)CCCC3)c3cc(Cl)ncc23)C1] | tert-butyl-(S)-(1-(1-((1-(((tert- butyldimethylsilyl)oxy) methyl)cyclopentyl)methyl)- 6-chloro-1H-pyrazolo[4,3- c]pyridin-3-yl)pyrrolidin- 3-yl)carbamate | 564 |
| 4-19 | 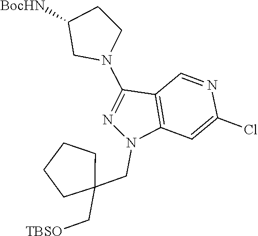 [SMILES: CC(C)(C)OC(=O)N[C@@H]1CCN(c2nn(CC3(CO[Si](C)(C)C(C)(C)C)CCCC3)c3cc(Cl)ncc23)C1] | tert-butyl-(R)-(1-(1-((1-(((tert- butyldimethylsilyl)oxy) methyl)cyclopentyl)methyl)- 6-chloro-IH-pyrazolo[4,3- c]pyridin-3-yl)pyrrolidin- 3-yl)carbamate | 564 |
| 4-20 | 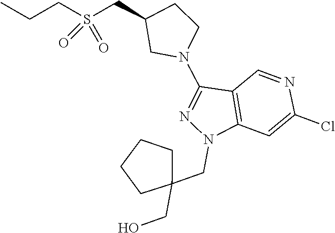 [SMILES: CCCS(=O)(=O)C[C@H]1CCN(c2nn(CC3(CO)CCCC3)c3cc(Cl)ncc23)C1] | (S)-(1-((6-chloro-3-(3-((pro- pylsulfonyl)methyl)pyrro- lidin-1-yl)-1H-pyrazolo[4,3- c]pyridin-1-yl)methyl)cy- clopentyl)methanol | 455 |
| 4-21 | 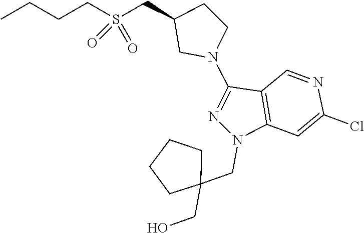 [SMILES: CCCCS(=O)(=O)C[C@H]1CCN(c2nn(CC3(CO)CCCC3)c3cc(Cl)ncc23)C1] | (S)-(1-((6-chloro-3-(3-((bu- tylsulfonyl)methyl)pyrroli- din-1-yl)-1H-pyrazolo[4,3- c]pyridin-1-yl)methyl)cyclo- pentyl)methanol | 469 |
| 4-22 | 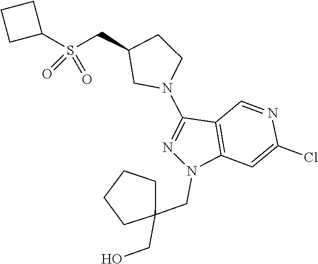 [SMILES: O=S(=O)(C[C@H]1CCN(c2nn(CC3(CO)CCCC3)c3cc(Cl)ncc23)C1)C1CCC1] | (S)-(1-((6-chloro-3-(3-((cy- clobutylsulfonyl)methyl)pyr- rolidin-1-yl)-1H-pyrazolo [4,3-c]pyridin-1-yl)methyl) cyclopentyl)methanol | 467 |
| 4-23 | 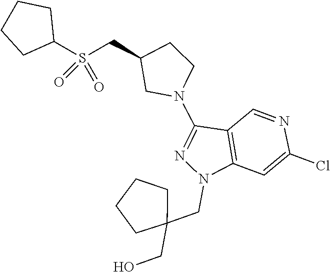 [SMILES: O=S(=O)(C[C@H]1CCN(c2nn(CC3(CO)CCCC3)c3cc(Cl)ncc23)C1)C1CCCC1] | (S)-(1-((6-chloro-3-(3-((cy- clopentylsulfonyl)methyl) pyrrolidin-1-yl)-1H-pyrazo- lo[4,3-c]pyridin-1-yl)meth- yl)cyclopentyl)methanol | 481 |

### Intermediate 5-1: Preparation of 4-(4-aminopyrimidin-2-yl)-2-methyl-1-((2-(trimethylsilyl)ethoxy)methyl) 1,2-dihydro-3H-pyrazol-3-one

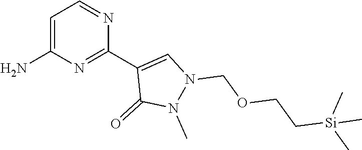 [SMILES: Cn1c(=O)c(-c2nccc(N)n2)cn1COCC[Si](C)(C)C]

### Step: Synthesis of 2-methyl-1-((2-(trimethylsilyl)ethoxy)methyl)-1,2-dihydro-3H-pyrazol-3-one

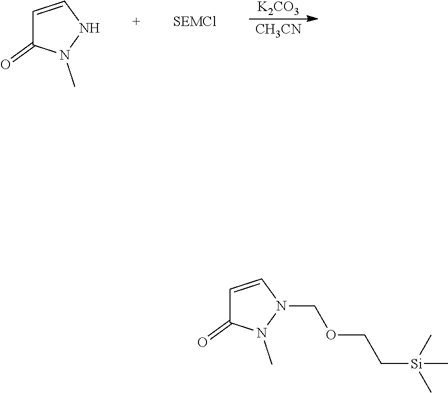

2-methyl-1,2,3-dihydro-1H-pyrazol-3-one (6.0 g, 61.16 mmol) was dissolved in CH₃CN (80 mL), and then K₂CO₃ (36.0 g, 260.52 mmol) and SEMCl (22.0 mL, 124.15 mmol) were added. The reaction was stirred at room temperature for 18 hours. It was filtered, and the filterate was concentrated. The solid was added to a PE/EA (10:1) mixture for slurrying. The mixture was filtered, and the solid was dried in vacuum to obtain 2-methyl-1-((2-(trimethylsilyl)ethoxy)methyl)-1,2-dihydro-3H-pyrazol-3-one (12.1 g, yield: 82.3%).

### Step 2: Synthesis of 4-bromo-2-methyl-1-((2-(trimethylsilyl)ethoxy)methyl)-1,2-dihydro-3H-pyrazol-3-one

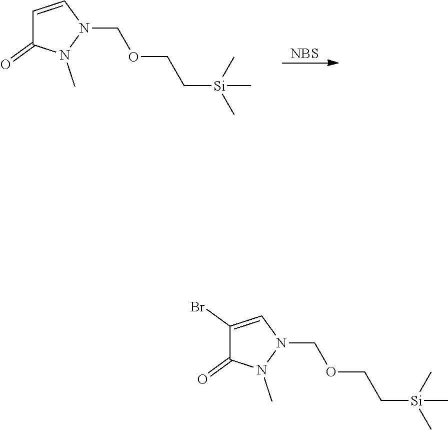

2-methyl-1-((2-(trimethylsilyl)ethoxy)methyl)-1,2-dihydro-3H-pyrazol-3-one (12.1 g, 52.98 mmol) was dissolved in CH₃CN (250 mL). The mixture was cooled to 0° C., and NBS (14.15 g, 79.48 mmol) was added. The mixture was stirred at room temperature for 3 hours. After the reaction was completed, saturated aqueous Na₂S₂O₃ solution was added to quench the reaction, and the mixture was extracted with CH₂Cl₂. The organic phase was washed with saturated aqueous NaCl solution, dried over anhydrous Na₂SO₄, filtered and concentrated. The residue was added to the PE/EA (10:1) mixture and slurried, filtered, and the solid was dried in vacuum to obtain 4-bromo-2-methyl-1-((2-(trimethicone)ethoxy)methyl)-1,2-dihydro-3H-pyrazol-3-one (12.00 g, yield: 70%).

### Step 3: Synthesis of tert-butyl (tert-butyloxycarbonyl)(2-chloropyrimidin-4-yl)carbamate

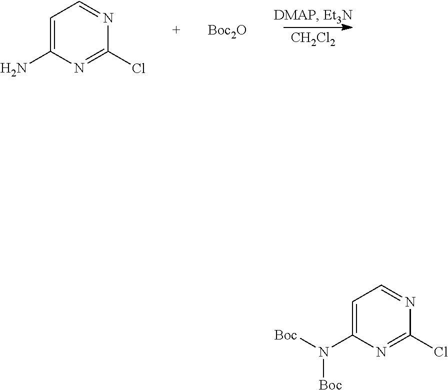

2-chloropyrimidin-4-amine (8.00 g, 61.75 mmol) was dissolved in THF (100 mL), and DMAP (0.75 g, 6.18 mmol), Et₃N (34.3 mL, 247.0 mmol) and Boc₂O (40.43 g, 185.26 mmol) were added. The mixture was stirred at room temperature and reacted for 18 hours. After the reaction was completed, EtOAc and water were added for dilution and extraction, and the organic phase was washed with saturated aqueous NaCl solution, dried over anhydrous Na₂SO₄, filtered and concentrated. The residue was slurried in petroleum ether, filtered, and the filter cake was dried in vacuum to obtain tert-butyl (tert-butoxycarbonyl)(2-chloropyrimidin-4-yl)carbamate (19.72 g, yield: 92%).

### Step 4: Synthesis of tert-butyl (tert-butyloxycarbonyl)(2-(2-methyl-3-oxy-1-((2-(trimethylsilyl)ethoxy)methyl)-2,3-dihydro-1H-pyrazol-4-yl)pyrimidin-4-yl)carbamate

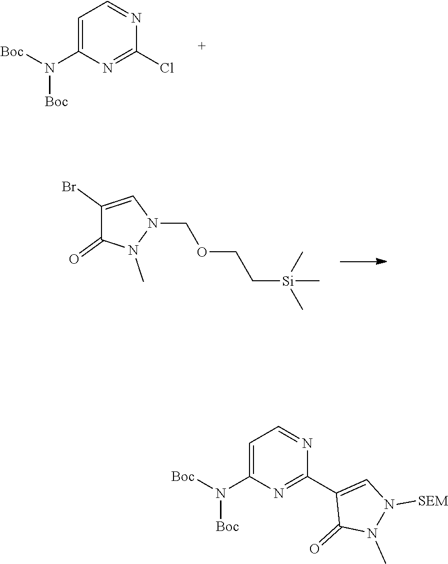 [SMILES: CC(C)(C)OC(=O)N(C(=O)OC(C)(C)C)c1ccnc(Cl)n1 ; Cn1c(=O)c(Br)cn1COCC[Si](C)(C)C ; Cn1c(=O)c(-c2nccc(N(C(=O)OC(C)(C)C)C(=O)OC(C)(C)C)n2)cn1COCC[Si](C)(C)C]

4-bromo-2-methyl-1-((2-(trimethylsilyl)ethoxy)methyl)-1,2-dihydro-3H-pyrazol-3-one (0.5 g, 1.63 mmol) was dissolved in a CH₃CN/H₂O mixture (25/2.5 mL), and tert-butyl (tert-butyloxycarbonyl)(2-chloropyrimidin-4-yl)carbamate (2.68 g, 8.14 mmol), B₂Pin₂ (2.11 mL, 8.14 mmol), Na₂CO₃ (0.86 g, 8.14 mmol) and Pd(Amphos)₂Cl₂ (0.23 g, 0.33 mmol) were added. Under nitrogen protection, the mixture was heated to 100° C., and reacted for 4 hours. After the reaction was completed, the reaction solution was cooled to room temperature, and EtOAc and H₂O were added for extraction. The organic phase was washed with saturated aqueous NaCl solution, dried over anhydrous Na₂SO₄, filtered, and concentrated. The residue was separated by using column chromatography to obtain tert-butyl (tert-butoxycarbonyl)(2-(2-methyl-3-oxy-1-((2-(trimethylsilyl)ethoxy)methyl)-2,3-dihydro-1H-pyrazol-4-yl)pyrimidin-4-yl)carbamate (580 mg, yield: 47.8%).

### Step 5: Synthesis of 4-(4-aminopyrimidin-2-yl)-2-methyl-1-((2-(trimethylsilyl)ethoxy)methyl)-1,2-dihydro-3H-pyrazol-3-one

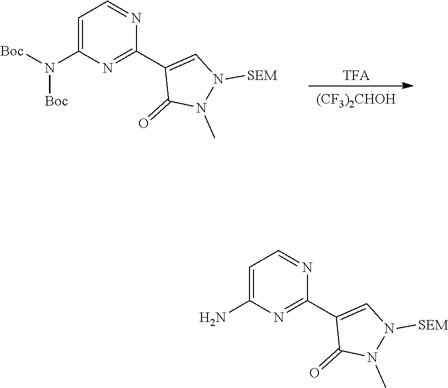 [SMILES: Cn1c(=O)c(-c2nccc(N(C(=O)OC(C)(C)C)C(=O)OC(C)(C)C)n2)cn1COCC[Si](C)(C)C ; "(CF3)2CHOH" ; Cn1c(=O)c(-c2nccc(N)n2)cn1COCC[Si](C)(C)C ; "TFA"]

Tert-butyl (tert-butoxycarbonyl)(2-(2-methyl-3-oxy-1-((2-(trimethylsilyl)ethoxy)methyl)-2,3-dihydro-1H-pyrazol-4-yl)pyrimidin-4-yl)carbamate (1.4 g, 2.68 mmol) was dissolved in hexafluoroisopropanol (15 mL), and TFA (2.8 mL, 37.57 mmol) was added. The mixture was stirred at room temperature and reacted for 3 hours. Saturated aqueous NaHCO₃ solution was added for neutralization, and the mixture was extracted with EtOAc. The organic phase was washed with saturated aqueous NaCl solution, dried over anhydrous Na₂SO₄, filtered and concentrated. The residue was separated by using column chromatography to obtain 4-(4-aminopyrimidin-2-yl)-2-methyl-1-((2-(trimethylsilyl)ethoxy)methyl)-1,2-dihydro-3H-pyrazol-3-one (0.60 g, yield: 69.6%).

### II. Preparation of Compounds of Examples

### Example A1: Preparation of (S)-1¹,5-dimethyl-4³-(prop-1-yn-1-yl)-1¹H,4¹H-8-oxa-3-aza-4(6,1)-pyrazolo[4,3-c]pyridina-2(2,4)-pyrimidina-1(4,5)-pyrazolacyclooctaphane

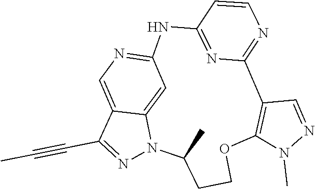 [SMILES: CC#Cc1nn2c3cc(ncc13)Nc1ccnc(n1)-c1cnn(C)c1OCC[C@@H]2C]

### Step 1: Synthesis of (S)-4-(4-((1-(4-((tert-butyldimethylsilyl)oxy)butan-2-yl)-3-(prop-1-yn-1-yl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one

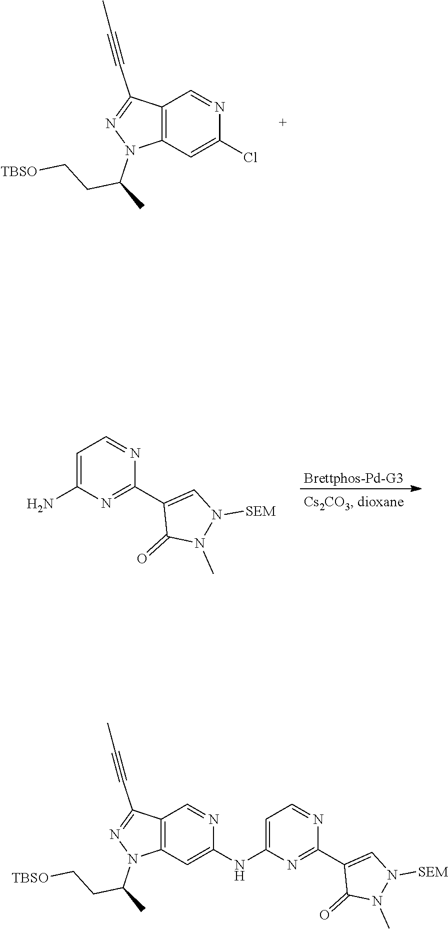 [SMILES: CC#Cc1nn([C@@H](C)CCO[Si](C)(C)C(C)(C)C)c2cc(Cl)ncc12 ; Cn1c(=O)c(-c2nccc(N)n2)cn1COCC[Si](C)(C)C ; "Brettphos-Pd-G3" ; "Cs2CO3, dioxane" ; CC#Cc1nn([C@@H](C)CCO[Si](C)(C)C(C)(C)C)c2cc(Nc3ccnc(-c4cn(COCC[Si](C)(C)C)n(C)c4=O)n3)ncc12]

(S)-1-(4-((tert-butyldimethylsilyl)oxy)butan-2-yl)-6-chloro-, prop-1-yn-1-yl)-1H-pyrazolo[4,3-c]pyridine (353 mg, 0.93 mmol) and 4-(4-aminopyrimidin-2-yl)-2-methyl-1-((2-(trimethylsilyl)ethoxy)methyl)-1,2-dihydro-3H-pyrazol-3-one (300 mg, 0.93 mmol) were dissolved in 1,4-dioxane (10 mL), and Cs₂CO₃ (608 mg, 1.86 mmol) and XantPhos Pd-G3 (169 mg, 0, 19 mmol) were added. Under nitrogen protection, the mixture was heated to 120° C., and stirred overnight. After the reaction was completed, the mixture was cooled to room temperature. Ethyl acetate was added to dilute the solution, and then the solution was washed with saturated brine and dried over anhydrous sodium sulfate. It was filtered and the filtrate was concentrated and separated by using column chromatography to obtain (S)-4-(4-((1-(4-((tert-butyldimethylsilyl)oxy)butan-2-yl)-3-(prop-1-yn-1-yl)-1H-pyrazolo[4,3-c]pyridin-6-yl)amino)pyrimidin-2-yl)-2-methyl-1-((2-(trimethylsilyl)ethoxy)meth yl)-1,2-dihydro-3H-pyrazol-3-one (0.32 g, yield: 51%). MS m/z (ESI): 663.4 [M+H]⁺.

### Step 2: Synthesis of (S)-4-(4-((1-(4-hydroxybutan-2-yl)-3-(prop-1-yn-1-yl)-1H-pyrazolo[4,3-c]pyridin-6-yl)amino)pyrimidin-2-yl)-2-methyl-1,2-dihydro-3H-pyrazol-3-one

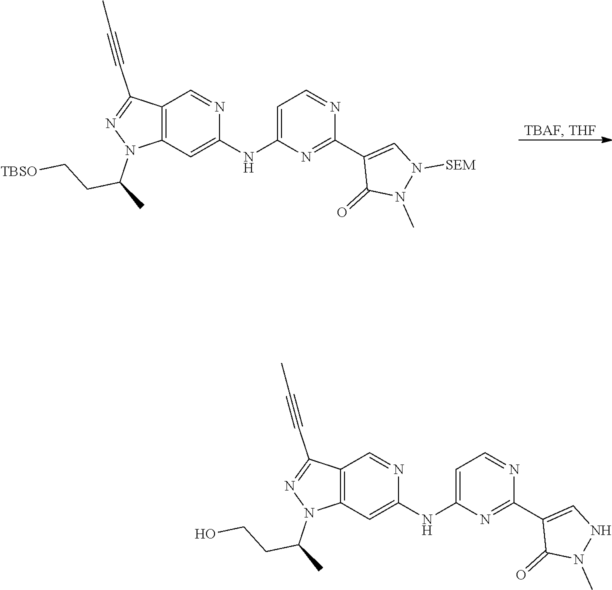

(S)-4-(4-((1-(4-((tert-butyldimethylsilyl)oxy)butan-2-yl)-3-(prop-1-yn-1-yl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one (320 mg, 0.48 mmol) was dissolved in THF (5 mL), and then IM TBAF/THF solution (2.9 mL, 2.90 mmol) was added. Under nitrogen protection, the mixture was heated to 70° C., and stirred for 2 hours. After the reaction was completed, the reaction solution was cooled to room temperature and concentrated. The residue was separated by using column chromatography to obtain (S)-4-(4-((1-(4-hydroxybutan-2-yl)-3-(prop-1-yn-1-yl)-1H-pyrazolo[4,3-c]pyridin-6-yl)amino)pyrimidin-2-yl)-2-methyl-1,2-dihydro-3H-pyrazol-3-one (98 mg, yield: 48%). MS m/z (ESI): 419.2 [M+H]⁺.

### Step 3: Synthesis of (S)-1¹,5-dimethyl-4³-(prop-1-yn-1-yl)-1¹H,4¹H-8-oxa-3-aza-4(6,1)-pyrazolo[4,3-c]pyridina-2(2,4)-pyrimidina-1(4,5)-pyrazolacyclooctaphane

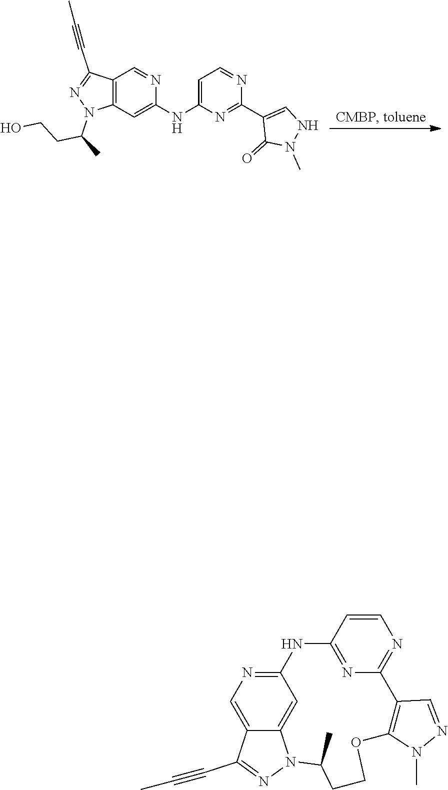 [SMILES: CC#Cc1nn([C@@H](C)CCO)c2cc(Nc3ccnc(-c4c[nH]n(C)c4=O)n3)ncc12 ; "CMBP, toluene" ; CC#Cc1nn2c3cc(ncc13)Nc1ccnc(n1)-c1cnn(C)c1OCC[C@@H]2C]

(S)-4-(4-((1-(4-hydroxybutan-2-yl)-3-(prop-1-yn-1-yl)-1H-pyrazolo[4,3-c]pyridin-6-yl)amino)pyrimidin-2-yl)-2-methyl-1,2-dihydro-3H-pyrazol-3-one (98 mg, 0.23 mmol) was dissolved in toluene (5 mL), and CMBP (339 mg, 1.41 mmol) was added. Under nitrogen protection, the mixture was heated to 130° C., and reacted for 24 hours. After the reaction was completed, the reaction solution was cooled to room temperature, diluted with CH₂Cl₂, washed with saturated aqueous NaHCO₃, dried over anhydrous sodium sulfate, filtered, and the filtrate was concentrated. The residue was separated by using column chromatography to obtain (S)-1¹,5-dimethyl-4³-(prop-1-yn-1-yl)-1¹H,4¹H8-oxa-3-aza-4(6,1)-pyrazolo[4,3-c]pyridina-2(2,4)-pyrimidina-1(4,5)-pyrazolacyclooctaphane-(8.2 mg, yield: 9%). MS m/z (ESI): 401.2 [M+H]⁺.

¹HNMR (400 MHz, DMSO-d₆) δ 10.33 (s, 1H), 9.09 (d, J=1.1 Hz, 1H), 8.75 (d, J=1.0 Hz, 1H), 8.31 (d, J=5.8 Hz, 1H), 7.93 (s, 1H), 6.74 (d, J=5.8 Hz, 1H), 5.14 (dd, J=10.4, 6.8 Hz, 1H), 4.37 (t, J=11.4 Hz, 1H), 4.01-3.87 (m, 1H), 3.76 (s, 3H), 2.17 (s, 3H), 1.99 (t, J=7.6 Hz, 1H), 1.75 (d, J=6.8 Hz, 3H).

Examples A2 to A3 can be prepared by selecting the corresponding raw materials with reference to the synthesis methods of Example A1:

| Example | | | MS: m/z |
|---|---|---|---|
| No. | Structural Formula | Chemical Name | [M + H]⁺ |
| A2 | 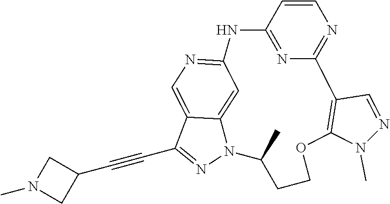 [SMILES: C[C@H]1CCOc2c(cnn2C)-c2nccc(n2)Nc2cc3c(cn2)c(C#CC2CN(C)C2)nn31] | (S)-1¹,5-dimethyl-4³-((1-meth- ylazetidin-3-yl)ethynyl)-1¹H,4¹H- 8-oxa-3-aza-4(6,1)-py- razolo[4,3-c]pyridina-2(2,4)- pyrimidina-1(4,5)-pyrazola- cyclooctaphane | 456.2 |
| A3 | 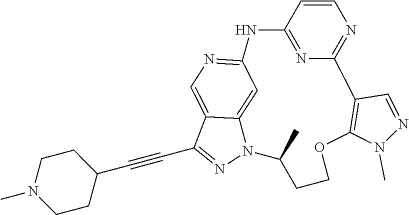 [SMILES: C[C@H]1CCOc2c(cnn2C)-c2nccc(n2)Nc2cc3c(cn2)c(C#CC2CCN(C)CC2)nn31] | (S)-1¹,5-dimethyl-4³-((1-meth- ylpiperidin-4-yl)ethynyl)- 1¹H,4¹H-8-oxa-3-aza-4(6,1)- pyrazolo[4,3-c]pyridina-2(2,4)- pyrimidina-1(4,5)-pyra- zolacyclooctaphane | 484.2 |

### Example 1: Preparation of 1′-methyl-3′-((1-methylpiperidin-4-yl)ethynyl)spiro(cyclopentane-1,6′-8-oxa-3-aza-4(6,1)-pyrazolo[4,3-c]pyridina-2(2,4)-pyrimidina-1(4,5)-pyrazolacyclooctaphane)

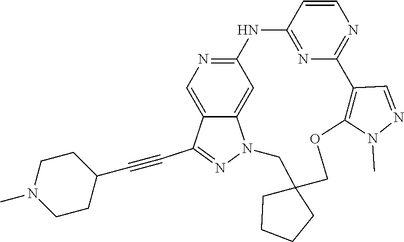 [SMILES: CN1CCC(C#Cc2nn3c4cc(ncc24)Nc2ccnc(n2)-c2cnn(C)c2OCC2(CCCC2)C3)CC1]

### Step 1: Synthesis of 4-(4-(1-((1-(hydroxymethyl)cyclopentyl)methyl)-3-((1-methylpiperidin-4-yl)ethynyl)-1H-pyrazol[4,3-c]pyridin-6-yl)amino)pyrimidin-2-yl)-2-methyl-1-((2-(trimethylsilyl)ethoxy)methyl)-1,2-dihydro-3H-pyrazol-3-one

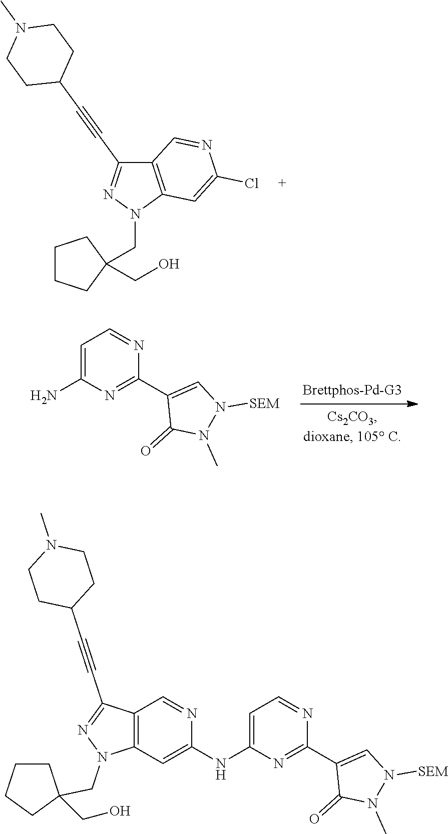

(1-((6-chloro-3-((1-methylpiperidin-4-yl)ethynyl)-1H-pyrazolo[4,3-c]pyridin-1-yl)methyl)cyclopentyl)methanol (245 mg, 0.633 mmol) was dissolved in 1,4-dioxane (15 mL), and then 4-(4-aminopyrimidin-2-yl)-2-methyl-1-((2-(trimethylsilyl)ethoxy)methyl)-1,2-dihydro-3H-pyrazol-3-one (224 mg, 0.697 mmol), Cs₂CO₃ (413 mg, 1.266 mmol) and Brettphos-Pd-G3 (115 mg, 0.127 mmol) were added. The mixture was heated to 105° C. and stirred overnight under nitrogen protection. After the reaction was completed, the reaction solution was cooled to room temperature, concentrated, and separated by using column chromatography to obtain 4-(4-((1-((1-(hydroxymethyl)cyclopentyl)methyl)-3-((1-methylpiperidin-4-yl)ethynyl)-1H-pyrazolo[4,3-c]pyridin-6-yl)amino)pyrimidin-2-yl)-2-methyl-1-((2-(trimethylsilyl)eth oxy)methyl)-1,2-dihydro-3H-pyrazol-3-one (210 mg, 0.219 mmol, yield: 34.55%). MS m/z (ESI): 672.6 [M+H]⁺.

### Step 2: Synthesis of 4-(4-((1-((1-(hydroxymethyl)cyclopentyl)methyl)-3-((1-methylpiperidin-4-yl)ethynyl)-1H-pyrazolo[4,3-c]pyridin-6-yl)amino)pyrimidin-2-yl)-2-methyl-1,2-dihydro-3H-pyrazol-3-one

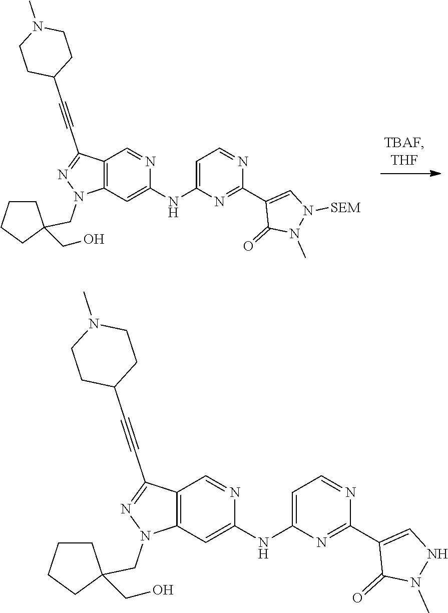 [SMILES: CN1CCC(C#Cc2nn(CC3(CO)CCCC3)c3cc(Nc4ccnc(-c5cn(COCC[Si](C)(C)C)n(C)c5=O)n4)ncc23)CC1 ; CN1CCC(C#Cc2nn(CC3(CO)CCCC3)c3cc(Nc4ccnc(-c5c[nH]n(C)c5=O)n4)ncc23)CC1]

4-(4-((1-((1-(hydroxymethyl)cyclopentyl)methyl)-3-((1-methylpiperidin-4-yl)ethynyl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one (210 mg, 0.219 mmol) was dissolved in THF (12 mL), and TBAF/THF solution (0.46 mL, 1 M, 0.459 mmol) was added. The mixture was heated to 70° C., and stirred for 1 hour. After the reaction was completed, the reaction solution was concentrated, the crude product was slurried with methanol, filtered and dried to obtain 4-(4-((1-((1-(hydroxymethyl)cyclopentyl)methyl)-3-((1-methylpiperidin-4-yl)ethynyl)-1H-pyrazolo[4,3-c]pyridin-6-yl)amino)pyrimidin-2-yl)-2-methyl-1,2-dihydro-3H-pyrazol-3-one (65 mg, 0.120 mmol, yield: 54.9%). MS m/z (ESI): 542.4 [M+H]⁺.

### Step 3: Synthesis of 1′-methyl-3′-((1-methylpiperidin-4-yl)ethynyl)spiro(cyclopentane-1,6′-8-oxa-3-aza-4(6,1)-pyrazolo[4,3-c]pyridina-2(2,4)-pyrimidina-1(4,5)-pyrazolacyclooctaphane)

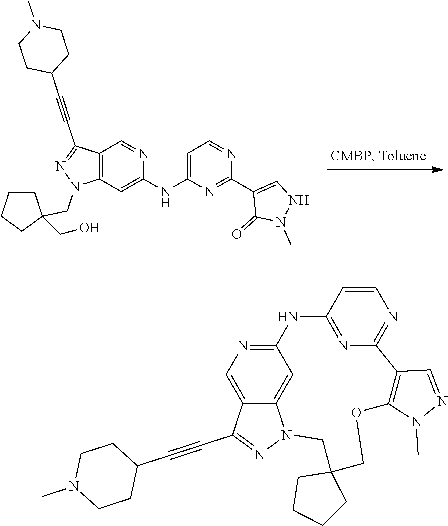 [SMILES: CN1CCC(C#Cc2nn(CC3(CO)CCCC3)c3cc(Nc4ccnc(-c5c[nH]n(C)c5=O)n4)ncc23)CC1 ; CN1CCC(C#Cc2nn3c4cc(ncc24)Nc2ccnc(n2)-c2cnn(C)c2OCC2(CCCC2)C3)CC1]

4-(4-((1-((1-(hydroxymethyl)cyclopentyl)methyl)-3-((1-methylpiperidin-4-yl)ethynyl)-1H-pyrazolo[4,3-c]pyridin-6-yl)amino)pyrimidin-2-yl)-2-methyl-1,2-dihydro-3H-pyrazol-3-one (60 mg, 0.111 mmol) was dissolved in toluene (15 mL), and then CMBP (214 mg, 0.886 mmol) was added. Under nitrogen protection, the mixture was heated to 130° C., and reacted overnight. After the reaction was completed, the mixture was cooled to room temperature, quenched by adding saturated aqueous sodium bicarbonate solution, and then extracted twice with dichloromethane. The organic phase was concentrated and separated by using column chromatography to obtain a crude product, which was then separated by preparative HPLC to obtain 1′-methyl-3′-((1-methylpiperidin-4-yl)ethynyl)spiro(cyclopentane-1,6′-8-oxa-3-aza-4(6,1)-pyrazolo[4,3-c]pyridina-2(2,4)-pyrimidina-1(4,5)-pyrazolacyclooctaphane) (14 mg, 0.024 mmol, yield: 21.7%). MS m/z (ESI): 524.4 [M+H]⁺.

¹H NMR (400 MHz, DMSO-d₆) δ 10.51 (s, 1H), 9.54 (s, 1H), 8.74 (s, 1H), 8.34 (d, J=5.8 Hz, 1H), 8.02 (s, 1H), 6.80 (d, J=5.8 Hz, 1H), 3.82 (s, 3H), 2.77-2.60 (m, 4H), 2.18 (s, 3H), 2.15-2.08 (m, 3H), 1.96-1.83 (m, 6H), 1.75-1.67 (m, 4H).

Examples 2 to 12 can be prepared by selecting the corresponding raw materials with reference to the synthesis methods of Example 1 or A1:

| Example | | | MS: m/z |
|---|---|---|---|
| No. | Structural Formula | Chemical Name | [M + H]⁺ |
| 2 | 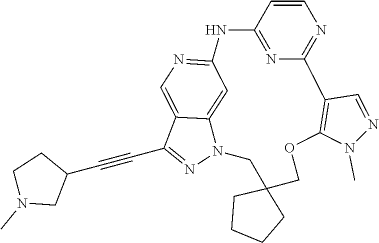 [SMILES: CN1CCC(C#Cc2nn3c4cc(ncc24)Nc2ccnc(n2)-c2cnn(C)c2OCC2(CCCC2)C3)C1] | l′-methyl-3′-((1-methylpyrro- lidin-3-yl)ethynyl)spiro(cyclo- pentane-1,6′-8-oxa-3-aza- 4(6,1)-pyrazolo[4,3-c]pyridi- na-2(2,4)-pyrimidina-1(4,5)- pyrazolacyclooctaphane) | 510.2 |
| 3 | 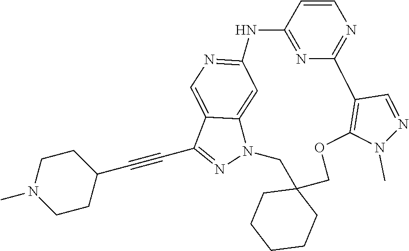 [SMILES: CN1CCC(C#Cc2nn3c4cc(ncc24)Nc2ccnc(n2)-c2cnn(C)c2OCC2(CCCCC2)C3)CC1] | 1′-methyl-3′-((1-methylpiper- idin-4-yl)ethynyl)spiro(cyclo- hexane-1,6′-8-oxa-3-aza-4(6,1)- pyrazolo[4,3-c]pyridina- 2(2,4)-pyrimidina-1(4,5)-py- razolacyclooctaphane) | 538.4 |
| 4 | 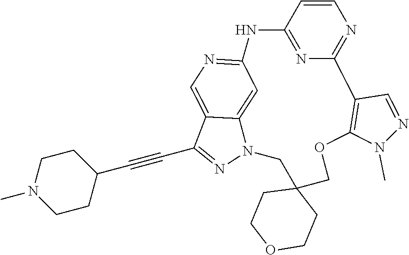 [SMILES: CN1CCC(C#Cc2nn3c4cc(ncc24)Nc2ccnc(n2)-c2cnn(C)c2OCC2(CCOCC2)C3)CC1] | 1′-methyl-3′-((1-methylpiper- idin-4-yl)ethynyl)-2,3,5,6-tetra- hydrospiro(pyran-4,6′-8-oxa- 3-aza-4(6,1)-pyrazolo[4,3- c]pyridina-2(2,4)-pyrimidina- 1(4,5)-pyrazolacyclooctaphane) | 540.2 |
| 5 | 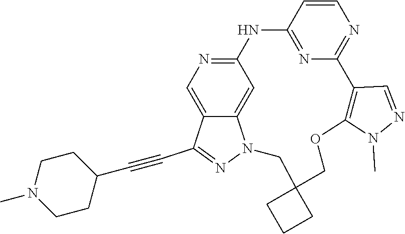 [SMILES: CN1CCC(C#Cc2nn3c4cc(ncc24)Nc2ccnc(n2)-c2cnn(C)c2OCC2(CCC2)C3)CC1] | 1′-methyl-3′-((1-methylpiper- idin-4-yl)ethynyl)spiro(cyclo- butane-1,6′-8-oxa-3-aza-4(6,1)- pyrazolo[4,3-c]pyridina- 2(2,4)-pyrimidina-1(4,5)-py- razolacyclooctaphane) | 510.4 |
| 6 | 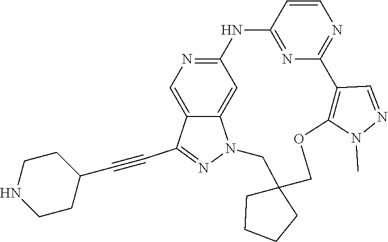 [SMILES: Cn1ncc2c1OCC1(CCCC1)Cn1nc(C#CC3CCNCC3)c3cnc(cc31)Nc1ccnc-2n1] | 1′-methyl-3′-(piperidin-4-yl- ethynyl)spiro(cyclopentane- 1,6′-8-oxa-3-aza-4(6,1)-pyra- zolo[4,3-c]pyridina-2(2,4)-py- rimidina-1(4,5)-pyrazolacy- clooctaphane) | 510.4 |
| 7 | 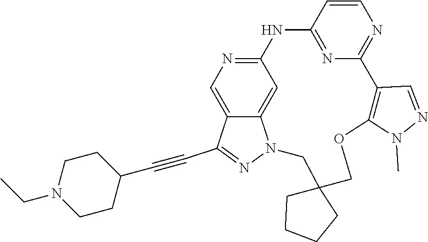 [SMILES: CCN1CCC(C#Cc2nn3c4cc(ncc24)Nc2ccnc(n2)-c2cnn(C)c2OCC2(CCCC2)C3)CC1] | 3′-((1-ethylpiperidin-4-yl)eth- ynyl)-1′-methylspiro(cyclo- pentane-1,6′-8-oxa-3-aza-4(6,1)- pyrazolo[4,3-c]pyridina- 2(2,4)-pyrimidina-1(4,5)-py- razolacyclooctaphane) | 538.4 |
| 8 | 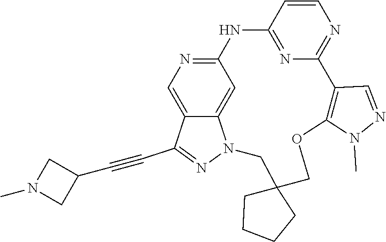 [SMILES: CN1CC(C#Cc2nn3c4cc(ncc24)Nc2ccnc(n2)-c2cnn(C)c2OCC2(CCCC2)C3)C1] | l′-methyl-3′-((1-methylazeti- din-3-yl)ethyny1)spiro(cyclo- pentane-1,6′-8-oxa-3-aza-4(6,1)- pyrazolo[4,3-c]pyridina- 2(2,4)-pyrimidina-1(4,5)-py- razolacyclooctaphane) | 496.4 |
| 9 | 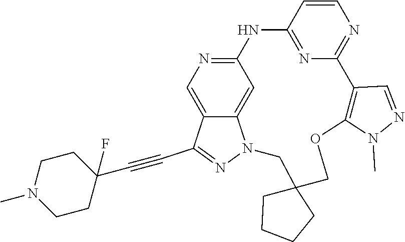 [SMILES: CN1CCC(F)(C#Cc2nn3c4cc(ncc24)Nc2ccnc(n2)-c2cnn(C)c2OCC2(CCCC2)C3)CC1] | 3′-((4-fluoro-1-methylpiperi- din-4-yl)ethynyl)-1′-methylspi- ro(cyclopentane-1,6′-8-ox- a-3-aza-4(6,1)-pyrazolo[4,3- c]pyridina-2(2,4)-pyrimidina- 1(4,5)-pyrazolacyclooctaphane) | 542.4 |
| 10 | 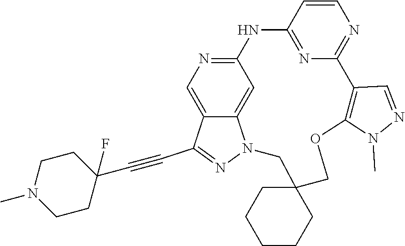 [SMILES: CN1CCC(F)(C#Cc2nn3c4cc(ncc24)Nc2ccnc(n2)-c2cnn(C)c2OCC2(CCCCC2)C3)CC1] | 3′-((4-fluoro-1-methylpiperi- din-4-yl)ethynyl)-1′-methylspi- ro(cyclohexane-1,6′-8-oxa- 3-aza-4(6,1)-pyrazolo[4,3-c] pyridina-2(2,4)-pyrimidina- 1(4,5)-pyrazolacyclooctaphane) | 556.4 |
| 11 | 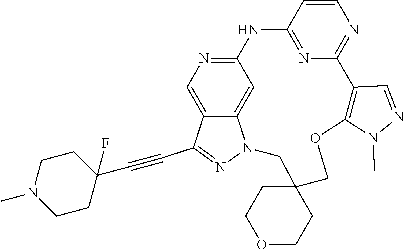 [SMILES: CN1CCC(F)(C#Cc2nn3c4cc(ncc24)Nc2ccnc(n2)-c2cnn(C)c2OCC2(CCOCC2)C3)CC1] | 3′-((4-fluoro-1-methylpiperi- din-4-yl)ethynyl)-1′-methyl- 2,3,5,6-tetrahydrospiro(pyran- 4,6′-8-oxa-3-aza-4(6,1)-py- razolo[4,3-c]pyridina-2(2,4)- pyrimidina-1(4,5)-pyrazolacy- clooctaphane) | 558.4 |
| 12 | 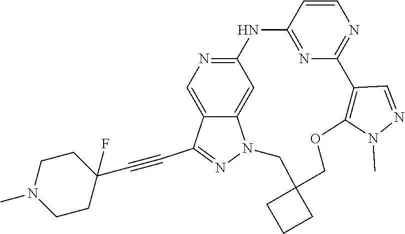 [SMILES: CN1CCC(F)(C#Cc2nn3c4cc(ncc24)Nc2ccnc(n2)-c2cnn(C)c2OCC2(CCC2)C3)CC1] | 3′-((4-fluoro-1-methylpiperi- din-4-yl)ethynyl)-1′-methylspi- ro(cyclobutane-1,6′-8-oxa- 3-aza-4(6,1)-pyrazolo[4,3-c] pyridina-2(2,4)-pyrimidina-1 (4,5)-pyrazolacyclooctaphane | 528.4 |

### Example 13: Preparation of (R)-2-(1-(1′-methylspiro[cyclopentane-1,6′-8-oxa-3-aza-4(6,1)-pyrazolol[4,3-c]pyridina-2(2,4)-pyrimidina-1(4,5)-pyrazolacyclooctaphane]-3′-yl)pyrrolidin-3-yl)propan-2-ol

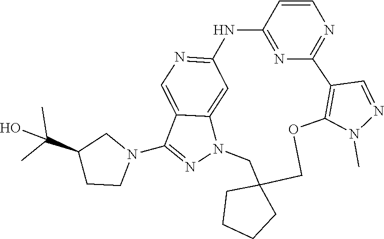 [SMILES: Cn1ncc2c1OCC1(CCCC1)Cn1nc(N3CC[C@@H](C(C)(C)O)C3)c3cnc(cc31)Nc1ccnc-2n1]

### Step 1: Synthesis of (R)-2-(1-(6-chloro-1-((1-(hydroxymethyl)cyclopentyl)methyl)-1H-pyrazolo[4,3-c]pyridin-3-yl)pyrrolidin-3-yl)propan-2-ol

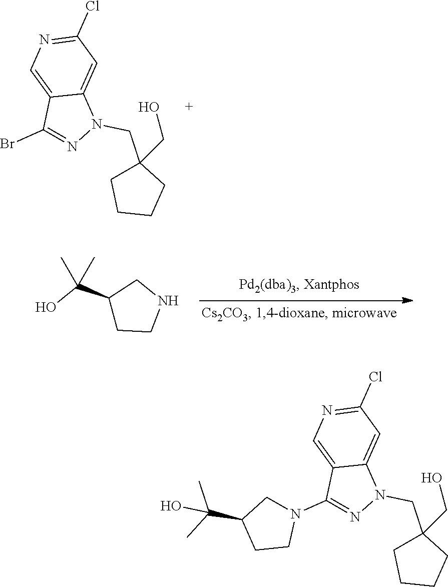

(R)-2-(pyrrolidin-3-yl)propan-2-ol hydrochloride (0.31 g, 1.86 mmol) and (1-((3-bromo-6-chloro-1H-pyrazolo[4,3-c]pyridin-1-yl)methyl)cyclopentyl)methanol (0.40 g, 1.16 mmol) were dissolved in 1,4-dioxane (8 mL), and then Cs₂C₀₃ (1.13 g, 3.48 mmol), Pd₂(dba)₃ (0.16 g, 0.17 mmol) and Xantphos (0.20 g, 0.35 mmol) were added. After the nitrogen substitution was carried out, the mixture was heated in a microwave to 80° C., and reacted for 2 hours. After the reaction was completed, ethyl acetate was added to dilute the reaction solution, and it was washed once with water and once with saturated brine respectively. The organic layer was separated, washed with saturated brine, and dried over anhydrous sodium sulfate. It was filtered and concentrated, and the resulting crude product was separated by using column chromatography (eluent: CH₂Cl₂/MeOH 0-5%) to obtain (R)-2-(1-(6-chloro-1-((1-(hydroxymethyl)cyclopentyl)methyl)-1H-pyrazolo[4,3-c]pyridin-3-yl)pyrrolidin-3-yl)propan-2-ol (0.28 g, purity: 72.4%, yield: 44.1%). MS m/z (ESI): 393.2 [M+H]⁺.

### Step 2: Synthesis of (R)-4-(4-((1-((1-(hydroxymethyl)cyclopentyl)methyl)-3-(3-(2-hydroxypropan-2-yl)pyrrolidin-1-yl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one

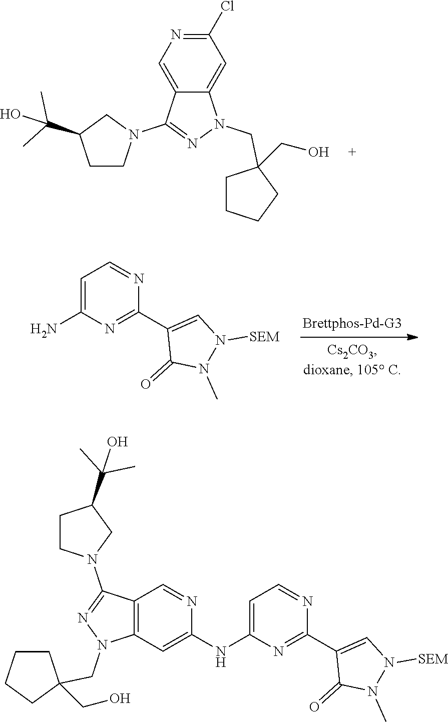 [SMILES: CC(C)(O)[C@@H]1CCN(c2nn(CC3(CO)CCCC3)c3cc(Cl)ncc23)C1 ; Cn1c(=O)c(-c2nccc(N)n2)cn1COCC[Si](C)(C)C ; Cn1c(=O)c(-c2nccc(Nc3cc4c(cn3)c(N3CC[C@@H](C(C)(C)O)C3)nn4CC3(CO)CCCC3)n2)cn1COCC[Si](C)(C)C]

(R)-2-(1-(6-chloro-1-((1-(hydroxymethyl)cyclopentyl)methyl)-1H-pyrazolo[4,3-c]pyridin-3-yl)pyrrolidin-3-yl)propan-2-ol (0.40 g, 0.74 mmol) and 4-(4-aminopyrimidin-2-yl)-2-methyl-1-((2-(trimethylsilyl)ethoxy)methyl)-1,2-dihydro-3H-pyrazol-3-one (0.24 g, 0.74 mmol) were dissolved in 1,4-dioxane (8 mL), and then Cs₂CO₃ (0.48 g, 1.47 mmol) and Brettphos-Pd-G3 (0.13 g, 0.15 mmol) were added. After the nitrogen substitution was carried out, the mixture was heated in a microwave to 130° C., and reacted for 2 hours. After the reaction solution was cooled, ethyl acetate was added to dilute it, and the mixture was washed once with water and once with saturated brine, respectively. The organic layer was separated, dried over anhydrous sodium sulfate and then concentrated. The resulting crude product was separated by using column chromatography (eluent: CH₂C₂/MeOH 0-6%) to obtain (R)-4-(4-((1-((1-(hydroxymethyl)cyclopentyl)methyl)-3-(3-(2-hydroxypropan-2-yl)pyrrolidin-1-yl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one (0.38 g, purity: 62.6%, yield: 47.6%). MS m/z (ESI): 678.6 [M+H]⁺.

### Step 3: Synthesis of (R)-4-(4-((1-((1-(hydroxymethyl)cyclopentyl)methyl)-3-(3-(2-hydroxypropan-2-yl)pyrrolidin-1-yl)-1H-pyrazolo[4,3-c]pyridin-6-yl)amino)pyrimidin-2-yl)-1-methyl-1H-pyrazol-5-ol

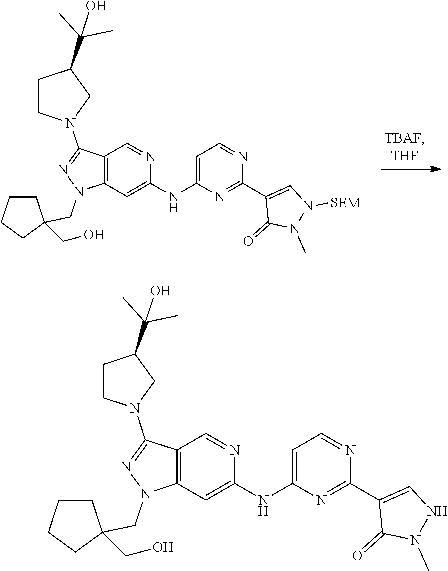 [SMILES: Cn1c(=O)c(-c2nccc(Nc3cc4c(cn3)c(N3CC[C@@H](C(C)(C)O)C3)nn4CC3(CO)CCCC3)n2)cn1COCC[Si](C)(C)C ; Cn1[nH]cc(-c2nccc(Nc3cc4c(cn3)c(N3CC[C@@H](C(C)(C)O)C3)nn4CC3(CO)CCCC3)n2)c1=O]

(R)-4-(4-((1-((1-(hydroxymethyl)cyclopentyl)methyl)-3-(3-(2-hydroxypropan-2-yl) pyrrolidin-1-yl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one (370 mg, purity: 62.6%, 0.35 mmol) was dissolved in THF (5.0 mL), and TBAF/THF solution (2.11 mL, 1.0 M, 2.11 mmol) was added. The mixture was heated to 70° C., and reacted for 1 hour. After the reaction was completed, the reaction solution was cooled to room temperature, and then concentrated and separated by using column chromatography to obtain (R)-4-(4-((1-((1-(hydroxymethyl)cyclopentyl)methyl)-3-(3-(2-hydroxypropan-2-yl)pyrrolidin-1-yl)-1H-pyrazolo[4,3-c]pyridin-6-yl)amino)pyrimidin-2-yl)-1-methyl-1H-pyrazol-5-ol (200 mg, purity: 85.4%, yield: 89.9%). MS m/z (ESI): 548.4 [M+H]⁺.

### Step 4: Synthesis of (R)-2-(1-(1′-methylspiro[cyclopentane-1,6′-8-oxa-3-aza-4(6,1)-pyrazolo[4,3-c]pyridina-2(2,4)-pyrimidina-1(4,5)-pyrazolacyclooctaphane]-3′-yl)pyrrolidin-3-yl)propan-2-ol

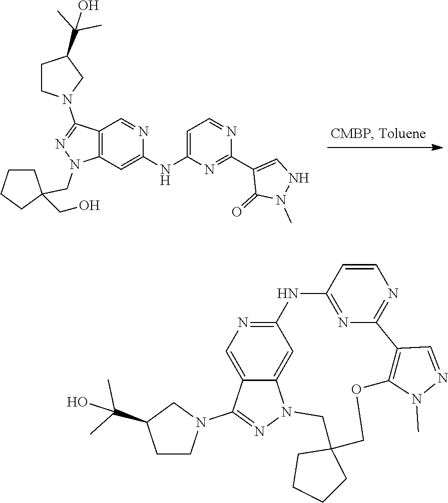 [SMILES: Cn1[nH]cc(-c2nccc(Nc3cc4c(cn3)c(N3CC[C@@H](C(C)(C)O)C3)nn4CC3(CO)CCCC3)n2)c1=O ; Cn1ncc2c1OCC1(CCCC1)Cn1nc(N3CC[C@@H](C(C)(C)O)C3)c3cnc(cc31)Nc1ccnc-2n1]

(R)-4-(4-((1-((1-(hydroxymethyl)cyclopentyl)methyl)-3-(3-(2-hydroxypropan-2-yl) pyrrolidin-1-yl)-1H-pyrazolo[4,3-c]pyridin-6-yl)amino)pyrimidin-2-yl)-1-methyl-1H-pyrazol-5-ol (180 mg, purity: 85.4%, 0.28 mmol) was dissolved in toluene (5 mL), and CMBP (542 mg, 2.25 mmol) was added. The mixture was heated to 130° C., and reacted for 24 hours. After the reaction was completed, the reaction solution was cooled to room temperature and saturated aqueous NaHCO₃ solution was added to quench the reaction. The mixture was extracted twice with ethyl acetate, and the organic layer was combined, washed with saturated brine, and dried over anhydrous sodium sulfate. It was filtered, concentrated, and separated by using column chromatography (eluent: CH₂Cl₂/MeOH 0-5%) to obtain (R)-2-(1-(1′-methylspiro[cyclopentane-1,6′-8-oxa-3-aza-4(6,1)-pyrazolo[4,3-c]pyridina-2(2,4)-pyrimidina-1(4,5)-pyrazolacyclooctaphane]-3′-yl)pyrrolidin-3-yl)propan-2-ol (34.4 mg, yield: 22.9%). MS m/z (ESI): 530.4 [M+H]⁺. ¹HNMR (400 MHz, DMSO-d₆) δ 10.26 (s, 1H), 9.18 (s, 1H), 8.71 (s, 1H), 8.29 (d, J=5.8 Hz, 1H), 8.00 (s, 1H), 6.77 (d, J=5.9 Hz, 1H), 4.37 (s, 1H), 3.80 (s, 3H), 3.71-3.61 (m, 1H), 3.60-3.38 (m, 3H), 2.41-2.28 (m, 11H), 1.92 (q, J=9.4 Hz, 4H), 1.70 (brs, 2H), 1.17 (brs, 3H), 1.15 (s, 3H).

### Example 14: Preparation of (S)-1′-methyl-3′-(3-(((trifluoromethyl)sulfonyl)methyl)pyrrolidin-1-yl)spiro[cyclopentane-1,6′-8-oxa-3-aza-4(6,1)-pyrazolo[4,3-c]pyridina-2(2,4)-pyrimidina-1(4,5)-pyrazolacyclooctaphane]

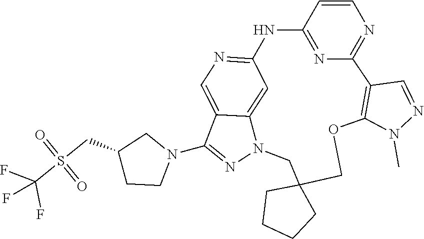 [SMILES: Cn1ncc2c1OCC1(CCCC1)Cn1nc(N3CC[C@H](CS(=O)(=O)C(F)(F)F)C3)c3cnc(cc31)Nc1ccnc-2n1]

### Step 1: Synthesis of (S)-4-(4-((1-((1-(hydroxymethyl)cyclopentyl)methyl)-3-(3-(((trifluoromethyl)sulfon yl)methyl)pyrrolidin-1-yl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one

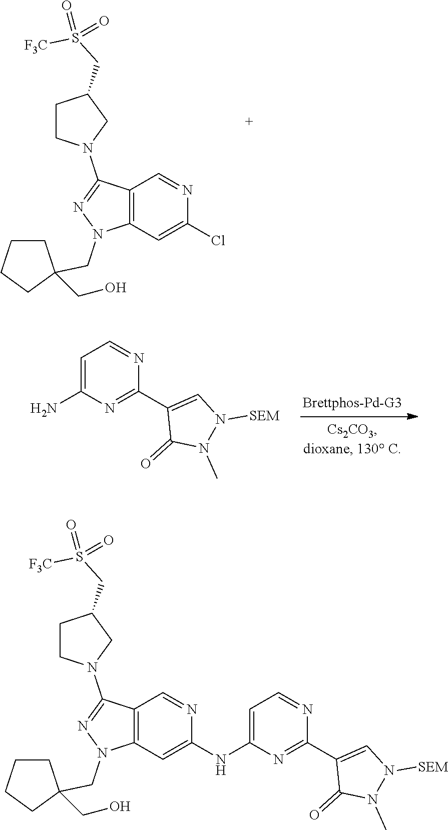 [SMILES: O=S(=O)(C[C@H]1CCN(c2nn(CC3(CO)CCCC3)c3cc(Cl)ncc23)C1)C(F)(F)F ; Cn1c(=O)c(-c2nccc(N)n2)cn1COCC[Si](C)(C)C ; Cn1c(=O)c(-c2nccc(Nc3cc4c(cn3)c(N3CC[C@H](CS(=O)(=O)C(F)(F)F)C3)nn4CC3(CO)CCCC3)n2)cn1COCC[Si](C)(C)C]

(S)-(1-((6-chloro-3-(3-(((trifluoromethyl)sulfonyl)methyl)pyrrolidin-1-yl)-1H-pyrazolo[4,3-c]pyridin-1-yl)methyl)cyclopentyl)methanol (0.48 g, 0.98 mmol) and 4-(4-aminopyrimidin-2-yl)-2-methyl-1-((2-(trimethylsilyl)ethoxy)methyl)-1,2-dihydro-3H-pyrazol-3-one (330 mg, 1.03 mmol) were dissolved in 1,4-dioxane (8 mL), and then Cs₂CO₃ (0.64 g, 1.95 mmol) and Brettphos-Pd-G3 (0.18 g, 0.195 mmol) were added. After the nitrogen substitution was carried out, the mixture was heated in a microwave to 130° C., and reacted for 2 hours. The reaction solution was cooled and diluted with ethyl acetate, and then washed once with water and saturated brine in sequence. The organic layer was separated and dried over anhydrous sodium sulfate. It was filtered and concentrated, and the resulting crude product was separated by using column chromatography to obtain (S)-4-(4-((1-((1-(hydroxymethyl)cyclopentyl)methyl)-3-(3-(((trifluoromethyl)sulfonyl)methyl)pyrrolidin-1-yl)-1H-pyrazolo[4,3-c]pyridin-6-yl)amino)pyrimidin-2-yl)-2-methyl-1-((2-(trimethylsilyl)ethoxy)methyl)-1,2-dihydro-3H-pyrazol-3-one (0.32 g, purity: 73.6%, yield: 31.5%). MS m/z (ESI): 766.4 [M+H]⁺.

### Step 2: Synthesis of (S)-4-(4-((1-((1-(hydroxymethyl)cyclopentyl)methyl)-3-(3-(((trifluoromethyl)sulfon yl)methyl)pyrrolidin-1-yl)-1H-pyrazolo[4,3-c]pyridin-6-yl)amino)pyrimidin-2-yl)-2-methyl-1,2-dihydro-3H-pyrazol-3-one

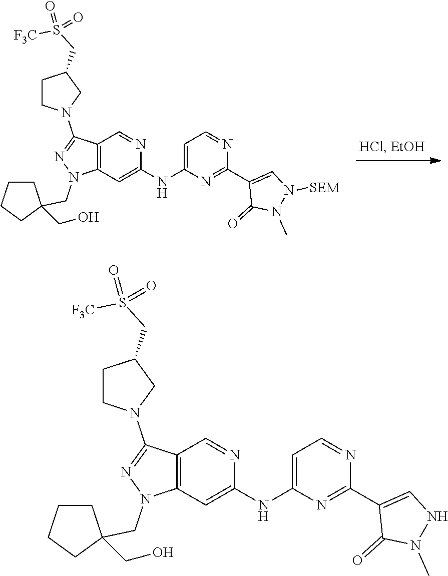 [SMILES: Cn1c(=O)c(-c2nccc(Nc3cc4c(cn3)c(N3CC[C@H](CS(=O)(=O)C(F)(F)F)C3)nn4CC3(CO)CCCC3)n2)cn1COCC[Si](C)(C)C ; Cn1[nH]cc(-c2nccc(Nc3cc4c(cn3)c(N3CC[C@H](CS(=O)(=O)C(F)(F)F)C3)nn4CC3(CO)CCCC3)n2)c1=O]

(S)-4-(4-((1-((1-(hydroxymethyl)cyclopentyl)methyl)-3-(3-(((trifluoromethyl)sulfonyl)methyl)pyrrolidin-1-yl)-1H-pyrazolo[4,3-c]pyridin-6-yl)amino)pyrimidin-2-yl)-2-m ethyl-1-((2-(trimethylsilyl)ethoxy)methyl)-1,2-dihydro-3H-pyrazol-3-one (320 mg, purity: 73.6%, 0.31 mmol) was dissolved in ethanol (5.0 mL), and concentrated hydrochloric acid (0.50 mL, 12 M, 6.00 mmol) was added. The mixture was heated to 70° C., and reacted for 2 hours. After the reaction was completed, it was cooled to room temperature. The reaction solution was concentrated and separated by using column chromatography (eluent: CH₂Cl₂/MeOH 0-5%) to obtain (S)-4-(4-((1-((1-(hydroxymethyl)cyclopentyl)methyl)-3-(3-(((trifluoromethyl)sulfonyl)methyl)pyrrolidin-1-yl)-1H-pyrazolo[4,3-c]pyridin-6-yl)amino)pyrimidin-2-yl)-2-methyl-1,2-dihydro-3H-pyrazol-3-one (150 mg, purity: 86.8%, yield: 65.1%). MS m/z (ESI): 636.4 [M+H]⁺.

### Step 3: Synthesis of (S)-1′-methyl-3′-(3-(((trifluoromethyl)sulfonyl)methyl)pyrrolidin-1-yl)spiro[cyclopentane-1,6′-8-oxa-3-aza-4(6,1)-pyrazolo[4,3-c]pyridina-2(2,4)-pyrimidina-1(4,5)-pyrazolacyclooctaphane]

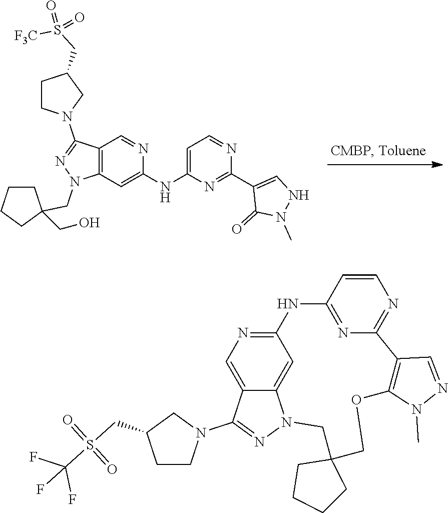 [SMILES: Cn1[nH]cc(-c2nccc(Nc3cc4c(cn3)c(N3CC[C@H](CS(=O)(=O)C(F)(F)F)C3)nn4CC3(CO)CCCC3)n2)c1=O ; Cn1ncc2c1OCC1(CCCC1)Cn1nc(N3CC[C@H](CS(=O)(=O)C(F)(F)F)C3)c3cnc(cc31)Nc1ccnc-2n1]

(S)-4-(4-((1-((1-(hydroxymethyl)cyclopentyl)methyl)-3-(3-(((trifluoromethyl)sulfonyl)methyl)pyrrolidin-1-yl)-1H-pyrazolo[4,3-c]pyridin-6-yl)amino)pyrimidin-2-yl)-2-methyl-1,2-dihydro-3H-pyrazol-3-one (150 mg, purity: 86.8%, 0.21 mmol) was dissolved in dry toluene (20 mL), and CMBP (400 mg, 1.64 mmol) was added. The mixture was heated to 130° C., and reacted for 24 hours. After the reaction was completed, the reaction solution was cooled to room temperature, quenched by adding saturated aqueous NaHCO₃ solution, and extracted twice with ethyl acetate. The organic phase was combined, washed sequentially with saturated brine, and dried over anhydrous sodium sulfate. It was filtered and concentrated. The resulting crude product was separated by preparative HPLC to obtain (S)-1′-methyl-3′-(3-(((trifluoromethyl)sulfonyl)methyl)pyrrolidin-1-yl)spiro[cyclopentane-1,6′-8-oxa-3-aza-4(6,1)-pyrazolo[4,3-c]pyridina-2(2,4)-pyrimidina-1(4,5)-pyrazolacyclooctaphane] (26.8 mg, yield: 20.7%). MS m/z (ESI): 618.4 [M+H]⁺.

¹HNMR (400 MHz, DMSO-d₆) δ 10.28 (s, 1H), 9.21 (s, 1H), 8.69 (s, 1H), 8.30 (d, J=5.8 Hz, 1H), 8.00 (s, 1H), 6.76 (d, J=5.9 Hz, 1H), 4.60 (brs, 2H), 4.11 (d, J=6.7 Hz, 2H), 3.89-3.69 (m, 5H), 3.61 (q, J=8.3 Hz, 1H), 3.38 (t, J=9.0 Hz, 1H), 2.90 (p, J=7.6 Hz, 1H), 2.33-2.29 (m, 1H), 2.05-1.84 (m, 4H), 1.70 (brs, 2H).

Examples 15 to 47 and Example A4 can be prepared by selecting the corresponding raw materials with reference to the routes of Example A1, 1, 13 or 14:

| Example | | | MS: m/z |
|---|---|---|---|
| No. | Structural Formula | Chemical Name | [M + H]⁺ |
| 15 | 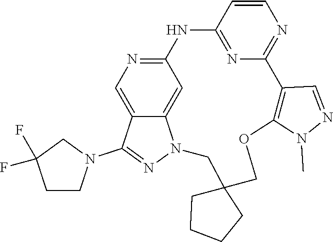 [SMILES: Cn1ncc2c1OCC1(CCCC1)Cn1nc(N3CCC(F)(F)C3)c3cnc(cc31)Nc1ccnc-2n1] | 3′-(3,3-difluoropyrrolidin-1- yl)-1′-methylspiro(cyclopen- tane-1,6′-8-oxa-3-aza-4(6,1)- pyrazolo[4,3-c]pyridina- 2(2,4)-pyrimidina-1(4,5)-py- razolacyclooctaphane) | 508.2 |
| 16 | 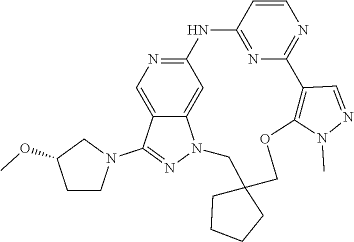 [SMILES: CO[C@H]1CCN(c2nn3c4cc(ncc24)Nc2ccnc(n2)-c2cnn(C)c2OCC2(CCCC2)C3)C1] | (S)-3′-(3-methoxypyrrolidin- 1-yl)-l′-methylspiro(cyclo- pentane-1,6′-8-oxa-3-aza-4(6,1)- pyrazolo[4,3-c]pyridina- 2(2,4)-pyrimidina-1(4,5)- pyrazolacyclooctaphane) | 502.2 |
| 17 | 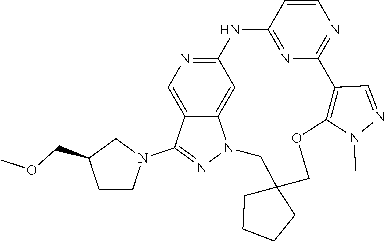 [SMILES: COC[C@@H]1CCN(c2nn3c4cc(ncc24)Nc2ccnc(n2)-c2cnn(C)c2OCC2(CCCC2)C3)C1] | (R)-3′-(3-(methoxymethyl) pyrrolidin-1-yl)-1′-methylspi- ro(cyclopentane-1,6′-8-oxa- 3-aza-4(6,1)-pyrazolo[4,3- c]pyridina-2(2,4)-pyrimidi- na-1(4,5)-pyrazolacycloocta- phane) | 516.4 |
| 18 | 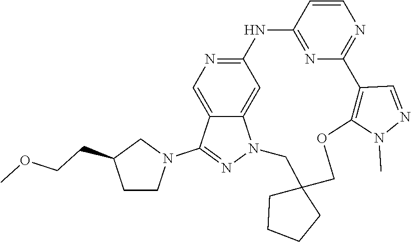 [SMILES: COCC[C@@H]1CCN(c2nn3c4cc(ncc24)Nc2ccnc(n2)-c2cnn(C)c2OCC2(CCCC2)C3)C1] | (S)-3′-(3-(2-methoxyethyl)pyr- rolidin-1-yl)-1′-methylspi- ro(cyclopentane-1,6′-8-oxa- 3-aza-4(6,1)-pyrazolo[4,3-c] pyridina-2(2,4)-pyrimidina- 1(4,5)-pyrazolacyclooctaphane) | 530.4 |
| 19 | 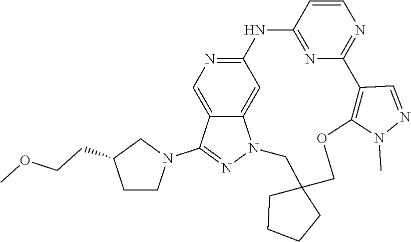 [SMILES: COCC[C@H]1CCN(c2nn3c4cc(ncc24)Nc2ccnc(n2)-c2cnn(C)c2OCC2(CCCC2)C3)C1] | (R)-3′-(3-(2-methoxyethyl) pyrrolidin-1-yl)-1′-methylspi- ro(cyclopentane-1,6′-8-oxa- 3-aza-4(6,1)-pyrazolo[4,3- c]pyridina-2(2,4)-pyrimidi- na-1(4,5)-pyrazolacycloocta- phane) | 530.4 |
| 20 | 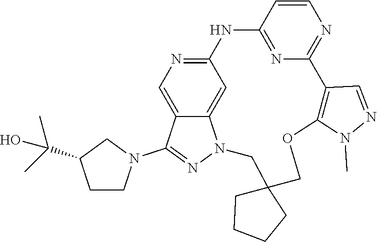 [SMILES: Cn1ncc2c1OCC1(CCCC1)Cn1nc(N3CC[C@H](C(C)(C)O)C3)c3cnc(cc31)Nc1ccnc-2n1] | (S)-2-(1-(1′-methylspiro[cy- clopentane-1,6′-8-oxa-3-aza- 4(6,1)-pyrazolo[4,3-c]pyridi- na-2(2,4)-pyrimidina-1(4,5)- pyrazolacyclooctaphane]- 3′-yl)pyrrolidin-3-yl)propan- 2-ol | 530.4 |
| 21 | 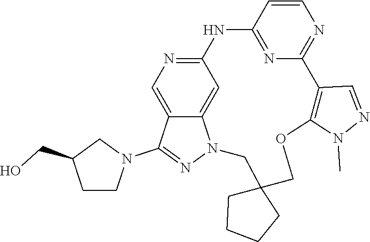 [SMILES: Cn1ncc2c1OCC1(CCCC1)Cn1nc(N3CC[C@@H](CO)C3)c3cnc(cc31)Nc1ccnc-2n1] | (R)-(1-(1′-methylspiro(cyclo- pentane-1,6′-8-oxa-3-aza- 4(6,1)-pyrazolo[4,3-c]pyridi- na-2(2,4)-pyrimidina-1(4,5)- pyrazolacyclooctaphane)-3′- yl)pyrrolidin-3-yl)methanol | 502.4 |
| 22 | 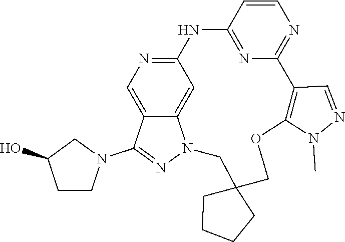 [SMILES: Cn1ncc2c1OCC1(CCCC1)Cn1nc(N3CC[C@@H](O)C3)c3cnc(cc31)Nc1ccnc-2n1] | (R)-1-(1′-methylspiro(cyclo- pentane-1,6′-8-oxa-3-aza-4(6,1)- pyrazolo[4,3-c]pyridina- 2(2,4)-pyrimidina-1(4,5)- pyrazolacy clooctaphane)-3′- yl)pyrrolidin-3-ol | 488.4 |
| 23 | 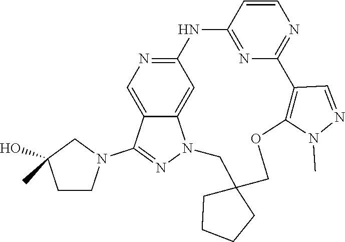 [SMILES: Cn1ncc2c1OCC1(CCCC1)Cn1nc(N3CC[C@](C)(O)C3)c3cnc(cc31)Nc1ccnc-2n1] | (S)-3-methyl-1-(1′-methylspi- ro(cyclopentane-1,6′-8-oxa- 3-aza-4(6,1)-pyrazolo[4,3- c]pyridina-2(2,4)-pyrimidina- 1(4,5)-pyrazolacycloocta- phane)-3′-yl)pyrrolidin-3-ol | 502.4 |
| 24 | 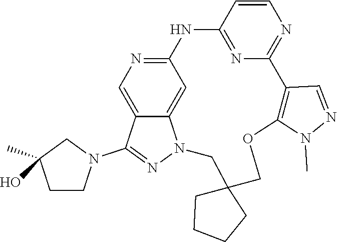 [SMILES: Cn1ncc2c1OCC1(CCCC1)Cn1nc(N3CC[C@@](C)(O)C3)c3cnc(cc31)Nc1ccnc-2n1] | (R)-3-methyl-1-(1′-methylspi- ro(cyclopentane-1,6′-8-oxa- 3-aza-4(6,1)-pyrazolo[4,3- c]pyridina-2(2,4)-pyrimidi- na-1(4,5)-pyrazolacycloocta- phane)-3′-yl)pyrrolidin-3-ol | 502.4 |
| 25 | 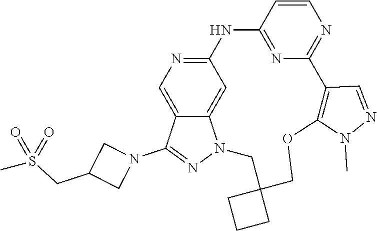 [SMILES: Cn1ncc2c1OCC1(CCC1)Cn1nc(N3CC(CS(C)(=O)=O)C3)c3cnc(cc31)Nc1ccnc-2n1] | l′-methyl-3′-(3-((methylsul- fonyl)methyl)azetidin-1-yl)spi- ro(cyclobutane-1,6′-8-oxa- 3-aza-4(6,1)-pyrazolo[4,3- c]pyridina-2(2,4)-pyrimidina- 1(4,5)-pyrazolacyclooctaphane) | 536.2 |
| 26 | 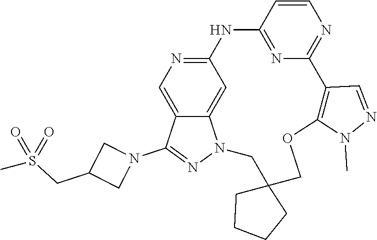 [SMILES: Cn1ncc2c1OCC1(CCCC1)Cn1nc(N3CC(CS(C)(=O)=O)C3)c3cnc(cc31)Nc1ccnc-2n1] | l′-methyl-3′-(3-((methylsul- fonyl)methyl)azetidin-1-yl)spi- ro(cyclopentane-1,6′-8-oxa- 3-aza-4(6,1)-pyrazolo[4,3- c]pyridina-2(2,4)-pyrimidi- na-1(4,5)-pyrazolacycloocta- phane) | 550.2 |
| 27 | 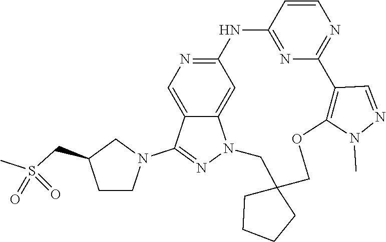 [SMILES: Cn1ncc2c1OCC1(CCCC1)Cn1nc(N3CC[C@@H](CS(C)(=O)=O)C3)c3cnc(cc31)Nc1ccnc-2n1] | (R)-1′-methyl-3′-(3-((meth- ylsulfonyl)methyl)pyrrolidin- 1-yl)spiro(cyclopentane-1,6′- 8-oxa-3-aza-4(6,1)-pyra- zolo[4,3-c]pyridina-2(2,4)-py- rimidina-1(4,5)-pyrazolacy- clooctaphane) | 564.3 |
| 28 | 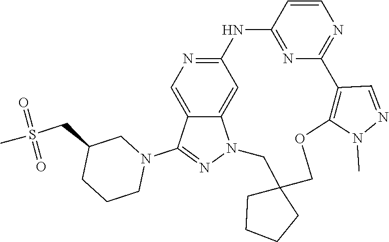 [SMILES: Cn1ncc2c1OCC1(CCCC1)Cn1nc(N3CCC[C@@H](CS(C)(=O)=O)C3)c3cnc(cc31)Nc1ccnc-2n1] | (R)-1′-methyl-3′-(3-((meth- ylsulfonyl)methyl)piperidin- 1-yl)spiro(cyclopentane-1,6′- 8-oxa-3-aza-4(6,1)-pyrazo- lo[4,3-c]pyridina-2(2,4)-py- rimidina-1(4,5)-pyrazolacyclo- octaphane) | 578.4 |
| 29 | 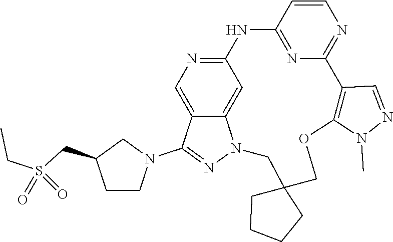 [SMILES: CCS(=O)(=O)C[C@@H]1CCN(c2nn3c4cc(ncc24)Nc2ccnc(n2)-c2cnn(C)c2OCC2(CCCC2)C3)C1] | (R)-3′-(3-((ethylsulfonyl)meth- yl)pyrrolidin-1-yl)-1′-meth- ylspiro(cyclopentane-1,6′- 8-oxa-3-aza-4(6,1)-pyrazolo [4,3-c]pyridina-2(2,4)-pyri- midina-1(4,5)-pyrazolacyclo- octaphane) | 578.4 |
| 30 | 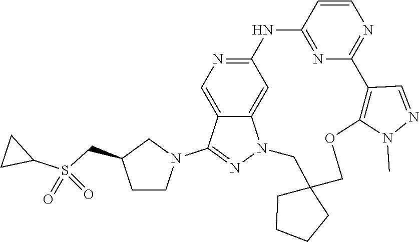 [SMILES: Cn1ncc2c1OCC1(CCCC1)Cn1nc(N3CC[C@@H](CS(=O)(=O)C4CC4)C3)c3cnc(cc31)Nc1ccnc-2n1] | R)-3′-(3-((cyclopropylsulfo- nyl)methyl)pyrrolidin-1-yl)- 1′-methylspiro(cyclopentane- 1,6′-8-oxa-3-aza-4(6,1)-py- razolo[4,3-c]pyridina-2(2,4)- pyrimidina-1(4,5)-pyrazo- lacyclooctaphane) | 590.4 |
| 31 | 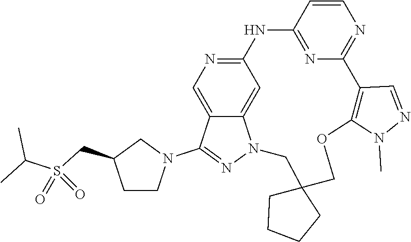 [SMILES: CC(C)S(=O)(=O)C[C@@H]1CCN(c2nn3c4cc(ncc24)Nc2ccnc(n2)-c2cnn(C)c2OCC2(CCCC2)C3)C1] | (R)-3′-(3-((isopropylsulfonyl) methyl)pyrrolidin-1-yl)-1′- methylspiro(cyclopentane- 1,6′-8-oxa-3-aza-4(6,1)-pyra- zolo[4,3-c]pyridina-2(2,4)- pyrimidina-1(4,5)-pyrazolacy- clooctaphane) | 592.4 |
| 32 | 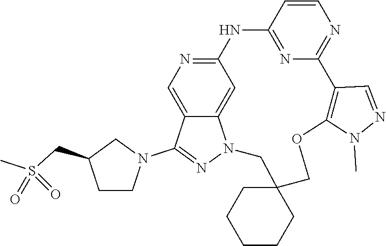 [SMILES: Cn1ncc2c1OCC1(CCCCC1)Cn1nc(N3CC[C@@H](CS(C)(=O)=O)C3)c3cnc(cc31)Nc1ccnc-2n1] | (R)-1′-methyl-3′-(3-((meth- ylsulfonyl)methyl)pyrrolidin- 1-yl)spiro(cyclohexane-1,6′- 8-oxa-3-aza-4(6,1)-pyrazo- lo[4,3-c]pyridina-2(2,4)-py- rimidina-1(4,5)-pyrazolacyclo- octaphane) | 578.4 |
| 33 | 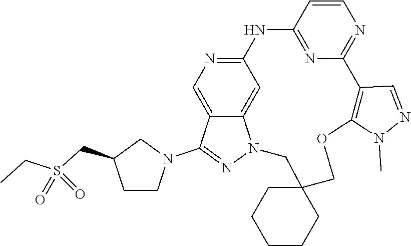 [SMILES: CCS(=O)(=O)C[C@@H]1CCN(c2nn3c4cc(ncc24)Nc2ccnc(n2)-c2cnn(C)c2OCC2(CCCCC2)C3)C1] | (R)-1′-methyl-3′-(3-((ethylsul- fonyl)methyl)pyrrolidin-1- yl)spiro(cyclohexane-1,6′- 8-oxa-3-aza-4(6,1)-pyrazolo [4,3-c]pyridina-2(2,4)-pyri- midina-1(4,5)-pyrazolacyclo- octaphane) | 592.4 |
| 34 | 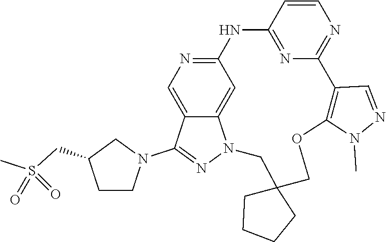 [SMILES: Cn1ncc2c1OCC1(CCCC1)Cn1nc(N3CC[C@H](CS(C)(=O)=O)C3)c3cnc(cc31)Nc1ccnc-2n1] | (S)-1′methyl-3′-(3-((methyl- sulfonyl)methyl)pyrrolidin- 1-yl)spiro(cyclopentane-1,6-′ 8-oxa-3-aza-4(6,1)-pyrazo- lo[4,3-c]pyridina-2(2,4)-pyri- midina-1(4,5)-pyrazolacyclo- octaphane) | 564.4 |
| 35 | 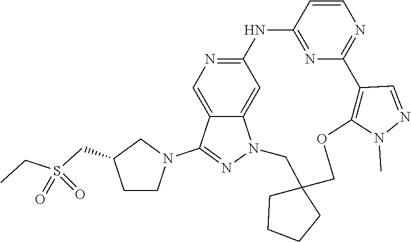 [SMILES: CCS(=O)(=O)C[C@H]1CCN(c2nn3c4cc(ncc24)Nc2ccnc(n2)-c2cnn(C)c2OCC2(CCCC2)C3)C1] | (S)-3′-(3-((ethylsulfonyl)meth- yl)pyrrolidin-1-yl)-1′-meth- ylspiro(cyclopentane-1,6′- 8-oxa-3-aza-4(6,1)-pyrazolo [4,3-c]pyridina-2(2,4)-pyri- midina-1(4,5)-pyrazolacyclo- octaphane) | 578.4 |
| 36 | 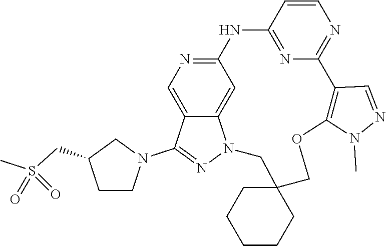 [SMILES: Cn1ncc2c1OCC1(CCCCC1)Cn1nc(N3CC[C@H](CS(C)(=O)=O)C3)c3cnc(cc31)Nc1ccnc-2n1] | (S)-1′-methyl-3′-(3-((methyl- sulfonyl)methyl)pyrrolidin- 1-yl)spiro(cyclohexane-1,6′- 8-oxa-3-aza-4(6,1)-pyrazolo [4,3-c]pyridina-2(2,4)-pyri- midina-1(4,5)-pyrazolacyclo- octaphane) | 578.4 |
| 37 | 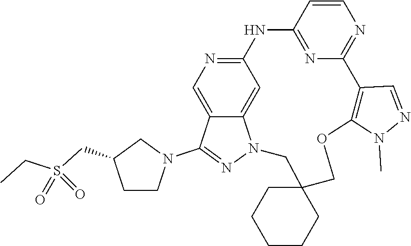 [SMILES: CCS(=O)(=O)C[C@H]1CCN(c2nn3c4cc(ncc24)Nc2ccnc(n2)-c2cnn(C)c2OCC2(CCCCC2)C3)C1] | (S)-1′-methyl-3′-(3-((ethylsul- fonyl)methyl)pyrrolidin-1- yl)spiro(cyclohexane-1,6′- 8-oxa-3-aza-4(6,1)-pyrazolo [4,3-c]pyridina-2(2,4)-pyri- midina-1(4,5)-pyrazolacyclo- octaphane) | 592.4 |
| 38 | 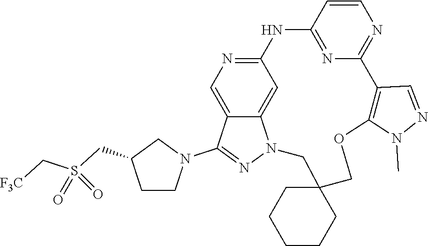 [SMILES: Cn1ncc2c1OCC1(CCCCC1)Cn1nc(N3CC[C@H](CS(=O)(=O)CC(F)(F)F)C3)c3cnc(cc31)Nc1ccnc-2n1] | (S)-1′-methyl-3′-(3-(((trifluoro- methyl)sulfonyl)methyl) pyrrolidin-1-yl)spiro(cyclohex- ane-1,6′-8-oxa-3-aza-4(6,1)- pyrazolo[4,3-c]pyridina- 2(2,4)-pyrimidina-1(4,5)-py- razolacyclooctaphane) | 632.4 |
| 39 | 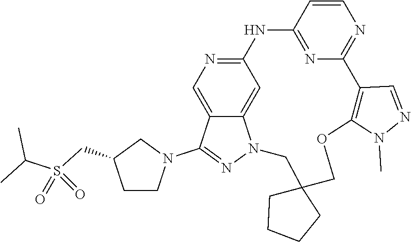 [SMILES: CC(C)S(=O)(=O)C[C@H]1CCN(c2nn3c4cc(ncc24)Nc2ccnc(n2)-c2cnn(C)c2OCC2(CCCC2)C3)C1] | (S)-3′-(3-((isopropylsulfonyl) methyl)pyrrolidin-1-yl)-1′- methylspiro(cyclopentane- 1,6′-8-oxa-3-aza-4(6,1)-pyra- zolo[4,3-c]pyridina-2(2,4)- pyrimidina-1(4,5)-pyrazolacy- clooctaphane) | 592.4 |
| 40 | 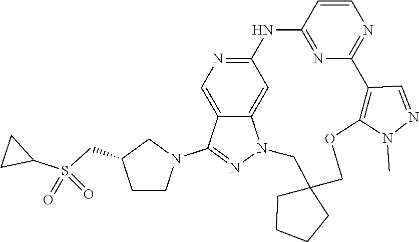 [SMILES: Cn1ncc2c1OCC1(CCCC1)Cn1nc(N3CC[C@H](CS(=O)(=O)C4CC4)C3)c3cnc(cc31)Nc1ccnc-2n1] | (S)-3′-(3-((cyclopropylsulfo- nyl)methyl)pyrrolidin-1-yl)- 1′-methylspiro(cyclopentane- 1,6′-8-oxa-3-aza-4(6,1)-py- razolo[4,3-c]pyridina-2(2,4)- pyrimidina-1(4,5)-pyrazo- lacyclooctaphane) | 590.4 |
| 41 | 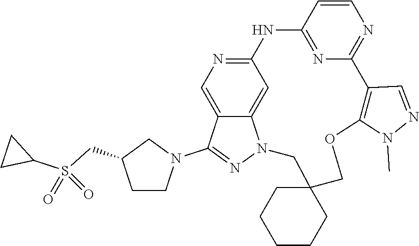 [SMILES: Cn1ncc2c1OCC1(CCCCC1)Cn1nc(N3CC[C@H](CS(=O)(=O)C4CC4)C3)c3cnc(cc31)Nc1ccnc-2n1] | (S)-3′-(3-((cyclopropylsulfo- nyl)methyl)pyrrolidin-1-yl)- 1′-methylspiro(cyclohexane- 1,6′-8-oxa-3-aza-4(6,1)-py- razolo[4,3-c]pyridina-2(2,4)- pyrimidina-1(4,5)-pyrazola- cyclooctaphane) | 604.4 |
| 42 | 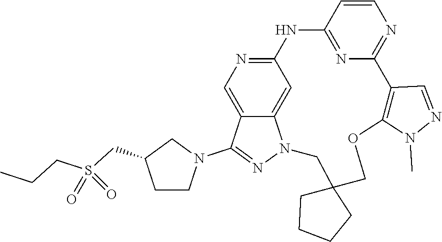 [SMILES: CCCS(=O)(=O)C[C@H]1CCN(c2nn3c4cc(ncc24)Nc2ccnc(n2)-c2cnn(C)c2OCC2(CCCC2)C3)C1] | (S)-3′-(3-((propylsulfonyl) methyl)pyrrolidin-1-yl)-1′- methylspiro(cyclopentane-1,6′- 8-oxa-3-aza-4(6,1)-pyra- zolo[4,3-c]pyridina-2(2,4)-py- rimidina-1(4,5)-pyrazolacy- clooctaphane) | 592.4 |
| 43 | 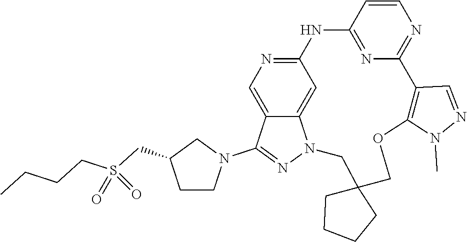 [SMILES: CCCCS(=O)(=O)C[C@H]1CCN(c2nn3c4cc(ncc24)Nc2ccnc(n2)-c2cnn(C)c2OCC2(CCCC2)C3)C1] | (S)-3′-(3-((butylsulfonyl)meth- yl)pyrrolidin-1-yl)-l′-meth- ylspiro(cyclopentane-1,6′- 8-oxa-3-aza-4(6,1)-pyrazolo [4,3-c]pyridina-2(2,4)-pyri- midina-1(4,5)-pyrazolacyclo- octaphane) | 606.4 |
| 44 | 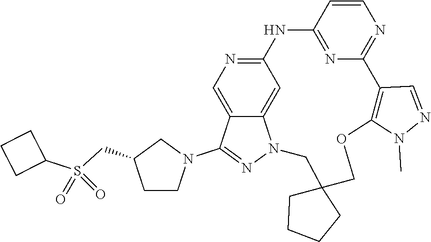 [SMILES: Cn1ncc2c1OCC1(CCCC1)Cn1nc(N3CC[C@H](CS(=O)(=O)C4CCC4)C3)c3cnc(cc31)Nc1ccnc-2n1] | (S)-3′-(3-((cyclobutylsulfonyl) methyl)pyrrolidin-1-yl)-1′- methylspiro(cyclopentane- 1,6′-8-oxa-3-aza-4(6,1)-pyra- zolo[4,3-c]pyridina-2(2,4)- pyrimidina-1(4,5)-pyrazolacy- clooctaphane) | 604.4 |
| 45 | 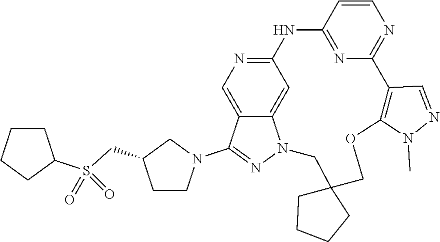 [SMILES: Cn1ncc2c1OCC1(CCCC1)Cn1nc(N3CC[C@H](CS(=O)(=O)C4CCCC4)C3)c3cnc(cc31)Nc1ccnc-2n1] | (S)-3′-(3-((cyclopentylsulfo- nyl)methyl)pyrrolidin-1-yl)- 1′-methylspiro(cyclopentane- 1,6′-8-oxa-3-aza-4(6,1)-py- razolo[4,3-c]pyridina-2(2,4)- pyrimidina-1(4,5)-pyrazo- lacyclooctaphane) | 618.4 |
| 46 | 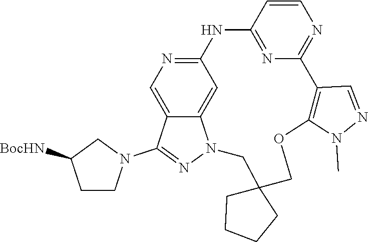 [SMILES: Cn1ncc2c1OCC1(CCCC1)Cn1nc(N3CC[C@@H](NC(=O)OC(C)(C)C)C3)c3cnc(cc31)Nc1ccnc-2n1] | Tert-butyl (R)-(1-(1′-methylspiro(cyclo- pentane-1,6′-8-oxa-3-aza- 4(6,1)-pyrazolo[4,3-c]pyridi- na-2(2,4)-pyrimidina-1(4,5)- pyrazolacyclooctaphane)-3- yl)pyrrolidin-3-yl)carbamate | 587.4 |
| 47 | 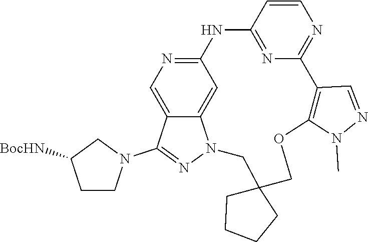 [SMILES: Cn1ncc2c1OCC1(CCCC1)Cn1nc(N3CC[C@H](NC(=O)OC(C)(C)C)C3)c3cnc(cc31)Nc1ccnc-2n1] | Tert-butyl (S)-(1-(1′-methylspiro(cyclo- pentane-1,6′-8-oxa-3-aza- 4(6,1)-pyrazolo[4,3-c]pyridi- na-2(2,4)-pyrimidina-1(4,5)- pyrazolacyclooctaphane)-3- yl)pyrrolidin-3-yl)carbamate | 587.4 |
| A4 | 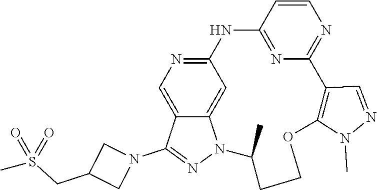 [SMILES: C[C@H]1CCOc2c(cnn2C)-c2nccc(n2)Nc2cc3c(cn2)c(N2CC(CS(C)(=O)=O)C2)nn31] | (S)-1¹,5-dimethyl-4³-(3-((meth- ylsulfony1)methyl)azetidin- 1-yl)-1¹H,4¹H-8-oxa-3-aza- 4(6,1)-pyrazolo[4,3-c]pyri- dina-2(2,4)-pyrimidina-1(4,5)- pyrazolacyclooctaphane | 510.4 |

### Example 48: Preparation of (R)—N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-yl)methanesulfonamide

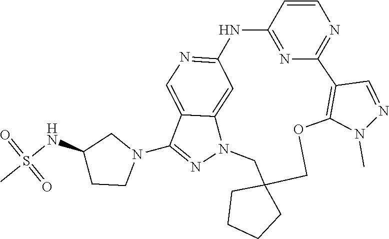 [SMILES: Cn1ncc2c1OCC1(CCCC1)Cn1nc(N3CC[C@@H](NS(C)(=O)=O)C3)c3cnc(cc31)Nc1ccnc-2n1]

### Step 1: Synthesis of (R)-1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-amine

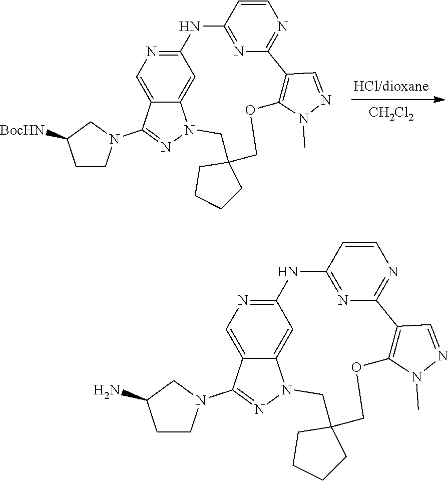

Tert-butyl (R)-(1-(1′-methylspiro(cyclopentane-1,6′-8-oxa-3-aza-4(6,1)-pyrazolo[4,3-c]pyridin-2(2,4)-pyrimidin-1(4,5)-pyrazolyl)-3′-yl)pyrrolidin-3-yl)carbamate (prepared with reference to Example 1) (70 mg, purity: 77.1%, 0.10 mmol) was dissolved in CH₂Cl₂ (3.0 mL), and HCl/dioxane solution (0.46 mL, 4.0 M, 1.84 mmol) was added under cooling in an ice-water bath, and the mixture was reacted at room temperature for 1 hour. After the reaction was completed, the reaction solution was concentrated to obtain (R)-1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-amine, which was directly used for the next reaction. MS m/z (ESI): 487.3 [M+H]⁺.

### Step 2: Synthesis of (R)—N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-yl)methanesulfonamide

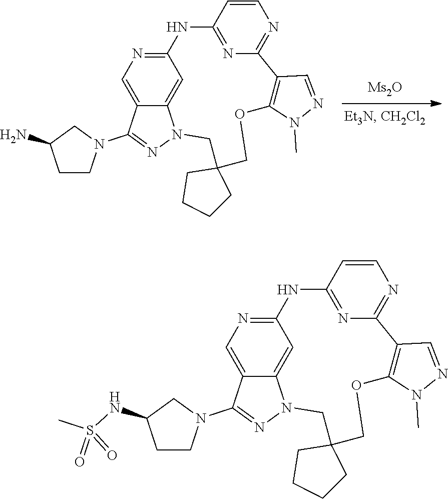

The crude product obtained in the previous step was dissolved in dry CH₂Cl₂ (3.0 mL), and triethylamine (0.10 mL, 0.74 mmol) and methanesulfonic anhydride (32 mg, 0.18 mmol) were added under cooling in an ice-water bath, and the mixture was reacted at room temperature for 1 hour. The reaction was quenched by adding saturated aqueous NaHCO₃ solution, and then extracted twice with CH₂Cl₂. The organic phase was combined, washed sequentially with saturated brine, and dried over anhydrous sodium sulfate. It was filtered, concentrated and separated by using column chromatography to obtain (R)—N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-yl)methanesulfon amide (7.3 mg, yield: 13%). MS m/z (ESI): 565.3 [M+H]⁺.

¹H NMR (400 MHz, DMSO-d₆) δ 10.26 (s, 1H), 9.21 (s, 1H), 8.71 (d, J=0.9 Hz, 1H), 8.30 (d, J=5.8 Hz, 1H), 8.00 (s, 1H), 6.77 (dd, J=5.8, 1.3 Hz, 1H), 4.61 (brs, 2H), 4.08 (p, J=6.3 Hz, 1H), 3.80 (s, 4H), 3.68 (dd, J=8.7, 5.9 Hz, 1H), 3.58 (q, J=7.8 Hz, 1H), 3.41 (dd, J=9.9, 5.5 Hz, 1H), 3.00 (s, 3H), 2.35-2.24 (m, 2H), 2.04-1.85 (m, 4H), 1.70 (s, 2H).

### Example 49: Preparation of (R)—N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane-3′-yl)pyrrolidin-3-yl)acetamide

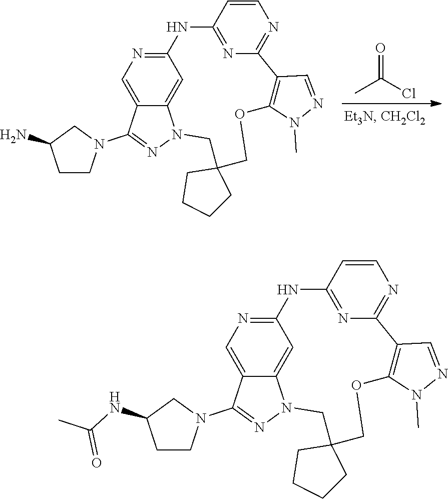

(R)-1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-amine (0.010 mmol) was dissolved in CH₂Cl₂ (3.0 mL). Triethylamine (0.10 mL, 0.74 mmol) was added under cooling in an ice-water bath, and acetyl chloride (0.03 mL, 0.46 mmol) was slowly added dropwise. The mixture was maintained at 0° C., and reacted for 1 hour. After the reaction was completed, saturated aqueous NaHCO₃ solution was added to quench the reaction, and the mixture was extracted twice with CH₂Cl₂. The organic phase was combined, washed successively with saturated brine, and dried over anhydrous sodium sulfate. It was filtered, concentrated and separated by using column chromatography to obtain (R)—N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-yl)acetamide (14.1 mg, yield: 26%). MS m/z (ESI): 529.4 [M+H]⁺.

¹HNMR (400 MHz, DMSO-d₆) δ 10.26 (s, 1H), 9.21 (d, J=1.0 Hz, 1H), 8.71 (d, J=1.0 Hz, 1H), 8.30 (d, J=5.8 Hz, 1H), 8.18 (d, J=6.7 Hz, 1H), 8.00 (s, 1H), 6.76 (dd, J=5.8, 1.5 Hz, 1H), 4.65 (brs, 2H), 4.37 (q, J=5.8 Hz, 1H), 3.80 (s, 3H), 3.75 (dd, J=10.0, 6.5 Hz, 1H), 3.67 (q, J=5.6 Hz, 1H), 3.59 (q, J=8.1 Hz, 1H), 3.37-3.35 (m, 1H), 2.20 (q, J=6.1 Hz, 2H), 1.94-1.87 (m, 3H), 1.82 (s, 3H), 1.70 (s, 2H).

### Example 50: Preparation of (R)-2,2,2-trifluoro-N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane-3′-yl)pyrrolidin-3-yl)acetamide

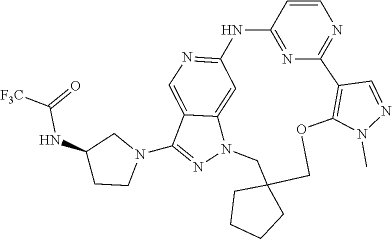 [SMILES: Cn1ncc2c1OCC1(CCCC1)Cn1nc(N3CC[C@@H](NC(=O)C(F)(F)F)C3)c3cnc(cc31)Nc1ccnc-2n1]

### Step 1: Synthesis of (R)-1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-amine

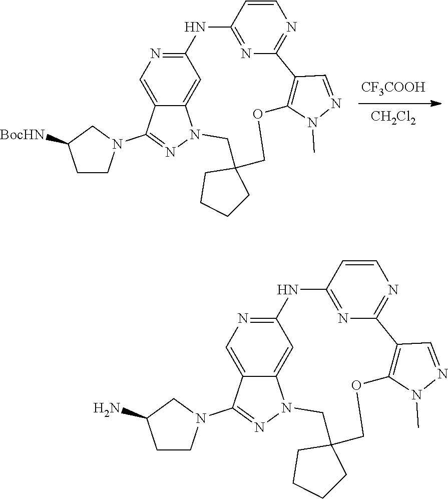

Tert-butyl (R)-(1-(1′-methylspiro(cyclopentane-1,6′-8-oxa-3-aza-4(6,1)-pyrazolo[4,3-c]pyridina-2 (2,4)-pyrimidina-1(4,5)-pyrazolacyclooctaphane)-3′-yl)pyrrolidin-3-yl)carbamate (50 mg, 0.08 mmol) was dissolved in CH₂Cl₂ (3.0 mL). CF₃COOH (0.24 mL, 3.28 mmol) was added under cooling in an ice-water bath. After the mixture was stirred for 1 hour, it was heated to room temperature and reacted for 3 hours. After it was concentrated to remove the solvent, the resulting crude product was directly used for the next reaction. MS m/z (ESI): 487.2 [M+H]⁺.

### Step 2: Synthesis of (R)-2,2,2-trifluoro-N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane-3′-yl)pyrrolidin-3-yl)acetamide

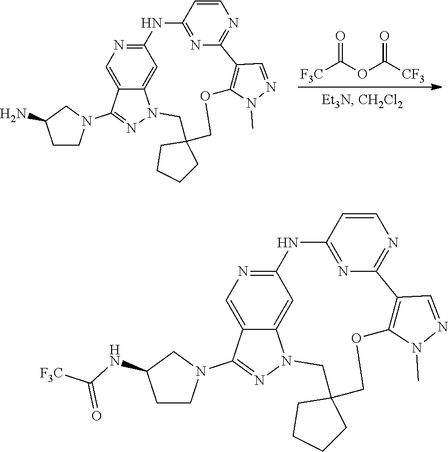

(R)-1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3′-yl)pyrrolidin-3-amine (0.08 mmol) was dissolved in CH₂Cl₂ (3.0 mL), triethylamine (0.03 mL, 0.25 mmol) was added under cooling in an ice-water bath, and TFAA (0.02 mL, 0.12 mmol) was slowly added dropwise. The mixture was maintained at 0° C., and reacted for 1 hour. After the reaction was completed, saturated aqueous NaHCO₃ solution was added to quench the reaction, and the mixture was extracted twice with CH₂C₂. The organic phase was combined, washed successively with saturated brine, and dried over anhdrous sodium sulfate. It was filtered, concentrated and separated by using column chromatography to obtain (R)-2,2,2-trifluoro-N-(1-(1′-methylspiro(cyclopentane-1,6′-8-oxa-3-aza-4(6,1)-pyrazolo[4,3-c]pyridina-2(2,4)-pyrimidina-1(4,5)-pyrazolacyclooctaphane)-3-yl)pyrrolidin-3-yl)acetamide (13.3 mg, yield: 27%). MS m/z (ESI): 583.4 [M+H]⁺.

¹H NMR (400 MHz, DMSO-d₆) δ 10.29 (s, 1H), 9.75 (d, J=6.8 Hz, 1H), 9.22 (s, 1H), 8.74 (s, 1H), 8.30 (d, J=5.8 Hz, 1H), 8.00 (s, 1H), 6.76 (dd, J=5.9, 1.5 Hz, 1H), 4.65 (brs, 2H), 4.51 (s, 1H), 3.87-3.81 (m, 1H), 3.80 (s, 3H), 3.77-3.67 (m, 1H), 3.61 (d, J=7.6 Hz, 1H), 3.53 (dd, J=10.2, 4.6 Hz, 1H), 2.37-2.23 (m, 2H), 2.05 (dq, J=13.0, 6.6 Hz, 2H), 1.91 (s, 3H), 1.70 (s, 2H).

Examples 51 to 58 can be prepared by selecting the corresponding raw materials with reference to the synthesis methods of Example 48, 49 or 50.

| Ex- | | | MS: |
|---|---|---|---|
| am- | | | m/z |
| ple | | | [M + |
| No. | Structural Formula | Chemical Name | H]⁺ |
| 51 | 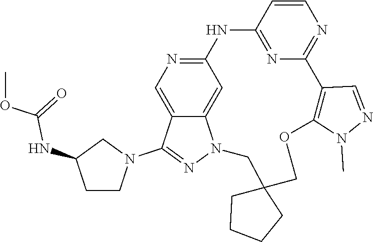 [SMILES: COC(=O)N[C@@H]1CCN(c2nn3c4cc(ncc24)Nc2ccnc(n2)-c2cnn(C)c2OCC2(CCCC2)C3)C1] | Methyl (R)-(1-(1′-methylspiro(cyclo- pentane-1,6′-8-oxa-3-aza-4 (6,1)-pyrazolo[4,3-c]pyridina- 2(2.4)-pyrimidina-1(4,5)-py- razolacyclooctaphane)-3′-yl) pyrrolidin-3-yl)carbamate | 545.4 |
| 52 | 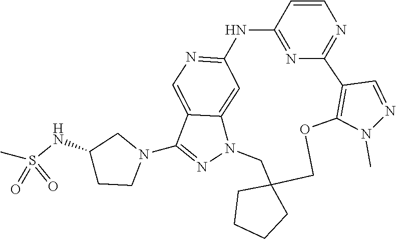 [SMILES: Cn1ncc2c1OCC1(CCCC1)Cn1nc(N3CC[C@H](NS(C)(=O)=O)C3)c3cnc(cc31)Nc1ccnc-2n1] | (R)-N-(1-(1′-methylspiro(cy- clopentane-1,6′-8-oxa-3-aza- 4(6,1)-pyrazolo[4,3-c]pyridi- na-2(2,4)-pyrimidina-1(4,5) pyrazolacyclooctaphane)-3′- yl)pyrrolidin-3-yl)methanesul- fonamide | 565.3 |
| 53 | 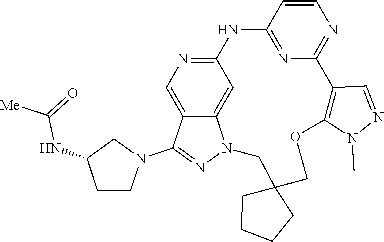 [SMILES: CC(=O)N[C@H]1CCN(c2nn3c4cc(ncc24)Nc2ccnc(n2)-c2cnn(C)c2OCC2(CCCC2)C3)C1] | (S)-N-(1-(1′-methylspiro(cy- clopentane-1,6′-8-oxa-3-aza- 4(6,1)-pyrazolo[4,3-c]pyridi- na-2(2,4)-pyrimidina-1(4,5)- pyrazolacyclooctaphane-3′- yl)pyrrolidin-3-yl)acetamide | 529.4 |
| 54 | 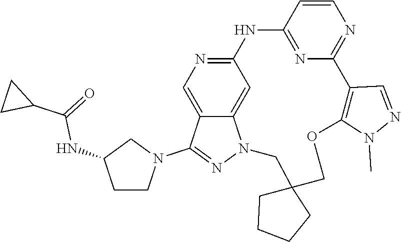 [SMILES: Cn1ncc2c1OCC1(CCCC1)Cn1nc(N3CC[C@H](NC(=O)C4CC4)C3)c3cnc(cc31)Nc1ccnc-2n1] | (S)-N-(1-(1′-methylspiro(cy- clopentane-1,6′-8-oxa-3-aza- 4(6,1)-pyrazolo[4,3-c]pyridi- na-2(2,4)-pyrimidina-1(4,5)- pyrazolacyclooctaphane)-3′- yl)pyrrolidin-3-yl)cyclopro- panecarboxamide | 555.4 |
| 55 | 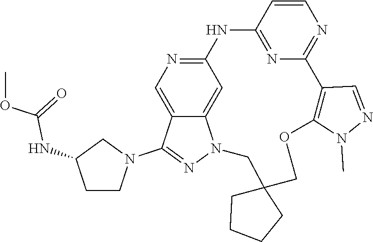 [SMILES: COC(=O)N[C@H]1CCN(c2nn3c4cc(ncc24)Nc2ccnc(n2)-c2cnn(C)c2OCC2(CCCC2)C3)C1] | Methyl (S)-(1-(1′-methylspiro(cyclo- pentane-1,6′-8-oxa-3-aza-4(6,1)- pyrazolo[4,3-c]pyridina- 2(2,4)-pyrimidina-1(4,5)-py- razolacyclooctaphane)-3′-yl) pyrrolidin-3-yl)carbamate | 545.4 |
| 56 | 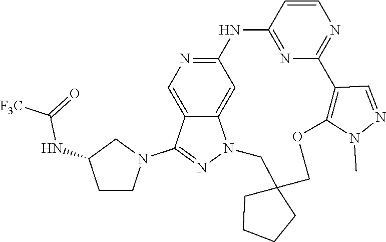 [SMILES: Cn1ncc2c1OCC1(CCCC1)Cn1nc(N3CC[C@H](NC(=O)C(F)(F)F)C3)c3cnc(cc31)Nc1ccnc-2n1] | (S)-2,2,2-trifluoro-N-(1-(1′- methylspiro(cyclopentane-1,6′- 8-oxa-3-aza-4(6,1)-pyrazo- lo[4,3-c]pyridina-2(2,4)-py- rimidina-1(4,5)-pyrazolacyclo- octaphane-3′-y1)pyrrolidin- 3-yl)acetamide | 583.4 |
| 57 | 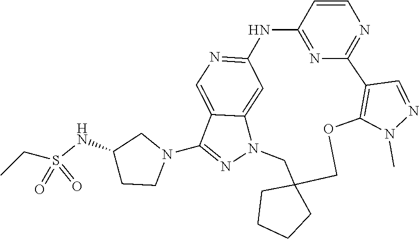 [SMILES: CCS(=O)(=O)N[C@H]1CCN(c2nn3c4cc(ncc24)Nc2ccnc(n2)-c2cnn(C)c2OCC2(CCCC2)C3)C1] | (S)-N-(1-(1′-methylspiro(cy- clopentane-1,6′-8-oxa-3-aza- 4(6,1)-pyrazolo[4,3-c]pyridi- na-2(2,4)-pyrimidina-1(4,5)- pyrazolacyclooctaphane-3′- yl)pyrrolidin-3-yl)ethanesulfona- mide | 579.4 |
| 58 | 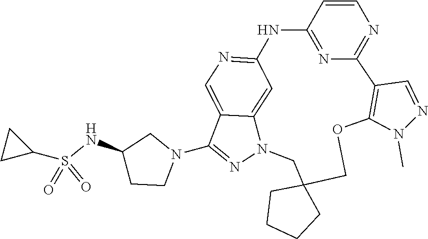 [SMILES: Cn1ncc2c1OCC1(CCCC1)Cn1nc(N3CC[C@@H](NS(=O)(=O)C4CC4)C3)c3cnc(cc31)Nc1ccnc-2n1] | (R)-N-(1-(1′-methylspiro(cy- clopentane-1,6′-8-oxa-3-aza- 4(6,1)-pyrazolo[4,3-c]pyridi- na-2(2,4)-pyrimidina-1(4,5)- pyrazolacyclooctaphane)-3′- yl)pyrrolidin-3-yl)cyclopro- panesulfonamide | 591.7 |

¹HNMR data for the compounds prepared in the above examples are as follows:

| Example | |
|---|---|
| No. | ¹H NMR (400 MHz) |
| 2 | ¹H NMR (400 MHz, DMSO-d₆) δ 10.47 (s, 1H), 9.49 (s, 1H), 8.73 (s, 1H), |
| | 8.31 (d, J = 8.0 Hz, 1H), 7.99 (s, 1H), 6.77 (d, J = 4.0 Hz, 1H), 4.79 (s, |
| | 1H), 3.87-3.72 (m, 4H), 2.98-2.81 (m, 3H), 2.32 (s, 3H), 2.27 (s, 3H), |
| | 1.98-1.72 (m, 6H), 1.59-1.27 (m, 3H), 0.94-0.73 (m, 2H). |
| 3 | ¹H NMR (400 MHz, DMSO-d₆) δ 10.51 (s, 1H), 9.55 (s, 1H), 8.72 (s, |
| | 1H), 8.33 (d, J = 5.8 Hz, 1H), 8.02 (s, 1H), 6.79 (d, J = 5.8 Hz, 1H), 4.93- |
| | 4.23 (m, 3H), 3.81 (s, 3H), 2.78-2.56 (m, 3H), 2.17 (s, 3H), 2.10 (s, |
| | 2H), 1.97-1.89 (m, 2H), 1.85-1.53 (m, 7H), 1.24 (s, 2H), 0.64 (brs, |
| | 2H). |
| 4 | ¹H NMR (400 MHz, DMSO-d₆) δ 10.53 (s, 1H), 9.54 (s, 1H), 8.73 (s, |
| | 1H), 8.34 (d, J = 5.8 Hz, 1H), 8.02 (s, 1H), 6.80 (d, J = 5.8 Hz, 1H), 4.68 |
| | (brs, 3H), 3.81 (s, 7H), 3.60 (d, J = 27.1 Hz, 1H), 2.91 (brs, 1H), 2.79- |
| | 2.61 (m, 3H), 2.18 (s, 3H), 2.16-2.06 (m, 2H), 1.99-1.88 (m, 3H), 1.70 |
| | (dtd, J = 13.0, 9.5, 3.5 Hz, 2H), 1.00 (brs, 1H), 0.42 (brs, 1H). |
| 5 | ¹H NMR (400 MHz, DMSO-d₆) δ 10.47 (s, 1H), 9.45 (s, 1H), 8.75 (s, |
| | 1H), 8.33 (d, J = 5.8 Hz, 1H), 8.01 (s, 1H), 6.79 (d, J = 5.9 Hz, 1H), 4.77 |
| | (brs, 2H), 4.30 (brs, 2H), 3.81 (s, 3H), 2.94 (brs, 4H), 2.43 (s, 3H), 2.26- |
| | 2.12 (m, 2H), 2.11-1.98 (m, 3H), 1.98-1.76 (m, 5H). |
| 6 | ¹H NMR (400 MHz, DMSO-d₆) δ 10.52 (s, 1H), 9.54 (s, 1H), 8.77 (s, |
| | 1H), 8.35-8.32 (m, 2H), 8.02 (s, 1H), 6.80 (d, J = 5.9 Hz, 1H), 3.82 (s, |
| | 3H), 3.17-3.08 (m, 2H), 3.04-2.96 (m, 1H), 2.84 (t, J = 10.3 Hz, 2H), |
| | 2.48-2.42 (m, 8H), 2.05-1.97 (m, 2H), 1.92-1.83 (m, 3H), 1.79-1.70 |
| | (m, 3H). |
| 7 | ¹H NMR (400 MHz, DMSO-d₆) δ 10.51 (s, 1H), 9.53 (s, 1H), 8.74 (s, |
| | 1H), 8.34 (d, J = 5.8 Hz, 1H), 8.02 (s, 1H), 6.80 (d, J = 5.8 Hz, 1H), 3.82 |
| | (s, 4H), 2.81-2.71 (m, 4H), 2.67 (s, 1H), 2.39-2.31 (m, 4H), 2.21-2.11 |
| | (m, 2H), 2.01-1.92 (m, 3H), 1.91 (s, 1H), 1.87 (s, 3H), 1.75-1.64 (m, |
| | 4H), 1.01 (t, J = 7.2 Hz, 3H). |
| 8 | ¹H NMR (400 MHz, DMSO-d₆) δ 10.50 (s, 1H), 9.51 (s, 1H), 8.77 (s, |
| | 1H), 8.33 (d, J = 5.8 Hz, 1H), 8.01 (s, 1H), 6.79 (d, J = 5.8 Hz, 1H), 3.80 |
| | (s, 3H), 3.66 (t, J = 7.1 Hz, 3H), 3.58-3.52 (m, 2H), 3.47-3.39 (m, 2H), |
| | 3.23 (t, J = 6.8 Hz, 3H), 2.30 (s, 3H), 1.87 (s, 3H), 1.80-1.61 (m, 2H), |
| | 1.03-0.62 (s, 2H). |
| 9 | ¹H NMR (400 MHz, DMSO-d₆) δ 10.56 (s, 1H), 9.58 (s, 1H), 8.82 (s, |
| | 1H), 8.34 (d, J = 5.8 Hz, 1H), 8.02 (s, 1H), 6.80 (d, J = 5.9 Hz, 1H), 5.23- |
| | 4.01 (m, 5H), 3.82 (s, 3H), 2.93 (s, 1H), 2.22 (s, 3H), 2.21-2.07 (m, |
| | 4H), 1.88-1.72 (m, 5H). |
| 11 | ¹H NMR (400 MHz, DMSO-d₆) δ 10.58 (s, 1H), 9.59 (s, 1H), 8.81 (d, J = |
| | 1.0 Hz, 1H), 8.35 (d, J = 5.8 Hz, 1H), 8.03 (s, 1H), 6.81 (d, J = 5.8 Hz, |
| | 1H), 4.74 (s, 2H), 3.82-3.57 (m, 9H), 2.94-2.83 (m, 4H), 2.22 (s, 3H), |
| | 2.20-1.80 (m, 8H). |
| 12 | ¹H NMR (400 MHz, DMSO-d₆) δ 10.53 (d, J = 5.3 Hz, 1H), 9.49 (d, J = |
| | 5.3 Hz, 1H), 8.81 (d, J = 5.5 Hz, 1H), 8.34 (d, J = 5.7 Hz, 1H), 8.01 (d, J = |
| | 5.4 Hz, 1H), 6.79 (d, J = 5.8 Hz, 1H), 4.81 (brs, 2H), 4.29 (brs, 3H), 3.81 |
| | (s, 3H), 2.93 (s, 1H), 2.26-2.07 (m, 9H), 1.93 (brs, 5H), 1.23 (s, 1H). |
| 15 | ¹H NMR (400 MHz, DMSO-d₆) δ 10.31 (s, 1H), 9.23 (d, J = 1.0 Hz, 1H), |
| | 8.79 (d, J = 1.0 Hz, 1H), 8.30 (d, J = 5.9 Hz, 1H), 8.00 (s, 1H), 6.77 |
| | (d, J = 5.8 Hz, 1H), 3.95 (t, J = 13.2 Hz, 2H), 3.80 (s, 3H), 3.77 (d, J = 7.2 |
| | Hz, 2H), 2.57 (td, J = 14.5, 7.3 Hz, 2H), 1.90 (s, 3H), 1.70 (s, 3H). |
| 16 | ¹H NMR (400 MHz, DMSO-d₆) δ 10.25 (s, 1H), 9.20 (d, J = 1.0 Hz, 1H), |
| | 8.74 (d, J = 0.9 Hz, 1H), 8.30 (d, J = 5.8 Hz, 1H), 8.00 (s, 1H), 6.77 (d, J = |
| | 5.9 Hz, 1H), 4.11 (s, 1H), 3.80 (s, 3H), 3.68 (dd, J = 10.8, 4.8 Hz, 1H), |
| | 3.63-3.53 (m, 3H), 3.28 (s, 3H), 2.13-2.04 (m, 2H), 1.91 (s, 2H), 1.70 |
| | (s, 2H), 1.62-1.53 (m, 1H), 1.35 (dd, J = 14.9, 7.7 Hz, 1H), 0.88 (t, J = |
| | 7.1 Hz, 1H). |
| 17 | ¹H NMR (400 MHz, DMSO-d₆) δ 10.26 (s, 1H), 9.19 (s, 1H), 8.70 (s, |
| | 1H), 8.30 (d, J = 5.8 Hz, 1H), 8.00 (s, 1H), 6.76 (d, J = 5.8 Hz, 1H), 3.80 |
| | (s, 3H), 3.69-3.59 (m, 2H), 3.54 (q, J = 8.0 Hz, 1H), 3.39 (dd, J = 6.8, |
| | 2.9 Hz, 3H), 3.29 (s, 5H), 2.60 (p, J = 7.1 Hz, 1H), 2.09 (h, J = 7.0 Hz, |
| | 1H), 1.91 (s, 3H), 1.81-1.66 (m, 3H). |
| 18 | ¹H NMR (400 MHz, DMSO-d₆) δ 10.22 (s, 1H), 9.18 (s, 1H), 8.71 (s, |
| | 1H), 8.29 (d, J = 5.8 Hz, 1H), 8.00 (s, 1H), 6.76 (d, J = 5.8 Hz, 1H), 4.50 |
| | (brs, 2H), 3.79 (s, 3H), 3.72 (dd, J = 9.5, 7.5 Hz, 1H), 3.62 (dt, J = 8.8, |
| | 5.1 Hz, 1H), 3.51 (q, J = 8.5 Hz, 1H), 3.42 (t, J = 6.5 Hz, 2H), 3.26 (s, |
| | 3H), 3.14 (t, J = 8.8 Hz, 1H), 2.92 (s, 1H), 2.34 (p, J = 7.6 Hz, 1H), 2.13 |
| | (d, J = 11.8 Hz, 1H), 1.90 (s, 2H), 1.81-1.48 (m, 6H). |
| 19 | ¹H NMR (400 MHz, DMSO-d₆) δ 10.23 (s, 1H), 9.18 (s, 1H), 8.71 (s, |
| | 1H), 8.29 (d, J = 5.9 Hz, 1H), 8.00 (s, 1H), 6.76 (d, J = 5.8 Hz, 1H), 4.50 |
| | (brs, 2H), 3.79 (s, 3H), 3.71 (dd, J = 9.4, 7.4 Hz, 1H), 3.63 (td, J = 8.7, |
| | 3.1 Hz, 1H), 3.56-3.46 (m, 1H), 3.42 (t, J = 6.5 Hz, 2H), 3.26 (s, 3H), |
| | 3.14 (t, J = 8.8 Hz, 1H), 2.89 (s, 1H), 2.33 (p, J = 7.4 Hz, 1H), 2.12 (s, |
| | 1H), 1.90 (s, 2H), 1.79-1.45 (m, 6H). |
| 20 | ¹H NMR (400 MHz, DMSO-d₆) δ 10.25 (s, 1H), 9.19 (s, 1H), 8.71 (s, |
| | 1H), 8.30 (d, J = 5.8 Hz, 1H), 8.00 (s, 1H), 6.77 (d, J = 5.9 Hz, 1H), 4.38 |
| | (s, 1H), 3.80 (s, 3H), 3.66 (t, J = 8.6 Hz, 1H), 3.58-3.39 (m, 3H), 2.45- |
| | 2.27 (m, 2H), 1.92 (m, 5H), 1.70 (s, 3H), 1.16 (d, J = 7.6 Hz, 6H). |
| 21 | ¹H NMR (400 MHz, DMSO-d₆) δ 10.25 (s, 1H), 9.19 (s, 1H), 8.71 (s, |
| | 1H), 8.30 (d, J = 5.8 Hz, 1H), 8.00 (s, 1H), 6.76 (d, J = 5.9 Hz, 1H), 4.72 |
| | (t, J = 5.3 Hz, 1H), 3.80 (s, 3H), 3.61 (dt, J = 15.5, 8.2 Hz, 2H), 3.52 |
| | (d, J = 8.5 Hz, 1H), 3.46 (q, J = 5.6 Hz, 2H), 2.05 (dd, J = 13.5, 6.6 Hz, |
| | 1H), 1.91 (s, 2H), 1.81-1.59 (m, 4H). |
| 22 | ¹H NMR (400 MHz, DMSO-d₆) δ 10.24 (s, 1H), 9.19 (s, 1H), 8.71 (s, |
| | 1H), 8.30 (d, J = 5.8 Hz, 1H), 8.00 (s, 1H), 6.76 (d, J = 5.8 Hz, 1H), 4.98 |
| | (s, 1H), 4.43 (s, 1H), 3.80 (s, 3H), 3.74-3.53 (m, 3H), 3.40 (d, J = 10.1 |
| | Hz, 1H), 2.15-1.99 (m, 1H), 1.99-1.83 (m, 4H), 1.71 (s, 3H). |
| 23 | ¹H NMR (400 MHz, DMSO-d₆) δ 10.24 (s, 1H), 9.18 (s, 1H), 8.69 (s, |
| | 1H), 8.29 (d, J = 5.8 Hz, 1H), 8.00 (s, 1H), 6.76 (d, J = 5.9 Hz, 1H), 4.80 |
| | (s, 1H), 4.50 (brs, 3H), 3.80 (s, 3H), 3.74-3.54 (m, 3H), 3.52-3.41 (m, |
| | 2H), 2.93 (brs, 1H), 1.93 (dd, J = 12.8, 5.8 Hz, 4H), 1.70 (brs, 2H), 1.38 |
| | (s, 3H). |
| 24 | ¹H NMR (400 MHz, DMSO-d₆) δ 10.24 (s, 1H), 9.19 (s, 1H), 8.69 (s, |
| | 1H), 8.29 (d, J = 5.8 Hz, 1H), 8.00 (s, 1H), 6.76 (d, J = 5.8 Hz, 1H), 4.80 |
| | (s, 1H), 3.80 (s, 3H), 3.72-3.57 (m, 2H), 3.52-3.42 (m, 2H), 1.98- |
| | 1.87 (m, 4H), 1.70 (s, 2H), 1.38 (s, 3H). |
| 25 | ¹H NMR (400 MHz, DMSO-d₆) δ 10.27 (s, 1H), 9.19 (d, J = 1.1 Hz, 1H), |
| | 8.55 (d, J = 1.0 Hz, 1H), 8.30 (d, J = 5.8 Hz, 1H), 7.99 (s, 1H), 6.76 |
| | (d, J = 5.9 Hz, 1H), 4.54 (s, 2H), 4.32 (t, J = 7.7 Hz, 2H), 3.96 (t, J = 7.0 |
| | Hz, 2H), 3.80 (s, 3H), 3.57 (d, J = 7.5 Hz, 2H), 3.00 (s, 3H), 2.24-2.05 |
| | (m, 2H), 1.94 (s, 4H). |
| 26 | ¹H NMR (400 MHz, DMSO-d₆) δ 10.30 (s, 1H), 9.26 (d, J = 1.0 Hz, 1H), |
| | 8.57 (s, 1H), 8.30 (d, J = 5.8 Hz, 1H), 8.00 (s, 1H), 6.77 (dd, J = 5.9, 1.5 |
| | Hz, 1H), 4.30 (t, J = 7.6 Hz, 2H), 3.94 (t, J = 7.0 Hz, 2H), 3.80 (s, 3H), |
| | 3.57 (d, J = 7.5 Hz, 2H), 3.33 (m, 8H), 3.00 (s, 3H), 1.89 (m, 2H), 1.70 |
| | (m, 2H). |
| 27 | ¹H NMR (400 MHz, DMSO-d₆) δ10.26 (s, 1H), 9.20 (s, 1H), 8.68 (d, J = |
| | 0.9 Hz, 1H), 8.30 (d, J = 5.8 Hz, 1H), 8.00 (s, 1H), 6.76 (dd, J = 5.8, 1.5 |
| | Hz, 1H), 4.50 (brs, 3H), 3.87-3.81 (m, 1H), 3.79 (s, 3H), 3.68 (td, J = |
| | 8.8, 3.1 Hz, 1H), 3.62-3.51 (m, 1H), 3.41 (h, J = 6.7 Hz, 2H), 3.05 (s, |
| | 3H), 2.81 (dt, J = 14.7, 7.5 Hz, 1H), 2.33-2.22 (m, 1H), 1.98-1.78 (m, |
| | 4H), 1.69 (s, 2H). |
| 28 | ¹H NMR (400 MHz, DMSO-d₆) δ 10.29 (s, 1H), 9.21 (s, 1H), 8.82 (s, |
| | 1H), 8.30 (d, J = 5.8 Hz, 1H), 8.00 (s, 1H), 6.77 (d, J = 5.9 Hz, 1H), 4.75 |
| | (brs, 2H), 4.03 (s, 1H), 3.79 (s, 3H), 3.75 (s, 1H), 3.31-3.25 (m, 2H), |
| | 3.23-3.13 (m, 1H), 3.04 (s, 3H), 3.02-2.82 (m, 3H), 2.34 (s, 2H), 1.91 |
| | (s, 4H), 1.81-1.53 (m, 5H), 1.46-1.30 (m, 2H). |
| 29 | ¹H NMR (400 MHz, DMSO-d₆) δ 10.26 (s, 1H), 9.20 (d, J = 1.0 Hz, 1H), |
| | 8.68 (d, J = 0.9 Hz, 1H), 8.30 (d, J = 5.8 Hz, 1H), 8.00 (s, 1H), 6.76 |
| | (dd, J = 5.9, 1.4 Hz, 1H), 4.67 (brs, 3H), 3.87-3.82 (m, 1H), 3.79 (s, |
| | 3H), 3.68 (s, 1H), 3.57 (p, J = 8.2 Hz, 1H), 3.35 (d, J = 13.6 Hz, 5H), 3.15 |
| | (q, J = 7.4 Hz, 2H), 2.80 (dt, J = 14.7, 7.4 Hz, 1H), 2.34-2.24 (m, 1H), |
| | 2.00-1.79 (m, 4H), 1.70 (s, 2H), 1.25 (t, J = 7.4 Hz, 3H). |
| 30 | ¹H NMR (400 MHz, DMSO-d₆) δ 10.26 (s, 1H), 9.20 (d, J = 1.0 Hz, 1H), |
| | 8.68 (d, J = 0.9 Hz, 1H), 8.30 (d, J = 5.8 Hz, 1H), 8.00 (s, 1H), 6.76 |
| | (dd, J = 5.9, 1.5 Hz, 1H), 4.63 (brs, 3H), 3.87-3.82 (m, 1H), 3.79 (s, |
| | 3H), 3.69 (dt, J = 9.8, 4.9 Hz, 1H), 3.58 (p, J = 7.7 Hz, 1H), 3.49-3.39 |
| | (m, 2H), 3.34 (s, 4H), 2.91-2.73 (m, 2H), 2.33-2.25 (m, 1H), 1.88 |
| | (dt, J = 12.4, 8.4 Hz, 4H), 1.70 (s, 2H), 1.10-0.97 (m, 4H). |
| 31 | ¹H NMR (400 MHz, DMSO-d₆) δ 10.26 (s, 1H), 9.20 (s, 1H), 8.70-8.66 |
| | (m, 1H), 8.30 (d, J = 5.8 Hz, 1H), 8.00 (s, 1H), 6.77 (dd, J = 5.8, 1.5 Hz, |
| | 1H), 4.64 (brs, 3H), 3.80 (s, 4H), 3.73-3.64 (m, 1H), 3.57 (q, J = 8.3 Hz, |
| | 1H), 3.33 (s, 8H), 2.80 (p, J = 7.4 Hz, 1H), 2.33-2.26 (m, 1H), 1.98- |
| | 1.79 (m, 4H), 1.28 (d, J = 6.8 Hz, 6H). |
| 32 | ¹H NMR (400 MHz, DMSO-d₆) δ 10.35 (s, 1H), 9.21 (s, 1H), 8.68 (s, |
| | 1H), 8.30 (d, J = 5.9 Hz, 1H), 8.03 (s, 1H), 6.78 (d, J = 5.8 Hz, 1H), 4.46- |
| | 4.25 (m, 3H), 3.80 (s, 4H), 3.68 (s, 1H), 3.56 (d, J = 8.4 Hz, 1H), 3.41 |
| | (q, J = 6.9 Hz, 3H), 3.05 (s, 3H), 2.82 (q, J = 7.7 Hz, 1H), 2.29 (t, J = 10.5 |
| | Hz, 1H), 1.85 (dd, J = 12.1, 8.7 Hz, 2H), 1.65-1.52 (m, 3H), 1.37-1.25 |
| | (m, 3H), 1.24 (d, J = 7.2 Hz, 2H). |
| 34 | ¹H NMR (400 MHz, DMSO-d₆) δ 10.26 (s, 1H), 9.21 (s, 1H), 8.69 (s, |
| | 1H), 8.30 (dd, J = 5.8, 1.5 Hz, 1H), 8.00 (d, J = 1.5 Hz, 1H), 6.77 (d, J = |
| | 5.9 Hz, 1H), 4.58 (brs, 3H), 3.84 (d, J = 8.9 Hz, 1H), 3.80 (s, 3H), 3.68 |
| | (t, J = 5.3 Hz, 1H), 3.57 (q, J = 8.3 Hz, 1H), 3.40 (t, J = 6.5 Hz, 2H), 3.30 |
| | (s, 3H), 3.05 (s, 3H), 2.81 (p, J = 7.5 Hz, 1H), 2.30 (d, J = 18.7 Hz, 1H), |
| | 1.97-1.80 (m, 4H), 1.70 (s, 3H). |
| 35 | ¹H NMR (400 MHz, DMSO-d₆) δ10.26 (s, 1H), 9.20 (s, 1H), 8.68 (s, 1H), |
| | 8.29 (d, J = 5.8 Hz, 1H), 8.00 (s, 1H), 6.76 (dd, J = 5.8, 1.5 Hz, 1H), 4.62 |
| | (brs, 2H), 3.79 (s, 4H), 3.73-3.63 (m, 1H), 3.56 (q, J = 8.4 Hz, 1H), 3.37 |
| | (s, 1H), 3.32 (s, 6H), 3.15 (q, J = 7.4 Hz, 2H), 2.80 (p, J = 7.3 Hz, 1H), |
| | 2.29 (td, J = 10.0, 5.4 Hz, 1H), 2.01-1.79 (m, 4H), 1.69 (s, 2H), 1.25 |
| | (t, J = 7.4 Hz, 3H). |
| 36 | ¹H NMR (400 MHz, DMSO-d₆) δ 10.27 (s, 1H), 9.23 (s, 1H), 8.68 (s, |
| | 1H), 8.30 (d, J = 5.9 Hz, 1H), 8.01 (s, 1H), 6.77 (d, J = 5.9 Hz, 1H), 3.81 |
| | (s, 3H), 3.69 (m, 1H), 3.61-3.53 (m, 1H), 3.19-3.10 (m, 2H), 3.05 (s, |
| | 3H), 2.95 (m, 2H), 2.81 (p, J = 7.5 Hz, 2H), 2.28 (m, 2H), 2.03-1.96 (m, |
| | 1H), 1.86 (dd, J = 12.3, 8.8 Hz, 3H), 1.62 (s, 3H). |
| 37 | ¹H NMR (400 MHz, DMSO-d₆) δ 10.28 (s, 1H), 9.23 (s, 1H), 8.68 (s, |
| | 1H), 8.30 (d, J = 5.8 Hz, 1H), 8.01 (s, 1H), 6.77 (d, J = 5.7 Hz, 1H), 4.51 |
| | (brs, 2H), 3.81 (s, 4H), 3.69 (s, 1H), 3.58 (s, 1H), 3.38 (s, 1H), 3.31 (s, |
| | 6H), 3.15 (q, J = 7.4 Hz, 2H), 2.81 (q, J = 7.9 Hz, 1H), 1.88 (d, J = 10.5 |
| | Hz, 2H), 1.62 (s, 1H), 1.37 (d, J = 6.3 Hz, 2H), 1.24 (d, J = 7.4 Hz, 6H), |
| | 0.86 (dt, J = 12.2, 7.5 Hz, 3H). |
| 39 | ¹H NMR (400 MHz, DMSO-d₆) δ10.25 (s, 1H), 9.20 (s, 1H), 8.68 (s, 1H), |
| | 8.30 (d, J = 5.8 Hz, 1H), 8.00 (s, 1H), 6.77 (dd, J = 5.8, 1.4 Hz, 1H), 4.50 |
| | (brs, 3H), 3.80 (s, 4H), 3.73-3.64 (m, 1H), 3.57 (d, J = 8.3 Hz, 1H), 3.34 |
| | (d, J = 7.2 Hz, 3H), 2.80 (t, J = 7.9 Hz, 1H), 2.29 (dq, J = 7.4, 3.5 Hz, 1H), |
| | 1.89 (q, J = 10.4 Hz, 3H), 1.70 (s, 2H), 1.28 (d, J = 6.8 Hz, 6H). |
| 40 | ¹H NMR (400 MHz, DMSO-d₆) δ 10.27 (s, 1H), 9.20 (s, 1H), 8.69 (s, |
| | 1H), 8.30 (d, J = 5.9 Hz, 1H), 8.00 (s, 1H), 6.77 (d, J = 5.9 Hz, 1H), 4.65 |
| | (brs, 3H), 3.83 (dd, J = 9.7, 7.3 Hz, 1H), 3.80 (s, 3H), 3.69 (t, J = 8.7 Hz, |
| | 1H), 3.57 (q, J = 8.4 Hz, 1H), 3.44 (d, J = 7.0 Hz, 2H), 2.89-2.75 (m, |
| | 2H), 2.34-2.25 (m, 1H), 1.98-1.81 (m, 4H), 1.70 (s, 2H), 1.07-0.98 |
| | (m, 4H). |
| 41 | 1H NMR (400 MHz, CDCl₃) δ 9.34 (s, 1H), 8.59 (s, 1H), 8.36 (d, J = 5.7 |
| | Hz, 2H), 8.21 (s, 1H), 6.48 (d, J = 5.8 Hz, 1H), 4.50 (s, 2H), 3.99 (t, J = |
| | 8.5 Hz, 1H), 3.87 (s, 3H), 3.82-3.74 (m, 1H), 3.66 (q, J = 8.2 Hz, 1H), |
| | 3.47 (t, J = 8.7 Hz, 1H), 3.27 (d, J = 7.0 Hz, 2H), 3.06 (p, J = 7.3 Hz, 1H), |
| | 2.46 (tt, J = 8.0, 4.7 Hz, 2H), 2.06-1.92 (m, 2H), 1.81 (s, 2H), 1.37- |
| | 1.24 (m, 4H), 1.13-1.07 (m, 2H) |
| 42 | ¹H NMR (400 MHz, DMSO-d₆) δ 10.26 (s, 1H), 9.20 (s, 1H), 8.68 (s, |
| | 1H), 8.30 (d, J = 5.8 Hz, 1H), 8.00 (s, 1H), 6.77 (dd, J = 5.9, 1.4 Hz, 1H), |
| | 4.50 (brs, 3H), 3.87-3.81 (m, 1H), 3.79 (s, 3H), 3.68 (dt, J = 9.0, 4.9 Hz, |
| | 1H), 3.56 (q, J = 8.4 Hz, 1H), 3.35 (d, J = 7.0 Hz, 2H), 3.17-3.09 (m, |
| | 2H), 2.80 (p, J = 7.3 Hz, 1H), 2.29 (td, J = 10.1, 3.5 Hz, 1H), 1.99-1.58 |
| | (m, 8H), 1.01 (t, J = 7.4 Hz, 3H). |
| 43 | ¹H NMR (400 MHz, DMSO-d₆) δ 10.27 (s, 1H), 9.22-9.18 (m, 1H), 8.68 |
| | (d, J = 0.9 Hz, 1H), 8.29 (d, J = 5.8 Hz, 1H), 8.00 (s, 1H), 6.76 (dd, J = |
| | 5.8, 1.4 Hz, 1H), 4.62 (brs, 3H), 3.87-3.81 (m, 1H), 3.79 (s, 3H), 3.72- |
| | 3.64 (m, 1H), 3.56 (q, J = 8.3 Hz, 1H), 3.41-3.34 (m, 2H), 3.19-3.11 |
| | (m, 2H), 2.80 (p, J = 7.4 Hz, 1H), 2.34-2.21 (m, 1H), 2.02-1.81 (m, |
| | 4H), 1.69 (ddd, J = 12.1, 10.3, 6.6 Hz, 4H), 1.41 (hept, J = 7.1 Hz, 3H), |
| | 0.91 (t, J = 7.3 Hz, 3H). |
| 44 | ¹H NMR (400 MHz, DMSO-d₆) δ 11.05 (s, 1H), 9.10 (s, 1H), 8.75 (s, |
| | 1H), 8.33 (d, J = 6.4 Hz, 1H), 8.17 (s, 1H), 6.89 (d, J = 6.5 Hz, 1H), 4.61 |
| | (brs, 3H), 4.05 (p, J = 8.2 Hz, 2H), 3.82 (s, 4H), 3.68 (t, J = 8.7 Hz, 1H), |
| | 3.56 (q, J = 8.5 Hz, 1H), 3.35-3.19 (m, 3H), 2.76 (p, J = 7.8 Hz, 1H), |
| | 2.40-2.21 (m, 6H), 2.07-1.80 (m, 6H), 1.70 (s, 2H). |
| 45 | ¹H NMR (400 MHz, DMSO-d₆) δ 10.35 (s, 1H), 9.18 (s, 1H), 8.68 (s, |
| | 1H), 8.30 (d, J = 5.8 Hz, 1H), 8.02 (s, 1H), 6.78 (d, J = 5.9 Hz, 1H), 4.49 |
| | (brs, 3H), 3.80 (s, 5H), 3.62 (ddt, J = 23.7, 16.0, 9.5 Hz, 4H), 2.85-2.77 |
| | (m, 1H), 2.36-2.20 (m, 2H), 1.90 (dq, J = 20.7, 9.1 Hz, 8H), 1.78-1.54 |
| | (m, 8H). |
| 47 | ¹H NMR (400 MHz, CDCl₃) δ 9.32 (s, 1H), 8.59 (s, 1H), 8.35 (d, J = 5.8 |
| | Hz, 1H), 8.20 (s, 1H), 8.00 (s, 1H), 6.46 (d, J = 5.8 Hz, 1H), 4.81 (d, J = |
| | 7.5 Hz, 1H), 4.43 (s, 1H), 3.87 (s, 3H), 3.85 (m, 1H), 3.76 (dt, J = 9.7, 7.4 |
| | Hz, 1H), 3.67 (td, J = 8.7, 5.3 Hz, 1H), 3.49 (dd, J = 10.0, 4.0 Hz, 1H), |
| | 2.35 (dq, J = 13.5, 6.8 Hz, 1H), 1.98-2.04 (m, 4H), 1.47 (s, 9H). |
| 51 | ¹H NMR (400 MHz, DMSO-d₆) δ 10.26 (s, 1H), 9.19 (s, 1H), 8.70 (s, |
| | 1H), 8.29 (d, J = 5.6 Hz, 1H), 8.00 (d, J = 1.1 Hz, 1H), 7.53 (d, J = 6.6 |
| | Hz, 1H), 6.76 (d, J = 5.7 Hz, 1H), 4.60 (brs, 2H), 4.23-4.15 (m, 1H), |
| | 3.79 (s, 3H), 3.76-3.64 (m, 2H), 3.61-3.52 (m, 4H), 2.20 (dq, J = 13.1, |
| | 6.7 Hz, 1H), 1.91 (s, 4H), 1.69 (s, 2H). |
| 52 | ¹H NMR (400 MHz, DMSO-d₆) δ 10.21 (s, 1H), 9.14 (s, 1H), 8.65 (s, |
| | 1H), 8.23 (d, J = 5.8 Hz, 1H), 7.93 (s, 1H), 7.40 (d, J = 6.8 Hz, 1H), 6.70 |
| | (dd, J = 5.8, 1.5 Hz, 1H), 4.04-3.98 (m, 1H), 3.73 (m, 5H), 3.65-3.59 |
| | (m, 1H), 3.51 (q, J = 7.7 Hz, 1H), 3.37-3.33 (m, 1H), 2.94 (s, 3H), 2.23- |
| | 2.16 (m, 2H), 1.95-1.88 (m, 2H), 1.84 (s, 3H), 1.63 (s, 3H). |
| 54 | ¹H NMR (400 MHz, DMSO-d₆) δ 10.27 (s, 1H), 9.21 (s, 1H), 8.72 (s, |
| | 1H), 8.41 (d, J = 6.8 Hz, 1H), 8.30 (d, J = 5.8 Hz, 1H), 8.00 (s, 1H), 6.76 |
| | (d, J = 5.9 Hz, 1H), 4.41 (q, J = 5.8 Hz, 1H), 3.80 (s, 3H), 3.78-3.68 (m, |
| | 2H), 3.60 (q, J = 7.9 Hz, 1H), 3.40-3.35 (m, 1H), 2.20 (dt, J = 13.9, 6.9 |
| | Hz, 1H), 1.92 (d, J = 6.0 Hz, 3H), 1.70 (s, 2H), 1.57 (td, J = 7.6, 3.8 Hz, |
| | 1H), 0.71-0.62 (m, 4H). |
| 55 | ¹H NMR (400 MHz, DMSO-d₆) δ 10.27 (s, 1H), 9.20 (s, 1H), 8.70 (s, |
| | 1H), 8.30 (d, J = 5.8 Hz, 1H), 8.00 (s, 1H), 7.54 (d, J = 6.7 Hz, 1H), 6.76 |
| | (d, J = 5.8 Hz, 1H), 4.20 (q, J = 6.1 Hz, 1H), 3.80 (s, 3H), 3.76 (dd, J = |
| | 9.9, 6.6 Hz, 1H), 3.68 (q, J = 7.6 Hz, 1H), 3.59 (t, J = 7.9 Hz, 1H), 3.55 |
| | (s, 3H), 3.39 (d, J = 4.8 Hz, 1H), 2.19 (dt, J = 13.7, 6.6 Hz, 1H), 2.04- |
| | 1.94 (m, 1H), 1.92 (d, J = 7.5 Hz, 3H), 1.70 (s, 2H). |
| 56 | ¹H NMR (400 MHz, DMSO-d₆) δ 10.28 (s, 1H), 9.75-9.73 (d, 1H), 9.22 |
| | (s, 1H), 8.74 (s, 1H), 8.30 (d, J = 5.8 Hz, 1H), 8.00 (s, 1H), 6.77 (d, J = |
| | 5.7 Hz, 1H), 4.51 (q, J = 5.9 Hz, 1H), 3.88-3.82 (m, 1H), 3.80 (s, 3H), |
| | 3.72 (q, J = 7.9 Hz, 1H), 3.61 (q, J = 7.9 Hz, 1H), 3.53 (dd, J = 10.0, 4.7 |
| | Hz, 1H), 3.34 (s, 3H), 2.36-2.24 (m, 2H), 2.05 (dp, J = 11.5, 5.8 Hz, |
| | 2H), 1.91 (d, J = 7.7 Hz, 3H), 1.70 (s, 2H). |
| 57 | ¹H NMR (400 MHz, DMSO-d₆) δ 10.28 (s, 1H), 9.21 (s, 1H), 8.72 (s, |
| | 1H), 8.30 (d, J = 5.9 Hz, 1H), 8.00 (s, 1H), 7.51-7.49 (d, 1H), 6.77 (d, J = |
| | 5.8 Hz, 1H), 4.06-4.02 (m, 1H), 3.81 (m, J = 2.1 Hz, 4H), 3.70 (m, 1H), |
| | 3.58 (m, 1H), 3.41 (m, 1H), 3.09 (t, J = 7.3 Hz, 2H), 2.29-2.24 (m, 2H), |
| | 1.99 (m, 3H), 1.92 (s, 3H), 1.71 (s, 2H), 1.24 (m, 5H). |
| 58 | ¹H NMR (400 MHz, DMSO-d₆) δ 10.26 (s, 1H), 9.20 (s, 1H), 8.71 (s, |
| | 1H), 8.30 (d, J = 5.8 Hz, 1H), 8.00 (s, 1H), 6.77 (d, J = 5.8 Hz, 1H), 5.32 |
| | (t, J = 4.8 Hz, 1H), 4.66 (brs, 2H), 4.15-4.08 (m, 1H), 3.80 (s, 4H), 3.70 |
| | (q, J = 7.9 Hz, 1H), 3.59 (t, J = 8.0 Hz, 1H), 3.44 (dd, J = 10.0, 5.5 Hz, |
| | 1H), 2.70-2.64 (m, 1H), 2.33-2.24 (m, 2H), 2.02-1.91 (m, 4H), 1.74- |
| | 1.68 (m, 1H), 1.46 (d, J = 7.7 Hz, 1H), 0.97 (1, J = 6.9 Hz, 4H). |
| A2 | ¹H NMR (400 MHz, DMSO-d₆) δ 10.35 (s, 1H), 9.09 (s, 1H), 8.77 |
| | (s, 1H), 8.31 (d, J = 5.8 Hz, 1H), 7.93 (s, 1H), 6.74 (d, J = 5.8 Hz, 1H), |
| | 5.14 (dt, J = 14.0, 7.0 Hz, 1H), 4.37 (t, J = 11.3 Hz, 1H), 3.95 (d, J = 10.2 |
| | Hz, 1H), 3.76 (s, 3H), 3.64-3.55 (m, 2H), 3.49 (p, J = 7.2 Hz, 1H), 3.12 |
| | (td, J = 6.2, 1.4 Hz, 2H), 2.24 (s, 3H), 2.00 (t, J = 12.9 Hz, 1H), 1.76 |
| | (d, J = 6.8 Hz, 3H). |
| A3 | ¹H NMR (400 MHz, DMSO-d₆) δ 10.27 (s, 1H), 9.01 (s, 1H), 8.67 (s, |
| | 1H), 8.24 (d, J = 5.8 Hz, 1H), 7.86 (s, 1H), 6.67 (d, J = 5.9 Hz, 1H), 5.07 |
| | (dd, J = 10.4, 6.6 Hz, 1H), 4.30 (t, J = 11.4 Hz, 1H), 3.88 (d, J = 10.2 Hz, |
| | 1H), 3.69 (s, 3H), 2.66 (m, 3H), 2.11 (s, 3H), 2.05 (m, 2H), 1.94 (t, J = |
| | 12.8 Hz, 2H), 1.89-1.79 (m, 2H), 1.69 (d, J = 6.9 Hz, 3H), 1.66-1.56 |
| | (m, 2H). |
| A4 | ¹H NMR (400 MHz, DMSO-d₆) δ 8.67 (d, J = 1.0 Hz, 1H), 8.42 (d, J = |
| | 6.1 Hz, 1H), 8.27 (d, J = 1.1 Hz, 1H), 7.87 (s, 1H), 6.79 (d, J = 6.1 Hz, |
| | 1H), 4.96-4.82 (m, 1H), 4.37-4.28 (m, 2H), 4.03-3.94 (m, 2H), 3.84 |
| | (d, J = 8.3 Hz, 2H), 3.70 (s, 3H), 3.61-3.53 (m, 5H), 3.42-3.35 (m, 1H), |
| | 3.00 (s, 3H), 2.42-2.31 (m, 1H), 1.91 (t, J = 12.9 Hz, 1H), 1.67 (d, J = |
| | 6.7 Hz, 3H). |

### III. Biological Test Evaluation

### Cell Proliferation Assay

### (I) Reagents and Consumables

DMEM Medium (Gibco, Cat #11965118)
RPMI1640 Medium (Gibco, Cat #11875119)
Fetal Bovine Serum (FBS) (GBICO, Cat #10099-141)
CellTiter-Glo® Luminescent Cell Viability Assay Kit (Promega, Cat #G7572)
96-well, Black, Clear Flat Bottom Plate (Corning®, Cat #3603)

### (II) Instruments

SpectraMax Multimode Microplate Reader MD, 2104-0010A;
CO2 Incubator, Thermo Scientific 3100 Series;
Biosafety Cabinet, Thermo Scientific, 1300 Series A2;
Inverted Microscope, Olympus, CKX41SF;
Siemens Refrigerator, KK25E76TI.

### (III) Cell Lines and Culture Conditions

| | | | Cell |
|---|---|---|---|
| No. | Cell Line | Cell Medium | Density |
| 1 | A431 WT | DMEM + 15% FBS | 5000 |
| 2 | Ba/F3 EGFR-L858R- | RPMI1640 + 10% FBS | 3000 |
| | C797S (LC) | | |
| 3 | Ba/F3 EGFR-DEL19- | RPMI1640 + 10% FBS | 3000 |
| | C797S (DC) | | |
| 4 | Ba/F3 EGFR-L858R- | RPMI1640 + 10% FBS | 3000 |
| | T790M-C797S (LTC) | | |
| 5 | Ba/F3 EGFR-DEL19- | RPMI1640 + 10% FBS | 3000 |
| | T790M-C797S (DTC) | | |

### (IV) Experiment Steps

### 1. Cell Culture and Inoculation:

(1) Harvest cells in a logarithmic growth phase and count the cells using a platelet counter. Test cell viability using a trypan blue exclusion method to ensure that cell viability is above 90%.
(2) Adjust the cell concentration to desired final density; and add 90 μL of cell suspension to a 96-well plate.
(3) Incubate the cells overnight at 37° C., under 5% CO₂ and 95% humidity in the 96-well plate.

### 2. T0 Reference Data:

(1) Add 10 μL PBS to each well of the TO plate containing cells.
(2) Thaw the CTG reagent and equilibrate the plate to room temperature for 30 minutes.
(3) Add an equal volume of CTG solution to each well.
(4) Lyse cells by shaking on an orbital shaker for 5 minutes.
(5) Leave the plate at room temperature for 20 minutes to stabilize the luminescence signal.
(6) Read the TO fluorescence signal.

### 3. Compound Dilution and Addition

(1) Add a corresponding volume of DMSO to a corresponding compound powder according to the compound information sheet to prepare a 10 mM stock solution.
(2) Prepare 1000-fold and 3.16-fold diluted compound solutions.
(3) Dilute the 1000× diluted compound solution 100-fold with PBS to prepare a 10-fold compound solution, with a maximum concentration of 10 μM, nine concentrations and 3.16-fold dilution. Then add 10 μL of the drug solution concentration to individual wells of the 96-well plate seeded with cells. Test each compound concentration in triplicate wells, and the final DMSO concentration is 0.1%.
(4) Incubate the cells in the 96-well plate containing the drug compounds at 37° C., under 5% CO₂, and 95% humidity for an additional 72 hour period prior to performing the CTG analysis.

### 4. Fluorescence Signal Reading

(1) Thaw the CTG reagent and equilibrate the plate to room temperature for 30 minutes.
(2) Add an equal volume of CTG solution to each well.
(3) Lyse cells by shaking on an orbital shaker for 5 minutes.
(4) Leave the plate at room temperature for 20 minutes to stabilize the fluorescence signal.
(5) Read fluorescence value.

### 5. Data Processing

Analyze the data by using GraphPad Prism 7.0 software, and use Non-linear S-curve regression to fit the dose-response curves, to calculate the IC₅₀ values (in nM). The specific experiment results are presented in Table 1:

$$Cell survival rate ( \% ) = ( Lum test drug - Lum culture medium control ) \text{} / ( Lum cell control - Lum culture medium control ) \times 100 \% .$$

TABLE 1
Biology Test Results

| Example | A431 | BaF3 | BaF3 | BaF3 | BaF3 |
|---|---|---|---|---|---|
| No. | WT | LTC | DTC | LC | DC |
| 1 | 322.8 | 10.5 | 7.2 | 17.4 | 7.9 |
| 2 | 602.9 | 12.8 | 17.5 | 21.1 | 30.4 |
| 3 | 439.4 | 10.4 | 6.8 | 30.6 | 19.4 |
| 4 | 822.1 | 12.4 | 15.6 | 50.3 | 26.8 |
| 5 | 1084.3 | 9.2 | 4.7 | 14.0 | 19.7 |
| 6 | 1358.0 | 28.3 | NT | 35.6 | NT |
| 7 | 1660.0 | 24.6 | NT | 43.5 | NT |
| 8 | 499.2 | 39.4 | 27.8 | 63.5 | 31.5 |
| 9 | 1923.4 | 47.0 | 18.4 | 62.7 | 32.8 |
| 11 | 806.9 | 133.2 | 92.2 | 187.2 | 127.7 |
| 12 | 629.0 | 61.9 | NT | 123.5 | NT |
| 13 | 794.4 | 14.9 | 8.4 | 25.5 | 14.8 |
| 14 | 1716 | 3.6 | 3.5 | 5.0 | 2.8 |
| 15 | 1047.0 | 32.4 | NT | 84.4 | NT |
| 16 | 481.6 | 13.3 | NT | 23.8 | NT |
| 17 | 390.9 | 7.8 | NT | 32.1 | NT |
| 18 | 765.0 | 10.9 | NT | 25.4 | NT |
| 19 | 770.9 | 11.3 | NT | 19.5 | NT |
| 20 | 511.5 | 16.0 | NT | 38.0 | NT |
| 21 | 171.4 | 4.7 | 3.4 | 15.8 | 7.1 |
| 22 | 408.8 | 5.9 | NT | 13.2 | NT |
| 23 | 770.6 | 8.1 | 3.5 | 19.5 | 7.8 |
| 24 | 378.1 | 6.1 | 0.7 | NT | 4.4 |
| 25 | 570.7 | 106 | 42.6 | 123 | 66.5 |
| 26 | 645.4 | 28.5 | 16.3 | 33.5 | 11.4 |
| 27 | 587.2 | 5.2 | 1.0 | 13.4 | 1.1 |
| 28 | 682.4 | 86.9 | 12.4 | 45.7 | 12.8 |
| 29 | 621.8 | 4.0 | 1.8 | 9.6 | 1.6 |
| 30 | 743.5 | 5.8 | 2.4 | 11.8 | 2.5 |
| 31 | 366.3 | 4.3 | 4.8 | 10.8 | 3.5 |
| 32 | 543.0 | 12.1 | 10.5 | 14.9 | 5.1 |
| 34 | 375.3 | 1.4 | 1.6 | 2.7 | 0.7 |
| 35 | 345.0 | 1.0 | 0.7 | 2.2 | 0.9 |
| 36 | 614.0 | 2.0 | 1.5 | 5.1 | 2.5 |
| 37 | 553.9 | 6.4 | 3.2 | 15.8 | 6.1 |
| 39 | 445.7 | 0.8 | 1.5 | 2.1 | 2.3 |
| 40 | 320.5 | 0.7 | NT | 2.1 | NT |
| 41 | 564.0 | 3.1 | 5.2 | 12.9 | 15.9 |
| 42 | 592.9 | 1.2 | 2.3 | 1.9 | 3.5 |
| 43 | 699.6 | 1.3 | 3.6 | 2.9 | 5.0 |
| 44 | 1132.0 | 1.9 | 4.0 | 5.3 | 10.1 |
| 45 | 738.1 | 1.1 | 3.9 | 2.9 | 4.0 |
| 47 | 2574.0 | 35.2 | NT | 82.0 | NT |
| 48 | 357.0 | 4.5 | 2.0 | 7.6 | 2.5 |
| 49 | 926.0 | 29.6 | NT | 59.9 | NT |
| 50 | 1061.0 | 20.7 | NT | 86.5 | NT |
| 51 | 424.1 | 9.0 | 10.1 | 17.6 | 15.0 |
| 52 | 260.9 | 16.4 | NT | 31.0 | NT |
| 54 | 654.2 | 12.0 | 8.7 | 31.6 | 12.1 |
| 55 | 329.2 | 12.0 | 8.5 | 33.1 | 12.1 |
| 56 | 842.9 | 12.5 | NT | 36.5 | NT |
| 57 | 251.2 | 7.9 | 3.1 | 22.8 | 5.3 |
| 58 | 911.4 | 2.1 | 2.8 | 6.9 | 7.2 |
| A1 | 2701 | 262.0 | 64.9 | 299 | 40.9 |
| A2 | 2492 | 167.3 | NT | 176.5 | NT |
| A3 | 1843 | 62.1 | NT | 53.3 | NT |
| A4 | 2681.0 | 186.4 | 243.8 | 379.7 | 175.9 |
| Positive | 449.5 | 8.0 | 4.2 | 11.5 | 4.9 |
| Compound | | | | | |

Notes
“NT” is an abbreviation of “Not Tested”, which means that it has not been tested for the time being.

The “positive compound” used in Biological Test Evaluation and Pharmacokinetic Assay in Mice of the present invention is compound 101 from WO2021168074A1, which is one of the most potent active compounds described in that prior art. The chemical structure of compound 101 is as follows:

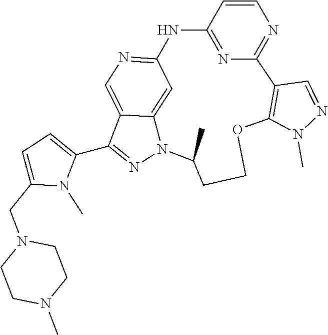 [SMILES: C[C@H]1CCOc2c(cnn2C)-c2nccc(n2)Nc2cc3c(cn2)c(-c2ccc(CN4CCN(C)CC4)n2C)nn31]

From the bioactivity data of the compounds in specific examples, it can be seen that the series of compounds of the present invention had a strong inhibitory effect on various resistant EGFR C797S mutations at the cellular level, including the EGFR L858R/C797S double mutation, EGFR Del19/C797S double mutation. L858R/T790 M/C797S triple mutation and Del19/T790 M/C797S triple mutation, and had high selectivity for EGFR WT.

### IV. Pharmacokinetic Experiment in Mice

### (I) Study Purpose

The purpose of this trial is to study the pharmacokinetic behavior of some compounds of the present invention, and the administration method is: Single oral administration (PO) to ICR mice.

### (II) Trial Protocol

### 1. Test Drug

The compounds used in this trial are from the compounds in the specific examples of the present invention and the listed positive compounds.

### 2. Test Animals

ICR mice male N=9 original source: Shanghai Sippr-Bk Lab Animal Co., Ltd.

### 3. Drug Preparation and Administration

The compounds are weighed and dissolved in the corresponding solution (10% PG+10% Solutol®+80% pH3 citrate buffer). The mixture is then shaken well and sonicated to prepare a 1 mg/mL stock solution. Nine mice are orally administered at a dose of 10 mg/kg (or IV 2 mg/kg) after an overnight fast.

### 4. Sample Collection

About 90 μL of blood is collected via the submandibular vein per time point, heparin sodium anticoagulant is added, the sample is placed on ice after collection, and plasma is centrifuged within 1 hour (centrifugation conditions: 8,000 rpm, 6 minutes, 2-8° C.). The blood collection timepoints are 0, 0.25, 0.5, 1, 2, 4, 6, 8 and 24 h. The samples are stored in a −20° C. refrigerator.

The plasma sample is 40 μL, 160 μL of ice-cold acetonitrile containing internal standard is added, vortexed for 3 minutes, and centrifuged at 11,000 rpm for 5 minutes. 100 μL of the supernatant is taken and added to 100 μL of water, 5 μL of the sample is taken and injected into LC/MS/MS for analysis.

### 5. Test Results

TABLE 2
Pharmacokinetic Experiment Results

| Example | | Cₘₐₓ | AUCₗₐₛₜ | T_{1/2} |
|---|---|---|---|---|
| No. | Dose | (ng/mL) | (hr*ng/mL) | (h) |
| Example 1 | 10 mg/kg | 88.2 | 1029.9 | 3.5 |
| Example 3 | 10 mg/kg | 307.1 | 3916.8 | 5.6 |
| Example 7 | 10 mg/kg | 193.4 | 2515.9 | 5.6 |
| Example 13 | 10 mg/kg | 1542.3 | 8867.0 | 2.2 |
| Example 14 | 10 mg/kg | 882.3 | 5321.2 | 2.4 |
| Example 22 | 10 mg/kg | 502.8 | 2336.1 | 3.0 |
| Example 29 | 10 mg/kg | 1399.1 | 4540.4 | 0.66 |
| Example 34 | 10 mg/kg | 693.6 | 2388.8 | 4.7 |
| Example 35 | 10 mg/kg | 1498.7 | 5574.9 | 2.6 |
| | | | (Oral F, 81.5%) | |
| Example 37 | 10 mg/kg | 2071.5 | 10058.2 | 3.1 |
| Example 39 | 10 mg/kg | 1559.2 | 6082.1 | 3.0 |
| Example 40 | 10 mg/kg | 1352.4 | 4943.3 | 3.5 |
| Example 41 | 10 mg/kg | 1918.8 | 7105.9 | 2.3 |
| Example 42 | 10 mg/kg | 1059.2 | 4286.4 | 3.0 |
| Example 51 | 10 mg/kg | 595.5 | 2691.9 | 2.8 |
| Example 54 | 10 mg/kg | 412.3 | 1730.4 | 1.3 |
| Positive | 10 mg/kg | 32.7 | 204.4 | 4.8 |
| Compounds | | | | |

From the pharmacokinetic data of the compounds in the above specific examples, this series of compounds of the present invention were absorbed into the mice body via a single oral dose (10 mg/kg), and showed very good PK data. Compared to the positive compounds, the AUC values of this series of compounds of the present invention were increased by at least 5-fold. Furthermore, some of the compounds in the examples exhibited AUC increases of up to 10-fold or more. The experiment results demonstrate that this series of compounds of the present invention exhibit significantly increased oral exposure, have good oral bioavailability, and are likely to solve the problem that pharmacokinetic limitations that make the positive compounds unsuitable for oral administration.

All documents mentioned in the present invention are incorporated by reference in this application, just as each document is cited separately as a reference. In addition, it should be understood that various modifications or changes may be made by those skilled in the art after reading the above disclosed content of the present invention, and these equivalent forms also fall within the scope defined by the claims appended hereto.

1. A compound of formula (I), or a stereoisomer or pharmaceutically acceptable salt thereof: 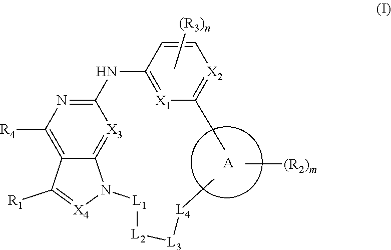
wherein
X₁ is CRₐ or N;
X₂ is CR_{b} or N;
X₃ is CR_{c} or N;
X₄ is CR_{d} or N;
L₁ is CR₅ₐR_{5b};
L₂ is (CR_{5c}R_{5d})ₚ, NR₆ₐ, O, S, S(O), S(O)₂, or C(O);
L₃ is a bond, CR₅ₑR_{5f}, NR_{6b}, O, S, S(O), S(O)₂, or C(O);
L₂ and L₃ together form a C₄₋₁₂ cycloalkyl, 4-12 membered heterocyclyl, C₆₋₁₀ aryl, or 5-10 membered heteroaryl, and the C₄₋₁₂ cycloalkyl, 4-12 membered heterocyclyl, C₆₋₁₀ aryl, or 5-10 membered heteroaryl is independently optionally further substituted by one or more substituents independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂;
L₄ is CR_{5g}R₅ₕ, O, S, S(O), S(O)₂, NR_{6c}, or C(O);
ring A is C₄₋₁₂ cycloalkyl, 4-12 membered heterocyclyl, C₆₋₁₀ aryl, or 5-10 membered heteroaryl;
R₁ is selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₃)—C(O)R₁₂, the above R₁ groups each optionally further substituted by one or more substituents independently selected from the group consisting of deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₁₋₁₀ alkyl substituted by C₃₋₁₂ cycloalkyl, C₁₋₁₀ alkyl substituted by 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-N(R₁₃)—S(O)₂R₁₀, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂, the above one or more substituents each optionally further substituted by one or more substitutents independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-N(R₁)—S(O)₂R₁₀, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂;
each R₂ is independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₃)—C(═NR₁₄)R₁₂, —C₀₋₈ alkyl-C(O)NR₁₃R₁₄ and —C₀₋₈ alkyl-N(R₁₃)—C(O)R₁₂, the above R₂ groups each optionally further substituted by one or more substituents independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)R₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₃)—C(O)R₁₂;
each R₃, each Rₐ, and each R_{b} are independently selected from the group consisting of hydrogen, deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂;
R₄ and R_{c} are each independently selected from the group consisting of hydrogen, deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂;
R_{d} is selected from the group consisting of hydrogen, deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)R₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂;
R₅ₐ and R_{5b} are each independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁)—C(O)R₁₂, or, R₅ₐ and R_{5b}, together with the carbon atom to which R₅ₐ and R_{5b} are directly attached, form a C(O), C₃₋₆ cycloalkyl, 4-10 membered heterocyclyl, C₆₋₁₀ aryl, or 5-10 membered heteroaryl, the above R₅ₐ and R_{5b} groups each optionally further substituted by one or more substituents independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₄, and —C₀₋₈ alkyl-N(R₁₃)—C(O)R₁₂;
each R_{5c} and each R_{5d} are independently selected form the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂, or, R_{5c} and R_{5d}, together with the carbon atom to which R_{5c} and R_{5d} are directly attached, form a C₃₋₆ cycloalkyl, 4-10 membered heterocyclyl, C₆₋₁₀ aryl, or 5-10 membered heteroaryl, the above R_{5c} and R_{5d} groups each optionally further substituted by one or more substituents independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁)—C(O)R₁₂, provided that when R_{5c} or R_{5d} is H or C₁₋₄ alkyl, R₁ is optionally substituted C₂₋₁₀ alkynyl, or, L₃ is O, S, S(O), or S(O)₂;
R₅ₑ and R_{5f} are each independently selected from the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)R₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂, or, R₅ₑ and R_{5f}, together with the carbon atom to which R₅ₑ and R_{5f} are directly attached, form a C₃₋₆ cycloalkyl, 4-10 membered heterocyclyl, C₆₋₁₀ aryl, or 5-10 membered heteroaryl, the above R₅ₑ and R_{5f} groups each optionally further substituted by one or more substituents independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂;
R_{5g} and R₅ₕ are each independently selected form the group consisting of hydrogen, deuterium, halogen, cyano, nitro, azido, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂, or, R_{5g} and R₅ₕ, together with the carbon atom to which R_{5g} and R₅ₕ are directly attached, form a C₃₋₆ cycloalkyl, 4-10 membered heterocyclyl, C₆₋₁₀ aryl, or 5-10 membered heteroaryl, the above R_{5g} and R₅ₕ groups each optionally further substituted by one or more substituents independently selected from the group consisting of deuterium, halogen, cyano, nitro, azido, C₁₋₁₀ alkyl, C₁₋₁₀ haloalkyl, C₁₋₁₀ deuteroalkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, ═O, ═S, —C₀₋₈ alkyl-SF₅, —C₀₋₈ alkyl-S(O)(═N—R₇)R₈, —C₀₋₈ alkyl-N═S(O)R₈R₉, —C₀₋₈ alkyl-N═SR₈R₉, —C₀₋₈ alkyl-O—S(O)₂R₁₀, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)SR₁₁, —C₀₋₈ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₈ alkyl-N(R₁₃)—C(O)R₁₂;
R₆ₐ is selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl, and —O—R₁₁, above R₆ₐ groups each optionally further substituted by one or more substituents independently selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy, and —NR₁₃R₁₄;
R_{6b} is selected from the group consisting of hydrogen, deuterium, C₁₋₄ alkyl, C₂₋₄ alkenyl, C₂₋₄ alkynyl, C₃₋₄ cycloalkyl, 3-6 membered heterocyclyl, and —O—R₁₁, the above R_{6b} groups each optionally further substituted by one or more substituents independently selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy, and —NR₁₃R₁₄;
R_{6c} is selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl, and —O—R₁₁, above R_{6c} groups each optionally further substituted by one or more substituents independently selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy, and —NR₁₃R₁₄;
each R₇ is independently selected from the group consisting of hydrogen, deuterium, C₁₋₁₀ alkyl, C₂₋₁₀ alkenyl, C₂₋₁₀ alkynyl, C₃₋₁₀ cycloalkyl, 3-10 membered heterocyclyl, C₆₋₁₀ aryl, 5-10 membered heteroaryl, —C₀₋₈ alkyl-S(O)ᵣR₁₀, —C₀₋₈ alkyl-C(O)OR₁₁, —C₀₋₈ alkyl-C(O)R₁₂, or —C₀₋₈ alkyl-C(O)NR₁₃R₁₄, the above R₇ groups optionally further substituted by one or more substituents independently selected from the group consisting of deuterium, halogen, cyano, nitro, azido, C₁₋₁₀ alkyl, C₂₋₁₀ alkenyl, C₂₋₁₀ alkynyl, C₁₋₁₀ haloalkyl, C₁₋₁₀ deuteroalkyl, C₃₋₁₀ cycloalkyl, 3-10 membered heterocyclyl, C₆₋₁₀ aryl, 5-10 membered heteroaryl, —C₀₋₈ alkyl-S(O)R₁₀, —C₀₋₈ alkyl-O—R₁₁, —C₀₋₈ alkyl-C(O)OR₁₁, —C₀₋₈ alkyl-C(O)R₁₂, —C₀₋₈ alkyl-(—C(O)R₁₂, —C₀₋₈ alkyl-NR₁₃R₁₄, —C₀₋₈ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, or —C₀₋₈ alkyl-N(R₁₃)—C(O)R₁₂;
each R₈ and each R₉ are independently selected from the group consisting of hydrogen, deuterium, hydroxy, C₁₋₁₀ alkyl, C₂₋₁₀ alkenyl, C₂₋₁₀ alkynyl, C₃₋₁₀ cycloalkyl, 3-10 membered heterocyclyl, C₆₋₁₀ aryl, and 5-10 membered heteroaryl, or, R₈ and R₉, together with the sulfur atom to which R₈ and R₉ are directly attached, form a 3-10 membered heterocyclyl, the above R₈ and R₉ groups optionally further substituted by one or more substituents independently selected from the group consisting of deuterium, halogen, cyano, nitro, azido, C₁₋₁₀ alkyl, C₂₋₁₀ alkenyl, C₂₋₁₀ alkynyl, C₁₋₁₀ haloalkyl, C₁₋₁₀ deuteroalkyl, C₃₋₁₀ cycloalkyl, 3-10 membered heterocyclyl, C₆₋₁₀ aryl, 5-10 membered heteroaryl, —C₀₋₈ alkyl-S(O)ᵣR₁₀, —C₀₋₈ alkyl-O—R₁₁, —C₀₋₈ alkyl-C(O)OR₁₁, —C₀₋₈ alkyl-C(O)R₁₂, —C₀₋₈ alkyl-O—C(O)R₁₂, —C₀₋₈ alkyl-NR₁₃R₁₄, —C₀₋₈ alkyl-C(═NR₁₃)R₁₂, —C₀₋₈ alkyl-N(R₃)—C(═NR₁₄)R₁₂, —C₀₋₈ alkyl-C(O)NR₁₃R₁₄, and —C₀₋₈ alkyl-N(R₁₃)—C(O)R₁₂;
each R₁₀ is independently selected from the group consisting of hydrogen, deuterium, hydroxy, C₁₋₁₀ alkyl, C₂₋₁₀ alkenyl, C₃₋₁₂ cycloalkyl, 3-12 membered heterocyclyl, C₆₋₁₀ aryl, 5-10 membered heteroaryl, and —NR₁₃R₁₄, the above R₁₀ groups each optionally further substituted by one or more substituents independently selected from the group consisting of deuterium, halogen, hydroxy, ═O, C₁₋₁₀ alkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy, and —NR₁₃R₁₄;
each R₁₁ is independently selected from the group consisting of hydrogen, deuterium, C₁₋₁₀ alkyl, C₂₋₁₀ alkenyl, C₃₋₁₂ cycloalkyl, 3-12 membered heterocyclyl, C₆₋₁₀ aryl, and 5-10 membered heteroaryl, and the above R₁₁ groups are each independently optionally further substituted by one or more substituents independently selected from the group consisting of deuterium, halogen, hydroxy, ═O, cyano, C₁₋₁₀ alkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy, and —NR₁₃R₁₄;
each R₁₂ is independently selected from the group consisting of hydrogen, deuterium, hydroxy, C₁₋₁₀ alkyl, C₁₋₁₀ alkoxy, C₂₋₁₀ alkenyl, C₂₋₁₀ alkynyl, C₃₋₁₂ cycloalkyl, C₃₋₁₂ cycloalkoxy, 3-12 membered heterocyclyl, 3-12 member heterocyclyloxy, C₆₋₁₀ aryl, C₆₋₁₀ aryloxy, 5-10 membered heteroaryl, 5-10 membered heteroaryloxy, and —NR₁₃R₁₄, the above R₁₂ groups each optionally further substituted by one or more substituents independently selected from the group consisting of deuterium, halogen, hydroxy, ═O, cyano, C₁₋₁₀ alkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy, and —NR₁₃R₁₄;
each R₁₃ and each R₁₄ are independently selected from the group consisting of hydrogen, deuterium, hydroxy, C₁₋₁₀ alkyl, C₂₋₁₀ alkenyl, C₂₋₁₀ alkynyl, C₃₋₁₂ cycloalkyl, 3-12 membered heterocyclyl, C₆₋₁₀ aryl, 5-10 membered heteroaryl, sulfinyl, sulfonyl, methanesulfonyl, isopropylsulfonyl, cyclopropylsulfonyl, p-toluenesulfonyl, aminosulfonyl, dimethylaminosulfonyl, and C₁₋₁₀ alkanoyl, the above R₁₃ and R₁₄ groups each optionally further substituted by one or more substituents independently selected from the group consisting of deuterium, halogen, hydroxy, ═O, C₁₋₁₀ alkyl, C₂₋₁₀ alkenyl, C₂₋₁₀ alkynyl, C₁₋₁₀ haloalkyl, C₁₋₁₀ deuteroalkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy, amino, monoC₁₋₁₀ alkylamino, diC₁₋₁₀ alkylamino, and C₁₋₁₀ alkanoyl; or
R₁₃ and R₁₄, together with the nitrogen atom to which they are directly attached, form a 4-10 membered heterocyclyl or 5-10 membered heteroaryl, the 4-10 membered heterocyclyl or 5-10 membered heteroaryl optionally further substituted by one or more substituents independently selected from the group consisting of deuterium, halogen, hydroxy, ═O, C₁₋₁₀ alkyl, C₂₋₁₀ alkenyl, C₂₋₁₀ alkynyl, C₁₋₁₀ haloalkyl, C₁₋₁₀ deuteroalkyl, C₁₋₁₀ alkoxy, C₃₋₁₂ cycloalkyl, C₃₋₁₂ cycloalkoxy, 3-12 membered heterocyclyl, 3-12 membered heterocyclyloxy, C₆₋₁₀ aryl, C₆₋₁₀ aryloxy, 5-10 membered heteroaryl, 5-10 membered heteroaryloxy, methanesulfonylmethyl, amino, monoC₁₋₁₀ alkylamino, diC₁₋₁₀ alkylamino, and C₁₋₁₀ alkanoyl;
m is 0, 1, 2, 3 or 4;
n is 0, 1, or 2;
p is 1 or 2; and
each r is independently 0, 1, or 2.

2. The compound of formula (I), or the stereoisomer or pharmaceutically acceptable salt thereof, according to claim 1, wherein X₁ is CRₐ or N;
X₂ is CR_{b} or N;
X₃ is CR_{c} or N;
X₄ is CR_{d} or N;
L₁ is CR₅ₐR_{5b};
L₂ is (CR_{5c}R_{5d})ₚ, NR₆ₐ, O, S, S(O), S(O)₂ or C(O);
L₃ is a bond, CR₅ₑR_{5f}, NR_{6b}, O, S, S(O), S(O)₂, or C(O); or
L₂ and L₃ together form a C₄₋₆ cycloalkyl, 4-6 membered heterocyclyl, C₆₋₈ aryl, or 5-8 membered heteroaryl, and the C₄₋₆ cycloalkyl, 4-6 membered heterocyclyl, C₆₋₈ aryl, or 5-8 membered heteroaryl is optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂;
L₄ is CR_{5g}R₅ₕ, O, S, S(O), S(O)₂, NR_{6c}, or C(O);
ring A is C₄₋₆ cycloalkyl, 4-6 membered heterocyclyl, C₆₋₈ aryl, or 5-8 membered heteroaryl;
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₈ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₄ alkyl-N(R₁₃)—C(O)R₁₂, the above R₁ groups each optionally further substituted by one or more substituents independently selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-N(R₁₃)—S(O)₂R₁₀, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂, the above one or more substituents each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-N(R₁₃)—S(O)₂R₁₀, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₃)—C(═NR₁₄)R₁₂, —C₀₋₄ alkyl-C(O)NR₁₃R₁₄, and —C₀₋₄ alkyl-N(R₁₃)—C(O)R₁₂;
each R₂ is independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁)R₁₂, —C₀₋₄ alkyl-N(R₃)—C(═NR₁₄)R₁₂, —C₀₋₄ alkyl-C(O)NR₁₃R₁₄, and —C₀₋₄ alkyl-N(R₁)—C(O)R₁₂, the above R₂ groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂;
each R₃, each Rₐ, and each R_{b} are independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heterocyclyl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂;
R₄ and R_{c} are each independently selected form the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂;
R_{d} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂;
R₅ₐ and R_{5b} are each independently selected form the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₈ alkyl-O—C(O)R₁₂, —C₀₋₄ alkyl-P(O)(R₁₂)₂, —C₀₋₄ alkyl-NR₁₃R₁₄, —C₀₋₈ alkyl-C(═NR₁₃)R₁₂, —C₀₋₈ alkyl-N(R₃)—C(═NR₁₄)R₁₂, —C₀₋₄ alkyl-C(O)NR₁₃R₁₄, and —C₀₋₈ alkyl-N(R₁₃)—C(O)R₁₂, or, R₅ₐ and R_{5b}, together with the carbon atom to which R₅ₐ and R_{5b} are directly attached, form a C(O), C₃₋₆ cycloalkyl, 4-6 membered heterocyclyl, C₆₋₈ aryl, or 5-8 membered heteroaryl, the above R₅ₐ and R_{5b}, groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁3R₁₄ and —C₀₋₄ alkyl-N(R₁₃)—C(O)R₁₂;
each R_{5c} and each R_{5d} are independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂, or, R_{5c} and R_{5d}, together with the carbon atom to which R_{5c}, and R_{5d} are directly attached, form a C₃₋₆ cycloalkyl, 4-6 membered heterocyclyl, C₆₋₈ aryl, or 5-8 membered heteroaryl, the above R_{5c} and R_{5d} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₈ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₃, —C₀₋₄ alkyl-C(O)NR₁₃R₁₄, and —C₀₋₄ alkyl-N(R₁₃)—C(O)R₁₂, provided that when R_{5c} or R_{5d} is H or C₁₋₄ alkyl, R₁ is optionally substituted C₂₋₁₀ alkynyl, or, L₃ is O, S, S(O), or S(O)₂;
R₅ₑ and R_{5f} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)R₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁)R₁₂, —C₀₋₄ alkyl-N(R₃)—C(═NR₁₄)R₁₂, —C₀₋₄ alkyl-C(O)NR₁₃R₁₄, and —C₀₋₄ alkyl-N(R₁₃)—C(O)R₁₂, or, R₅ₑ and R_{5f}, together with the carbon atom to which R₅ₑ and R_{5f} are directly attached, form a C₃₋₆ cycloalkyl, 4-6 membered heterocyclyl, C₆₋₈ aryl, or 5-8 membered heteroaryl, the above R₅ₑ and R_{5f} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂; and
R_{5g} and R₅ₕ are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)R₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₂, or, R_{5g} and R₅ₕ, together with the carbon atom to which R_{5g} and R₅ₕ are directly attached, form a C₃₋₆ cycloalkyl, 4-6 membered heterocyclyl, C₆₋₈ aryl, or 5-8 membered heteroaryl, the above R_{5g} and R₅ₕ groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₃)—C(O)R₁₂.

3. The compound of formula (I), or the stereoisomer or pharmaceutically acceptable salt thereof, according to claim 1, wherein each R₇ is independently selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —C₀₋₄ alkyl-S(O)ᵣR₁₀, —C₀₋₈ alkyl-C(O)OR₁₁, —C₀₋₄ alkyl-C(O)R₁₂ and —C₀₋₄ alkyl-C(O)NR₁₃R₁₄, the above R₇ groups optionally further substituted by one or more substituents independently selected from the group consisting of deuterium, halogen, cyano, C₁₋₄ alkyl, C₂₋₄ alkenyl, C₂₋₄ alkynyl, C₁₋₄ haloalkyl, C₁₋₄ deuteroalkyl, C₃₋₆ cycloalkyl, 3-6 membered heterocyclyl, C₆₋₈ aryl, 5-8 membered heteroaryl, —C₀₋₈ alkyl-S(O)ᵣR₁₀, —C₀₋₄ alkyl-O—R₁₁, —C₀₋₈ alkyl-C(O)OR₁₁, —C₀₋₄ alkyl-C(O)R₁₂, —C₀₋₄ alkyl-O—C(O)R₁₂, —C₀₋₄ alkyl-NR₁₃R₁₄, —C₀₋₈ alkyl-C(═NR₁₃)R₁₂, —C₀₋₈ alkyl-N(R₃)—C(═NR₁₄)R₁₂, —C₀₋₄ alkyl-C(O)NR₁₃R₁₄, and —C₀₋₈ alkyl-N(R₁₃)—C(O)R₁₂;
each R₈ and each R₉ are independently selected from the group consisting of hydrogen, deuterium, hydroxy, C₁₋₄ alkyl, C₂₋₄ alkenyl, C₂₋₄ alkynyl, C₃₋₆ cycloalkyl, 3-6 membered heterocyclyl, C₆ aryl, and 5-8 membered heteroaryl, or, R₈ and R₉, together with the sulfur atom to which R₈ and R₉ are directly attached, form a 3-6 membered heterocyclyl, the above R₈ and R₉ groups optionally further substituted by one or more substituents independently selected from the group consisting of deuterium, halogen, cyano, C₁₋₄ alkyl, C₂₋₄ alkenyl, C₂₋₄ alkynyl, C₁₋₄ haloalkyl, C₁₋₄ deuteroalkyl, C₃₋₆ cycloalkyl, 3-6 membered heterocyclyl, C₆₋₈ aryl, 5-8 membered heteroaryl, —C₀₋₄ alkyl-S(O)ᵣR₁₀, —C₀₋₈ alkyl-O—R₁₁, —C₀₋₄ alkyl-C(O)OR₁₁, —C₀₋₄ alkyl-C(O)R₁₂, —C₀₋₄ alkyl-O—C(O)R₁₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₄, and —C₀₋₄ alkyl-N(R₁₃)—C(O)R₁₂;
each R₁₀ is independently selected from the group consisting of hydrogen, deuterium, C₁₋₄ alkyl, C₂₋₄ alkenyl, C₃₋₆ cycloalkyl, 3-6 membered heterocyclyl, C₆₋₈ aryl, 5-8 membered heteroaryl, and —NR₁₃R₁₄, the above R₁₀ groups each optionally further substituted by one or more substituents independently selected from the group consisting of deuterium, halogen, hydroxy, ═O, C₁₋₄ alkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, and —NR₁₃R₁₄;
each R₁₁ is independently selected from the group consisting of hydrogen, deuterium, C₁₋₄ alkyl, C₂₋₄ alkenyl, C₃₋₄ cycloalkyl, 3-6 membered heterocyclyl, C₆₋₈ aryl, and 5-8 membered heteroaryl, the above R₁₁ groups each optionally further substituted by one or more substituents independently selected from the group consisting of deuterium, halogen, hydroxy, ═O, cyano, C₁₋₄ alkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, and —NR₁₃R₁₄;
each R₁₂ is independently selected from the group consisting of hydrogen, deuterium, hydroxy, C₁₋₄ alkyl, C₁₋₄ alkoxy, C₂₋₄ alkenyl, C₂₋₄ alkynyl, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, and —NR₁₃R₁₄, the above R₁₁ groups each optionally further substituted by one or more substituents independently selected from the group consisting of deuterium, halogen, hydroxy, ═O, cyano, C₁₋₄ alkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, and —NR₁₃R₁₄;
each R₁₃ and each R₁₄ are independently selected from the group consisting of hydrogen, deuterium, hydroxy, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, sulfinyl, sulfonyl, methanesulfonyl, isopropylsulfonyl, cyclopropylsulfonyl, p-toluenesulfonyl, aminosulfonyl, dimethylaminosulfonyl, and C₁₋₄ alkanoyl, the above R₁₃ and R₁₄ groups each optionally further substituted by one or more substituents independently selected from the group consisting of deuterium, halogen, hydroxy, ═O, C₁₋₄ alkyl, C₂₋₄ alkenyl, C₂₋₄ alkynyl, C₁₋₄ haloalkyl, C₁₋₄ deuteroalkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, amino, mono C₁₋₄ alkylamino, diC₁₋₄ alkylamino, and C₁₋₄ alkanoyl; or
R₁₃ and R₁₄, together with the nitrogen atom to which R₁₃ and R₁₄ are directly attached, form a 4-6 membered heterocyclyl or 5-8 membered heteroaryl, the 4-6 membered heterocyclyl or 5-8 membered heteroaryl optionally further substituted by one or more substituents independently selected from the group consisting of deuterium, halogen, hydroxy, ═O, C₁₋₄ alkyl, C₂₋₄ alkenyl, C₂₋₄ alkynyl, C₁₋₄ haloalkyl, C₁₋₄ deuteroalkyl, C₁₋₄ alkoxy, C₃₋₆ cycloalkyl, C₃₋₆ cycloalkoxy, 3-6 membered heterocyclyl, 3-6 membered heterocyclyloxy, C₆₋₈ aryl, C₆₋₈ aryloxy, 5-8 membered heteroaryl, 5-8 membered heteroaryloxy, methanesulfonylmethyl, amino, monoC₁₋₄ alkylamino, diC₁₋₄ alkylamino, and C₁₋₄ alkanoyl; and
each r is independently 0, 1 or 2.

4. The compound of formula (I), or the stereoisomer or pharmaceutically acceptable salt thereof, according to claim 1, wherein the compound of formula (I) is a compound of formula (II) as shown below: 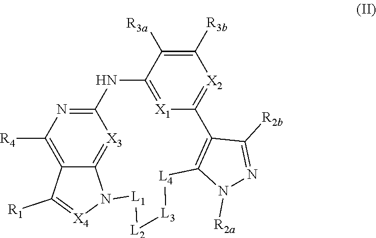
wherein:
X₁ is CRₐ or N;
X₂ is CR_{b} or N;
X₃ is CR_{c} or N;
X₄ is CR₅ or N;
L₁ is CR₅ₐR_{5b};
L₂ is (CR_{5c}R_{5d})ₚ, NR₆ₐ, O, S, S(O), S(O)₂ or C(O);
L₃ is a bond, CR₅ₑR_{5f}, or C(O);
L₄ is CR_{5g}R₅ₕ, O, S, S(O), S(O)₂, NR_{6c}, or C(O);
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, the above R₁ groups each optionally further substituted by one or more substituents independently selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₄ cycloalkyl, C₁₋₄ alkyl substituted by 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₁₃)—S(O)₂R₁₀, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, the above one or more substituents each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-N(R₁₃)—S(O)₂R₁₀, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₈ alkyl-C(O)SR₁₁, —C₀₋₄ alkyl-S—C(O)R₁₂, —C₀₋₈ alkyl-C(O)R₁₂, —C₀₋₈ alkyl-O—C(O)R₁₂, —C₀₋₈ alkyl-P(O)(R₁₂)₂, —C₀₋₄ alkyl-NR₁₃R₁₄, —C₀₋₄ alkyl-C(═NR₁₃)R₁₂, —C₀₋₄ alkyl-N(R₁₃)—C(═NR₁₄)R₁₂, —C₀₋₄ alkyl-C(O)NR₁₃R₁₄, and —C₀₋₄ alkyl-N(R₁₃)—C(O)R₁₂;
R₂ₐ is selected from the group consisting of hydrogen, deuterium, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —S(O)ᵣR₁₀, —O—R₁₁, —C(O)OR₁₁, —C(O)SR₁₁, —C(O₈R)R₁₂, and —C(O)NR₁₃R₁₄, the above R₂ₐ groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R_{2b} is selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, the above R_{2b} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R₃ₐ, R_{3b}, Rₐ, and R_{b} are each independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₄ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂;
R₄ and R_{c} are each independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₄ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
R_{d} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂;
each R_{5c} and each R_{5d} are independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄ and —N(R₁₃)—C(O)R₁₂, or, together with the carbon atom to which R_{5c} and R_{5d} are directly attached, form a C₃₋₄ cycloalkyl, 4-6 membered heterocyclyl, C₆₋₈ aryl, or 5-8 membered heteroaryl, the above R_{5c} and R_{5d} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂), —C(O)NR₁₃R₁₄, and —N(R₁₃)—C(O)R₁₂, provided that when R_{5c} or R_{5d} is H or C₁₋₄ alkyl, R₁ is optionally substituted C₂₋₁₀ alkynyl;
R₅ₑ and R_{5f} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)R₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, or, R₅ₑ and R_{5f}, together with the carbon atom to which R₅ₑ and R_{5f} are directly attached, form a C₃₋₆ cycloalkyl, 4-6 membered heterocyclyl, C₆₋₈ aryl, or 5-8 membered heteroaryl, the above R₅ₑ and R_{5f} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃))—C(O)R₁₂; and
R_{5g} and R₅ₕ are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, or, R_{5g} and R₅ₕ, together with the carbon atoms to which R_{5g} and R₅ₕ are directly attached, form a C₃₋₄ cycloalkyl, 4-6 membered heterocyclyl, C₆₋₈ aryl, or 5-8 membered heteroaryl, the above R_{5g} and R₅ₕ each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂.

5. The compound of formula (I), or the stereoisomer or pharmaceutically acceptable salt thereof, according to claim 1, wherein the compound of formula (I) is a compound of formula (III) as shown below: 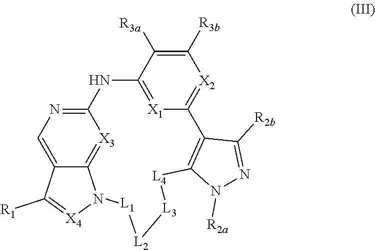
wherein, X₁ is CRₐ or N;
X₂ is CR_{b} or N;
X₃ is CR_{c} or N;
X₄ is CR_{d} or N;
L₁ is CR₅ₐR_{5b};
L₂ is (CR_{5c}R_{5d})ₚ;
L₃ is a bond, CR₅ₑR_{5f}, or C(O);
L₄ is CR_{5g}R_{5f}, O, NR_{6c}, or C(O);
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, the above R₁ groups each optionally further substituted by one or more substituents independently selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₁₃)—S(O)₂R₁₀, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, the above one or more substituents each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —C₀₋₄ alkyl-SF₅, —C₀₋₄ alkyl-S(O)(═N—R₇)R₈, —C₀₋₄ alkyl-N═S(O)R₈R₉, —C₀₋₄ alkyl-N═SR₈R₉, —C₀₋₄ alkyl-N(R₁₃)—S(O)₂R₁₀, —C₀₋₄ alkyl-O—S(O)₂R₁₀, —C₀₋₄ alkyl-S(O)ᵣR₁₀, —C₀₋₄ alkyl-O—R₁₁, —C₀₋₄ alkyl-C(O)OR₁₁, —C₀₋₄ alkyl-C(O)SR₁₁, —C₀₋₄ alkyl-S—C(O)R₁₂, —C₀₋₄ alkyl-C(O)R₁₂, —C₀₋₄ alkyl-O—C(O)R₁₂, —C₀₋₄ alkyl-P(O)(R₁₂)₂, —C₀₋₄ alkyl-NR₁₃R₁₄, —C₀₋₄ alkyl-C(═NR₁₃)R₁₂, —C₀₋₈ alkyl-N(R₁₃)—C(═NR₁₄)R₁₂, —C₀₋₈ alkyl-C(O)NR₁₃R₁₄, and —C₀₋₄ alkyl-N(R₁)—C(O)R₁₂;
R₂ₐ is selected from the group consisting of hydrogen, deuterium, hydroxy, C₁₋₄ alkyl, C₂₋₄ alkenyl, C₂₋₄ alkynyl, C₃₋₆ cycloalkyl, and 3-6 membered heterocyclyl, the above R₂ₐ groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂;
R_{2b} is selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, and —SF₅, the above R_{2b} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂;
R₃ₐ, R_{3b}, Rₐ and R_{b} are each independently selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₄ cycloalkyl, 3-6 membered heterocyclyl, C₆₋₈ aryl, 5-8 membered heteroaryl, and —SF₅;
R_{c} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, and —SF₅;
R_{d} is selected from the group consisting of hydrogen, deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, and —SF₅;
R₅ₐ and R_{5b} are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, and —SF₅, or, R₅ₐ and R_{5b}, together with the carbon atom to which R₅ₐ and R_{5b} are directly attached, form a C(O), C₃₋₆ cycloalkyl, 4-6 membered heterocyclyl, C₆₋₈ aryl, or 5-8 membered heteroaryl, and the above R₅ₐ and R_{5b} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₄ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂;
when p is 1, R_{5c} and R_{5d}, together with the carbon atom to which R_{5c} and R_{5d} are directly attached, form a C₃₋₆ cycloalkyl or 4-6 membered heterocyclyl, the above R_{5c} and R_{5d} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₄ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, or
when p is 2, one group of R_{5c} and R_{5d}, together with the carbon atom to which the one group of R_{5c} and R_{5d} are directly attached, form a C₃₋₄ cycloalkyl or 4-6 membered heterocyclyl, and the other group of R_{5c} and R_{5d} are each independently optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂;
R_{5g} and R₅ₕ are each independently selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, C₆₋₈ aryl, 5-8 membered heteroaryl, and —SF₅, or, R_{5g} and R₅ₕ, together with the carbon atom to which they R₅, and R₅ h are directly attached, form a C₃₋₆ cycloalkyl, 4-6 membered heterocyclyl, C₆₋₈ aryl, or 5-8 membered heteroaryl, and the above R_{5g} and R₅ₕ groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₄ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂; and
R_{6c} is selected from the group consisting of hydrogen, deuterium, C₁₋₄ alkyl, C₃₋₆ cycloalkyl, 3-6 membered heterocyclyl, and —O—R₁₁, the above R_{6c} groups each optionally further substituted by one or more substituents independently selected from the group consisting of deuterium, halogen, cyano, hydroxy, ═O, C₁₋₄ alkyl, C₁₋₄ haloalkyl, C₁₋₄ deuteroalkyl, C₂₋₄ alkenyl, C₂₋₄ alkynyl, C₁₋₄ alkoxy, C₃₋₆ cycloalkyl, C₃₋₆ cycloalkoxy, 3-6 membered heterocyclyl, 3-6 membered heterocyclyloxy, and —NR₁₃R₁₄.

6. The compound of formula (I), or the stereoisomer or pharmaceutically acceptable salt thereof, according to claim 1, wherein the compound of formula (I) is a compound of formula (IV) as shown below: 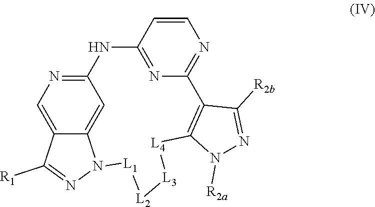
wherein,
L₁ is CR₅ₐR_{5b};
L₂ is CR_{5c}R_{5d};
L₃ is CR₅ₑR_{5f};
L₄ is O;
R₁ is selected from the group consisting of hydrogen, deuterium, halogen, cyano, C₁₋₄ alkyl, C₂₋₄ alkenyl, C₂₋₄ alkynyl, C₃₋₆ cycloalkyl, 3-8 membered heterocyclyl, C₆₋₈ aryl, 5-8 membered heteroaryl, and —SF₅, the above R₁ groups each optionally further substituted by one or more substituents independently selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ alkyl substituted by C₃₋₆ cycloalkyl, C₁₋₄ alkyl substituted by 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₁₃)—S(O)₂R₁₀, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁₃)—C(O)R₁₂, the above one or more substituents each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —S(O)(═N—R₇)R₈, —N═S(O)R₈R₉, —N═SR₈R₉, —N(R₃)—S(O)₂R₁₀, —O—S(O)₂R₁₀, —S(O)ᵣR₁₀, —O—R₁₁, —C(O)OR₁₁, —C(O)SR₁₁, —S—C(O)R₁₂, —C(O)R₁₂, —O—C(O)R₁₂, —P(O)(R₁₂)₂, —NR₁₃R₁₄, —C(═NR₁₃)R₁₂, —N(R₁₃)—C(═NR₁₄)R₁₂, —C(O)NR₁₃R₁₄, and —N(R₁)—C(O)R₁₂;
R₂ₐ is selected from the group consisting of hydrogen, deuterium, hydroxy, C₁₋₄ alkyl, C₂₋₄ alkenyl, C₂₋₄ alkynyl, C₃₋₆ cycloalkyl, and 3-6 membered heterocyclyl, the above R₂ₐ groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S, and —SF₅;
R_{2b} is selected from the group consisting of hydrogen, deuterium, halogen, cyano, C₁₋₄ alkyl, C₂₋₄ alkenyl, C₂₋₄ alkynyl, C₃₋₆ cycloalkyl, 3-6 membered heterocyclyl, and —SF₅, the above R_{2b} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S, and —SF₅;
R₅ₐ and R_{5b} are each independently selected form the group consisting of hydrogen, deuterium, halogen, cyano, C₁₋₄ alkyl, C₃₋₆ cycloalkyl, and 3-6 membered heterocyclyl, or, R₅ₐ and R_{5b}, together with the carbon atom to which R₅ₐ and R_{5b} are directly attached, form a C₃₋₆ cycloalkyl or 4-6 membered heterocyclyl, and the above R₅ₐ and R_{5g} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S, and —SF₅;
R_{5c} and R_{5d}, together with the carbon atom to which R_{5c} and R_{5d} are directly attached, form a C₃₋₆ cycloalkyl or 4-6 membered heterocyclyl, the above R_{5c} and R_{5d} groups are each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S, and —SF₅;
R₅ₑ and R_{5f} are each independently selected from the group consisting of hydrogen, deuterium, halogen, cyano, C₁₋₄ alkyl, C₃₋₆ cycloalkyl, and 3-6 membered heterocyclyl, or, R₅ₑ and R_{5f}, together with the carbon atom to which R₅ₑ and R_{5f} are directly attached, form a C₃₋₆ cycloalkyl or 4-6 membered heterocyclyl, the above R₅ₑ and R_{5f} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S, and —SF₅.

7. The compound of formula (I), or the stereoisomer or pharmaceutically acceptable salt thereof, according to claim 6, wherein L₁ is CR₅ₐR_{5b};
L₂ is CR_{5c}R_{5d};
L₃ is CR₅ₑR_{5f};
L₄ is O;
R₅ₐ and R_{5b} are each independently selected from the group consisting of hydrogen, deuterium, fluorine, chlorine, bromine, cyano, methyl, ethyl, propyl, isopropyl, cyclopropyl, cyclobutyl, oxacyclopropyl, oxacyclobutyl, azacyclopropyl, and azacyclobutyl, the above R₅ₐ and R_{5b} groups each optionally further substituted by one or more substituents independently selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S, and —SF₅;
R_{5c} and R_{5d}, together with the carbon atom to which R_{5c} and R_{5d} are directly attached, form a C₃₋₆ cycloalkyl or 4-6 membered heterocyclyl, the above R_{5c} and R_{5d} groups each optionally further substituted by one or more substituents independently selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S, and —CF₅; and
R₅ₑ and R_{5f} are each independently selected from the group consisting of hydrogen, deuterium, fluorine, chlorine, bromine, cyano, methyl, ethyl, propyl, isopropyl, cyclopropyl, cyclobutyl, oxacyclopropyl, oxacyclobutyl, azacyclopropyl, and azacyclobutyl, the above R₅ₑ and R_{5f} groups each optionally further substituted by one or more substituents independently selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S, and —SF₅.

8. The compound of formula (I), or the stereoisomer or pharmaceutically acceptable salt thereof, according to claim 6, wherein R₂ₐ is selected from the group consisting of hydrogen, deuterium, hydroxy, methyl, ethyl, propyl, isopropyl, vinyl, ethynyl, cyclopropyl, cyclobutyl, oxacyclopropyl, oxacyclobutyl, azacyclopropyl, and azacyclobutyl, the above R₂ₐ groups each optionally further substituted by one or more substituents independently selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S, and —SF₅; and
R_{2b} is selected from the group consisting of hydrogen, deuterium, fluorine, chlorine, bromine, cyano, methyl, ethyl, propyl, isopropyl, vinyl, ethynyl, cyclopropyl, cyclobutyl, oxacyclopropyl, oxacyclobutyl, azacyclopropyl, azacyclobutyl, and —SF₅, the above R_{2b} groups each optionally further substituted by one or more substituents independently selected from the group consisting of deuterium, fluorine, chlorine, bromine, cyano, methyl, ethyl, propyl, isopropyl, monofluoromethyl, difluoromethyl, trifluoromethyl, monodeuteriomethyl, dideuteriomethyl, trideuteriomethyl, vinyl, ethynyl, cyclopropyl, cyclobutyl, oxacyclopropyl, oxacyclobutyl, azacyclopropyl, azacyclobutyl, ═O, ═S, and —SF₅.

9. The compound of formula (I), or the stereoisomer or pharmaceutically acceptable salt thereof, according to claim 1, wherein the compound of formula (I) is a compound of formula (V) as shown below: 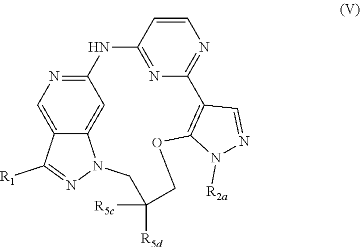
wherein,
R₁ is selected from C₂₋₄ alkenyl, C₂₋₄ alkynyl, and 3-8 membered heterocyclyl, the above R₁ groups each optionally further substituted by one or more substituents independently selected from the group consisting of deuterium, halogen, cyano, C₁₋₄ alkyl, C₂₋₄ alkenyl, C₂₋₄ alkynyl, C₃₋₆ cycloalkyl, 3-6 membered heterocyclyl, C₁₋₄ substituted by C₃₋₆ cycloalkyl, C₁₋₄ alkyl substituted by 3-6 membered heterocyclyl, ═O, ═S, —SF₅, —N(R₁₃)—S(O)₂R₁₀, —O—S(O)₂R₁₀, —S(O)ᵣR₁₀, —O—R₁₁, —C(O)OR₁₁, —S—C(O)R₁₂, —C(O)R₁₂, —O—C(O)R₁₂, —NR₁₃R₁₄, —C(O)NR₁₃R₁₄, and —N(R₁₃)—C(O)R₁₂, the above one or more substituents each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, C₆₋₈ aryl, 5-8 membered heteroaryl, ═O, ═S, —SF₅, —N(R₁₃)—S(O)₂R₁₀, —O—S(O)₂R₁₀, —S(O)ᵣR₁₀, —O—R₁₁, —C(O)OR₁₁, —C(O)R₁₂, —O—C(O)R₁₂, —NR₁₃R₁₄, —C(O)NR₁₃R₁₄, and —N(R₁₃)—C(O)R₁₂; and
R₂ₐ is selected from the group consisting of hydrogen, deuterium, C₁₋₄ alkyl, C₃₋₆ cycloalkyl, and 3-6 membered heterocyclyl, the above R₂ₐ groups each optionally further substituted by one or more substituents independently selected from the group consisting of deuterium, halogen, cyano, C₁₋₄ alkyl, C₁₋₄ haloalkyl, C₁₋₄ deuteroalkyl, C₃₋₄ cycloalkyl, and 3-6 membered heterocyclyl;
R_{5c} and R_{5d}, together with the carbon atom to which they R_{5c} and R_{5d} are directly attached, form a C₃₋₆ cycloalkyl or 4-6 membered heterocyclyl, the above R_{5c} and R_{5d} groups each optionally further substituted by one or more substituents independently selected from the group consisting of deuterium, halogen, cyano, C₁₋₄ alkyl, C₁₋₄ haloalkyl, C₁₋₄ deuteroalkyl, C₂₋₄ alkenyl, C₂₋₄ alkynyl, C₃₋₆ cycloalkyl, 3-6 membered heterocyclyl, ═O, ═S, and —SF₅.

10. The compound of formula (I), or the stereoisomer or pharmaceutically acceptable salt thereof, according to claim 9, wherein R_{5c} and R_{5d}, together with the carbon atom to which R_{5c} and R_{5d} are directly attached, form a C₃₋₆ cycloalkyl or 4-6 membered heterocyclyl.

11. The compound of formula (I), or the stereoisomer or pharmaceutically acceptable salt thereof, according to claim 9, wherein R_{5c} and R_{5d}, together with the carbon atom to which R_{5c} and R_{5d} are directly attached, form a cyclopropyl, cyclobutyl, cyclopentyl, cyclohexyl, oxacyclobutyl, oxocyclopentyl, or oxocyclohexyl.

12. The compound of formula (I), or the stereoisomer or pharmaceutically acceptable salt thereof, according to claim 9, wherein R₁ is C₂₋₄ alkynyl, and the C₂₋₄ alkynyl is optionally further substituted by one or more substituents selected from the group consisting of deuterium, C₁₋₄ alkyl, C₃₋₆ cycloalkyl, and 3-6 membered heterocyclyl, the above R₁ groups each optionally further substituted by one or more substituents independently selected from the group consisting of deuterium, halogen, cyano, C₁₋₄ alkyl, C₁₋₄ haloalkyl, C₁₋₄ deuteroalkyl, C₃₋₆ cycloalkyl, and 3-6 membered heterocyclyl.

13. The compound of formula (I), or the stereoisomer or pharmaceutically acceptable salt thereof, according to claim 9, wherein R₁ is ethynyl, the ethynyl optionally further substituted by one or more substituents selected from the group consisting of deuterium, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl, and azacyclohexyl, the above one or more substituents each optionally further substituted by one or more substituents independently selected from the group consisting of deuterium, fluorine, bromine, cyano, methyl, ethyl, propyl, isopropyl, butyl, monofluoromethyl, difluoromethyl, trifluoromethyl, monodeuteriomethyl, dideuteriomethyl, trideuteriomethyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl, and azacyclohexyl.

14. The compound of formula (I), or the stereoisomer or pharmaceutically acceptable salt thereof, according to claim 9, wherein R₁ is ethynyl, the ethynyl optionally further substituted by one or more substituents selected from the group consisting of cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl, and azacyclohexyl, the above R₁ groups each optionally further substituted by one or more substituents independently selected from the group consisting of deuterium, fluorine, bromine, cyano, methyl, ethyl, propyl, isopropyl, monofluoromethyl, difluoromethyl, trifluoromethyl, monodeuteriomethyl, dideuteriomethyl, and trideuteriomethyl.

15. The compound of formula (I), or the stereoisomer or pharmaceutically acceptable salt thereof, according to claim 9, wherein R₁ is 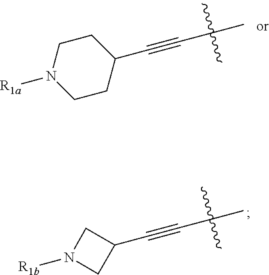
R₁ₐ is selected from the group consisting of hydrogen, methyl, ethyl, propyl, isopropyl, monofluoromethyl, difluoromethyl, trifluoromethyl, monodeuteriomethyl, dideuteriomethyl, and trideuteriomethyl; and
R_{1b} is selected from the group consisting of hydrogen, methyl, ethyl, propyl, isopropyl, monofluoromethyl, difluoromethyl, trifluoromethyl, monodeuteriomethyl, dideuteriomethyl, and trideuteriomethyl.

16. The compound of formula (I), or the stereoisomer or pharmaceutically acceptable salt thereof, according to claim 9, wherein R₁ is 4-8 membered nitrogen-containing heterocyclyl, the 4-8 membered nitrogen-containing heterocyclyl optionally further substituted by one or more substituents selected from the group consisting of deuterium, halogen, cyano, C₁₋₄ alkyl, ═O, —N(R₁₃)—S(O)₂R₁₀, —S(O)ᵣR₁₀, —O—R₁₁, —O—C(O)R₁₂, —NR₁₃R₁₄, and —N(R₁₃)—C(O)R₁₂, the C₁₋₄ alkyl optionally further substituted by one or more substituents selected from the group consisting of deuterium, halogen, cyano, C₁₋₄ alkyl, C₁₋₄ haloalkyl, C₁₋₄ deuteroalkyl, —N(R₁₃)—S(O)₂R₁₀, —O—S(O)₂R₁₀, —S(O)R₁₀, —O—R₁₁, —O—C(O)R₁₂, —NR₁₃R₁₄, and —N(R₁₃)—C(O)R₁₂.

17. The compound of formula (I), or the stereoisomer or pharmaceutically acceptable salt thereof, according to claim 9, wherein R₁ is of the following structure; 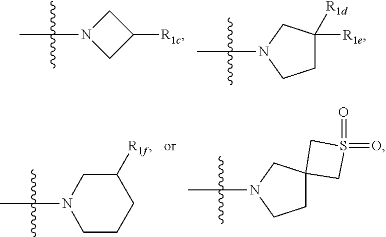
wherein,
R_{1c} is selected from the group consisting of hydrogen, deuterium, halogen, C₁₋₄ alkyl, —N(R₁₃ₐ)—S(O)₂R₁₀ₐ, —O—R₁₁ₐ, and —N(R₁₃ₐ)—C(O)R₁₂ₐ, the C₁₋₄ alkyl optionally further substituted by one or more substituents selected from the group consisting of deuterium, C₁₋₄ alkyl, C₁₋₄ haloalkyl, C₁₋₄ deuteroalkyl, —S(O)ᵣR_{11b}, and —O—R₁₁ₐ;
R_{1d} is selected from the group consisting of hydrogen, deuterium, halogen, C₁₋₄ alkyl, —N(R₁₃ₐ)—S(O)₂R₁₀ₐ, —O—R₁₁ₐ, and —N(R₁₃ₐ)—C(O)R₁₂ₐ;
R₁ₑ is selected from the group consisting of hydrogen, deuterium, halogen, C₁₋₄ alkyl, —N(R₁₃ₐ)—S(O)₂R₁₀ₐ, —O—R₁₁ₐ, and —N(R₁₃ₐ)—C(O)R₁₂ₐ, the C₁₋₄ alkyl optionally further substituted by one or more substituents selected from the group consisting of deuterium, C₁₋₄ alkyl, C₁₋₄ haloalkyl, C₁₋₄ deuteroalkyl, —S(O)ᵣR_{10b}, and —O—R₁₁ₐ;
R_{1f} is selected from the group consisting of hydrogen, deuterium, halogen, C₁₋₄ alkyl, —N(R₁₃ₐ)—S(O)₂R₁₀ₐ, —O—R₁₁ₐ, and —N(R₁₃ₐ)—C(O)R₁₁ₐ, the C₁₋₄ alkyl optionally further mem substituted by one or more substituents selected from the group consisting of deuterium, C₁₋₄ alkyl, C₁₋₄ haloalkyl, C₁₋₄ deuteroalkyl, —S(O)ᵣR_{10b}, and —O—R₁₁ₐ;
each R₁₀ₐ is independently selected from the group consisting of hydrogen, C₁₋₄ alkyl, C₃₋₆ cycloalkyl, and 3-6 membered heterocyclyl;
each R_{10b} is independently selected from the group consisting of hydrogen, C₁₋₄ alkyl, C₃₋₆ cycloalkyl, and 3-6 membered heterocyclyl, the above R_{10b} groups each optionally further substituted by one or more substituents independently selected from the group consisting of deuterium, halogen, hydroxy, C₁₋₄ alkyl, and C₁₋₄ alkoxy;
each R₁₁ₐ is independently hydrogen or C₁₋₄ alkyl;
each R₁₂ₐ is independently selected from the group consisting of hydrogen, C₁₋₄ alkyl, C₁₋₄ alkoxy, and C₃₋₆ cycloalkyl, the above R₁₂ₐ groups each optionally further substituted by one or more substituents independently selected from the group consisting of deuterium, halogen, hydroxy, and cyano;
each R₁₃ₐ is independently hydrogen or C₁₋₄ alkyl; and
each r is independently 0, 1 or 2.

18. The compound of formula (I), or the stereoisomer or pharmaceutically acceptable salt thereof, according to claim 9, wherein R₁ is of the structure: 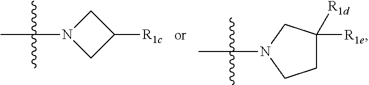
wherein,
R_{1c} is selected from the group consisting of hydrogen, deuterium, fluorine, bromine, methyl, ethyl, propyl, isopropyl, butyl, —N(R₁₃ₐ)—S(O)₂R₁₀ₐ, —O—R₁₁ₐ, and —N(R₁₃ₐ)—C(O)R₁₂ₐ, the methyl, ethyl, propyl, isopropyl, or butyl optionally further substituted by one or more substituents independently selected from the group consisting of deuterium, methyl, ethyl, propyl, isopropyl, butyl, monofluoromethyl, difluoromethyl, trifluoromethyl, monodeuteriomethyl, dideuteriomethyl, trideuteriomethyl, —S(O)ᵣR_{10b}, and —O—R₁₁ₐ;
R_{1d} is selected from the group consisting of hydrogen, deuterium, fluorine, bromine, methyl, ethyl, propyl, isopropyl, and butyl;
R₁ₑ is selected from the group consisting of hydrogen, deuterium, fluorine, bromine, methyl, ethyl, propyl, isopropyl, butyl, —N(R₁₃ₐ)—S(O)₂R₁₀ₐ, —O—R₁₁ₐ, and —N(R₁₃ₐ)—C(O)R₁₂ₐ, the methyl, ethyl, propyl, isopropyl, or butyl optionally further substituted by one or more substituents independently selected from the group consisting of deuterium, methyl, ethyl, propyl, isopropyl, butyl, monofluoromethyl, difluoromethyl, trifluoromethyl, monodeuteriomethyl, dideuteriomethyl, trideuteriomethyl, —S(O)ᵣR_{10b}, and —O—R₁₁ₐ;
each R₁₀ₐ is independently selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl, and azacyclohexyl;
each R_{10b} is independently selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl, and azacyclohexyl, the above R_{10b} groups each optionally further substituted by one or more substituents independently selected from the group consisting of deuterium, fluorine, chlorine, hydroxy, methyl, ethyl, propyl, isopropyl, butyl, methoxy, ethoxy, and propoxy;
each R₁₁ₐ is independently selected from the group consisting of hydrogen, methyl, ethyl, propyl, isopropyl, and butyl;
each R₁₂ₐ is independently selected from the group consisting of hydrogen, methyl, ethyl, propyl, isopropyl, butyl, methoxy, ethoxy, propoxy, tert-butoxy, cyclopropyl, cyclobutyl, cyclopentyl, and cyclohexyl, the above R₁₂ₐ groups each optionally further substituted by one or more substituents independently selected from the group consisting of deuterium, fluorine, chlorine, hydroxy, and cyano;
each R₁₃ₐ is independently hydrogen; and
each r is independently 0, 1, or 2.

19. The compound of formula (I), or the stereoisomer or pharmaceutically acceptable salt thereof, according to claim 9, wherein R₁ is of the structure: 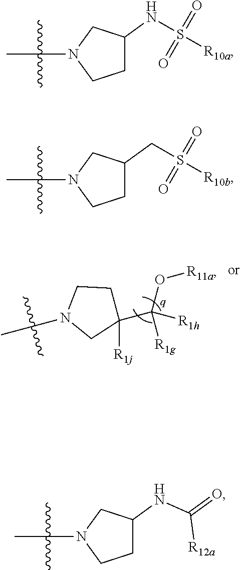
wherein,
R_{1g} is hydrogen, deuterium, or methyl; R₁ₕ is hydrogen, deuterium, or methyl; R₁ⱼ is hydrogen, deuterium, or methyl; q is 0, 1, or 2;
R₁₀ₐ is selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl, and azacyclohexyl;
R_{10b} is selected from the group consisting of hydrogen, methyl, ethyl, propyl, isopropyl, butyl, cyclopropyl, cyclobutyl, cyclopentyl, cyclohexyl, oxacyclopropyl, oxacyclobutyl, oxacyclopentyl, oxacyclohexyl, azacyclopropyl, azacyclobutyl, azacyclopentyl, and azacyclohexyl, the above R_{10b} groups each optionally further substituted by one or more substituents independently selected from the group consisting of deuterium, fluorine, chlorine, hydroxy, methyl, ethyl, propyl, isopropyl, butyl, methoxy, ethoxy, and propoxy;
each R₁₁ₐ is independently selected from the group consisting of hydrogen, methyl, ethyl, propyl, isopropyl, and butyl; and
R₁₂ₐ is selected from the group consisting of hydrogen, methyl, ethyl, propyl, isopropyl, butyl, methoxy, ethoxy, propoxy, tert-butoxy, cyclopropyl, cyclobutyl, cyclopentyl, and cyclohexyl, the above R₁₂ₐ groups each optionally further substituted by one or more substituents independently selected from the group consisting of deuterium, fluorine, chlorine, hydroxy, and cyano.

20. The compound of formula (I), or the stereoisomer or pharmaceutically acceptable salt thereof, according to claim 9, wherein R₂ₐ is selected from the group consisting of hydrogen, deuterium, C₁₋₄ alkyl, C₁₋₄ haloalkyl, C₁₋₄ deuteroalkyl, C₃₋₆ cycloalkyl, and 3-6 membered heterocyclyl.

21. The compound of formula (I), or the stereoisomer or pharmaceutically acceptable salt thereof, according to claim 9, wherein R₂ₐ is selected from the group consisting of hydrogen, deuterium, methyl, ethyl, propyl, isopropyl, butyl, monofluoromethyl, difluoromethyl, trifluoromethyl, monodeuteriomethyl, dideuteriomethyl, trideuteriomethyl, cyclopropyl, cyclobutyl, oxacyclopropyl, oxacyclobutyl, azacyclopropyl, and azacyclobutyl.

22. The compound of formula (I), or the stereoisomer or pharmaceutically acceptable salt thereof, according to claim 1, wherein the compound of formula (I) is selected from the following compounds:

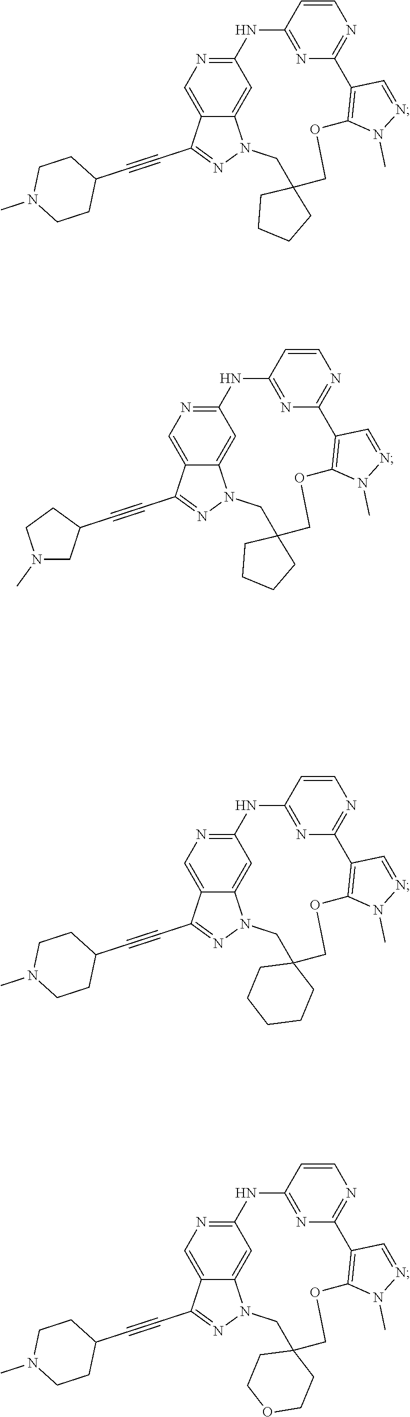 [SMILES: CN1CCC(C#Cc2nn3c4cc(ncc24)Nc2ccnc(n2)-c2cnn(C)c2OCC2(CCCC2)C3)CC1 ; CN1CCC(C#Cc2nn3c4cc(ncc24)Nc2ccnc(n2)-c2cnn(C)c2OCC2(CCCC2)C3)C1 ; CN1CCC(C#Cc2nn3c4cc(ncc24)Nc2ccnc(n2)-c2cnn(C)c2OCC2(CCCCC2)C3)CC1 ; CN1CCC(C#Cc2nn3c4cc(ncc24)Nc2ccnc(n2)-c2cnn(C)c2OCC2(CCOCC2)C3)CC1]

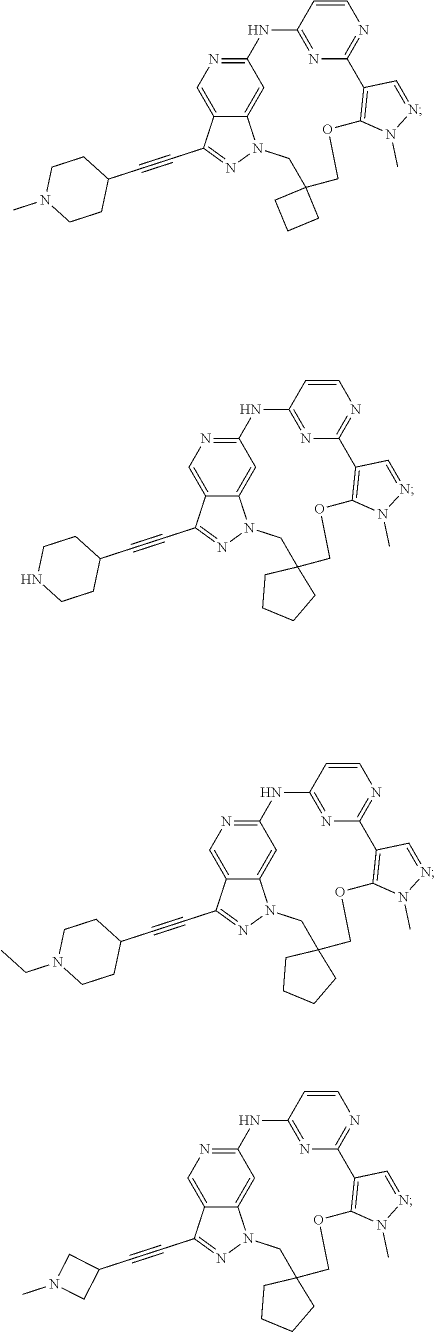 [SMILES: CN1CCC(C#Cc2nn3c4cc(ncc24)Nc2ccnc(n2)-c2cnn(C)c2OCC2(CCC2)C3)CC1 ; Cn1ncc2c1OCC1(CCCC1)Cn1nc(C#CC3CCNCC3)c3cnc(cc31)Nc1ccnc-2n1 ; CCN1CCC(C#Cc2nn3c4cc(ncc24)Nc2ccnc(n2)-c2cnn(C)c2OCC2(CCCC2)C3)CC1 ; CN1CC(C#Cc2nn3c4cc(ncc24)Nc2ccnc(n2)-c2cnn(C)c2OCC2(CCCC2)C3)C1]

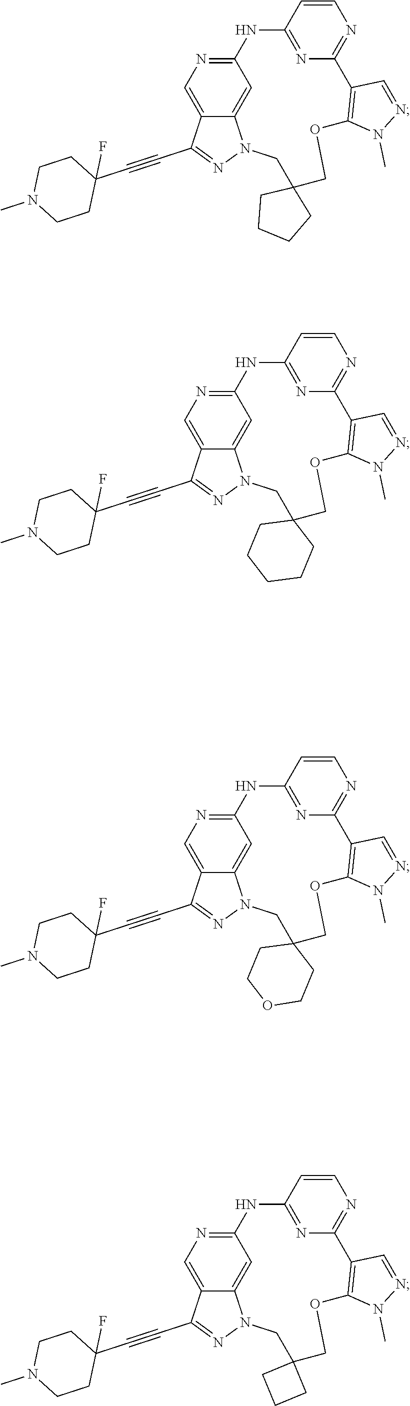 [SMILES: CN1CCC(F)(C#Cc2nn3c4cc(ncc24)Nc2ccnc(n2)-c2cnn(C)c2OCC2(CCCC2)C3)CC1 ; CN1CCC(F)(C#Cc2nn3c4cc(ncc24)Nc2ccnc(n2)-c2cnn(C)c2OCC2(CCCCC2)C3)CC1 ; CN1CCC(F)(C#Cc2nn3c4cc(ncc24)Nc2ccnc(n2)-c2cnn(C)c2OCC2(CCOCC2)C3)CC1 ; CN1CCC(F)(C#Cc2nn3c4cc(ncc24)Nc2ccnc(n2)-c2cnn(C)c2OCC2(CCC2)C3)CC1]

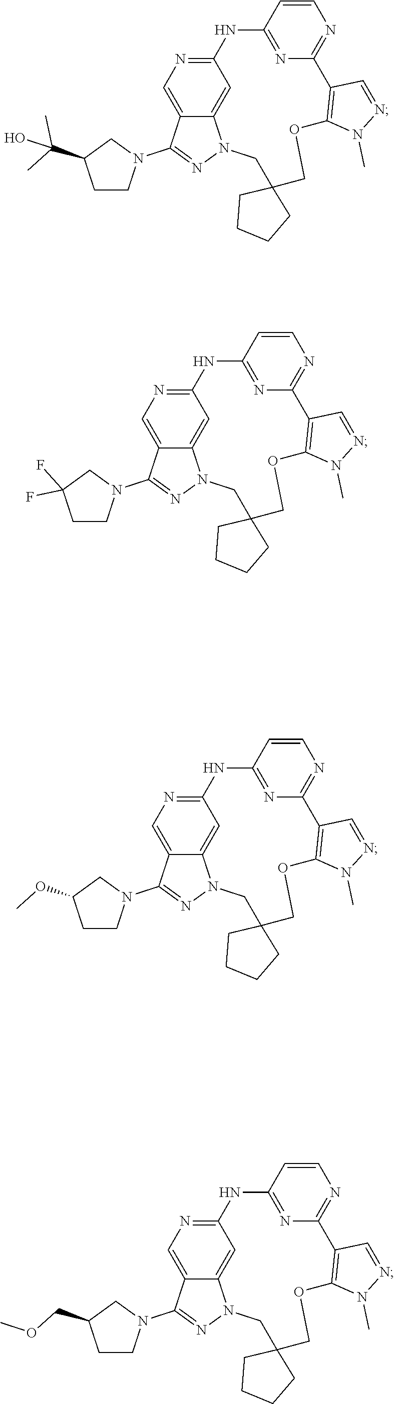 [SMILES: Cn1ncc2c1OCC1(CCCC1)Cn1nc(N3CC[C@@H](C(C)(C)O)C3)c3cnc(cc31)Nc1ccnc-2n1 ; Cn1ncc2c1OCC1(CCCC1)Cn1nc(N3CCC(F)(F)C3)c3cnc(cc31)Nc1ccnc-2n1 ; CO[C@H]1CCN(c2nn3c4cc(ncc24)Nc2ccnc(n2)-c2cnn(C)c2OCC2(CCCC2)C3)C1 ; COC[C@@H]1CCN(c2nn3c4cc(ncc24)Nc2ccnc(n2)-c2cnn(C)c2OCC2(CCCC2)C3)C1]

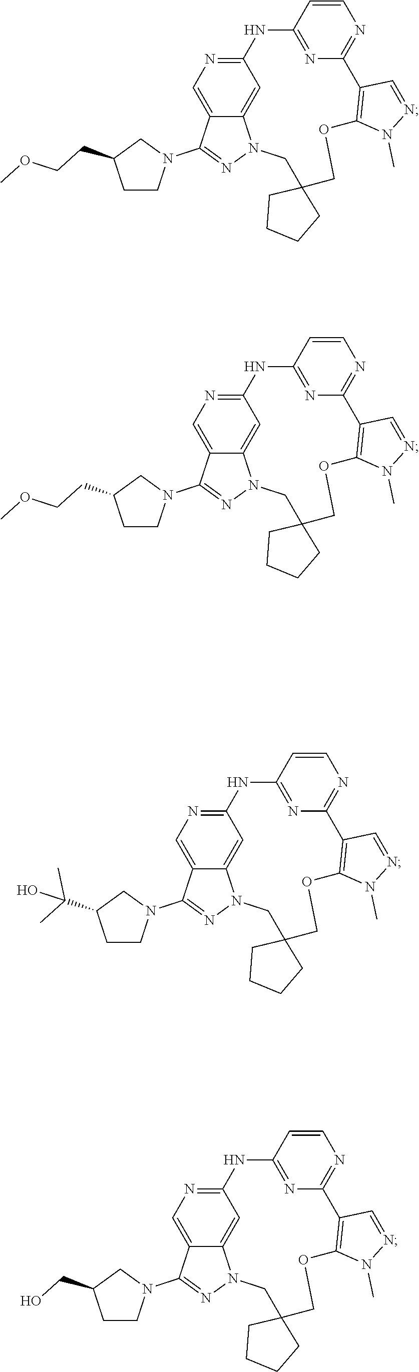 [SMILES: COCC[C@@H]1CCN(c2nn3c4cc(ncc24)Nc2ccnc(n2)-c2cnn(C)c2OCC2(CCCC2)C3)C1 ; COCC[C@H]1CCN(c2nn3c4cc(ncc24)Nc2ccnc(n2)-c2cnn(C)c2OCC2(CCCC2)C3)C1 ; Cn1ncc2c1OCC1(CCCC1)Cn1nc(N3CC[C@H](C(C)(C)O)C3)c3cnc(cc31)Nc1ccnc-2n1 ; Cn1ncc2c1OCC1(CCCC1)Cn1nc(N3CC[C@@H](CO)C3)c3cnc(cc31)Nc1ccnc-2n1]

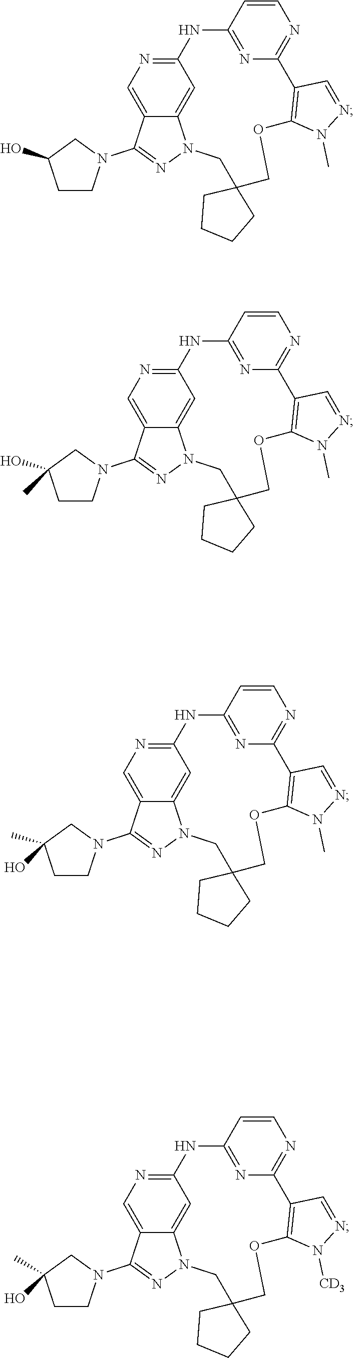 [SMILES: Cn1ncc2c1OCC1(CCCC1)Cn1nc(N3CC[C@@H](O)C3)c3cnc(cc31)Nc1ccnc-2n1 ; Cn1ncc2c1OCC1(CCCC1)Cn1nc(N3CC[C@](C)(O)C3)c3cnc(cc31)Nc1ccnc-2n1 ; Cn1ncc2c1OCC1(CCCC1)Cn1nc(N3CC[C@@](C)(O)C3)c3cnc(cc31)Nc1ccnc-2n1 ; [2H]C([2H])([2H])n1ncc2c1OCC1(CCCC1)Cn1nc(N3CC[C@@](C)(O)C3)c3cnc(cc31)Nc1ccnc-2n1]

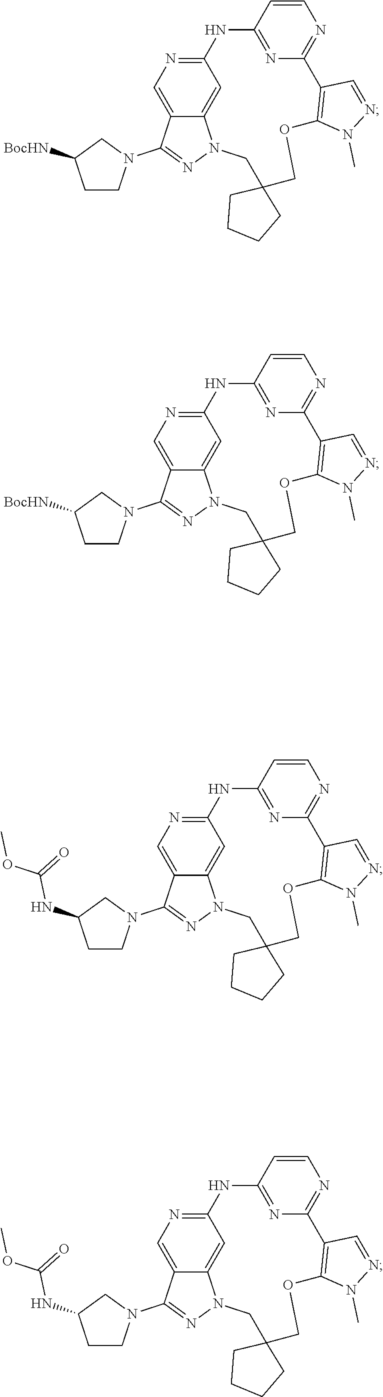 [SMILES: Cn1ncc2c1OCC1(CCCC1)Cn1nc(N3CC[C@@H](NC(=O)OC(C)(C)C)C3)c3cnc(cc31)Nc1ccnc-2n1 ; Cn1ncc2c1OCC1(CCCC1)Cn1nc(N3CC[C@H](NC(=O)OC(C)(C)C)C3)c3cnc(cc31)Nc1ccnc-2n1 ; COC(=O)N[C@@H]1CCN(c2nn3c4cc(ncc24)Nc2ccnc(n2)-c2cnn(C)c2OCC2(CCCC2)C3)C1 ; COC(=O)N[C@H]1CCN(c2nn3c4cc(ncc24)Nc2ccnc(n2)-c2cnn(C)c2OCC2(CCCC2)C3)C1]

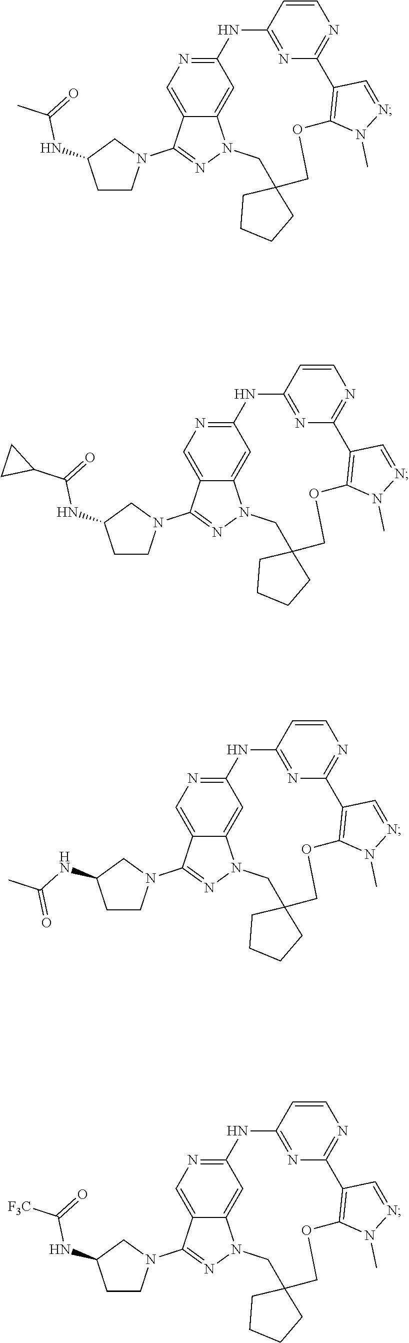 [SMILES: CC(=O)N[C@H]1CCN(c2nn3c4cc(ncc24)Nc2ccnc(n2)-c2cnn(C)c2OCC2(CCCC2)C3)C1 ; Cn1ncc2c1OCC1(CCCC1)Cn1nc(N3CC[C@H](NC(=O)C4CC4)C3)c3cnc(cc31)Nc1ccnc-2n1 ; CC(=O)N[C@@H]1CCN(c2nn3c4cc(ncc24)Nc2ccnc(n2)-c2cnn(C)c2OCC2(CCCC2)C3)C1 ; Cn1ncc2c1OCC1(CCCC1)Cn1nc(N3CC[C@@H](NC(=O)C(F)(F)F)C3)c3cnc(cc31)Nc1ccnc-2n1]

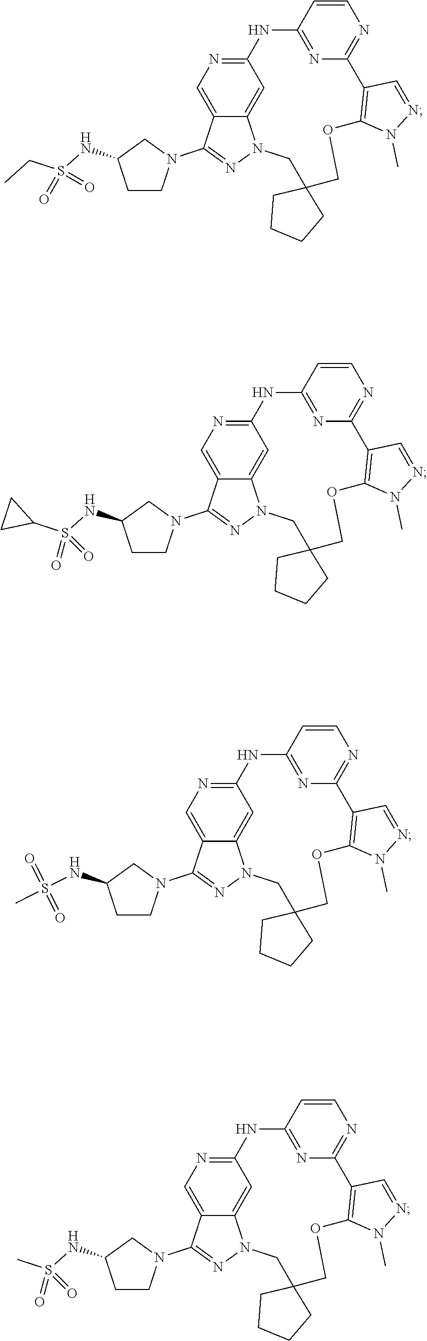 [SMILES: CCS(=O)(=O)N[C@H]1CCN(c2nn3c4cc(ncc24)Nc2ccnc(n2)-c2cnn(C)c2OCC2(CCCC2)C3)C1 ; Cn1ncc2c1OCC1(CCCC1)Cn1nc(N3CC[C@@H](NS(=O)(=O)C4CC4)C3)c3cnc(cc31)Nc1ccnc-2n1 ; Cn1ncc2c1OCC1(CCCC1)Cn1nc(N3CC[C@@H](NS(C)(=O)=O)C3)c3cnc(cc31)Nc1ccnc-2n1 ; Cn1ncc2c1OCC1(CCCC1)Cn1nc(N3CC[C@H](NS(C)(=O)=O)C3)c3cnc(cc31)Nc1ccnc-2n1]

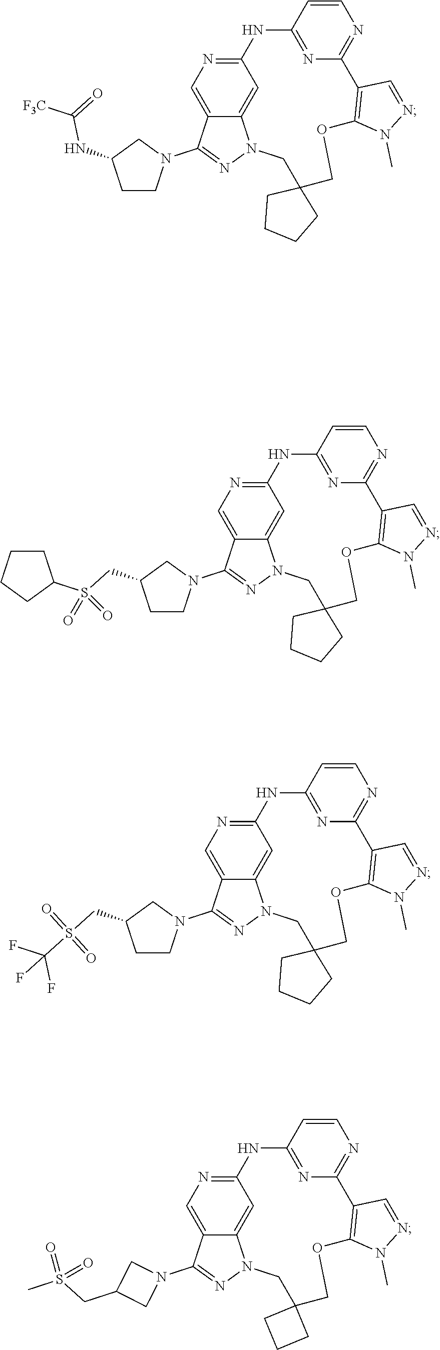 [SMILES: Cn1ncc2c1OCC1(CCCC1)Cn1nc(N3CC[C@H](NC(=O)C(F)(F)F)C3)c3cnc(cc31)Nc1ccnc-2n1 ; Cn1ncc2c1OCC1(CCCC1)Cn1nc(N3CC[C@H](CS(=O)(=O)C4CCCC4)C3)c3cnc(cc31)Nc1ccnc-2n1 ; Cn1ncc2c1OCC1(CCCC1)Cn1nc(N3CC[C@H](CS(=O)(=O)C(F)(F)F)C3)c3cnc(cc31)Nc1ccnc-2n1 ; Cn1ncc2c1OCC1(CCC1)Cn1nc(N3CC(CS(C)(=O)=O)C3)c3cnc(cc31)Nc1ccnc-2n1]

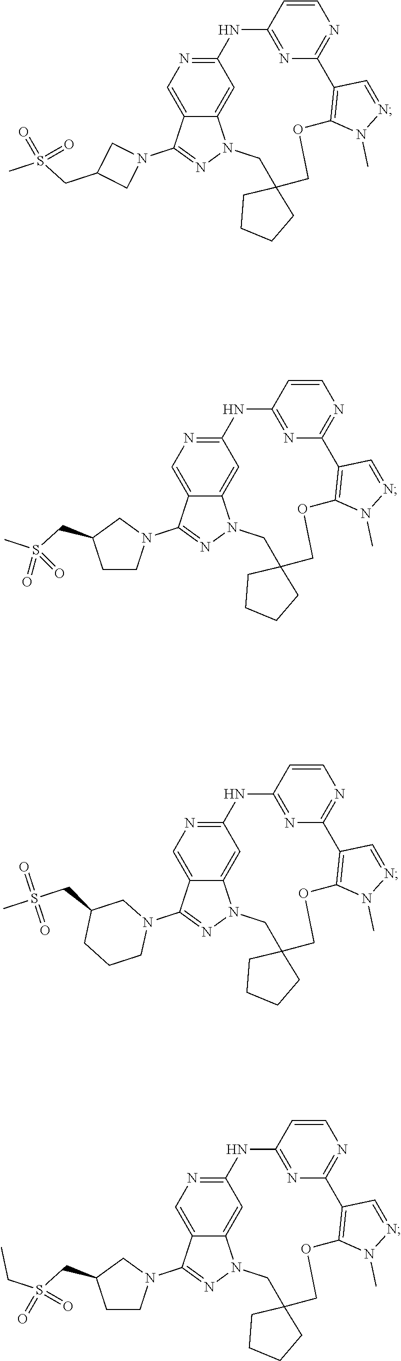 [SMILES: Cn1ncc2c1OCC1(CCCC1)Cn1nc(N3CC(CS(C)(=O)=O)C3)c3cnc(cc31)Nc1ccnc-2n1 ; Cn1ncc2c1OCC1(CCCC1)Cn1nc(N3CC[C@@H](CS(C)(=O)=O)C3)c3cnc(cc31)Nc1ccnc-2n1 ; Cn1ncc2c1OCC1(CCCC1)Cn1nc(N3CCC[C@@H](CS(C)(=O)=O)C3)c3cnc(cc31)Nc1ccnc-2n1 ; CCS(=O)(=O)C[C@@H]1CCN(c2nn3c4cc(ncc24)Nc2ccnc(n2)-c2cnn(C)c2OCC2(CCCC2)C3)C1]

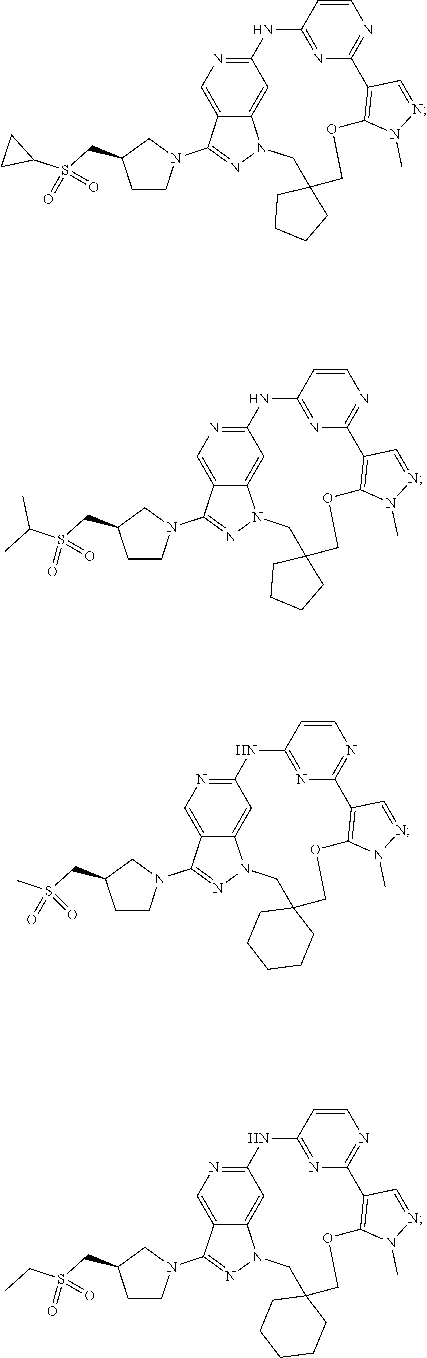 [SMILES: Cn1ncc2c1OCC1(CCCC1)Cn1nc(N3CC[C@@H](CS(=O)(=O)C4CC4)C3)c3cnc(cc31)Nc1ccnc-2n1 ; CC(C)S(=O)(=O)C[C@@H]1CCN(c2nn3c4cc(ncc24)Nc2ccnc(n2)-c2cnn(C)c2OCC2(CCCC2)C3)C1 ; Cn1ncc2c1OCC1(CCCCC1)Cn1nc(N3CC[C@@H](CS(C)(=O)=O)C3)c3cnc(cc31)Nc1ccnc-2n1 ; CCS(=O)(=O)C[C@@H]1CCN(c2nn3c4cc(ncc24)Nc2ccnc(n2)-c2cnn(C)c2OCC2(CCCCC2)C3)C1]

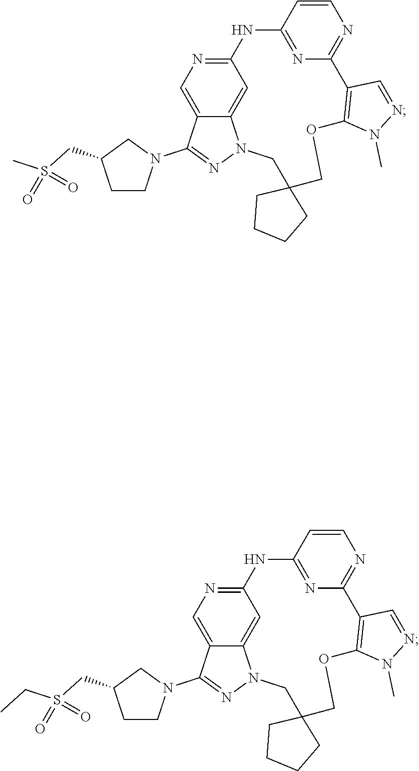 [SMILES: Cn1ncc2c1OCC1(CCCC1)Cn1nc(N3CC[C@H](CS(C)(=O)=O)C3)c3cnc(cc31)Nc1ccnc-2n1 ; CCS(=O)(=O)C[C@H]1CCN(c2nn3c4cc(ncc24)Nc2ccnc(n2)-c2cnn(C)c2OCC2(CCCC2)C3)C1]

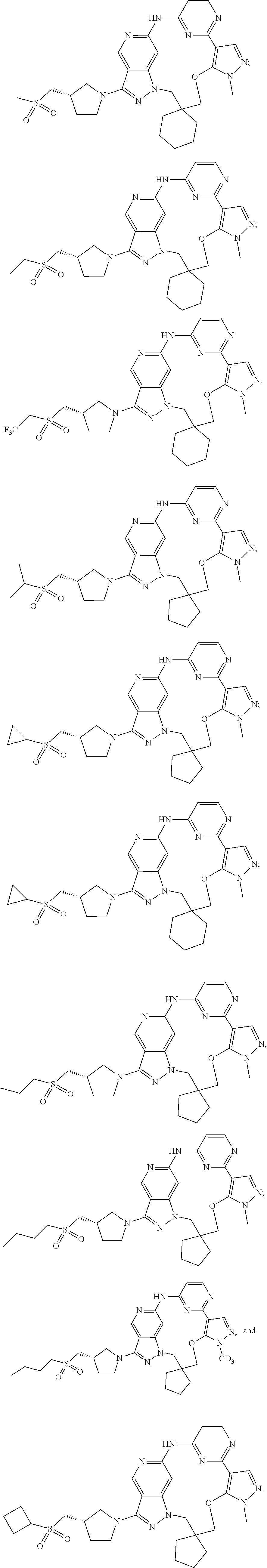

23. A pharmaceutical composition comprising the compound of formula (I), or the stereoisomer or pharmaceutically acceptable salt thereof, according to claim 1, and a pharmaceutically acceptable carrier.

24. A method for treating tumors or metastatic diseases in a subject, the tumors or metastatic diseases associated at least partially with EGFR L858R/C797S double mutation, EGFR Del19/C797S double mutation, L858R/T790 M/C797S triple mutation, and Del19/T790 M/C797S triple mutation, comprising administering to the subject an effective amount of a medicament comprising the compound of formula (I), or the stereoisomer or pharmaceutically acceptable salt thereof, according to claim 1.

25. The method according to claim 24, wherein the tumors or metastatic diseases are tumors and/or metastatic diseases caused by hyperproliferation and dysfunction in cell death induction.

26. The method according to claim 24, wherein the tumors or metastatic diseases are selected from the group consisting of lung cancer, colon cancer, pancreatic cancer, head and neck cancer, breast cancer, ovarian cancer, uterine cancer, gastric cancer, non-small cell lung cancer, leukemia, myelodysplastic syndrome, malignant lymphoma, head and neck tumor, thoracic tumor, gastrointestinal tumor, endocrine tumor, breast and other gynecological tumor, urological tumor, skin tumor, sarcoma, sinonasal inverted papilloma or sinonasal squamous cell carcinoma associated with sinonasal inverted papilloma.

27. (canceled)
